(19) United States
(12) Patent Application Publication
(10) Pub. No.: US 2024/0018245 A1
(43) Pub. Date: Jan. 18, 2024

(54) DOSING FOR TREATMENT WITH ANTI-FCRH5/ANTI-CD3 BISPECIFIC ANTIBODIES

(71) Applicant: Genentech, Inc., South San Francisco, CA (US)

(72) Inventors: Bernard Martin FINE, South San Francisco, CA (US); Teiko SUMIYOSHI, San Francisco, CA (US); Mengsong LI, South San Francisco, CA (US); James Niall COOPER, South San Francisco, CA (US)

(21) Appl. No.: 18/295,539

(22) Filed: Apr. 4, 2023

Related U.S. Application Data
(63) Continuation of application No. PCT/US21/53636, filed on Oct. 5, 2021.
(60) Provisional application No. 63/116,597, filed on Nov. 20, 2020.
(60) Provisional application No. 63/229,019, filed on Aug. 3, 2021.
(60) Provisional application No. 63/087,623, filed on Oct. 5, 2020.
(60) Provisional application No. 63/239,859, filed on Sep. 1, 2021.

(51) Int. Cl. C07K 16/28; A61P 35/00; A61K 31/573; A61K 39/395; A61K 31/167; A61K 31/137

(52) U.S. Cl. CPC C07K 16/283; A61P 35/00; C07K 16/2809; C07K 16/2866; A61K 2039/505; A61K 39/3955; A61K 31/167; A61K 31/137; A61K 31/573

(57) ABSTRACT

The invention provides methods of dosing for the treatment of cancers, such as multiple myelomas, with anti-fragment crystallizable receptor-like 5 (FcRH5)/anti-cluster of differentiation 3 (CD3) bispecific antibodies.

### SEQUENCE LISTING

The instant application contains a Sequence Listing which has been submitted electronically in XML format and is hereby incorporated by reference in its entirety. Said XML copy, created on Mar. 31, 2023, is named 50474-213006_Sequence_Listing_3_31_23 and is 41,598 bytes in size.

### FIELD OF THE INVENTION

The present invention relates to the treatment of cancers, such as B cell proliferative disorders. More specifically, the invention concerns the specific treatment of human patients having multiple myeloma (MM) using anti-fragment crystallizable receptor-like 5 (FcRH5)/anti-cluster of differentiation 3 (CD3) bispecific antibodies.

### BACKGROUND

Cancer remains one of the most deadly threats to human health. In the U.S., cancer affects more than 1.7 million new patients each year and is the second leading cause of death after heart disease, accounting for approximately one in four deaths.

Hematologic cancers, in particular, are the second leading cause of cancer-related deaths. Hematologic cancers include multiple myeloma (MM), a neoplasm characterized by the proliferation and accumulation of malignant plasma cells. Worldwide, approximately 110,000 people are diagnosed with MM annually. MM remains incurable despite advances in treatment, with an estimated median survival of 8-10 years for standard-risk myeloma and 2-3 years for high-risk disease, despite receipt of an autologous stem-cell transplant. Despite the significant improvement in patient's survival over the past 20 years, only 10-15% of patients achieve or exceed expected survival compared with the matched general population. Increased survival has been achieved with the introduction of proteasome inhibitors, immunomodulatory drugs (IMiDs), and monoclonal antibodies. Nevertheless, most patients (if not all) eventually relapse, and the outcome of patients with MM after they become refractory, or ineligible to receive a proteasome inhibitor or an IMiD, is quite poor, with survival less than 1 year. Therefore, relapsed or refractory (R/R) MM, in particular, continues to constitute a significant unmet medical need, and novel therapeutic agents are needed. For such patients, alternative or secondary treatment modalities, such as bispecific antibody-based immunotherapies, may be particularly efficacious. There is an unmet need in the field for the development of efficacious methods of dosing therapeutic bispecific antibodies (e.g., anti-FcRH5/anti-CD3 bispecific antibodies) for the treatment of cancers (e.g., MM, e.g., R/R MM) that achieve a more favorable benefit-risk profile.

### SUMMARY OF THE INVENTION

In one aspect, the disclosure features a method of treating a subject having a multiple myeloma (MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.01 mg to about 2.9 mg, the C1D2 is between about 3 mg to about 19.9 mg, and the C1D3 is between about 20 mg to about 600 mg.

In some aspects, the C1D1 is between about 0.1 mg to about 1.5 mg; the C1D2 is between about 3.2 mg to about 10 mg; and the C1D3 is between about 80 mg to about 300 mg. In some aspects, the C1D1 is about 0.3 mg; the C1D2 is about 3.6 mg; and the C1D3 is about 160 mg.

In some aspects, the dosing regimen further comprises a second dosing cycle comprising a single dose (C2D1) of the bispecific antibody, wherein the C2D1 is equal to or greater than the C1D3 and is between about 20 mg to about 600 mg. In some aspects, the C2D1 is between about 80 mg to about 300 mg. In some aspects, the C2D1 is about 160 mg.

In another aspect, the disclosure features a method of treating a subject having a MM comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.2 mg to about 0.4 mg, the C1D2 is greater than the C1D1, and the C1D3 is greater than the C1D2.

In some aspects, the C1D1 is about 0.3 mg. In some aspects, the C1D2 is between about 3 mg to about 19.9 mg. In some aspects, the C1D2 is between about 3.2 mg to about 10 mg. In some aspects, the C1D2 is about 3.6 mg. In some aspects, the C1D3 is between about 20 mg to about 600 mg. In some aspects, the C1D3 is between about 80 mg to about 300 mg. In some aspects, the C1D3 is about 160 mg.

In some aspects, the dosing regimen further comprises a second dosing cycle comprising a single dose (C2D1) of the bispecific antibody, wherein the C2D1 is equal to or greater than the C1D3 and is between about 20 mg to about 600 mg. In some aspects, the C2D1 is between about 80 mg to about 300 mg. In some aspects, the C2D1 is about 160 mg.

In some aspects, the length of the first dosing cycle is 21 days. In some aspects, the method comprises administering to the subject the C1D1, the C1D2, and the C1D3 on or about Days 1, 8, and 15, respectively, of the first dosing cycle.

In some aspects, the length of the second dosing cycle is 21 days. In some aspects, the method comprises administering to the subject the C2D1 on or about Day 1 of the second dosing cycle.

In some aspects, the dosing regimen comprises one or more additional dosing cycles. In some aspects, the dosing regimen comprises four additional dosing cycles, wherein the length of each of the four additional dosing cycles is 21 days. In some aspects, the four additional dosing cycles each comprise a single dose of the bispecific antibody, wherein the single dose is between about 80 mg to about 300 mg, and wherein the method comprises administering to the subject the single dose on or about Day 1 of each of the four additional dosing cycles. In some aspects, the dosing regimen further comprises up to 17 additional dosing cycles, wherein the length of each of the additional dosing cycles is 21 days. In some aspects, the up to 17 additional dosing cycles each comprise a single dose of the bispecific antibody, wherein the single dose is between about 80 mg to about 300 mg, and wherein the method comprises administering to the subject the single dose on or about Day 1 of each of the up to 17 additional dosing cycles.

In some aspects, the median peak IL-6 level in a population of subjects treated according to the method does not exceed 125 pg/mL between the C1D1 and the C1D2. In some aspects, the median peak IL-6 level in a population of subjects treated according to the method does not exceed 100 pg/mL between the C1D1 and the C1D2. In some aspects, the median peak IL-6 level in a population of subjects treated according to the method does not exceed 125 pg/mL between the C1D2 and the C1D3. In some aspects, the median peak IL-6 level in a population of subjects treated according to the method does not exceed 100 pg/mL between the C1D2 and the C1D3. In some aspects, the median peak IL-6 level in a population of subjects treated according to the method does not exceed 125 pg/mL following the C1D3. In some aspects, the median peak IL-6 level in a population of subjects treated according to the method does not exceed 100 pg/mL following the C1D3. In some aspects, the IL-6 level is measured in a peripheral blood sample.

In some aspects, the peak level of CD8+ T cell activation in the subject in the first dosing cycle occurs between the C1D2 and the C1D3. In some aspects, the peak level of CD8+ T cell activation in the subject in the first dosing cycle occurs within 24 hours of the C1D2.

In another aspect, the disclosure features a method of treating a subject having a multiple myeloma (MM) comprising administering to the subject a bispecific antibody that binds to FcRH5 and CD3 in a dosing regimen comprising at least a first dosing cycle, wherein the first dosing cycle comprises a first dose (C1D1) and a second dose (C1D2) of the bispecific antibody, wherein the C1D1 is between about 0.5 mg to about 19.9 mg and the C1D2 is between about 20 mg to about 600 mg. In some aspects, the C1D1 is between about 1.2 mg to about 10.8 mg and the C1D2 is between about 80 mg to about 300 mg. In some aspects, the C1D1 is about 3.6 mg and the C1D2 is about 198 mg. In some aspects, the length of the first dosing cycle is 21 days. In some aspects, the method comprises administering to the subject the C1D1 and the C1D2 on or about Days 1 and 8, respectively, of the first dosing cycle. In some aspects, the dosing regimen further comprises a second dosing cycle comprising a single dose (C2D1) of the bispecific antibody, wherein the C2D1 is equal to or greater than the C1D2 and is between about 20 mg to about 600 mg. In some aspects, the C2D1 is between about 80 mg to about 300 mg. In some aspects, the C2D1 is about 198 mg. In some aspects, the length of the second dosing cycle is 21 days. In some aspects, the method comprises administering to the subject the C2D1 on Day 1 of the second dosing cycle. In some aspects, the dosing regimen comprises one or more additional dosing cycles. In some aspects, the dosing regimen comprises one to 17 additional dosing cycles. In some aspects, the length of each of the one or more additional dosing cycles is 21 days. In some aspects, each of the one or more additional dosing cycles comprises a single dose of the bispecific antibody. In some aspects, the method comprises administering to the subject the single dose of the bispecific antibody on Day 1 of the one or more additional dosing cycles.

In some aspects of any of the methods described herein, the bispecific antibody comprises an anti-FcRH5 arm comprising a first binding domain comprising the following six hypervariable regions (HVRs): (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some aspects, the bispecific antibody comprises an anti-FcRH5 arm comprising a first binding domain comprising (a) a heavy chain variable (VH) domain comprising an amino acid sequence having at least 95% sequence identity to the amino acid sequence of SEQ ID NO: 7; (b) a light chain variable (VL) domain comprising an amino acid sequence having at least 95% sequence identity to the amino acid sequence of SEQ ID NO: 8; or (c) a VH domain as in (a) and a VL domain as in (b). In some aspects, the first binding domain comprises a VH domain comprising an amino acid sequence of SEQ ID NO: 7 and a VL domain comprising an amino acid sequence of SEQ ID NO: 8. In some aspects, wherein the bispecific antibody comprises an anti-CD3 arm comprising a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some aspects, the bispecific antibody comprises an anti-CD3 arm comprising a second binding domain comprising (a) a VH domain comprising an amino acid sequence having at least 95% sequence identity to the amino acid sequence of SEQ ID NO: 15; (b) a VL domain comprising an amino acid sequence having at least 95% sequence identity to the amino acid sequence of SEQ ID NO: 16; or (c) a VH domain as in (a) and a VL domain as in (b). In some aspects, the second binding domain comprises a VH domain comprising an amino acid sequence of SEQ ID NO: 15 and a VL domain comprising an amino acid sequence of SEQ ID NO: 16. In some aspects, the bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein: (a) H1 comprises the amino acid sequence of SEQ ID NO: 35; (b) L1 comprises the amino acid sequence of SEQ ID NO: 36; (c) H2 comprises the amino acid sequence of SEQ ID NO: 37; and (d) L2 comprises the amino acid sequence of SEQ ID NO: 38.

In some aspects of any of the methods described herein, the bispecific antibody comprises an aglycosylation site mutation. In some aspects, the aglycosylation site mutation reduces effector function of the bispecific antibody. In some aspects, wherein the aglycosylation site mutation is a substitution mutation. In some aspects, the bispecific antibody comprises a substitution mutation in the Fc region that reduces effector function. In some aspects, the bispecific antibody is a monoclonal antibody. In some aspects, the bispecific antibody is a humanized antibody. In some aspects, the bispecific antibody is a chimeric antibody. In some aspects, the bispecific antibody is an antibody fragment that binds FcRH5 and CD3. In some aspects, the antibody fragment is selected from the group consisting of Fab, Fab′-SH, Fv, scFv, and (Fab′)₂ fragments. In some aspects, the bispecific antibody is a full-length antibody. In some aspects, the bispecific antibody is an IgG antibody. In some aspects, the IgG antibody is an IgG₁ antibody.

In some aspects of any of the methods described herein, the bispecific antibody comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some aspects, at least one of the one or more heavy chain constant domains is paired with another heavy chain constant domain. In some aspects, the CH3₁ and CH3₂ domains each comprise a protuberance or cavity, and wherein the protuberance or cavity in the CH3₁ domain is positionable in the cavity or protuberance, respectively, in the CH3₂ domain. In some aspects, the CH3₁ and CH3₂ domains meet at an interface between the protuberance and cavity. In some aspects, the CH2₁ and CH2₂ domains each comprise a protuberance or cavity, and wherein the protuberance or cavity in the CH2₁ domain is positionable in the cavity or protuberance, respectively, in the CH2₂ domain. In some aspects, the CH2₁ and CH2₂ domains meet at an interface between said protuberance and cavity. In some aspects, the anti-FcRH5 arm comprises the protuberance and the anti-CD3 arm comprises the cavity. In some aspects, a CH3 domain of the anti-FcRH5 arm comprises a protuberance comprising a T366W amino acid substitution mutation (EU numbering) and a CH3 domain of the anti-CD3 arm comprises a cavity comprising T366S, L368A, and Y407V amino acid substitution mutations (EU numbering).

In some aspects of any of the methods described herein, the bispecific antibody is administered to the subject as a monotherapy.

In some aspects of any of the methods described herein, the bispecific antibody is administered to the subject as a combination therapy. In some aspects, the bispecific antibody is administered to the subject concurrently with one or more additional therapeutic agents. In some aspects, the bispecific antibody is administered to the subject prior to the administration of one or more additional therapeutic agents. In some aspects, the bispecific antibody is administered to the subject subsequent to the administration of one or more additional therapeutic agents. In some aspects, the one or more additional therapeutic agents comprise an effective amount of tocilizumab. In some aspects, tocilizumab is administered to the subject by intravenous infusion. In some aspects, (a) the subject weighs 100 kg, and tocilizumab is administered to the subject at a dose of 800 mg; (b) the subject weighs 30 kg and <100 kg, and tocilizumab is administered to the subject at a dose of 8 mg/kg; or (c) the subject weighs <30 kg, and tocilizumab is administered to the subject at a dose of 12 mg/kg. In some aspects, tocilizumab is administered to the subject 2 hours before administration of the bispecific antibody. In some aspects, the one or more additional therapeutic agents comprise an effective amount of pomalidomide, daratumumab, or a B-cell maturation antigen (BCMA)-directed therapy.

In some aspects of any of the methods described herein, the bispecific antibody is administered to the subject by intravenous infusion.

In some aspects of any of the methods described herein, the bispecific antibody is administered to the subject subcutaneously.

In some aspects of any of the methods described herein, the subject has a cytokine release syndrome (CRS) event, and the method further comprises treating the symptoms of the CRS event while suspending treatment with the bispecific antibody. In some aspects, the method further comprises administering to the subject an effective amount of tocilizumab to treat the CRS event. In some aspects, tocilizumab is administered intravenously to the subject as a single dose of about 8 mg/kg. In some aspects, the CRS event does not resolve or worsens within 24 hours of treating the symptoms of the CRS event, and the method further comprising administering to the subject one or more additional doses of tocilizumab to manage the CRS event. In some aspects, the one or more additional doses of tocilizumab are administered intravenously to the subject at a dose of about 8 mg/kg. In some aspects, the one or more additional therapeutic agents comprise an effective amount of a corticosteroid. In some aspects, the corticosteroid is administered intravenously to the subject. In some aspects, the corticosteroid is methylprednisolone. In some aspects, methylprednisolone is administered at a dose of about 80 mg. In some aspects, the corticosteroid is dexamethasone. In some aspects, dexamethasone is administered at a dose of about 20 mg. In some aspects, the one or more additional therapeutic agents comprise an effective amount of acetaminophen or paracetamol. In some aspects, acetaminophen or paracetamol is administered at a dose of between about 500 mg to about 1000 mg. In some aspects, acetaminophen or paracetamol is administered orally to the subject. In some aspects, the one or more additional therapeutic agents comprise an effective amount of diphenhydramine. In some aspects, diphenhydramine is administered at a dose of between about 25 mg to about 50 mg. In some aspects, diphenhydramine is administered orally to the subject.

In some aspects of any of the methods described herein, the MM is a relapsed or refractory (R/R) MM. In some aspects, the individual has received at least three prior lines of treatment for the MM. In some aspects, the individual has received at least four prior lines of treatment for the MM. In some aspects, the individual has been exposed to a prior treatment comprising a proteasome inhibitor, an IMiD, and/or an anti-CD38 therapeutic agent. In some aspects, the proteasome inhibitor is bortezomib, carfilzomib, or ixazomib. In some aspects, the IMiD is thalidomide, lenalidomide, or pomalidomide. In some aspects, the anti-CD38 therapeutic agent is an anti-CD38 antibody. In some aspects, the anti-CD38 antibody is daratumumab, MOR202, or isatuximab. In some aspects, the anti-CD38 antibody is daratumumab. In some aspects, the individual has been exposed to a prior treatment comprising an anti-SLAMF7 therapeutic agent, a nuclear export inhibitor, a histone deacetylase (HDAC) inhibitor, an autologous stem cell transplant (ASCT), a bispecific antibody, an antibody-drug conjugate (ADC), a CAR-T cell therapy, or a BCMA-directed therapy. In some aspects, the anti-SLAMF7 therapeutic agent is an anti-SLAMF7 antibody. In some aspects, the anti-SLAMF7 antibody is elotuzumab. In some aspects, the nuclear export inhibitor is selinexor. In some aspects, the HDAC inhibitor is panobinostat. In some aspects, the BCMA-directed therapy is an antibody-drug conjugate targeting BCMA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing dose escalation schedules for Arm A (single-step dose escalation arm) and Arm B (multi-step dose escalation arm) of the GO39775 Phase I dose-escalation study. C: cycle; D: day; Q: every.

FIG. 2 is a schematic diagram showing a possible single-step dose-escalation scenario for Arm A of the GO39775 Phase I dose-escalation study. AE: adverse event; DLT; dose-limiting toxicity; ISC: Internal Safety Committee; MAD: maximum achieved dose; MTD: maximum tolerated dose; pts: patients. Dose levels are in milligrams. Dose levels and dose modifications are for illustrative purposes only. “AE” refers to adverse events not considered by the investigator to be attributable to another clearly identifiable cause (e.g., disease progression).

FIG. 3 is a schematic diagram showing a possible two-step dose-escalation scenario for Arm B of the GO39775 Phase I dose-escalation study. CRS: cytokine release syndrome. Dose levels are in milligrams. Dose levels and dose modifications are for illustrative purposes only.

FIG. 4A is a schematic diagram showing the progression of doses in the single-step dose-escalation arm (Arm A) and the single-step expansion arm (Arm C) of the GO39775 Phase I dose-escalation study. Dose levels are in milligrams.

FIG. 4B is a schematic diagram showing the progression of doses in the double-step dose-escalation arm (Arm B) and the double-step expansion arm (Arm D) of the GO39775 Phase I dose-escalation study. Dose levels are in milligrams.

FIG. 5 is a bar graph showing the best percent change from baseline (baseline level of M-protein or affected light chain for light chain multiple myeloma (LCMM) patients) for patients treated with 3.6 mg and 20 mg, 40 mg, 60 mg, or 90 mg cevostamab (BFCR4350A) on C1D1 (cycle 1, day 1) and C1D8 (cycle 1, day 8), respectively, and a table showing the best response (PD: progressive disease; SD: stable disease; MR: minimal response; PR: partial response; VGPR: very good partial response; SCR: stringent complete response; CR: complete response) and time to best response in days; treatment history (Dara: daratumumab. PI: proteasome inhibitor; IMiD: immunomodulatory drug; auto: autologous stem cell transplantation (ASCT); and cytology for each patient. High risk cytology (including 1q21, t(4;14), t(11;14), t(14;16), and del(17p)) is defined using the International Myeloma Working Group (IMWG) criteria, as shown in Table 1.

FIG. 6 is a table showing the best response; presence or absence of extra-medullary (ext med) disease; presence or absence of high-risk cytology; and prior daratumumab status for thirteen patients who showed a response to cevostamab therapy and a chart showing timelines of treatment for each patient. Dose levels, overall response (MRD: minimal residual disease), and events (adverse events, ongoing treatment, overall response, and disease progression) are shown.

FIG. 7 is a bar graph showing the frequency of clinical symptoms of Grade 1 and Grade 2+ CRS. The grade of each symptom (adverse event; AE) is indicated by shade.

FIG. 8 is a set of tables showing the best overall response (PD, progressive disease; SD/MR, stable disease/minimal response; PR, partial response; VGPR, very good partial response; CR/sCR, complete response/stringent complete response) and the frequency and severity of CRS in patients enrolled in Arm B or Arm A of the GO39775 Phase I dose-escalation study.

FIG. 9 is a set of box plots showing pharmacodynamic (PD) parameters at the indicated cevostamab dose levels in Arms A, B, and C of the GO39775 Phase I dose-escalation study. CRS grade (no CRS, Grade 1, Grade 2, or Grade 3) is indicated by shade. Dashed lines in the Peak IL-6 plots indicate an IL-6 level of 100-125 pg/mL, which is a rough threshold for clinical significance based on CAR-T data. All flow cytometry timepoints are predose.

FIG. 10A is a set of box plots showing baseline FcRH5 expression level (molecules of equivalent soluble fluorochrome (MESF)) for patients in Arm A (≥20 mg target dose) and Arm C (3.6/90 mg) of the GO39775 Phase I dose-escalation study.

FIG. 10B is a set of box plots showing baseline FcRH5 expression level (MESF) and response (R, response; NR, no response; NA, data not available) for patients in Arm A (≥20 mg target dose) and Arm C (3.6/90 mg) of the GO39775 Phase I dose-escalation study.

FIG. 11A is a schematic diagram showing an experimental protocol for the tocilizumab prophylaxis arm of the GO39775 Phase I dose-escalation study. 15 patients are treated with tocilizumab prophylaxis and cevostamab, the study is paused for a review of safety, and 20 additional patients are treated following the safety review.

FIG. 11B is a schematic diagram showing a 6+6 experimental protocol for the tocilizumab prophylaxis arm of the GO39775 Phase I dose-escalation study. An initial group of patients are treated with tocilizumab prophylaxis and cevostamab, safety is reviewed, and about 30 additional patients are treated following the safety review.

FIG. 11C is a schematic diagram showing guidelines for opening an arm of the tocilizumab prophylaxis study including a prophylactic tocilizumab treatment at C1D8. *: based on success criteria.

FIG. 12 is a scatter plot showing peak IL-6 levels (pg/mL) in patients in the GO39775 Phase I study having no CRS or Grade 1, Grade 2, or Grade 3 CRS.

FIG. 13 is a set of scatter plots showing FcRH5 expression levels (MESF) for all biomarker-evaluable patients (left panel) and for biomarker-evaluable patients in the patients in active dose cohorts (doses at or above 3.6 mg on Cycle 1, Day 1 and 20 mg on Cycle 1, Day 8) who had less than a partial response (<PR; includes progressive disease, minimal response, and stable disease) or at least a partial response (≥PR; includes partial response, very good partial response, and stringent complete response) (right panel) in the GO39775 Phase I study.

FIG. 14A is a set of scatter plots showing absolute counts of CD8+ T-cells and CD4+ T-cells measured in peripheral blood of patients in the GO39775 Phase I study at the indicated time points. EOI: end of infusion.

FIG. 14B is a set of scatter plots showing levels of T-cell activation (assessed as levels of CD8+CD69+ T-cells) and T-cell proliferation (assessed as levels of CD8+CD69+ T-cells) measured in peripheral blood of patients in the GO39775 Phase I study at the indicated time points.

FIG. 14C is a scatter plot showing levels of IFN-γ measured in plasma of patients in the active dose cohorts of the GO39775 Phase I study at the indicated time points.

FIG. 15A is a scatter plot showing IL-6 levels (pg/mL) as measured in plasma of patients in the active dose cohorts of the GO39775 Phase I study at the indicated time points.

FIG. 15B is a scatter plot showing peak IL-6 levels (pg/mL) as measured in plasma of patients in the active dose cohorts of the GO39775 Phase I study after the C1D1 dose (left panel) or C1D8 dose (right panel) who experienced no CRS or Grade 1, 2, or 3 CRS. Symbols indicate whether the patient received tocilizumab after the C1D1 dose as a part of CRS treatment.

FIG. 16A is a set of graphs showing the density of CD8+ tumor-infiltrating T-cells in the tumor region (cells/mm²) for patients in the GO39775 Phase I study who were non-responders or responders during Cycle 1 and a scatter plot showing the log fold change in CD8+ tumor-infiltrating T-cells in non-responders and responders. **: p<0.01; NS: non-significant.

FIG. 16B is a set of micrographs showing dual chromogenic immunohistochemistry (IHC) staining for CD8 and CD138 in formalin-fixed, decalcified, and paraffin-embedded sections of bone marrow biopsies from screening (left panel, labeled “A”) and on treatment (right panel, labeled “B”) in a patient having a stringent complete response. Images are shown at 200× magnification. At screening, numerous CD138+ plasma cells were observed, with scattered CD8+ T-cells. On treatment, a single CD138+ plasma cell was observed, surrounded by large numbers of CD8+ T-cells.

FIG. 17 is a bar graph showing the incidence (%) and severity of CRS events at the indicated cycle dates.

FIG. 18 is a bar graph showing response rates for patients treated with the indicated doses of cevostamab in the GO39775 Phase I study.

FIG. 19 is a chart showing timelines of treatment for patients treated with cevostamab at the indicated dose levels. Overall response (PD, SD, MR (minor response), PR, VGPR, CR, or sCR)), and events (treatment completed, adverse events, disease progression, physician decisions, and ongoing treatment) are indicated by colors and symbols.

FIG. 20 is a graph showing the mean PK concentration (ng/mL) of cevostamab in serum at the indicated days after infusion and at the indicated doses.

FIG. 21 is a bar graph showing the overall response rate (ORR) (%) for efficacy evaluable patients who received the indicated prior therapy and were treated at or above the 3.6/20 mg dose level of cevostamab in the GO39775 Phase I study. BCMA: B-cell maturation antigen; CAR-T: chimeric antigen receptor T cell therapy; ADC, antibody-drug conjugate; ASCT, autologous stem cell transplant.

FIG. 22A is a scatter plot showing FcRH5 expression on myeloma cells (MESF) in samples from patients who have received six or more lines (≥6 L) or five or fewer lines (≤5 L) of prior treatment for MM.

FIG. 22B is a pair of scatter plots showing FcRH5 expression on tumor cells (MESF) in samples from patients who are triple-refractory (left panel; Y: triple-refractory; N; not triple-refractory) or penta-refractory (right panel; Y: penta-refractory; N; not penta-refractory) to prior MM therapy.

FIG. 22C is a set of scatter plots showing FcRH5 expression on myeloma cells (MESF) in samples from patients who have received prior anti-CD8 antibody therapy (left panel; Y: received prior anti-CD8 antibody therapy; N; did not receive such therapy); patients who have received prior anti-BCMA therapy (center panel; Y: received prior anti-BCMA therapy; N; did not receive such therapy); and patients who have received prior ASCT therapy (center panel; Y: received prior ASCT therapy; N; did not receive such therapy).

FIG. 23A is a set of scatter plots showing FcRH5 expression on tumor cells (MESF) in samples from patients who have 2, 1, or 0 high-risk cytogenetic abnormalities (left panel) and in all patients having high risk cytogenetics (at least one high-risk cytogenetic abnormality) or standard risk cytogenetics (right panel). n.s.: not significant.

FIG. 23B is a set of scatter plots showing FcRH5 expression on tumor cells (MESF) in samples from patients having (Y) or not having (N) 1q21 gain (left panel); t(4;14) abnormalities (center panel); and del(17p) abnormalities (right panel).

FIG. 24 is a schematic diagram showing the chemical structure of cevostamab (BFCR4350A). Anti-CD3: anti-cluster of differentiation 3; anti-FcRH5: anti-fragment crystallizable receptor-like 5; TDB: T-cell-dependent bispecific antibody.

FIG. 25 is a bar graph showing the cytokine release syndrome (CRS) profile in single step-up (right) and double step-up (left) dosing regimens in the GO39775 study. TD: target dose.

FIG. 26 is an exposure-response (E-R) plot showing the exposure-safety relationship of cevostamab (probability of the occurrence of Grade ≥2 CRS events vs. target dose Cₘₐₓ in Cycle 1) following the target dose administration based on pooled data from the single step and double step regimens of Study GO39775. Filled circles at 0% and 100% probabilities of Grade ≥2 CRS represent the observed data using pooled data from the single step-up and double step-up regimens. The E-R plots are divided into intervals (dashed grey lines) indicating the quintiles of the corresponding exposure metric. Black filled circles at each quintile indicate the observed median exposure and the observed probability of patients having Grade ≥2 CRS. Shaded areas and black curves represent the 90% CIs and the median of fitted logistic regression model from 1000 bootstrap samples, respectively. Horizontal bars represent the population pharmacokinetic model predicted exposures (geometric mean and 90% CIs) at the planned dose cohorts of 500 simulations at each cohort. AIC=Akaike information criterion; Cₘₐₓ Cycle 1 target dose=maximum concentration following the target dose administration of cevostamab; CRS=cytokine release syndrome; E0=baseline estimate of efficacy; EC50=half maximal effective concentration; Emax=maximal effect; E-R=exposure-response; Gr=Grade 2.

FIG. 27A is an E-R plot showing the exposure-safety relationship of cevostamab for occurrence of grade ≥1 CRS events following the C1D1 step-up dose administration using pooled data from the single step-up and double step-up regimens.

FIG. 27B is an E-R plot showing the exposure-safety relationship of cevostamab for occurrence of grade ≥1 CRS events following the target dose administration using pooled data from the single step-up and double step-up regimens.

FIG. 28A is an E-R plot showing the exposure-safety relationship of cevostamab for occurrence of grade ≥1 ICANS events following the C1D1 step-up dose administration using pooled data from the single step-up and double step-up regimens.

FIG. 28B is an E-R plot showing the exposure-safety relationship of cevostamab for occurrence of grade ≥1 ICANS events following the target dose administration (C_{max,ss}) using pooled data from the single step-up and double step-up regimens. C_{max,ss}=maximum concentration following the target dose administration of cevostamab at steady-state in both the single step-up and double step-up regimen.

FIG. 29 is a pair of plots showing the exposure-efficacy relationship of cevostamab for probability of objective response following cevostamab administration using pooled data from the single-step and double-step dosing regimens of study GO39775 (left: AUCₛₛ; right: C_{min,ss}). E0=baseline estimate of efficacy; EC50=half maximal effective concentration; Emax=maximal effect.

FIG. 30A is a plot showing the exposure-efficacy relationship of cevostamab for probability of ≥VGPR following cevostamab administration using pooled data from the single-step and double-step dosing regimens of Study GO39775 (AUCₛₛ).

FIG. 30B is a plot showing the exposure-efficacy relationship of cevostamab for probability of ≥VGPR following cevostamab administration using pooled data from the single-step and double-step dosing regimens of Study GO39775 (C_{min,ss}).

FIG. 31 is a plot showing the exposure-efficacy relationship of cevostamab exposure (AUCₛₛ) for probability of an ORR of PR or better following cevostamab administration using pooled data from the single-step and double-step dosing regimens of Study GO39775.

FIG. 32 is a set of Sankey diagrams showing the proportion of patients experiencing no CRS or Grade 1, Grade 2, or Grade 3 CRS in the indicated cycles of the indicated dosing regimens.

FIG. 33A is a box-and-whisker plot showing peak interleukin 6 (IL-6) concentrations determined between C1D1 to C1D8 in patients who received an 0.3 mg dose of cevostamab in the double-step dosing schedule compared to patients who received 3.6 mg in the single-step dosing schedule. CRS grade and tocilizumab (toci) administration (yes or no) are also shown for each patient.

FIG. 33B is a box-and-whisker plot showing peak IL-6 concentrations determined between C1D8 to C1D15 in patients who received the 3.6 mg C1D8 dose of cevostamab following the 0.3 mg C1D1 dose (denoted as 0.3/3.6) in the double-step dosing schedule compared to peak IL-6 levels determined between C1D1 to C1D8 in patients who received the 3.6 mg C1D1 dose in single-step dosing schedule. CRS grade and tocilizumab (toci) administration (yes or no) are also shown for each patient.

FIG. 33C is a box-and-whisker plot showing peak IL-6 concentrations determined post-target dose on C1D15 in the double-step dosing schedule compared to those on C1D8 in the single-step dosing schedule. CRS grade and tocilizumab (toci) administration (yes or no) are also shown for each patient.

FIG. 34 is a pair of box-and-whisker plots showing IL-6 concentration and CD8 T-cell activation pharmacodynamic (PD) data that support 0.3 mg as the lowest C1D1 dose. CRS grade and tocilizumab (toci) administration (yes or no) are also shown for each patient. Trt: treatment.

FIG. 35 is a plot showing the exposure-safety relationship of cevostamab for occurrence of grade ≥2 CRS events following the C1D1 step dose administration using pooled data from the single step and double step dosing regimens in Study GO39775 (Step Dose Cₘₐₓ).

FIG. 36 is a stacked bar graph showing the time to onset of CRS after each Cycle 1 dose of the recommended phase II dose.

FIG. 37A is a plot showing the relationship between the target dose and AUC_{7-21d}, following the target dose administration of cevostamab on Cycle 1 Day 8 (ranging from 0.15 mg to 198 mg) in the single step-up dose cohort. Black solid line represents the best-fit regression line using the power model. Colored dots represent the observed data at the tested target doses. The black filled circles represent the geometric mean of the exposures, with black bars representing the 90% CIs of the exposures at the tested doses.

FIG. 37B is a plot showing the relationship between the target dose and Cₘₐₓ, following the target dose administration of cevostamab on Cycle 1 Day 8 (ranging from 0.15 mg to 198 mg) in single step-up dose cohorts and on Cycle 1 Day 14 (ranging from 60 mg to 160 mg) in double step-up dose cohorts.

FIG. 38 is a set of box-and-whisker plots showing peak interleukin 6 (IL-6) concentrations determined following C1D1 in patients who received an 0.3 mg, 0.6 mg, 1.2 mg, or 3.6 mg dose of cevostamab (left panel) and following C1D8 in patients who received 0.3/3.6 mg, 0.6/3.6 mg, or 1.2/3.6 mg C1D1/C1D8 doses in the double-step dosing schedule (right panel) compared to patients who received a 3.6 mg C1D1 in the single-step dosing schedule. CRS grade and tocilizumab (toci) administration (yes or no) are also shown for each patient.

FIG. 39 is a set of plots showing percent CD8+ T-cell activation at the indicated time points during treatment with the indicated dosing regimens of cevostamab.

FIG. 40A is a scatter plot showing the relationship between peak IL-6 level observed following the step-up dose of cevostamab and the probability of Grade 1+ CRS. A linear logistic regression analysis is shown. IL-6 data following tocilizumab administration were censored.

FIG. 40B is a scatter plot showing the relationship between peak IL-6 level observed following the step-up dose of cevostamab and the probability of Grade 2+ CRS. A linear logistic regression analysis is shown. IL-6 data following tocilizumab administration were censored.

FIG. 41A is a scatter plot showing the relationship between peak IL-6 level observed following the target dose of cevostamab and the probability of Grade 1+ CRS. A linear logistic regression analysis is shown. IL-6 data following tocilizumab administration were censored.

FIG. 41B is a scatter plot showing the relationship between peak IL-6 level observed following the target dose of cevostamab and the probability of Grade 2+ CRS. A linear logistic regression analysis is shown. IL-6 data following tocilizumab administration were censored.

FIG. 42 is a pair of scatter plots showing the relationship between the percent of CD8+ T-cell activation observed following the C1D1 step-up dose of cevostamab and the probability of Grade 1+(left panel) or Grade 2+(right panel) CRS. A linear logistic regression analysis is shown.

FIG. 43 is a pair of scatter plots showing the relationship between the percent of CD8+ T-cell activation observed following the target dose of cevostamab and the probability of Grade 1+(left panel) or Grade 2+(right panel) CRS. A linear logistic regression analysis is shown.

FIG. 44 is a scatter plot showing the relationship between the cevostamab C1D1 step dose Cₘₐₓ and peak IL-6 concentration following administration of the C1D1 step dose. Pooled data from the single step and double step regimens of Study GO39775 are shown.

FIG. 45 is a scatter plot showing the relationship between the cevostamab target dose Cₘₐₓ and peak IL-6 concentration following administration of the target dose. Pooled data from the single step and double step regimens of Study GO39775 are shown.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) aspects that are directed to that value or parameter per se.

It is understood that aspects of the invention described herein include “comprising,” “consisting,” and “consisting essentially of” aspects.

The term “FcRH5” or “fragment crystallizable receptor-like 5,” as used herein, refers to any native FcRH5 from any vertebrate source, including mammals such as primates (e.g. humans) and rodents (e.g., mice and rats), unless otherwise indicated, and encompasses “full-length,” unprocessed FcRH5, as well as any form of FcRH5 that results from processing in the cell. The term also encompasses naturally occurring variants of FcRH5, including, for example, splice variants or allelic variants. FcRH5 includes, for example, human FcRH5 protein (UniProtKB/Swiss-Prot ID: Q96RD9.3), which is 977 amino acids in length.

The terms “anti-FcRH5 antibody” and “an antibody that binds to FcRH5” refer to an antibody that is capable of binding FcRH5 with sufficient affinity such that the antibody is useful as a diagnostic and/or therapeutic agent in targeting FcRH5. In one embodiment, the extent of binding of an anti-FcRH5 antibody to an unrelated, non-FcRH5 protein is less than about 10% of the binding of the antibody to FcRH5 as measured, e.g., by a radioimmunoassay (RIA). In certain embodiments, an antibody that binds to FcRH5 has a dissociation constant (K_{D}) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-FcRH5 antibody binds to an epitope of FcRH5 that is conserved among FcRH5 from different species.

The term “cluster of differentiation 3” or “CD3,” as used herein, refers to any native CD3 from any vertebrate source, including mammals such as primates (e.g. humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤250 nM, ≤100 nM, ≤15 nM, ≤10 nM, ≤5 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

For the purposes herein, “cevostamab,” also referred to as BFCR4350A or RO7187797, is an Fc-engineered, humanized, full-length non-glycosylated IgG1 kappa T-cell-dependent bispecific antibody (TDB) that binds FcRH5 and CD3 and comprises an anti-FcRH5 arm comprising the heavy chain polypeptide sequence of SEQ ID NO: 35 and the light chain polypeptide sequence of SEQ ID NO: 36 and an anti-CD3 arm comprising the heavy chain polypeptide sequence of SEQ ID NO: 37 and the light chain polypeptide sequence of SEQ ID NO: 38. Cevostamab comprises a threonine to tryptophan amino acid substitution at position 366 on the heavy chain of the anti-FcRH5 arm (T366W) using EU numbering of Fc region amino acid residues and three amino acid substitutions (tyrosine to valine at position 407, threonine to serine at position 366, and leucine to alanine at position 368) on the heavy chain of the anti-CD3 arm (Y407V, T366S, and L368A) using EU numbering of Fc region amino acid residues to drive heterodimerization of the two arms (half-antibodies). Cevostamab also comprises an amino acid substitution (asparagine to glycine) at position 297 on each heavy chain (N297G) using EU numbering of Fc region amino acid residues, which results in a non-glycosylated antibody that has minimal binding to Fc (Fcγ) receptors and, consequently, prevents Fc-effector function. Cevostamab is also described in WHO Drug Information (International Nonproprietary Names for Pharmaceutical Substances), Recommended INN: List 84, Vol. 34, No. 3, published 2020 (see page 701).

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “full-length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab, Fab′-SH; F(ab′)₂; diabodies; linear antibodies; single-chain antibody molecules (e.g., scFv, ScFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “functional Fc region” possesses an “effector function” of a native sequence Fc region.

Exemplary “effector functions” include C1q binding; CDC; Fc receptor binding; ADCC; phagocytosis; down regulation of cell surface receptors (e.g., B cell receptor; BCR), etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG I Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, preferably at least about 90% homology therewith, or preferably at least about 95% homology therewith.

“Fc complex” as used herein refers to CH3 domains of two Fc regions interacting together to form a dimer or, as in certain aspects, two Fc regions interact to form a dimer, wherein the cysteine residues in the hinge regions and/or the CH3 domains interact through bonds and/or forces (e.g., Van der Waals, hydrophobic forces, hydrogen bonds, electrostatic forces, or disulfide bonds).

“Fc component” as used herein refers to a hinge region, a CH2 domain or a CH3 domain of an Fc region.

“Hinge region” is generally defined as stretching from about residue 216 to 230 of an IgG (EU numbering), from about residue 226 to 243 of an IgG (Kabat numbering), or from about residue 1 to 15 of an IgG (IMGT unique numbering).

The “lower hinge region” of an Fc region is normally defined as the stretch of residues immediately C-terminal to the hinge region, i.e., residues 233 to 239 of the Fc region (EU numbering).

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and preferably at least about 90% homology therewith, more preferably at least about 95% homology therewith.

“Fc receptor” or “FcR” describes a receptor that binds to the Fc region of an antibody. A preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “knob-into-hole” or “KnH” technology as mentioned herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc interaction interfaces, CL:CH1 interfaces or VH/VL interfaces of antibodies (e.g., US2007/0178552, WO 96/027011, WO 98/050431 and Zhu et al. (1997) Protein Science 6:781-788). This is especially useful in driving the pairing of two different heavy chains together during the manufacture of multispecific antibodies. For example, multispecific antibodies having KnH in their Fc regions can further comprise single variable domains linked to each Fc region, or further comprise different heavy chain variable domains that pair with identical, similar, or different light chain variable domains. KnH technology can also be used to pair two different receptor extracellular domains together or any other polypeptide sequences that comprise different target recognition sequences.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The “CH1 region” or “CH1 domain” comprises the stretch of residues from about residue 118 to residue 215 of an IgG (EU numbering), from about residue 114 to 223 of an IgG (Kabat numbering), or from about residue 1.4 to residue 121 of an IgG (IMGT unique numbering) (Lefranc M-P, Giudicelli V, Duroux P, Jabado-Michaloud J, Folch G, Aouinti S, Carillon E, Duvergey H, Houles A, Paysan-Lafosse T, Hadi-Saljoqi S, Sasorith S, Lefranc G, Kossida S. IMGT®, the international ImMunoGeneTics information System® 25 years on. Nucleic Acids Res. 2015 January; 43 (Database issue):D413-22).

The “CH2 domain” of a human IgG Fc region usually extends from about residues 244 to about 360 of an IgG (Kabat numbering), from about residues 231 to about 340 of an IgG (EU numbering), or from about residues 1.6 to about 125 of an IgG (IGMT unique numbering). The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22: 161-206 (1985).

The “CH3 domain” comprises the stretch of residues C-terminal to a CH2 domain in an Fc region (i.e., from about amino acid residue 361 to about amino acid residue 478 of an IgG (Kabat numbering), from about amino acid residue 341 to about amino acid residue 447 of an IgG (EU numbering), or from about amino acid residue 1.4 to about amino acid residue 130 of an IgG (IGMT unique numbering)).

The “CL domain” or “constant light domain” comprises the stretch of residues C-terminal to a light-chain variable domain (VL). The light chain of an antibody may be a kappa (κ) (“Cκ”) or lambda (A) (“Cλ”) light chain region. The Cκ region generally extends from about residue 108 to residue 214 of an IgG (Kabat or EU numbering) or from about residue 1.4 to residue 126 of an IgG (IMGT unique numbering). The Cλ residue generally extends from about residue 107a to residue 215 (Kabat numbering) or from about residue 1.5 to residue 127 (IMGT unique numbering) (Lefranc M-P, Giudicelli V, Duroux P, Jabado-Michaloud J, Folch G, Aouinti S, Carillon E, Duvergey H, Houles A, Paysan-Lafosse T, Hadi-Saljoqi S, Sasorith S, Lefranc G, Kossida S. IMGT®, the international ImMunoGeneTics information System® 25 years on. Nucleic Acids Res. 2015 January; 43 (Database issue):D413-22).

The light chain (LC) from any vertebrate species can be assigned to one of two clearly distinct types, called kappa and lambda, based on the amino acid sequences of their constant domains.

Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter. J. Mol. Biol. 227:381, 1991; Marks et al. J. Mol. Biol. 222:581, 1991. Also available for the preparation of human monoclonal antibodies are methods described in Cole et al. Monoclonal Antibodies and Cancer Therapy, Alan R. Liss, p. 77 (1985); Boerner et al. J. Immunol., 147(1):86-95, 1991. See also van Dijk and van de Winkel. Curr. Opin. Pharmacol. 5:368-74, 2001. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al. Proc. Nat. Acad. Sci. USA. 103:3557-3562, 2006 regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. In certain aspects in which all or substantially all of the FRs of a humanized antibody correspond to those of a human antibody, any of the FRs of the humanized antibody may contain one or more amino acid residues (e.g., one or more Vernier position residues of FRs) from non-human FR(s). A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed. W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al. J. Immunol. 150:880-887, 1993; Clarkson et al. Nature 352:624-628, 1991.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);
(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthun, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

By “targeting domain” is meant a part of a compound or a molecule that specifically binds to a target epitope, antigen, ligand, or receptor. Targeting domains include but are not limited to antibodies (e.g., monoclonal, polyclonal, recombinant, humanized, and chimeric antibodies), antibody fragments or portions thereof (e.g., bis-Fab fragments, Fab fragments, F(ab′)₂, scFab, scFv antibodies, SMIP, single-domain antibodies, diabodies, minibodies, scFv-Fc, affibodies, nanobodies, and VH and/or VL domains of antibodies), receptors, ligands, aptamers, peptide targeting domains (e.g., cysteine knot proteins (CKP)), and other molecules having an identified binding partner. A targeting domain may target, block, agonize, or antagonize the antigen to which it binds.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “multispecific antibody” is used in the broadest sense and specifically covers an antibody that has polyepitopic specificity. In one aspect, the multispecific antibody binds to two different targets (e.g., bispecific antibody). Such multispecific antibodies include, but are not limited to, an antibody comprising a heavy chain variable domain (VH) and a light chain variable domain (VL), where the VH/VL unit has polyepitopic specificity, antibodies having two or more VL and VH domains with each VH/VL unit binding to a different epitope, antibodies having two or more single variable domains with each single variable domain binding to a different epitope, full-length antibodies, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Monospecific” refers to the ability to bind only one antigen. In one aspect, the monospecific biepitopic antibody binds two different epitopes on the same target/antigen. In one aspect, the monospecific polyepitopic antibody binds to multiple different epitopes of the same target/antigen. According to one aspect, the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the term “immunoadhesin” designates molecules which combine the binding specificity of a heterologous protein (an “adhesin”) with the effector functions of immunoglobulin constant domains. Structurally, the immunoadhesins comprise a fusion of an amino acid sequence with a desired binding specificity, which amino acid sequence is other than the antigen recognition and binding site of an antibody (i.e., is “heterologous” compared to a constant region of an antibody), and an immunoglobulin constant domain sequence (e.g., CH2 and/or CH3 sequence of an IgG). The adhesin and immunoglobulin constant domains may optionally be separated by an amino acid spacer. Exemplary adhesin sequences include contiguous amino acid sequences that comprise a portion of a receptor or a ligand that binds to a protein of interest. Adhesin sequences can also be sequences that bind a protein of interest, but are not receptor or ligand sequences (e.g., adhesin sequences in peptibodies). Such polypeptide sequences can be selected or identified by various methods, include phage display techniques and high throughput sorting methods. The immunoglobulin constant domain sequence in the immunoadhesin can be obtained from any immunoglobulin, such as IgG1, IgG2, IgG3, or IgG4 subtypes, IgA (including IgA1 and IgA2), IgE, IgD, or IgM.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rHL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), interleukin 1 (IL-1) blockers such as anakinra (Kineret), T cell costimulation blockers such as abatacept (Orencia), interleukin 6 (IL-6) blockers such as tocilizumab (ACTEMRA®); interleukin 13 (IL-13) blockers such as lebrikizumab; interferon alpha (IFN) blockers such as Rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “disorder” is any condition that would benefit from treatment including, but not limited to, chronic and acute disorders or diseases including those pathological conditions which predispose a mammal to the disorder in question. In one aspect, the disorder is a cancer, e.g., a multiple myeloma (MM).

The terms “cell proliferative disorder” and “proliferative disorder” refer to disorders that are associated with some degree of abnormal cell proliferation. In one aspect, the cell proliferative disorder is cancer. In one aspect, the cell proliferative disorder is a tumor.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous” refer to or describe the physiological condition in mammals that is typically characterized by unregulated cell growth/proliferation. Aspects of cancer include solid tumor cancers and non-solid tumor cancers. Examples of cancer include, but are not limited to, B cell proliferative disorders, such as multiple myeloma (MM), which may be relapsed or refractory MM. The MM may be, e.g., typical MM (e.g., immunoglobulin G (IgG) MM, IgA MM, IgD MM, IgE MM, or IgM MM), light chain MM (LCMM) (e.g., lambda light chain MM or kappa light chain MM), or non-secretory MM. The MM may have one or more cytogenetic features (e.g., high-risk cytogenic features), e.g., t(4;14), t(11;14), t(14;16), and/or del(17p), as described in Table 1 and in the International Myeloma Working Group (IMWG) criteria provided in Sonneveld et al., Blood, 127(24): 2955-2962, 2016, and/or 1q21, as described in Chang et al., Bone Marrow Transplantation, 45: 117-121, 2010. Cytogenic features may be detected, e.g., using fluorescent in situ hybridization (FISH).

TABLE 1
Cytogenic features of MM

| Primary genetic events | | Secondary genetic events | |
|---|---|---|---|
| IgH translocation | Gene(s) | Deletion | Gene(s) |
| t(4;14) | FGFR3/MMSET | 1p | CDKN2C, FAF1, FAM46C |
| t(6;14) | CCND3 | 6q | |
| t(11;14) | CCND1 | 8p | |
| t(14;16) | MAF | 13 | RB1, DIS3 |
| t(14;20) | MAFB | 11q | BIRC2/BIRC3 |
| | | 14q | TRAF3 |
| | | 16q | WWOX, CYLD |
| | | 17p | TP53 |
| Hyperdiploidy | | Gain | |
| Trisomies of chromosomes | | 1q | CKS1B, ANP32E |
| 3, 5, 7, 9, 11, 15, 19, 21 | | | |

The term “B cell proliferative disorder” or “B cell malignancy” refers to a disorder that is associated with some degree of abnormal B cell proliferation and includes, for example, a lymphoma, leukemia, myeloma, and myelodysplastic syndrome. In one embodiment, the B cell proliferative disorder is a lymphoma, such as non-Hodgkin's lymphoma (NHL), including, for example, diffuse large B cell lymphoma (DLBCL) (e.g., relapsed or refractory DLBCL). In another embodiment, the B cell proliferative disorder is a leukemia, such as chronic lymphocytic leukemia (CLL). Other specific examples of cancer also include germinal-center B cell-like (GCB) diffuse large B cell lymphoma (DLBCL), activated B cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, γ heavy chain disease, p heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, primary mediastinal (thymic) large B cell lymphoma, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Examples of solid tumors include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, and mesothelioma.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, e.g., as described in Gazzano-Santoro et al., J. Immunol. Methods 202:163 (1996), can be performed.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxic agents. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet. Annu. Rev. Immunol. 9:457-92, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. Proc. Natl. Acad. Sci. USA. 95:652-656, 1998.

“Complex” or “complexed” as used herein refers to the association of two or more molecules that interact with each other through bonds and/or forces (e.g., Van der Waals, hydrophobic, hydrophilic forces) that are not peptide bonds. In one aspect, the complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

As used herein, “delaying progression” of a disorder or disease means to defer, hinder, slow, retard, stabilize, and/or postpone development of the disease or disorder (e.g., a cell proliferative disorder, e.g., cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” of a compound, for example, an anti-FcRH5/anti-CD3 T-cell-dependent bispecific antibody (TDB) of the invention or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “overall survival” or “OS” refers to the percentage of individuals in a group who are likely to be alive after a particular duration of time.

As used herein, “objective response rate” (ORR) refers to the sum of stringent complete response (sCR), complete response (CR), very good partial response (VGPR), and partial response (PR) rates as determined using the International Myeloma Working Group response criteria (Table 4).

The term “epitope” refers to the particular site on an antigen molecule to which an antibody binds. In some aspects, the particular site on an antigen molecule to which an antibody binds is determined by hydroxyl radical footprinting. In some aspects, the particular site on an antigen molecule to which an antibody binds is determined by crystallography.

A “growth inhibitory agent” when used herein refers to a compound or composition which inhibits growth of a cell either in vitro or in vivo. In one aspect, growth inhibitory agent is growth inhibitory antibody that prevents or reduces proliferation of a cell expressing an antigen to which the antibody binds. In another aspect, the growth inhibitory agent may be one which significantly reduces the percentage of cells in S phase. Aspects of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

An “immunoconjugate” is an antibody conjugated to one or more heterologous molecule(s), including but not limited to a cytotoxic agent.

The term “immunomodulatory agent” refers to a class of molecules that modifies the immune system response or the functioning of the immune system. Immunomodulatory agents include, but are not limited to, PD-L1 axis binding antagonists, thalidomide (α-N-phthalimido-glutarimide) and its analogues, OTEZLA® (apremilast), REVLIMID® (lenalidomide) and POMALYST® (pomalidomide), and pharmaceutically acceptable salts or acids thereof.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human.

An “isolated” protein or peptide is one which has been separated from a component of its natural environment. In some aspects, a protein or peptide is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “PD-L1 axis binding antagonist” refers to a molecule that inhibits the interaction of a PD-L1 axis binding partner with either one or more of its binding partners, so as to remove T cell dysfunction resulting from signaling on the PD-L1 signaling axis—with a result being to restore or enhance T cell function (e.g., proliferation, cytokine production, target cell killing). As used herein, a PD-L1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some aspects, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one aspect, a PD-1 binding antagonist reduces the negative co-stimulatory signal mediated by or through cell surface proteins expressed on T lymphocytes mediated signaling through PD-1 so as render a dysfunctional T cell less dysfunctional (e.g., enhancing effector responses to antigen recognition). In some aspects, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is AMP-224. In another specific aspect, a PD-1 binding antagonist is MED1-0680. In another specific aspect, a PD-1 binding antagonist is PDR001. In another specific aspect, a PD-1 binding antagonist is REGN2810. In another specific aspect, a PD-1 binding antagonist is BGB-108.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some aspects, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some aspects, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one aspect, a PD-L1 binding antagonist reduces the negative co-stimulatory signal mediated by or through cell surface proteins expressed on T lymphocytes mediated signaling through PD-L1 so as to render a dysfunctional T cell less dysfunctional (e.g., enhancing effector responses to antigen recognition). In some aspects, a PD-L1 binding antagonist is an anti-PD-L1 antibody. In still another specific aspect, an anti-PD-L1 antibody is MPDL3280A (atezolizumab, marketed as TECENTRIQ™ with a WHO Drug Information (International Nonproprietary Names for Pharmaceutical Substances), Recommended INN: List 74, Vol. 29, No. 3, 2015 (see page 387)). In a specific aspect, an anti-PD-L1 antibody is YW243.55. S70. In another specific aspect, an anti-PD-L1 antibody is MDX-1105. In another specific aspect, an anti PD-L1 antibody is MSB0015718C. In still another specific aspect, an anti-PD-L1 antibody is MEDI4736.

The term “PD-L2 binding antagonist” refers to a molecule that decreases, blocks, inhibits, abrogates or interferes with signal transduction resulting from the interaction of PD-L2 with either one or more of its binding partners, such as PD-1. In some aspects, a PD-L2 binding antagonist is a molecule that inhibits the binding of PD-L2 to one or more of its binding partners. In a specific aspect, the PD-L2 binding antagonist inhibits binding of PD-L2 to PD-1. In some aspects, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T cell less dysfunctional (e.g., enhancing effector responses to antigen recognition). In some aspects, a PD-L2 binding antagonist is an immunoadhesin.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention (e.g., anti-FcRH5/anti-CD3 TDBs of the invention) are used to delay development of a disease or to slow the progression of a disease.

By “reduce” or “inhibit” is meant the ability to cause an overall decrease, for example, of 20% or greater, of 50% or greater, or of 75%, 85%, 90%, 95%, or greater. In certain aspects, reduce or inhibit can refer to the effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP).

According to the invention, the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. A vaccine may be a cancer vaccine. A “cancer vaccine” as used herein is a composition that stimulates an immune response in a subject against a cancer. Cancer vaccines typically consist of a source of cancer-associated material or cells (antigen) that may be autologous (from self) or allogenic (from others) to the subject, along with other components (e.g., adjuvants) to further stimulate and boost the immune response against the antigen. Cancer vaccines can result in stimulating the immune system of the subject to produce antibodies to one or several specific antigens, and/or to produce killer T cells to attack cancer cells that have those antigens.

As used herein, “administering” is meant a method of giving a dosage of a compound (e.g., an anti-FcRH5/anti-CD3 TDB of the invention) to a subject. In some aspects, the compositions utilized in the methods herein are administered intravenously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

“CD38” as used herein refers to a CD38 glycoprotein found on the surface of many immune cells, including CD4+, CD8+, B lymphocytes, and natural killer (NK) cells, and includes any native CD38 from any vertebrate source, including mammals such as primates (e.g., humans) and rodents (e.g., mice and rats), unless otherwise indicated. CD38 is expressed at a higher level and more uniformly on myeloma cells as compared to normal lymphoid and myeloid cells. The term encompasses “full-length,” unprocessed CD38, as well as any form of CD38 that results from processing in the cell. The term also encompasses naturally occurring variants of CD38, e.g., splice variants or allelic variants. CD38 is also referred to in the art as cluster of differentiation 38, ADP-ribosyl cyclase 1, cADPr hydrolase 1, and cyclic ADP-ribose hydrolase 1. CD38 is encoded by the CD38 gene. The nucleic acid sequence of an exemplary human CD38 is shown under NCBI Reference Sequence: NM_001775.4 or in SEQ ID NO: 33. The amino acid sequence of an exemplary human CD38 protein encoded by CD38 is shown under UniProt Accession No. P28907 or in SEQ ID NO: 34.

The term “anti-CD38 antibody” encompasses all antibodies that bind CD38 with sufficient affinity such that the antibody is useful as a therapeutic agent in targeting a cell expressing the antigen, and does not significantly cross-react with other proteins such as a negative control protein in the assays described below. For example, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. An anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the anti-CD38 antibody may bind to both human CD38 and chimpanzee CD38. Anti-CD38 antibodies also include anti-CD38 antagonist antibodies. Bispecific antibodies wherein one arm of the antibody binds CD38 are also contemplated. Also encompassed by this definition of anti-CD38 antibody are functional fragments of the preceding antibodies. Examples of antibodies which bind CD38 include: daratumumab (DARZALEX®) (U.S. Pat. No. 7,829,673 and U.S. Pub. No: 20160067205 A1); “MOR202” (U.S. Pat. No. 8,263,746); and isatuximab (SAR-650984).

### II. Therapeutic Methods

The invention is based, in part, on methods of treating a subject having cancer (e.g., multiple myeloma (MM)) using fractionated, dose-escalation dosing regimens with anti-fragment crystallizable receptor-like 5 (FcRH5)/anti-cluster of differentiation 3 (CD3) bispecific antibodies. The methods are expected to reduce or inhibit unwanted treatment effects, which include cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, and/or elevated liver enzymes. Therefore, the methods are useful for treating the subject while achieving a more favorable benefit-risk profile.

The invention provides methods useful for treating a subject having a cancer (e.g., multiple myeloma) that include administering to the subject a bispecific antibody that binds to FcRH5 and CD3 (i.e., an anti-FcRH5/anti-CD3 antibody) in a fractionated, dose-escalation dosing regimen.

A. Dosing Regimens

Single Step-Up Dosing Regimens

In some aspects, the invention provides methods of treating a subject having a cancer (e.g., a multiple myeloma (MM)) comprising administering to the subject a bispecific antibody that binds to FcRH5 and CD3 in a single step-up dosing regimen.

In some aspects, the invention provides a method of treating a subject having a multiple myeloma (MM) comprising administering to the subject a bispecific antibody that binds to FcRH5 and CD3 in a dosing regimen comprising at least a first dosing cycle, wherein the first dosing cycle comprises a first dose (C1D1) and a second dose (C1D2) of the bispecific antibody, wherein the C1D1 is between about 0.05 mg to about 180 mg (e.g., between about 0.1 mg to about 160 mg, between about 0.5 mg to about 140 mg, between about 1 mg to about 120 mg, between about 1.5 mg to about 100 mg, between about 2.0 mg to about 80 mg, between about 2.5 mg to about 50 mg, between about 3.0 mg to about 25 mg, between about 3.0 mg to about 15 mg, between about 3.0 mg to about 10 mg, or between about 3.0 mg to about 5 mg) and the C1D2 is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 100 mg, between about 75 mg to about 100 mg, or between about 85 mg to about 100 mg) and the C1D2 is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 50 mg to about 250 mg, between about 100 mg to about 225 mg, or between about 150 mg to about 200 mg).

In some aspects, the invention provides a method of treating a subject having a cancer (e.g., a multiple myeloma) comprising administering to the subject a bispecific antibody that binds to FcRH5 and CD3 in a dosing regimen comprising at least a first dosing cycle and a second dosing cycle, wherein (a) the first dosing cycle comprises a first dose (C1D1; cycle 1, dose 1) and a second dose (C1D2; cycle 1, dose, 2) of the bispecific antibody, wherein the C1D1 is less than the C1D2, and wherein the C1D1 is between about 0.05 mg to about 180 mg (e.g., between about 0.1 mg to about 160 mg, between about 0.5 mg to about 140 mg, between about 1 mg to about 120 mg, between about 1.5 mg to about 100 mg, between about 2.0 mg to about 80 mg, between about 2.5 mg to about 50 mg, between about 3.0 mg to about 25 mg, between about 3.0 mg to about 15 mg, between about 3.0 mg to about 10 mg, or between about 3.0 mg to about 5 mg) and the C1D2 is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 100 mg, between about 75 mg to about 100 mg, or between about 85 mg to about 100 mg); and (b) the second dosing cycle comprises a single dose (C2D1; cycle 2, dose 1) of the bispecific antibody, wherein the C2D1 is equal to or greater than the C1D2 and is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 100 mg, between about 75 mg to about 100 mg, or between about 85 mg to about 100 mg).

In some aspects, (a) the C1D1 is between about 0.5 mg to about 19.9 mg (e.g., between about 1 mg to about 18 mg, between about 2 mg to about 15 mg, between about 3 mg to about 10 mg, between about 3.3 mg to about 6 mg, or between about 3.4 mg to about 4 mg, e.g., about 3 mg, 3.2 mg, 3.4 mg, 3.6 mg, 3.8 mg, 4 mg, 4.2 mg, 4.4 mg, 4.6 mg, 4.8 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg), and (b) the C1D2 is between about 20 mg to about 600 mg (e.g., between about 30 mg to 500 mg, 40 mg to 400 mg, 60 mg to 350 mg, 80 mg to 300 mg, 100 mg to 200 mg, or 140 mg to 180 mg, e.g., about 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg).

In some aspects, the C1D1 is between about 1.2 mg to about 10.8 mg and the C1D2 is between about 80 mg to about 300 mg. In some aspects, the C1D1 is about 3.6 mg and the C1D2 is about 198 mg. In some aspects, the C1D1 is between 1.2 mg to 10.8 mg and the C1D2 is between 80 mg to 300 mg. In some aspects, the C1D1 is 3.6 mg and the C1D2 is 198 mg.

In some instances, the methods described above may include a first dosing cycle of three weeks or 21 days. In some instances, the methods may include administering to the subject the C1D1 and the C1D2 on or about Days 1 and 8, respectively, of the first dosing cycle.

Double Step-Up Dosing Regimens

In other aspects, the invention provides methods of treating a subject having a cancer (e.g., a multiple myeloma (MM)) comprising administering to the subject a bispecific antibody that binds to FcRH5 and CD3 in a double step-up dosing regimen.

In some aspects, the disclosure features a method of treating a subject having a cancer (e.g., a MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.2 mg to about 0.4 mg (e.g., is about 0.20 mg, 0.21 mg, 0.22 mg, 0.23 mg, 0.24 mg, 0.25 mg, 0.26 mg, 0.27 mg, 0.28 mg, 0.29 mg, 0.30 mg, 0.31 mg, 0.32 mg, 0.33 mg, 0.34 mg, 0.35 mg, 0.36 mg, 0.37 mg, 0.38 mg, 0.39 mg, or 0.40 mg); the C1D2 is greater than the C1D1, and the C1D3 is greater than the C1D2. In some aspects, the C1D1 is about 0.3 mg.

In some aspects, the C1D1 is between 0.2 mg to and 0.4 mg (e.g., is 0.20 mg, 0.21 mg, 0.22 mg, 0.23 mg, 0.24 mg, 0.25 mg, 0.26 mg, 0.27 mg, 0.28 mg, 0.29 mg, 0.30 mg, 0.31 mg, 0.32 mg, 0.33 mg, 0.34 mg, 0.35 mg, 0.36 mg, 0.37 mg, 0.38 mg, 0.39 mg, or 0.40 mg). In some aspects, the C1D1 is 0.3 mg.

In some aspects, the disclosure provides a method of treating a subject having a cancer (e.g., a MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.01 mg to about 2.9 mg, the C1D2 is between about 3 mg to about 19.9 mg, and the C1D3 is between about 20 mg to about 600 mg.

In some aspects, the invention provides a method of treating a subject having a cancer (e.g., a MM) comprising administering to the subject a bispecific antibody that binds to FcRH5 and CD3 in a dosing regimen comprising at least a first dosing cycle and a second dosing cycle, wherein (a) the first dosing cycle comprises a first dose (C1D1), a second dose (C1D2), and a third dose (C1D3) of the bispecific antibody, wherein the C1D1 and the C1D2 are each less than the C1D3, and wherein the C1D1 is between about 0.01 mg to about 2.9 mg, the C1D2 is between about 3 mg to about 19.9 mg, and the C1D3 is between about 20 mg to about 600 mg; and (b) the second dosing cycle comprises a single dose (C2D1) of the bispecific antibody, wherein the C2D1 is equal to or greater than the C1D3 and is between about 20 mg to about 600 mg.

In some aspects, the C1D1 is between about 0.05 mg to about 2.5 mg, about 0.1 mg to about 2 mg, about 0.2 mg to about 1 mg, or about 0.2 mg to about 0.4 mg (e.g., about 0.01 mg, 0.05 mg, 0.1 mg, 0.2 mg, 0.3 mg, 0.4 mg, 0.5 mg, 0.6 mg, 0.7 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, or 2.9 mg). In some aspects, the C1D1 is about 0.3 mg.

In some aspects, the C1D1 is between 0.05 mg to 2.5 mg, 0.1 mg to 2 mg, 0.2 mg to 1 mg, or 0.2 mg to 0.4 mg (e.g., 0.01 mg, 0.05 mg, 0.1 mg, 0.2 mg, 0.3 mg, 0.4 mg, 0.5 mg, 0.6 mg, 0.7 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, or 2.9 mg). In some aspects, the C1D1 is 0.3 mg.

In some aspects, the C1D2 is between about 3 mg to about 19.9 mg (e.g., between about 3 mg to about 18 mg, between about 3.1 mg to about 15 mg, between about 3.2 mg to about 10 mg, between about 3.3 mg to about 6 mg, or between about 3.4 mg to about 4 mg, e.g., about 3 mg, 3.2 mg, 3.4 mg, 3.6 mg, 3.8 mg, 4 mg, 4.2 mg, 4.4 mg, 4.6 mg, 4.8 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg). In some aspects, the C1D2 is between about 3.2 mg to about 10 mg. In some aspects, the C1D2 is about 3.6 mg.

In some aspects, the C1D2 is between 3 mg to 19.9 mg (e.g., between 3 mg to 18 mg, between 3.1 mg to 15 mg, between 3.2 mg to 10 mg, between 3.3 mg to 6 mg, or between 3.4 mg to 4 mg, e.g., 3 mg, 3.2 mg, 3.4 mg, 3.6 mg, 3.8 mg, 4 mg, 4.2 mg, 4.4 mg, 4.6 mg, 4.8 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg). In some aspects, the C1D2 is between 3.2 mg to 10 mg. In some aspects, the C1D2 is 3.6 mg.

In some aspects, the C1D3 is between about 20 mg to about 600 mg (e.g., between about 30 mg to about 500 mg, about 40 mg to about 400 mg, about 60 mg to about 350 mg, about 80 mg to about 300 mg, about 100 mg to about 200 mg, or about 140 mg to about 180 mg, e.g., about 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the C1D3 is between about 80 mg to about 300 mg. In some aspects, the C1D3 is about 160 mg.

In some aspects, the C1D3 is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the C1D3 is between 80 mg to 300 mg. In some aspects, the C1D3 is 160 mg.

In some aspects, the method comprises only a single dosing cycle (e.g., a dosing cycle comprising a C1D1, a C1D2, and a C1D3). In other aspects, the dosing regimen further comprises a second dosing cycle comprising at least a single dose (C2D1) of the bispecific antibody. In some aspects, the C2D1 is equal to or greater than the C1D3 and is between about 20 mg to about 600 mg (e.g., between about 30 mg to about 500 mg, about 40 mg to about 400 mg, about 60 mg to about 350 mg, about 80 mg to about 300 mg, about 100 mg to about 200 mg, or about 140 mg to about 180 mg, e.g., about 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the C2D1 is between about 80 mg to about 300 mg. In some aspects, the C2D1 is about 160 mg.

In some aspects, the C2D1 is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the C2D1 is between 80 mg to 300 mg. In some aspects, the C2D1 is 160 mg. In some aspects, the C2D1 is 159 mg.

Alternatively, in any of the above embodiments, the C1D1 may be between about 0.01 mg to about 60 mg (e.g., between about 0.05 mg to about 50 mg, between about 0.01 mg to about 40 mg, between about 0.1 mg to about 20 mg, between about 0.1 mg to about 10 mg, between about 0.1 mg to about 5 mg, between about 0.1 mg to about 2 mg, between about 0.1 mg to about 1.5 mg, between about 0.1 mg to about 1.2 mg, between about 0.1 mg to about 0.5 mg, or between about 0.2 mg to about 0.4 mg, e.g., about 0.3 mg, e.g., 0.3 mg), the C1D2 may be between about 0.05 mg to about 180 mg (e.g., between about 0.1 mg to about 160 mg, between about 0.5 mg to about 140 mg, between about 1 mg to about 120 mg, between about 1.5 mg to about 100 mg, between about 2.0 mg to about 80 mg, between about 2.5 mg to about 50 mg, between about 3.0 mg to about 25 mg, between about 3.0 mg to about 15 mg, between about 3.0 mg to about 10 mg, between about 3.0 mg to about 5 mg, or between about 3.0 mg to about 4.0 mg, e.g., about 3.6 mg, e.g., 3.6 mg), and the C1D3 may be between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 190 mg, between about 140 mg to about 180 mg, or between about 150 mg to about 170 mg, e.g., about 160 mg, e.g., 160 mg); and in aspects comprising a second dosing cycle, the C2D1 may be between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 190 mg, between about 140 mg to about 180 mg, or between about 150 mg to about 170 mg, e.g., about 160 mg, e.g., 160 mg).

In some instances, the length of the first dosing cycle is three weeks or 21 days. In some instances, the methods may include administering to the subject the C1D1, the C1D2, and the C1D3 on or about Days 1, 8, and 15, respectively, of the first dosing cycle.

Further Dosing Cycles

In some instances, the methods described above may include a second dosing cycle of three weeks or 21 days. In some instances, the methods may include administering to the subject the C2D1 on or about Day 1 of the second dosing cycle.

In some instances in which the methods include at least a second dosing cycle, the methods may include one or more additional dosing cycles. In some instances, the dosing regimen comprises 1 to 17 additional dosing cycles (e.g., 1, 2, 3, 4, 5, 6, 7, 8, 9, 10, 11, 12, 13, 14, 15, 16, or 17 additional dosing cycles, e.g., 1-3 additional dosing cycles, 1-5 additional dosing cycles, 3-8 additional dosing cycles, 5-10 additional dosing cycles, 8-12 additional dosing cycles, 10-15 additional dosing cycles, 12-17 additional dosing cycles, or 15-17 additional dosing cycles, i.e., the dosing regimen includes one or more of additional dosing cycle(s) C3, C4, C5, C6, C7, C8, C9, C10, C11, C12, C13, C14, C15, C16, C17, C18, and C19. In some embodiments, the length of each of the one or more additional dosing cycles is 7 days, 14 days, 21 days, or 28 days. In some embodiments, the length of each of the one or more additional dosing cycles is between 5 days and 30 days, e.g., between 5 and 9 days, between 7 and 11 days, between 9 and 13 days, between 11 and 15 days, between 13 and 17 days, between 15 and 19 days, between 17 and 21 days, between 19 and 23 days, between 21 and 25 days, between 23 and 27 days, or between 25 and 30 days. In some instances, the length of each of the one or more additional dosing cycles is three weeks or 21 days. In some instances, each of the one or more additional dosing cycles comprises a single dose of the bispecific antibody. In some aspects, the dose of the bispecific antibody in the one or more additional dosing cycles is equal to the C2D1, e.g., is between about 20 mg to about 600 mg (e.g., between about 30 mg to about 500 mg, about 40 mg to about 400 mg, about 60 mg to about 350 mg, about 80 mg to about 300 mg, about 100 mg to about 200 mg, or about 140 mg to about 180 mg, e.g., about 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the dose of the bispecific antibody in the one or more additional dosing cycles is about 160 mg. In some aspects, the dose of the bispecific antibody in the one or more additional dosing cycles is about 198 mg. In some aspects, the dose of the bispecific antibody in the one or more additional dosing cycles is equal to the C2D1, e.g., is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the dose of the bispecific antibody in the one or more additional dosing cycles is 160 mg. In some aspects, the dose of the bispecific antibody in the one or more additional dosing cycles is 198 mg. In some instances, the method comprises administering to the subject the single dose of the bispecific antibody on or about Day 1 of the one or more additional dosing cycles.

In some aspects, the bispecific antibody is administered to the subject every 21 days (Q3W) until progressive disease is observed, for up to 18 cycles, or until minimal residual disease (MRD) is observed.

In some instances, the bispecific anti-FcRH5/anti-CD3 antibody is administered to the subject as a monotherapy.

B. IL-6 and CD8+ T Cell Activation Thresholds

Peak IL-6 Levels

In some aspects of the dosing regimens (e.g., double-step dosing regimens) described herein, the peak IL-6 level in a sample from a patient or population of patients does not exceed a threshold for clinical significance, e.g., a threshold associated with increased risk of cytokine release syndrome (CRS). Peak IL-6 is the highest measured or reported IL-6 value taken during the time period following a dose of the bispecific antibody that binds to FcRH5 and CD3 (e.g., a time period between end of infusion (EOI) of the dose and the administration of the next dose. IL-6 level may be measured in any appropriate sample. In some aspects, the IL-6 level is measured in a peripheral blood sample.

In some aspects, the peak IL-6 level in a subject or the median peak IL-6 level in a population of subjects treated according to a method provided herein does not exceed 125 pg/mL (e.g., does not exceed 124, 123, 122, 121, 120, 119, 118, 117, 116, 115, 114, 113, 112, 111, 110, 109, 108, 107, 106, 105, 104, 103, 102, 101, or 100 pg/mL) between the C1D1 and the C1D2. For example, in some aspects, in which the C1D1 is administered on Day 1 and the C1D2 is administered on Day 8 of a dosing cycle, the peak IL-6 level in the subject or the median peak IL-6 level in the population of subjects does not exceed 125 pg/mL on Day 1 following administration of the C1D1, on any of Days 2-7, or on Day 8 before the administration of the C1D2. In some aspects, the peak IL-6 level in a subject or the median peak IL-6 level in a population of subjects treated according to the method does not exceed 100 pg/mL between the C1D1 and the C1D2.

In some aspects, the peak IL-6 level in a subject or the median peak IL-6 level in a population of subjects treated according to a method provided herein does not exceed 125 pg/mL (e.g., does not exceed 124, 123, 122, 121, 120, 119, 118, 117, 116, 115, 114, 113, 112, 111, 110, 109, 108, 107, 106, 105, 104, 103, 102, 101, or 100 pg/mL) between the C1D2 and the C1D3. For example, in some aspects, in which the C1D2 is administered on Day 8 and the C1D3 is administered on Day 15 of a dosing cycle, the peak IL-6 level in the subject or the median peak IL-6 level in the population of subjects does not exceed 125 pg/mL on Day 8 following administration of the C1D1, on any of Days 9-14, or on Day 15 before the administration of the C1D3. In some aspects, the peak IL-6 level in a subject or the median peak IL-6 level in a population of subjects treated according to the method does not exceed 100 pg/mL between the C1D2 and the C1D3.

In some aspects, the peak IL-6 level in a subject or the median peak IL-6 level in a population of subjects treated according to a method provided herein does not exceed 125 pg/mL (e.g., does not exceed 124, 123, 122, 121, 120, 119, 118, 117, 116, 115, 114, 113, 112, 111, 110, 109, 108, 107, 106, 105, 104, 103, 102, 101, or 100 pg/mL) following the C1D3. For example, in some aspects, in which the C1D3 is administered on Day 15 of a 21-day dosing cycle, the peak IL-6 level in the subject or the median peak IL-6 level in the population of subjects does not exceed 125 pg/mL on Day 15 following administration of the C1D1 or on any of days 16-21 of the dosing cycle. In some aspects, the peak IL-6 level in a subject or the median peak IL-6 level in a population of subjects treated according to the method does not exceed 100 pg/mL following the C1D3.

In some aspects, the peak IL-6 level in a subject or the median peak IL-6 level in a population of subjects treated according to a method provided herein does not exceed 125 pg/mL (e.g., does not exceed 124, 123, 122, 121, 120, 119, 118, 117, 116, 115, 114, 113, 112, 111, 110, 109, 108, 107, 106, 105, 104, 103, 102, 101, or 100 pg/mL) at any point during the dosing cycle. In some aspects, the peak IL-6 level in a subject or the median peak IL-6 level in a population of subjects treated according to a method provided herein does not exceed 100 pg/mL at any point during treatment.

In some aspects, the disclosure features a method of treating a subject having a cancer (e.g., a MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.2 mg to about 0.4 mg (e.g., is about 0.20 mg, 0.21 mg, 0.22 mg, 0.23 mg, 0.24 mg, 0.25 mg, 0.26 mg, 0.27 mg, 0.28 mg, 0.29 mg, 0.30 mg, 0.31 mg, 0.32 mg, 0.33 mg, 0.34 mg, 0.35 mg, 0.36 mg, 0.37 mg, 0.38 mg, 0.39 mg, or 0.40 mg); the C1D2 is greater than the C1D1, and the C1D3 is greater than the C1D2, wherein the peak IL-6 level in the subject or the median peak IL-6 level in the population of subjects treated according to the method does not exceed 125 pg/mL (e.g., does not exceed 100 pg/mL) between the C1D1 and the C1D2; between the C1D2 and the C1D3; and/or following the C1D3.

In some aspects, the invention provides a method of treating a subject having a cancer (e.g., a MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 and the C1D2 are each less than the C1D3, and wherein the C1D1 is between about 0.01 mg to about 2.9 mg, the C1D2 is between about 3 mg to about 19.9 mg, and the C1D3 is between about 20 mg to about 600 mg, wherein the peak IL-6 level in the subject or the median peak IL-6 level in the population of subjects treated according to the method does not exceed 125 pg/mL (e.g., does not exceed 100 pg/mL) between the C1D1 and the C1D2; between the C1D2 and the C1D3; and/or following the C1D3.

T Cell Activation

In some aspects of the double-step dosing regimens described herein, the peak level of CD8+ T cell activation in the subject in the first dosing cycle occurs between the C1D2 and the C1D3. For example, in some aspects, in which the C1D2 is administered on Day 8 and the C1D3 is administered on Day 15 of a dosing cycle, the peak level of CD8+ T cell activation in the subject occurs on Day 8 following administration of the C1D2, on any of Days 9-14, or on Day 15 before the administration of the C1D3. In some aspects, the peak level of CD8+ T cell activation in the subject in the first dosing cycle occurs within 24 hours of the C1D2, e.g., occurs within 20 hours, 18 hours, 16 hours, 14 hours, or 12 hours of the C1D2.

In some aspects, the disclosure features a method of treating a subject having a cancer (e.g., a MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the peak level of CD8+ T cell activation in the subject in the first dosing cycle occurs between the C1D2 and the C1D3.

In some aspects, the invention provides a method of treating a subject having a cancer (e.g., a MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 and the C1D2 are each less than the C1D3, and wherein the peak level of CD8+ T cell activation in the subject in the first dosing cycle occurs between the C1D2 and the C1D3.

C. Combination Therapies

In some instances, the bispecific anti-FcRH5/anti-CD3 antibody is administered to the subject in a combination therapy. For example, the bispecific anti-FcRH5/anti-CD3 antibody may be co-administered with one or more additional therapeutic agents.

i. Tocilizumab and Treatment of CRS

In one instance, the additional therapeutic agent is an effective amount of tocilizumab (ACTEMRA®). In some instances, the subject has a cytokine release syndrome (CRS) event (e.g., has a CRS event following treatment with the bispecific antibody, e.g., has a CRS event following a C1D1, a C1D2, a C1D3, a C2D1, or an additional dose of the bispecific antibody), and the method further comprises treating the symptoms of the CRS event (e.g., treating the CRS event by administering to the subject an effective amount of tocilizumab) while suspending treatment with the bispecific antibody. In some aspects, tocilizumab is administered intravenously to the subject as a single dose of about 8 mg/kg. In some aspects, the CRS event does not resolve or worsens within 24 hours of treating the symptoms of the CRS event, and the method further comprising administering to the subject one or more additional doses of tocilizumab to manage the CRS event, e.g., administering one or more additional doses of tocilizumab intravenously to the subject at a dose of about 8 mg/kg.

In some aspects, treating the symptoms of the CRS event further comprises treatment with a high-dose vasopressor (e.g., norepinephrine, dopamine, phenylephrine, epinephrine, or vasopressin and norepinephrine), e.g., as described in Tables 5A, 5B, and 6.

In other instances, tocilizumab is administered as a premedication, e.g., is administered to the subject prior to the administration of the bispecific anti-FcRH5/anti-CD3 antibody. In some instances, tocilizumab is administered as a premedication in Cycle 1, e.g., is administered prior to a first dose (C1D1), a second dose (C1D2), and/or a third dose (C1D3) of the bispecific anti-FcRH5/anti-CD3 antibody. In some aspects, the tocilizumab is administered intravenously to the subject as a single dose of about 8 mg/kg.

CRS Symptoms and Grading

CRS may be graded according to the Modified Cytokine Release Syndrome Grading System established by Lee et al., Blood, 124: 188-195, 2014 or Lee et al., Biol Blood Marrow Transplant, 25(4): 625-638, 2019, as described in Table 5A. In addition to diagnostic criteria, recommendations on management of CRS based on its severity, including early intervention with corticosteroids and/or anti-cytokine therapy, are provided and referenced in Tables 5A and 5B.

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124:188-195, 2014; Maude et al., New Eng J Med, 371: 1507-1517, 2014).

As noted in Table 5A, even moderate presentations of CRS in subjects with extensive comorbidities should be monitored closely, with consideration given to intensive care unit admission and tocilizumab administration.

Administration of Tocilizumab as a Premedication

In some aspects, an effective amount of tocilizumab is administered as a premedication (prophylaxis), e.g., is administered to the subject prior to the administration of the bispecific antibody (e.g., administered about 2 hours prior to the administration of the bispecific antibody). Administration of tocilizumab as a premedication may reduce the frequency or severity of CRS. In some aspects, tocilizumab is administered as a premedication in Cycle 1, e.g., is administered prior to a first dose (C1D1; cycle 1, dose 1), a second dose (C1D2; cycle 1, dose, 2), and/or a third dose (C1D3; cycle 1, dose 3) of the bispecific antibody. In some aspects, the tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. In some aspects, the tocilizumab is administered intravenously to the subject as a single dose of about 8 mg/kg for patients weighing 30 kg or more (maximum 800 mg) and at a dose of about 12 mg/kg for patients weighing less than 30 kg. Other anti-IL-6R antibodies that could be used in combination with tocilizumab include sarilumab, vobarilizumab (ALX-0061), SA-237, and variants thereof.

For example, in one aspect, the bispecific antibody is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In some aspects, the incidence of CRS (e.g., Grade 1 CRS, Grade 2 CRS, and/or Grade 3+ CRS) is reduced in patients who are treated with tocilizumab as a premedication relative to patients who are not treated with tocilizumab as a premedication. In some aspects, less intervention to treat CRS (e.g., less need for additional tocilizumab, IV fluids, steroids, or O₂) is required in patients who are treated with tocilizumab as a premedication relative to patients who are not treated with tocilizumab as a premedication. In some aspects, CRS symptoms have decreased severity (e.g., are limited to fevers and rigors) in patients who are treated with tocilizumab as a premedication relative to patients who are not treated with tocilizumab as a premedication.

Tocilizumab Administered to Treat CRS

In some aspects, the subject experiences a CRS event during treatment with the therapeutic bispecific antibody and an effective amount of tocilizumab is administered to manage the CRS event.

In some aspects, the subject has a CRS event (e.g., has a CRS event following treatment with the bispecific antibody, e.g., has a CRS event following a first dose or a subsequent dose of the bispecific antibody), and the method further includes treating the symptoms of the CRS event while suspending treatment with the bispecific antibody.

In some aspects, the subject experiences a CRS event, and the method further includes administering to the subject an effective amount of an interleukin-6 receptor (IL-6R) antagonist (e.g., an anti-IL-6R antibody, e.g., tocilizumab (ACTEMRA®/ROACTEMRA®)) to manage the CRS event while suspending treatment with the bispecific antibody. In some aspects, the IL-6R antagonist (e.g., tocilizumab) is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

In some aspects, the CRS event does not resolve or worsens within 24 hours of treating the symptoms of the CRS event, and the method further includes administering to the subject one or more additional doses of the IL-6R antagonist (e.g., an anti-IL-6R antibody, e.g., tocilizumab) to manage the CRS event, e.g., administering one or more additional doses of tocilizumab intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the one or more additional doses of tocilizumab are administered intravenously to the subject as a single dose of about 8 mg/kg.

In some aspects, the method further includes administering to the subject an effective amount of a corticosteroid. The corticosteroid may be administered intravenously to the subject. In some aspects, the corticosteroid is methylprednisone (methylprednisolone). In some instances, the methylprednisone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

The subject may be administered a corticosteroid, such as methylprednisolone or dexamethasone, if the CRS event is not managed with administration of the IL-6R antagonist (e.g., tocilizumab) alone. In some aspects, treating the symptoms of the CRS event further includes treatment with a high-dose vasopressor (e.g., norepinephrine, dopamine, phenylephrine, epinephrine, or vasopressin and norepinephrine), e.g., as described in Tables 5A, 5B, and 6. Tables 2 and 5A provide details about tocilizumab treatment of severe or life-threatening CRS.

Management of CRS Events by Grade

Management of the CRS events may be tailored based on the grade of the CRS (Tables 2 and 5A) and the presence of comorbidities. Table 2 provides recommendations for the management of CRS syndromes by grade.

TABLE 2
Recommendations for management of cytokine release syndrome

| Event ^{a, b} | Action to be taken ^{b} |
|---|---|
| Grade 1 | Immediate actions: |
| Fever, constitutional | If infusion is still ongoing, slow the infusion rate up to 50% or |
| symptoms | interrupt infusion. |
| | Treat symptomatically as indicated, including antihistamines, |
| | antipyretics, and/or analgesics as needed. |
| | Treat fever and neutropenia if present. |
| | Monitor fluid balance; administer IV fluids as clinically indicated. |
| | Restarting infusion: |
| | If therapeutic bispecific antibody infusion was interrupted, wait |
| | until 30 minutes after the event has resolved before restarting the |
| | infusion at 50% of the original infusion rate. |
| Grade 2 | Immediate actions: |
| Hypotension: responds to | Follow all Grade 1 recommendations. |
| fluids or a single low-dose | Hold further bispecific antibody treatment until symptoms |
| pressor ^{c} | completely resolved. |
| Hypoxia: requires <40% FiO2 | Consider treatment with IV corticosteroids (such as |
| to maintain adequate | methylprednisolone 2 mg/kg/day or, if neurologic symptoms are |
| hemoglobin oxygen saturation | present, dexamethasone 0.5 mg/kg/day). ^{b} |
| Organ toxicity: Grade 2 | Consider administering tocilizumab 8 mg/kg IV as a single dose. |
| | Monitor cardiac and other organ function closely. |
| | Provide hemodynamic support as indicated. |
| | Provide oxygen for hypoxia. |
| | Admit to ICU as appropriate. |
| | If no improvement within 24 hours, manage as a Grade 3 event: |
| | Initiate workup and assess for signs and symptoms of MAS/HLH. |
| | May receive the next dose of bispecific antibody if symptoms |
| | resolve to Grade ≤1 for 3 consecutive days. |
| | Restarting infusion: |
| | Wait until 30 minutes after the event has resolved before |
| | restarting the infusion at up to 25% of the original infusion rate. |
| | If hypotension or hypoxia recurs, stop infusion immediately. |
| | Bispecific antibody should not be re-administered (restarted) |
| | again during this cycle. |
| | If hypotension or hypoxia recurs, manage as a Grade 3 event. |
| | Next cycle: |
| | May receive the next dose of bispecific antibody if symptoms |
| | resolve to Grade ≤1 for 3 consecutive days, as follows: |
| | Administer bispecific antibody at 50% of the initial infusion rate of |
| | the previous cycle if the event occurred during or within 24 hours of |
| | the infusion. ^{d} |
| | Subsequent cycles: |
| | If there is an occurrence of IRR or CRS Grade ≥3 in any of |
| | the subsequent cycles, permanently discontinue bispecific antibody |
| | regardless of recovery (see Grade 3 management guidelines). |
| | If there is an occurrence of a Grade ≤2 CRS in subsequent |
| | cycles, manage as indicated by severity (see Grade 1 or 2 |
| | management guidelines). |
| Grade 3 | Immediate actions: |
| Hypotension: requires multiple | Stop further infusion of bispecific antibody. |
| pressor or high-dose pressor ^{c} | Treat symptomatically as indicated, including antihistamines, |
| Hypoxia: requires ≥40% FiO2 | antipyretics, and/or analgesics as needed. |
| to maintain adequate | Provide other supportive care as clinically indicated (e.g., fever |
| hemoglobin oxygen saturation | and neutropenia, infection). |
| Organ toxicity: Grade 3 | Monitor fluid balance; administer IV fluids as clinically indicated. |
| (e.g., Grade 4 transaminitis) | Hospitalize patient for at least 24 hours. |
| | Treat with IV corticosteroids (such as methylprednisolone 2 |
| | mg/kg/day or, if neurologic symptoms are present, dexamethasone |
| | 0.5 mg/kg/day). |
| | Administer tocilizumab 8 mg/kg IV. |
| | If there is no improvement after 24 hours, repeat tocilizumab |
| | administration. |
| | Initiate work up and assess for signs and symptoms of MAS/HLH. |
| | Monitor cardiopulmonary and organ function in ICU. |
| | Provide oxygen for hypoxia. |
| | Admission to ICU is recommended. |
| | Restarting infusion: |
| | Bispecific antibody should not be administered again during this cycle. |
| | Next cycle: |
| | If the patient had a Grade ≥2 IRR or CRS in any previous cycle, |
| | permanently discontinue bispecific antibody. |
| | If patient does not recover (is febrile or still on |
| | vasopressors) within 8 hours after corticosteroid and tocilizumab |
| | treatment, permanently discontinue bispecific antibody. |
| | If patient recovers (is afebrile and off vasopressors) within 8 hours |
| | following corticosteroid and tocilizumab treatment, bispecific |
| | antibody can be administered in next cycle, as follows: |
| | Hospitalize patient for at least 24 hours. |
| | Administer bispecific antibody at 50% of the initial infusion rate of |
| | the previous cycle if the event occurred during or within 24 hours of |
| | the infusion. ^{d} |
| | Subsequent cycles: |
| | If a Grade ≥3 CRS recurs, permanently discontinue bispecific antibody. |
| | If there is an occurrence of a Grade ≤2 CRS in subsequent cycles, |
| | manage as indicated by severity (i.e., Grade 1 or 2 management |
| | guidelines). |
| Grade 4 | Follow all Grade 3 management guidelines. |
| Mechanical ventilation | Permanently discontinue bispecific antibody treatment. |
| required; | |
| Organ toxicity: Grade 4 | |
| (excluding transaminitis) | |

CRS = cytokine release syndrome; HLH = hemophagocytic lymphohistiocytosis; ICU = intensive care unit; IV = intravenous; MAS = macrophage activation syndrome.
Note:
CRS is a disorder characterized by nausea, headache, tachycardia, hypotension, rash, shortness of breath, and renal, coagulation, hepatic and neurologic disorders; it is caused by the release of cytokines from cells (Lee et al., Blood, 124: 188-195, 2014).
^{a} Refer to Table 5A for description of grading of symptoms.
^{b} Guidance for CRS management based on Lee et al., Blood, 124: 188-195, 2014.
^{c} Refer to Table 5B for a description and calculation of high-dose vasopressors.
^{d} If the patient does not experience CRS during the next infusion at the 50% reduced rate, the infusion rate can be increased to the initial rate in subsequent cycles. However, if this patient experiences another CRS event, the infusion rate should be reduced by 25%-50% depending on the severity of the event.

Management of Grade 2 CRS Events

If the subject has a grade 2 CRS event (e.g., a grade 2 CRS event in the absence of comorbidities or in the presence of minimal comorbidities) following administration of the therapeutic bispecific antibody, the method may further include treating the symptoms of the grade 2 CRS event while suspending treatment with the bispecific antibody. If the grade 2 CRS event then resolves to a grade ≤1 CRS event for at least three consecutive days, the method may further include resuming treatment with the bispecific antibody without altering the dose. On the other hand, if the grade 2 CRS event does not resolve or worsens to a grade 3 CRS event within 24 hours of treating the symptoms of the grade 2 CRS event, the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the grade 2 or grade ≥3 CRS event. In some instances, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

If the subject has a grade 2 CRS event in the presence of extensive comorbidities following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 2 CRS event while suspending treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 CRS event resolves to a grade ≤1 CRS event within two weeks, the method further includes resuming treatment with the bispecific antibody at a reduced dose. In some instances, the reduced dose is 50% of the initial infusion rate of the previous cycle if the event occurred during or within 24 hours of the infusion. If, on the other hand, the grade 2 CRS event does not resolve or worsens to a grade 3 CRS event within 24 hours of treating the symptoms of the grade 2 CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade 3 CRS event. In some particular instances, the grade 2 CRS event does not resolve or worsens to a grade 3 CRS event within 24 hours of treating the symptoms of the grade 2 CRS event, and the method may further include administering to the subject one or more additional doses of tocilizumab to manage the grade 2 or grade 3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

Management of Grade 3 CRS Events

If the subject has a grade 3 CRS event following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 3 CRS event while suspending treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, the subject recovers (e.g., is afebrile and off vasopressors) within 8 hours following treatment with the bispecific antibody, and the method further includes resuming treatment with the bispecific antibody at a reduced dose. In some instances, the reduced dose is 50% of the initial infusion rate of the previous cycle if the event occurred during or within 24 hours of the infusion. In other instances, if the grade 3 CRS event does not resolve or worsens to a grade 4 CRS event within 24 hours of treating the symptoms of the grade 3 CRS event, the method may further include administering to the subject one or more (e.g., one, two, three, four, or five or more) additional doses of an IL-6R antagonist (e.g., an anti-IL-6R antibody, e.g., tocilizumab) to manage the grade 3 or grade 4 CRS event. In some particular instances, the grade 3 CRS event does not resolve or worsens to a grade 4 CRS event within 24 hours of treating the symptoms of the grade 3 CRS event, and the method further includes administering to the subject one or more additional doses of tocilizumab to manage the grade 3 or grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

Management of Grade 4 CRS Events

If the subject has a grade 4 CRS event following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 4 CRS event and permanently discontinuing treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. The grade 4 CRS event may, in some instances, resolve within 24 of treating the symptoms of the grade 4 CRS event. If the grade 4 CRS event does not resolve within 24 hours of treating the symptoms of the grade 4 CRS event, the method may further include administering to the subject one or more additional doses of an IL-6R antagonist (e.g., an anti-IL-6R antibody, e.g., tocilizumab (ACTEMRA®/ROACTEMRA®)) to manage the grade 4 CRS event. In some particular instances, the grade 4 CRS event does not resolve within 24 hours of treating the symptoms of the grade 4 CRS event, and the method further includes administering to the subject one or more (e.g., one, two, three, four, or five or more) additional doses of tocilizumab to manage the grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

ii. Corticosteroids

In another instance, the additional therapeutic agent is an effective amount of a corticosteroid. The corticosteroid may be administered intravenously to the subject. In some aspects, the corticosteroid is methylprednisone. The methylprednisone may be administered to the subject at a dose of about 80 mg. In other aspects, the corticosteroid is dexamethasone. The dexamethasone may be administered to the subject at a dose of about 80 mg. In some aspects, the corticosteroid (e.g., methylprednisone or dexamethasone) is administered to the subject prior to the administration of the bispecific anti-FcRH5/anti-CD3 antibody, e.g., administered one hour prior to the administration of the bispecific anti-FcRH5/anti-CD3 antibody.

iii. Acetaminophen or Paracetamol

In another instance, the additional therapeutic agent is an effective amount of acetaminophen or paracetamol. The acetaminophen or paracetamol may be administered orally to the subject, e.g., administered orally at a dose of between about 500 mg to about 1000 mg. In some aspects, the acetaminophen or paracetamol is administered to the subject as a premedication, e.g., is administered prior to the administration of the bispecific anti-FcRH5/anti-CD3 antibody.

iv. Diphenhydramine

In another instance, the additional therapeutic agent is an effective amount of diphenhydramine. The diphenhydramine may be administered orally to the subject, e.g., administered orally at a dose of between about 25 mg to about 50 mg. In some aspects, the diphenhydramine is administered to the subject as a premedication, e.g., is administered prior to the administration of the bispecific anti-FcRH5/anti-CD3 antibody.

v. Anti-Myeloma Agents

In another instance, the additional therapeutic agent is an effective amount of an anti-myeloma agent, e.g., an anti-myeloma agent that augments and/or complements T-cell-mediated killing of myeloma cells. The anti-myeloma agent may be, e.g., pomalidomide, daratumumab, and/or a B-cell maturation antigen (BCMA)-directed therapy (e.g., an antibody-drug conjugate targeting BCMA (BCMA-ADC)). In some aspects, the anti-myeloma agent is administered in four-week cycles.

In some aspects, the anti-myeloma agent is pomalidomide. In some aspects, the pomalidomide is administered orally at a dose of 4 mg on days 1-28 of a 28-day cycle. In some aspects, the pomalidomide is administered in combination with dexamethasone, e.g., administered in combination with dexamethasone administered on days 1, 8, 15, and 22 of a 28-day cycle.

In some aspects, the anti-myeloma agent is daratumumab. In some aspects, the daratumumab is administered by intravenous infusion (e.g., infusion over 3-5 hours) at a dose of 16 mg/kg once every week, once every two weeks, or once every four weeks. In some aspects, the daratumumab is administered by intravenous infusion (e.g., infusion over 3-5 hours) at a dose of 16 mg/kg once every week for two 28-day cycles, once every two weeks for three 28-day cycles, and once every four weeks for one or more additional cycles.

vi. Other Combination Therapies

In some aspects, the one or more additional therapeutic agents comprise a PD-L1 axis binding antagonist, an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

PD-L1 Axis Binding Antagonists

In some aspects, the additional therapeutic agent is a PD-L1 axis binding antagonist. Exemplary PD-L1 axis binding antagonists include agents that inhibit the interaction of a PD-L1 axis binding partner with one or more of its binding partners, so as to remove T cell dysfunction resulting from signaling on the PD-1 signaling axis, with a result being to restore or enhance T cell function (e.g., proliferation, cytokine production, target cell killing). As used herein, a PD-L1 axis binding antagonist includes a PD-1 binding antagonist, a PD-L1 binding antagonist, and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, or PD-L2. In some aspects, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one aspect, a PD-1 binding antagonist reduces the negative co-stimulatory signal mediated by or through cell surface proteins expressed on T lymphocytes mediated signaling through PD-1 so as render a dysfunctional T cell less dysfunctional (e.g., enhancing effector responses to antigen recognition). In some aspects, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108.

The term “PD-L1 binding antagonist” refers to a molecule that decreases, blocks, inhibits, abrogates or interferes with signal transduction resulting from the interaction of PD-L1 with either one or more of its binding partners, such as PD-1 and B7-1. In some aspects, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some aspects, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and B7-1. In one aspect, a PD-L1 binding antagonist reduces the negative co-stimulatory signal mediated by or through cell surface proteins expressed on T lymphocytes mediated signaling through PD-L1 so as to render a dysfunctional T cell less dysfunctional (e.g., enhancing effector responses to antigen recognition). In some aspects, a PD-L1 binding antagonist is an anti-PD-L1 antibody. In still another specific aspect, an anti-PD-L1 antibody is MPDL3280A (atezolizumab, marketed as TECENTRIQ™ with a WHO Drug Information (International Nonproprietary Names for Pharmaceutical Substances), Recommended INN: List 74, Vol. 29, No. 3, 2015 (see page 387)). In a specific aspect, an anti-PD-L1 antibody is YW243.55. S70. In another specific aspect, an anti-PD-L1 antibody is MDX-1105. In another specific aspect, an anti PD-L1 antibody is MSB0015718C. In still another specific aspect, an anti-PD-L1 antibody is MEDI4736.

The term “PD-L2 binding antagonist” refers to a molecule that decreases, blocks, inhibits, abrogates or interferes with signal transduction resulting from the interaction of PD-L2 with either one or more of its binding partners, such as PD-1. In some aspects, a PD-L2 binding antagonist is a molecule that inhibits the binding of PD-L2 to one or more of its binding partners. In a specific aspect, the PD-L2 binding antagonist inhibits binding of PD-L2 to PD-1. In some aspects, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T cell less dysfunctional (e.g., enhancing effector responses to antigen recognition). In some aspects, a PD-L2 binding antagonist is an immunoadhesin.

Growth Inhibitory Agents

In some aspects, the additional therapeutic agent is a growth inhibitory agent. Exemplary growth inhibitory agents include agents that block cell cycle progression at a place other than S phase, e.g., agents that induce G1 arrest (e.g., DNA alkylating agents such as tamoxifen, prednisone, dacarbazine, mechlorethamine, cisplatin, methotrexate, 5-fluorouracil, or ara-C) or M-phase arrest (e.g., vincristine, vinblastine, taxanes (e.g., paclitaxel and docetaxel), doxorubicin, epirubicin, daunorubicin, etoposide, or bleomycin).

Radiation Therapies

In some aspects, the additional therapeutic agent is a radiation therapy. Radiation therapies include the use of directed gamma rays or beta rays to induce sufficient damage to a cell so as to limit its ability to function normally or to destroy the cell altogether. Typical treatments are given as a one-time administration and typical dosages range from 10 to 200 units (Grays) per day.

Cytotoxic Agents

In some aspects, the additional therapeutic agent is a cytotoxic agent, e.g., a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and antitumor or anticancer agents.

Anti-Cancer Therapies

In some instances, the methods include administering to the individual an anti-cancer therapy other than, or in addition to, a bispecific anti-FcRH5/anti-CD3 antibody (e.g., an anti-neoplastic agent, a chemotherapeutic agent, a growth inhibitory agent, an anti-angiogenic agent, a radiation therapy, or a cytotoxic agent).

In some instances, the methods further involve administering to the patient an effective amount of an additional therapeutic agent. In some instances, the additional therapeutic agent is selected from the group consisting of an anti-neoplastic agent, a chemotherapeutic agent, a growth inhibitory agent, an anti-angiogenic agent, a radiation therapy, a cytotoxic agent, and combinations thereof. In some instances, a bispecific anti-FcRH5/anti-CD3 antibody may be administered in conjunction with a chemotherapy or chemotherapeutic agent. In some instances, a bispecific anti-FcRH5/anti-CD3 antibody may be administered in conjunction with a radiation therapy agent. In some instances, a bispecific anti-FcRH5/anti-CD3 antibody may be administered in conjunction with a targeted therapy or targeted therapeutic agent. In some instances, a bispecific anti-FcRH5/anti-CD3 antibody may be administered in conjunction with an immunotherapy or immunotherapeutic agent, for example a monoclonal antibody. In some instances, the additional therapeutic agent is an agonist directed against a co-stimulatory molecule. In some instances, the additional therapeutic agent is an antagonist directed against a co-inhibitory molecule.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some instances, a bispecific anti-FcRH5/anti-CD3 antibody may be administered in conjunction with an agonist directed against a co-stimulatory molecule. In some instances, a co-stimulatory molecule may include CD40, CD226, CD28, OX40, GITR, CD137, CD27, HVEM, or CD127. In some instances, the agonist directed against a co-stimulatory molecule is an agonist antibody that binds to CD40, CD226, CD28, OX40, GITR, CD137, CD27, HVEM, or CD127. In some instances, a bispecific anti-FcRH5/anti-CD3 antibody may be administered in conjunction with an antagonist directed against a co-inhibitory molecule. In some instances, a co-inhibitory molecule may include CTLA-4 (also known as CD152), TIM-3, BTLA, VISTA, LAG-3, B7-H3, B7-H4, IDO, TIGIT, MICA/B, or arginase. In some instances, the antagonist directed against a co-inhibitory molecule is an antagonist antibody that binds to CTLA-4, TIM-3, BTLA, VISTA, LAG-3, B7-H3, B7-H4, IDO, TIGIT, MICA/B, or arginase.

In some instances, a bispecific anti-FcRH5/anti-CD3 antibody may be administered in conjunction with an antagonist directed against CTLA-4 (also known as CD152), e.g., a blocking antibody. In some instances, a bispecific anti-FcRH5/anti-CD3 antibody may be administered in conjunction with ipilimumab (also known as MDX-010, MDX-101, or YERVOY®). In some instances, a bispecific anti-FcRH5/anti-CD3 antibody may be administered in conjunction with tremelimumab (also known as ticilimumab or CP-675,206). In some instances, a bispecific anti-FcRH5/anti-CD3 antibody may be administered in conjunction with an antagonist directed against B7-H3 (also known as CD276), e.g., a blocking antibody. In some instances, a bispecific anti-FcRH5/anti-CD3 antibody may be administered in conjunction with MGA271. In some instances, a bispecific anti-FcRH5/anti-CD3 antibody may be administered in conjunction with an antagonist directed against a TGF-beta, e.g., metelimumab (also known as CAT-192), fresolimumab (also known as GC1008), or LY2157299.

In some instances, a bispecific anti-FcRH5/anti-CD3 antibody may be administered in conjunction with a treatment comprising adoptive transfer of a T-cell (e.g., a cytotoxic T-cell or CTL) expressing a chimeric antigen receptor (CAR). In some instances, bispecific anti-FcRH5/anti-CD3 antibody may be administered in conjunction with a treatment comprising adoptive transfer of a T-cell comprising a dominant-negative TGF beta receptor, e.g., a dominant-negative TGF beta type II receptor. In some instances, a bispecific anti-FcRH5/anti-CD3 antibody may be administered in conjunction with a treatment comprising a HERCREEM protocol (see, e.g., ClinicalTrials.gov Identifier NCT00889954).

In some instances, a bispecific anti-FcRH5/anti-CD3 antibody may be administered in conjunction with an agonist directed against CD137 (also known as TNFRSF9, 4-11BB, or ILA), e.g., an activating antibody. In some instances, a bispecific anti-FcRH5/anti-CD3 antibody may be administered in conjunction with urelumab (also known as BMS-663513). In some instances, a bispecific anti-FcRH5/anti-CD3 antibody may be administered in conjunction with an agonist directed against CD40, e.g., an activating antibody. In some instances, bispecific anti-FcRH5/anti-CD3 antibody may be administered in conjunction with CP-870893. In some instances, bispecific anti-FcRH5/anti-CD3 antibody may be administered in conjunction with an agonist directed against OX40 (also known as CD134), e.g., an activating antibody. In some instances, a bispecific anti-FcRH5/anti-CD3 antibody may be administered in conjunction with an anti-OX40 antibody (e.g., AgonOX). In some instances, a bispecific anti-FcRH5/anti-CD3 antibody may be administered in conjunction with an agonist directed against CD27, e.g., an activating antibody. In some instances, a bispecific anti-FcRH5/anti-CD3 antibody may be administered in conjunction with CDX-1127. In some instances, a bispecific anti-FcRH5/anti-CD3 antibody may be administered in conjunction with an antagonist directed against indoleamine-2,3-dioxygenase (IDO). In some instances, with the IDO antagonist is 1-methyl-D-tryptophan (also known as 1-D-MT).

In some instances, a bispecific anti-FcRH5/anti-CD3 antibody may be administered in conjunction with an antibody-drug conjugate. In some instances, the antibody-drug conjugate comprises mertansine or monomethyl auristatin E (MMAE). In some instances, a bispecific anti-FcRH5/anti-CD3 antibody may be administered in conjunction with an anti-NaPi2b antibody-MMAE conjugate (also known as DNIB0600A or RG7599). In some instances, a bispecific anti-FcRH5/anti-CD3 antibody may be administered in conjunction with trastuzumab emtansine (also known as T-DM1, ado-trastuzumab emtansine, or KADCYLA®, Genentech). In some instances, a bispecific anti-FcRH5/anti-CD3 antibody may be administered in conjunction with DMUC5754A. In some instances, a bispecific anti-FcRH5/anti-CD3 antibody may be administered in conjunction with an antibody-drug conjugate targeting the endothelin B receptor (EDNBR), e.g., an antibody directed against EDNBR conjugated with MMAE.

In some instances, a bispecific anti-FcRH5/anti-CD3 antibody may be administered in conjunction with an anti-angiogenesis agent. In some instances, a bispecific anti-FcRH5/anti-CD3 antibody may be administered in conjunction with an antibody directed against a VEGF, e.g., VEGF-A. In some instances, a bispecific anti-FcRH5/anti-CD3 antibody may be administered in conjunction with bevacizumab (also known as AVASTIN®, Genentech). In some instances, a bispecific anti-FcRH5/anti-CD3 antibody may be administered in conjunction with an antibody directed against angiopoietin 2 (also known as Ang2). In some instances, a bispecific anti-FcRH5/anti-CD3 antibody may be administered in conjunction with MEDI3617.

In some instances, a bispecific anti-FcRH5/anti-CD3 antibody may be administered in conjunction with an antineoplastic agent. In some instances, a bispecific anti-FcRH5/anti-CD3 antibody may be administered in conjunction with an agent targeting CSF-1R (also known as M-CSFR or CD115). In some instances, a bispecific anti-FcRH5/anti-CD3 antibody may be administered in conjunction with anti-CSF-1R (also known as IMC-CS4). In some instances, a bispecific anti-FcRH5/anti-CD3 antibody may be administered in conjunction with an interferon, for example interferon alpha or interferon gamma. In some instances, a bispecific anti-FcRH5/anti-CD3 antibody may be administered in conjunction with Roferon-A (also known as recombinant Interferon alpha-2a). In some instances, a bispecific anti-FcRH5/anti-CD3 antibody may be administered in conjunction with GM-CSF (also known as recombinant human granulocyte macrophage colony stimulating factor, rhu GM-CSF, sargramostim, or LEUKINE®). In some instances, a bispecific anti-FcRH5/anti-CD3 antibody may be administered in conjunction with IL-2 (also known as aldesleukin or PROLEUKIN®). In some instances, a bispecific anti-FcRH5/anti-CD3 antibody may be administered in conjunction with IL-12. In some instances, a bispecific anti-FcRH5/anti-CD3 antibody may be administered in conjunction with an antibody targeting CD20. In some instances, the antibody targeting CD20 is obinutuzumab (also known as GA101 or GAZYVA®) or rituximab. In some instances, a bispecific anti-FcRH5/anti-CD3 antibody may be administered in conjunction with an antibody targeting GITR. In some instances, the antibody targeting GITR is TRX518.

In some instances, a bispecific anti-FcRH5/anti-CD3 antibody may be administered in conjunction with a cancer vaccine. In some instances, the cancer vaccine is a peptide cancer vaccine, which in some instances is a personalized peptide vaccine. In some instances the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a bispecific anti-FcRH5/anti-CD3 antibody may be administered in conjunction with an adjuvant. In some instances, a bispecific anti-FcRH5/anti-CD3 antibody may be administered in conjunction with a treatment comprising a TLR agonist, e.g., Poly-ICLC (also known as HILTONOL®), LPS, MPL, or CpG ODN. In some instances, a bispecific anti-FcRH5/anti-CD3 antibody may be administered in conjunction with tumor necrosis factor (TNF) alpha. In some instances, a bispecific anti-FcRH5/anti-CD3 antibody may be administered in conjunction with IL-1. In some instances, a bispecific anti-FcRH5/anti-CD3 antibody may be administered in conjunction with HMGB1. In some instances, a bispecific anti-FcRH5/anti-CD3 antibody may be administered in conjunction with an IL-10 antagonist. In some instances, a bispecific anti-FcRH5/anti-CD3 antibody may be administered in conjunction with an IL-4 antagonist. In some instances, a bispecific anti-FcRH5/anti-CD3 antibody may be administered in conjunction with an IL-13 antagonist. In some instances, a bispecific anti-FcRH5/anti-CD3 antibody may be administered in conjunction with an HVEM antagonist. In some instances, a bispecific anti-FcRH5/anti-CD3 antibody may be administered in conjunction with an ICOS agonist, e.g., by administration of ICOS-L, or an agonistic antibody directed against ICOS. In some instances, a bispecific anti-FcRH5/anti-CD3 antibody may be administered in conjunction with a treatment targeting CX3CL1. In some instances, a bispecific anti-FcRH5/anti-CD3 antibody may be administered in conjunction with a treatment targeting CXCL9. In some instances, a bispecific anti-FcRH5/anti-CD3 antibody may be administered in conjunction with a treatment targeting CXCL10. In some instances, a bispecific anti-FcRH5/anti-CD3 antibody may be administered in conjunction with a treatment targeting CCL5. In some instances, a bispecific anti-FcRH5/anti-CD3 antibody may be administered in conjunction with an LFA-1 or ICAM1 agonist. In some instances, a bispecific anti-FcRH5/anti-CD3 antibody may be administered in conjunction with a Selectin agonist.

In some instances, a bispecific anti-FcRH5/anti-CD3 antibody may be administered in conjunction with a targeted therapy. In some instances, a bispecific anti-FcRH5/anti-CD3 antibody may be administered in conjunction with an inhibitor of B-Raf. In some instances, a bispecific anti-FcRH5/anti-CD3 antibody may be administered in conjunction with vemurafenib (also known as ZELBORAF®). In some instances, a bispecific anti-FcRH5/anti-CD3 antibody may be administered in conjunction with dabrafenib (also known as TAFINLAR®). In some instances, a bispecific anti-FcRH5/anti-CD3 antibody may be administered in conjunction with erlotinib (also known as TARCEVA®). In some instances, a bispecific anti-FcRH5/anti-CD3 antibody may be administered in conjunction with an inhibitor of a MEK, such as MEK1 (also known as MAP2K1) or MEK2 (also known as MAP2K2). In some instances, a bispecific anti-FcRH5/anti-CD3 antibody may be administered in conjunction with cobimetinib (also known as GDC-0973 or XL-518). In some instances, a bispecific anti-FcRH5/anti-CD3 antibody may be administered in conjunction with trametinib (also known as MEKINIST®). In some instances, a bispecific anti-FcRH5/anti-CD3 antibody may be administered in conjunction with an inhibitor of K-Ras. In some instances, a bispecific anti-FcRH5/anti-CD3 antibody may be administered in conjunction with an inhibitor of c-Met. In some instances, a bispecific anti-FcRH5/anti-CD3 antibody may be administered in conjunction with onartuzumab (also known as MetMAb). In some instances, a bispecific anti-FcRH5/anti-CD3 antibody may be administered in conjunction with an inhibitor of Alk. In some instances, a bispecific anti-FcRH5/anti-CD3 antibody may be administered in conjunction with AF802 (also known as CH5424802 or alectinib). In some instances, a bispecific anti-FcRH5/anti-CD3 antibody may be administered in conjunction with an inhibitor of a phosphatidylinositol 3-kinase (PI3K). In some instances, a bispecific anti-FcRH5/anti-CD3 antibody may be administered in conjunction with BKM120. In some instances, a bispecific anti-FcRH5/anti-CD3 antibody may be administered in conjunction with idelalisib (also known as GS-1101 or CAL-101). In some instances, a bispecific anti-FcRH5/anti-CD3 antibody may be administered in conjunction with perifosine (also known as KRX-0401). In some instances, a bispecific anti-FcRH5/anti-CD3 antibody may be administered in conjunction with an inhibitor of an Akt. In some instances, a bispecific anti-FcRH5/anti-CD3 antibody may be administered in conjunction with MK2206. In some instances, a bispecific anti-FcRH5/anti-CD3 antibody may be administered in conjunction with GSK690693. In some instances, a bispecific anti-FcRH5/anti-CD3 antibody may be administered in conjunction with GDC-0941. In some instances, a bispecific anti-FcRH5/anti-CD3 antibody may be administered in conjunction with an inhibitor of mTOR. In some instances, a bispecific anti-FcRH5/anti-CD3 antibody may be administered in conjunction with sirolimus (also known as rapamycin). In some instances, a bispecific anti-FcRH5/anti-CD3 antibody may be administered in conjunction with temsirolimus (also known as CCI-779 or TORISEL®). In some instances, a bispecific anti-FcRH5/anti-CD3 antibody may be administered in conjunction with everolimus (also known as RAD001). In some instances, a bispecific anti-FcRH5/anti-CD3 antibody may be administered in conjunction with ridaforolimus (also known as AP-23573, MK-8669, or deforolimus). In some instances, a bispecific anti-FcRH5/anti-CD3 antibody may be administered in conjunction with OSI-027. In some instances, a bispecific anti-FcRH5/anti-CD3 antibody may be administered in conjunction with AZD8055. In some instances, a bispecific anti-FcRH5/anti-CD3 antibody may be administered in conjunction with INK128. In some instances, a bispecific anti-FcRH5/anti-CD3 antibody may be administered in conjunction with a dual PI3K/mTOR inhibitor. In some instances, a bispecific anti-FcRH5/anti-CD3 antibody may be administered in conjunction with XL765. In some instances, a bispecific anti-FcRH5/anti-CD3 antibody may be administered in conjunction with GDC-0980. In some instances, a bispecific anti-FcRH5/anti-CD3 antibody may be administered in conjunction with BEZ235 (also known as NVP-BEZ235). In some instances, a bispecific anti-FcRH5/anti-CD3 antibody may be administered in conjunction with BGT226. In some instances, a bispecific anti-FcRH5/anti-CD3 antibody may be administered in conjunction with GSK2126458. In some instances, a bispecific anti-FcRH5/anti-CD3 antibody may be administered in conjunction with PF-04691502. In some instances, a bispecific anti-FcRH5/anti-CD3 antibody may be administered in conjunction with PF-05212384 (also known as PKI-587).

In some instances, a bispecific anti-FcRH5/anti-CD3 antibody may be administered in conjunction with a chemotherapeutic agent. A chemotherapeutic agent is a chemical compound useful in the treatment of cancer. Exemplary chemotherapeutic agents include, but are not limited to erlotinib (TARCEVA®, Genentech/OSI Pharm.), anti-hormonal agents that act to regulate or inhibit hormone action on tumors such as anti-estrogens and selective estrogen receptor modulators (SERMs), antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), or trastuzumab (HERCEPTIN®, Genentech), EGFR inhibitors (EGFR antagonists), tyrosine kinase inhibitors, and chemotherapeutic agents also include non-steroidal anti-inflammatory drugs (NSAIDs) with analgesic, antipyretic and anti-inflammatory effects.

In instances for which the methods described herein involve a combination therapy, such as a particular combination therapy noted above, the combination therapy encompasses the co-administration of the bispecific anti-FcRH5/anti-CD3 antibody with one or more additional therapeutic agents, and such co-administration may be combined administration (where two or more therapeutic agents are included in the same or separate formulations) or separate administration, in which case, administration of the bispecific anti-FcRH5/anti-CD3 antibody can occur prior to, simultaneously, and/or following, administration of the additional therapeutic agent or agents. In one embodiment, administration of the bispecific anti-FcRH5/anti-CD3 antibody and administration of an additional therapeutic agent or exposure to radiotherapy can occur within about one month, or within about one, two or three weeks, or within about one, two, three, four, five, or six days, of each other.

In some aspects, the subject does not have an increased risk of CRS (e.g., has not experienced Grade 3+ CRS during treatment with a bispecific antibody or CAR-T therapy; does not have detectable circulating plasma cells; and/or does not have extensive extramedullary disease).

D. Cancers

Any of the methods of the invention described herein may be useful for treating cancer, such as a B cell proliferative disorder, including multiple myeloma (MM), which may be relapsed or refractory (R/R) MM. In some aspects, the patient has received at least three prior lines of treatment for the B cell proliferative disorder (e.g., MM), e.g., is 4L+, e.g., has received three, four, five, six, or more than six prior lines of treatment. For example, the patient may have been exposed to a proteasome inhibitor (PI), an immunomodulatory drug (IMiD), an autologous stem cell transplant (ASCT), an anti-CD38 therapy (e.g., anti-CD38 antibody therapy, e.g., daratumumab therapy), a CAR-T therapy, or a therapy comprising a bispecific antibody. In some instances, the patient has been exposed to all three of PI, IMiD, and anti-CD38 therapy. Other examples of B cell proliferative disorders/malignancies amenable to treatment with a bispecific anti-FcRH5/anti-CD3 antibody in accordance with the methods described herein include, without limitation, non-Hodgkin's lymphoma (NHL), including diffuse large B cell lymphoma (DLBCL), which may be relapsed or refractory DLBCL, as well as other cancers including germinal-center B cell-like (GCB) diffuse large B cell lymphoma (DLBCL), activated B cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, Waldenström macroglobulinemia, heavy chain diseases, a heavy chain disease, γ heavy chain disease, p heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle centre lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, primary mediastinal (thymic) large B cell lymphoma, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of B cell proliferative disorders include, but are not limited to, multiple myeloma (MM); low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Further examples of cancer include, but are not limited to, carcinoma, lymphoma, blastoma, sarcoma, and leukemia or lymphoid malignancies, including B cell lymphomas. More particular examples of such cancers include, but are not limited to, low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Solid tumors that may by amenable to treatment with a bispecific anti-FcRH5/anti-CD3 antibody in accordance with the methods described herein include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, and mesothelioma.

E. Prior Anti-Cancer Therapy

In some aspects, the subject has previously been treated for the B cell proliferative disorder (e.g., MM). In some aspects, the subject has received at least one, two, three, four, five, six, seven, eight, nine, ten, eleven, twelve, thirteen, fourteen, fifteen, or more than fifteen lines of treatment for the B cell proliferative disorder, e.g., is 2L+, 3L+, 4L+, 5L+, 6L+, 7L+, 8L+, 9L+, 10L+, 11 L+, 12L+, 13L+, 14L+, or 15L+. In some aspects, the subject has received at least three prior lines of treatment for the B cell proliferative disorder (e.g., MM), e.g., is 4L+, e.g., has received three, four, five, six, seven, eight, nine, ten, eleven, twelve, thirteen, fourteen, fifteen, or more than fifteen lines of treatment. In some aspects, the subject has relapsed or refractory (R/R) multiple myeloma (MM), e.g., has 4L+R/R MM.

In some aspects, the prior lines of treatment include one or more of a proteasome inhibitor (PI), e.g., bortezomib, carfilzomib, or ixazomib; an immunomodulatory drug (IMiD), e.g., thalidomide, lenalidomide, or pomalidomide; an autologous stem cell transplant (ASCT); an anti-CD38 agent, e.g., daratumumab (DARZALEX®) (U.S. Pat. No. 7,829,673 and U.S. Pub. No: 20160067205 A1), “MOR202” (U.S. Pat. No. 8,263,746), isatuximab (SAR-650984); a CAR-T therapy; a therapy comprising a bispecific antibody; an anti-SLAMF7 therapeutic agent (e.g., an anti-SLAMF7 antibody, e.g., elotuzumab); a nuclear export inhibitor (e.g., selinexor); and a histone deacetylase (HDAC) inhibitor (e.g., panobinostat). In some aspects, the prior lines of treatment include an antibody-drug conjugate (ADC). In some aspects, the prior lines of treatment include a B-cell maturation antigen (BCMA)-directed therapy, e.g., an antibody-drug conjugate targeting BCMA (BCMA-ADC).

In some aspects, the prior lines of treatment include all three of a proteasome inhibitor (PI), an IMiD, and an anti-CD38 agent (e.g., daratumumab).

In some aspects, the B cell proliferative disorder (e.g., MM) is refractory to the lines of treatment, e.g., is refractory to one or more of daratumumab, a PI, an IMiD, an ASCT, an anti-CD38 agent, a CAR-T therapy, a therapy comprising a bispecific antibody, an anti-SLAMF7 therapeutic agent, a nuclear export inhibitor, a HDAC inhibitor, an ADC, or a BCMA-directed therapy. In some aspects, the B cell proliferative disorder (e.g., MM) is refractory to daratumumab.

F. Risk-Benefit Profile

The methods described herein may result in an improved benefit-risk profile for patients having cancer (e.g., a multiple myeloma (MM), e.g., a relapsed or refractory (R/R) MM), e.g., a 4L+R/R MM, being treated with a bispecific anti-FcRH5/anti-CD3 antibody. In some instances, treatment using the methods described herein that result in administering the bispecific anti-FcRH5/anti-CD3 antibody in the context of a fractionated, dose-escalation dosing regimen may result in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) or complete inhibition (100% reduction) of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with a bispecific anti-FcRH5/anti-CD3 antibody using the fractionated, dose-escalation dosing regimen of the invention relative to treatment with a bispecific anti-FcRH5/anti-CD3 antibody using an non-fractioned dosing regimen.

G. Safety and Efficacy

i. Safety

In some aspects, less than 15% (e.g., less than 14%, less than 13%, less than 12%, less than 11%, less than 10%, less than 9%, less than 8%, less than 7%, less than 6%, less than 5%, less than 4%, less than 3%, less than 2%, or less than 1%) of patients treated using the methods described herein experience Grade 3 or Grade 4 cytokine release syndrome (CRS). In some aspects, less than 5% of patients treated using the methods described herein experience Grade 3 or Grade 4 CRS.

In some aspects, less than 10% (e.g., less than 9%, less than 8%, less than 7%, less than 6%, less than 5%, less than 4%, less than 3%, less than 2%, or less than 1%) of patients treated using the methods described herein experience Grade 4+ CRS. In some aspects, less than 3% of patients treated using the methods described herein experience Grade 4+ CRS. In some aspects, no patients experience Grade 4+ CRS.

In some aspects, less than 10% (e.g., less than 9%, less than 8%, less than 7%, less than 6%, less than 5%, less than 4%, less than 3%, less than 2%, or less than 1%) of patients treated using the methods described herein experience Grade 3 CRS. In some aspects, less than 5% of patients treated using the methods described herein experience Grade 3 CRS. In some aspects, no patients experience Grade 3 CRS.

In some aspects, Grade 2+ CRS events occur only in the first cycle of treatment. In some aspects, Grade 2 CRS events occur only in the first cycle of treatment. In some aspects, Grade 2 CRS events do not occur.

In some aspects, less than 3% of patients treated using the methods described herein experience Grade 4+ CRS, less than 5% of patients treated using the methods described herein experience Grade 3 CRS, and Grade 2+ CRS events occur only in the first cycle of treatment.

In some aspects, no Grade 3+ CRS events occur and Grade 2 CRS events occur only in the first cycle of treatment.

In some aspects, symptoms of immune effector cell-associated neurotoxicity syndrome (ICANS) are limited to confusion, disorientation, and expressive aphasia and resolve with steroids.

In some aspects, less than 10% (e.g., less than 9%, less than 8%, less than 7%, less than 6%, less than 5%, less than 4%, less than 3%, less than 2%, or less than 1%) of patients treated using the methods described herein experience seizures or other Grade 3+ neurologic adverse events. In some aspects, less than 5% of patients experience seizures or other Grade 3+ neurologic adverse events. In some aspects, no patients experience seizures or other Grade 3+ neurologic adverse events.

In some aspects, all neurological symptoms are either self-limited or resolved with steroids and/or tocilizumab therapy.

ii. Efficacy

In some aspects, the overall response rate (ORR) for patients treated using the methods described herein is at least 25%, e.g., is at least 30%, 35%, 40%, 45%, 50%, 55%, 60%, 65%, 70%, 75%, 80%, 85%, 90%, 95%, 99%, or 100%. In some aspects, the ORR is at least 40%. In some aspects, the ORR is at least 45% (e.g., at least 45%, 45.5%, 46%, 46.5% 47%, 47.5%, 48%, 48.5%, 49%, 49.5%, or 50%) at least 55%, or at least 65%. In some aspects, the ORR is at least 47.2%. In some aspects, the ORR is about 47.2%. In some aspects, the ORR is 75% or greater. In some aspects, at least 1% of patients (e.g., at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of patients) have a complete response (CR) or a very good partial response (VGPR). In some aspects, the ORR is 40%-50%, and 10%-20% of patients have a CR or a VGPR. In some aspects, the ORR is at least 40%, and at least 20% of patients have a CR or a VGPR.

In some aspects, the average duration of response (DoR) for patients treated using the methods described herein is at least two months, e.g., at least three months, at least four months, at least five months, at least six months, at least seven months, at least eight months, at least nine months, at least ten months, at least eleven months, at least one year, or more than one year. In some aspects, the average DoR is at least four months. In some aspects, the average DoR is at least five months. In some aspects, the average DoR is at least seven months.

In some aspects, the six month progression-free survival (PFS) rate for patients treated using the methods described herein is at least 10%, e.g., is at least 15%, 20%, 25%, 30%, 35%, 40%, 45%, 50%, 55%, 60%, 65%, 70%, 75%, 80%, 85%, 90%, 95%, 99%, or 100%. In some aspects, the six month PFS rate is at least 25%. In some aspects, the six month PFS rate is at least 40%. In some aspects, the six month PFS rate is at least 55%.

H. Methods of Administration

The methods may involve administering the bispecific anti-FcRH5/anti-CD3 antibody (and/or any additional therapeutic agent) by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, the bispecific anti-FcRH5/anti-CD3 antibody is administered by intravenous infusion. In other instances, the bispecific anti-FcRH5/anti-CD3 antibody is administered subcutaneously.

In some instances, the bispecific anti-FcRH5/anti-CD3 antibody administered by intravenous injection exhibits a less toxic response (i.e., fewer unwanted effects) in a patient than the same bispecific anti-FcRH5/anti-CD3 antibody administered by subcutaneous injection, or vice versa.

In some aspects, the bispecific anti-FcRH5/anti-CD3 antibody is administered intravenously over 4 hours (±15 minutes), e.g., the first dose of the antibody is administered over 4 hours±15 minutes.

In some aspects, the first dose and the second dose of the antibody are administered intravenously with a median infusion time of less than four hours (e.g., less than three hours, less than two hours, or less than one hour) and further doses of the antibody are administered intravenously with a median infusion time of less than 120 minutes (e.g., less than 90 minutes, less than 60 minutes, or less than 30 minutes.

In some aspects, the first dose and the second dose of the antibody are administered intravenously with a median infusion time of less than three hours and further doses of the antibody are administered intravenously with a median infusion time of less than 90 minutes.

In some aspects, the first dose and the second dose of the antibody are administered intravenously with a median infusion time of less than three hours and further doses of the antibody are administered intravenously with a median infusion time of less than 60 minutes. In some aspects, the patient is hospitalized (e.g., hospitalized for 72 hours, 48 hours, 24 hours, or less than 24 hours) during one or more administrations of the anti-FcRH5/anti-CD3 antibody, e.g., hospitalized for the C1D1 (cycle 1, dose 1) or the C1D1 and the C1D2 (cycle 1, dose 2). In some aspects, the patient is hospitalized for 72 hours following administration of the C1D1 and the C1D2. In some aspects, the patient is hospitalized for 24 hours following administration of the C1D1 and the C1D2. In some aspects, the patient is not hospitalized following the administration of any dose of the anti-FcRH5/anti-CD3 antibody.

For all the methods described herein, the bispecific anti-FcRH5/anti-CD3 antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The bispecific anti-FcRH5/anti-CD3 antibody need not be, but is optionally formulated with, one or more agents currently used to prevent or treat the disorder in question. The effective amount of such other agents depends on the amount of the bispecific anti-FcRH5/anti-CD3 antibody present in the formulation, the type of disorder or treatment, and other factors discussed above. The bispecific anti-FcRH5/anti-CD3 antibody may be suitably administered to the patient over a series of treatments.

I. Anti-FcRH5/Anti-CD3 Bispecific Antibodies

The methods described herein include administering to a subject having a cancer (e.g., a multiple myeloma, e.g., an R/R multiple myeloma) a bispecific antibody that binds to FcRH5 and CD3 (i.e., a bispecific anti-FcRH5/anti-CD3 antibody).

In some instances, any of the methods described herein may include administering a bispecific antibody that includes an anti-FcRH5 arm having a first binding domain comprising at least one, two, three, four, five, or six hypervariable regions (HVRs) selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, any of the methods described herein may include administering a bispecific antibody that includes an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, the bispecific antibody comprises an anti-FcRH5 arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). Accordingly, in some instances, the first binding domain comprises a VH domain comprising an amino acid sequence of SEQ ID NO: 7 and a VL domain comprising an amino acid sequence of SEQ ID NO: 8.

In some instances, any of the methods described herein may include administering a bispecific anti-FcRH5/anti-CD3 antibody that includes an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, any of the methods described herein may include administering a bispecific anti-FcRH5/anti-CD3 antibody that includes an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, the bispecific antibody comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the second binding domain comprises a VH domain comprising an amino acid sequence of SEQ ID NO: 15 and a VL domain comprising an amino acid sequence of SEQ ID NO: 16.

In some instances, any of the methods described herein may include administering a bispecific antibody that includes (1) an anti-FcRH5 arm having a first binding domain comprising at least one, two, three, four, five, or six HVRs selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, any of the methods described herein may include administering a bispecific antibody that includes (1) an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, the anti-FcRH5/anti-CD3 bispecific antibody comprises (1) at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively, and (2) at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, the anti-FcRH5/anti-CD3 bispecific antibody comprises (1) all four of heavy chain framework regions FR-H1, FR-H2, FR-H3, and FR-H4 comprising the sequences of SEQ ID NOs: 17-20, respectively, and/or all four of the light chain framework regions FR-L1, FR-L2, FR-L3, and FR-L4 comprising the sequences of SEQ ID NOs: 21-24, respectively, and (2) all four of heavy chain framework regions FR-H1, FR-H2, FR-H3, and FR-H4 comprising the sequences of SEQ ID NOs: 25-28, respectively, and/or all four (e.g., 1, 2, 3, or 4) of the light chain framework regions FR-L1, FR-L2, FR-L3, and FR-L4 comprising the sequences of SEQ ID NOs: 29-32, respectively.

In some instances, the anti-FcRH5/anti-CD3 bispecific antibody comprises (1) an anti-FcRH5 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). In some instances, the anti-FcRH5/anti-CD3 bispecific antibody comprises (1) a first binding domain comprising a VH domain comprising an amino acid sequence of SEQ ID NO: 7 and a VL domain comprising an amino acid sequence of SEQ ID NO: 8 and (2) a second binding domain comprising a VH domain comprising an amino acid sequence of SEQ ID NO: 15 and a VL domain comprising an amino acid sequence of SEQ ID NO: 16.

In some instances, the anti-FcRH5/anti-CD3 bispecific antibody comprises an anti-FcRH5 arm comprising a heavy chain polypeptide (H1) and a light chain polypeptide (L1), wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35 and/or (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36.

In some instances, the anti-FcRH5/anti-CD3 bispecific antibody comprises an anti-FcRH5 arm comprising a heavy chain polypeptide (H1) and a light chain polypeptide (L1), wherein (a) H1 comprises the amino acid sequence of SEQ ID NO: 35 and/or (b) L1 comprises the amino acid sequence of SEQ ID NO: 36.

In some instances, the anti-FcRH5/anti-CD3 bispecific antibody comprises an anti-CD3 arm comprising a heavy chain polypeptide (H2) and a light chain polypeptide (L2), wherein (a) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37 and/or (b) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-CD3 arm comprising a heavy chain polypeptide (H2) and a light chain polypeptide (L2), wherein (a) H2 comprises the amino acid sequence of SEQ ID NO: 37; and (b) L2 comprises the amino acid sequence of SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35; (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36; (c) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37; and (d) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises the amino acid sequence of SEQ ID NO: 35; (b) L1 comprises the amino acid sequence of SEQ ID NO: 36; (c) H2 comprises the amino acid sequence of SEQ ID NO: 37; and (d) L2 comprises the amino acid sequence of SEQ ID NO: 38.

In some instances, the anti-FcRH5/anti-CD3 bispecific antibody is cevostamab.

In some instances, the anti-FcRH5/anti-CD3 bispecific antibody according to any of the above embodiments described above may incorporate any of the features, singly or in combination, as described in Sections 1-7 below.

1. Antibody Affinity

In certain embodiments, an antibody provided herein has a dissociation constant (K_{D}) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 pg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, NJ) is performed at 37° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 37° C. at a flow rate of approximately 25 μl/min. Association rates (kₒₙ, or kₐ) and dissociation rates (kₒₙ, or k_{d}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio kₒₙ/kₒₙ. See, for example, Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 37° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

2. Antibody Fragments

In certain embodiments, an antibody provided herein (e.g., an anti-FcRH5/anti-CD3 TDB) is an antibody fragment that binds FcRH5 and CD3. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Nat. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein (e.g., an anti-FcRH5/anti-CD3 TDB) is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof), for example, are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein (e.g., an anti-FcRH5/anti-CD3 TDB) is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Multispecific Antibodies

In any one of the above aspects, an anti-FcRH5/anti-CD3 antibody provided herein is a multispecific antibody, for example, a bispecific antibody. Multispecific antibodies are antibodies (e.g., monoclonal antibodies) that have binding specificities for at least two different sites, e.g., antibodies having binding specificities for an immune effector cell and for a cell surface antigen (e.g., a tumor antigen, e.g., FcRH5) on a target cell other than an immune effector cell. In some aspects, one of the binding specificities is for FcRH5 and the other is for CD3.

In some aspects, the cell surface antigen may be expressed in low copy number on the target cell. For example, in some aspects, the cell surface antigen is expressed or present at less than 35,000 copies per target cell. In some embodiments, the low copy number cell surface antigen is present between 100 and 35,000 copies per target cell; between 100 and 30,000 copies per target cell; between 100 and 25,000 copies per target cell; between 100 and 20,000 copies per target cell; between 100 and 15,000 copies per target cell; between 100 and 10,000 copies per target cell; between 100 and 5,000 copies per target cell; between 100 and 2,000 copies per target cell; between 100 and 1,000 copies per target cell; or between 100 and 500 copies per target cell. Copy number of the cell surface antigen can be determined, for example, using a standard Scatchard plot.

In some embodiments, a bispecific antibody may be used to localize a cytotoxic agent to a cell that expresses a tumor antigen, e.g., FcRH5. Bispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). “Knob-in-hole” engineering of multispecific antibodies may be utilized to generate a first arm containing a knob and a second arm containing the hole into which the knob of the first arm may bind. The knob of the multispecific antibodies of the invention may be an anti-CD3 arm in one embodiment. Alternatively, the knob of the multispecific antibodies of the invention may be an anti-target/antigen arm in one embodiment. The hole of the multispecific antibodies of the invention may be an anti-CD3 arm in one embodiment. Alternatively, the hole of the multispecific antibodies of the invention may be an anti-target/antigen arm in one embodiment.

Multispecific antibodies may also be engineered using immunoglobulin crossover (also known as Fab domain exchange or CrossMab format) technology (see, e.g., WO2009/080253; Schaefer et al., Proc. Natl. Acad. Sci. USA, 108:11187-11192 (2011)). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibodies, or antibody fragments thereof, may also include a “Dual Acting FAb” or “DAF” comprising an antigen binding site that binds to CD3 as well as another, different antigen (e.g., a second biological molecule) (see, e.g., US 2008/0069820).

6. Antibody Variants

In some aspects, amino acid sequence variants of the bispecific anti-FcRH5/anti-CD3 antibodies of the invention are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the CDRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norieucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact an antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b. Glycosylation Variants

In certain embodiments, bispecific anti-FcRH5/anti-CD3 antibodies of the invention can be altered to increase or decrease the extent to which the antibody is glycosylated. Addition or deletion of glycosylation sites to anti-FcRH5 antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, bispecific anti-FcRH5/anti-CD3 antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Bispecific anti-FcRH5/anti-CD3 antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c. Fc Region Variants

In certain embodiments, one or more amino acid modifications may be introduced into the Fc region of a bispecific anti-FcRH5/anti-CD3 antibody, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates a bispecific anti-FcRH5/anti-CD3 antibody variant that possesses some but not all effector functions, which make it a desirable candidate for applications in which the half-life of the antibody in vivo is important, yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain embodiments, the proline at position 329 of a wild-type human Fc region in the antibody is substituted with glycine or arginine or an amino acid residue large enough to destroy the proline sandwich within the Fc/Fc.gamma. receptor interface that is formed between the proline 329 of the Fc and tryptophan residues Trp 87 and Trp 110 of FcgRIII (Sondermann et al. Nature. 406, 267-273, 2000). In certain embodiments, the antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the anti-FcRH5 and/or anti-CD3 antibody (e.g., bispecific anti-FcRH5 antibody) comprises an Fc region comprising an N297G mutation (EU numbering). In some aspects, the anti-FcRH5 arm of the bispecific anti-FcRH5 antibody comprises a N297G mutation and/or the anti-CD3 arm of the bispecific anti-FcRH5 antibody comprises an Fc region comprising an N297G mutation.

In some embodiments, the anti-FcRH5 antibody comprising the N297G mutation comprises an anti-FcRH5 arm comprising a first binding domain comprising the following six HVRs (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6; and an anti-CD3 arm comprising an N297G mutation. In some embodiments, the anti-CD3 arm comprising the N297G mutation comprises the following six HVRs: (a) an HVR-H1 comprising the amino acid sequence of SEQ ID NO: 9; (b) an HVR-H2 comprising the amino acid sequence of SEQ ID NO: 10; (c) an HVR-H3 comprising the amino acid sequence of SEQ ID NO: 11; (d) an HVR-L1 comprising the amino acid sequence of SEQ ID NO: 12; (e) an HVR-L2 comprising the amino acid sequence of SEQ ID NO: 13; and (f) an HVR-L3 comprising the amino acid sequence of SEQ ID NO: 14.

In some embodiments, the anti-FcRH5 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm comprising an N297G mutation. In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

In some embodiments, the anti-FcRH5 antibody comprising the N297G mutation comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some aspects, at least one of the one or more heavy chain constant domains is paired with another heavy chain constant domain. In some aspects, the CH3₁ and CH3₂ domains each comprise a protuberance or cavity, and wherein the protuberance or cavity in the CH3₁ domain is positionable in the cavity or protuberance, respectively, in the CH3₂ domain. In some aspects, the CH3₁ and CH3₂ domains meet at an interface between said protuberance and cavity. In some aspects, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some aspects, the anti-FcRH5 antibody is an IgG₁ antibody.

In some embodiments, the anti-FcRH5 antibody comprising the N297G mutation comprises an anti-FcRH5 arm comprising a first binding domain comprising (a) a VH domain comprising the amino acid sequence of SEQ ID NO: 7 and (b) a VL domain comprising the amino acid sequence of SEQ ID NO: 8, and an anti-CD3 arm, wherein (a) the anti-FcRH5 arm comprises T366S, L368A, Y407V, and N297G amino acid substitution mutations (EU numbering) and (b) the anti-CD3 arm comprises T366W and N297G substitution mutations (EU numbering). In some embodiments, the anti-CD3 arm comprising the T366W and N297G mutations comprises comprising (a) a VH domain comprising an amino acid sequence of SEQ ID NO: 15 and (b) a VL domain comprising an amino acid sequence of SEQ ID NO: 16.

In other embodiments, the anti-FcRH5 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm, wherein (a) the anti-FcRH5 arm comprises T366W and N297G substitution mutations (EU numbering) and (b) the anti-CD3 arm comprises T366S, L368A, Y407V, and N297G mutations (EU numbering). In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

d. Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

e. Antibody Derivatives

In certain embodiments, a bispecific anti-FcRH5/anti-CD3 antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

7. Charged Regions

In some aspects, the binding domain that binds FcRH5 or CD3 comprises a VH1 comprising a charged region (CR₁) and a VL1 comprising a charged region (CR₂), wherein the CR, in the VH1 forms a charge pair with the CR₂ in the VL1. In some aspects, the CR, comprises a basic amino acid residue and the CR₂ comprises an acidic amino acid residue. In some aspects, the CR₁ comprises a Q39K substitution mutation (Kabat numbering). In some aspects, the CR₁ consists of the Q39K substitution mutation. In some aspects, the CR₂ comprises a Q38E substitution mutation (Kabat numbering). In some aspects, the CR₂ consists of the Q38E substitution mutation. In some aspects, the second binding domain that binds CD3 comprises a VH2 comprising a charged region (CR₃) and a VL2 comprising a charged region (CR₄), wherein the CR₄ in the VL2 forms a charge pair with the CR₃ in the VH2. In some aspects, the CR₄ comprises a basic amino acid residue and the CR₃ comprises an acidic amino acid residue. In some aspects, the CR₄ comprises a Q38K substitution mutation (Kabat numbering). In some aspects, the CR₄ consists of the Q38K substitution mutation. In some aspects, the CR₃ comprises a Q39E substitution mutation (Kabat numbering). In some aspects, the CR₃ consists of the Q39E substitution mutation. In some aspects, the VL1 domain is linked to a light chain constant domain (CL1) domain and the VH1 is linked to a first heavy chain constant domain (CH1), wherein the CL1 comprises a charged region (CR₅) and the CH1 comprises a charged region (CR₆), and wherein the CR₅ in the CL1 forms a charge pair with the CR₆ in the CH1₁. In some aspects, the CR₅ comprises a basic amino acid residue and the CR₆ comprises an acidic residue. In some aspects, the CR₅ comprises a V133K substitution mutation (EU numbering). In some aspects, the CR₅ consists of the V133K substitution mutation. In some aspects, the CR₆ comprises a S183E substitution mutation (EU numbering). In some aspects, the CR₆ consists of the S183E substitution mutation.

In other aspects, the VL2 domain is linked to a CL domain (CL2) and the VH2 is linked to a CH1 domain (CH1₂), wherein the CL2 comprises a charged region (CR₇) and the CH1₂ comprises a charged region (CR₈), and wherein the CR₈ in the CH1₂ forms a charge pair with the CR₇ in the CL2. In some aspects, the CR₈ comprises a basic amino acid residue and the CR₇ comprises an acidic amino acid residue. In some aspects, the CR₈ comprises a S183K substitution mutation (EU numbering). In some aspects, the CR₈ consists of the S183K substitution mutation. In some aspects, the CR₈ comprises a V133E substitution mutation (EU numbering). In some aspects, the CR₈ consists of the V133E substitution mutation.

In other aspects, the VL2 domain is linked to a CL domain (CL2) and the VH2 is linked to a CH1 domain (CH1₂), wherein (a) the CL2 comprises one or more mutations at amino acid residues F116, L135, S174, S176, and/or T178 (EU numbering) and (b) the CH1₂ comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH1₂ comprises one or more of the following substitution mutations: A141I, F170S, S181M, S183A, and/or V185A. In some aspects, the CH1₂ comprises the following substitution mutations: A141I, F170S, S181M, S183A, and V185A.

In other aspects, the binding domain that binds FcRH5 or CD3 comprises a VH domain (VH1) comprising a charged region (CR₁) and a VL domain (VL1) comprising a charged region (CR₂), wherein the CR₂ in the VL, forms a charge pair with the CR₁ in the VH1. In some aspects, the CR₂ comprises a basic amino acid residue and the CR, comprises an acidic amino acid residue. In some aspects, the CR₂ comprises a Q38K substitution mutation (Kabat numbering). In some aspects, the CR₂ consists of the Q38K substitution mutation. In some aspects, the CR, comprises a Q39E substitution mutation (Kabat numbering). In some aspects, the CR, consists of the Q39E substitution mutation. In some aspects, the second binding domain that binds CD3 comprises a VH domain (VH2) comprising a charged region (CR₃) and a VL domain (VL2) comprising a charged region (CR₄), wherein the CR₃ in the VH2 forms a charge pair with the CR₄ in the VL2. In some aspects, the CR₃ comprises a basic amino acid residue and the CR₄ comprises an acidic amino acid residue. In some aspects, the CR₃ comprises a Q39K substitution mutation (Kabat numbering). In some aspects, the CR₃ consists of the Q39K substitution mutation. In some aspects, the CR₄ comprises a Q38E substitution mutation (Kabat numbering). In some aspects, the CR₄ consists of the Q38E substitution mutation. In some aspects, the VL1 domain is linked to a light chain constant domain (CL1) and the VH1 is linked to a first heavy chain constant domain (CH1₁), wherein the CL1 comprises a charged region (CR₅) and the CH1 comprises a charged region (CR₆), and wherein the CR₆ in the CH1 forms a charge pair with the CR₅ in the CL1. In some aspects, the CR₆ comprises a basic amino acid residue and the CR₅ comprises an acidic amino acid residue. In some aspects, the CR₆ comprises a S183K substitution mutation (EU numbering). In some aspects, the CR₆ consists of the S183K substitution mutation. In some aspects, the CR₅ comprises a V133E substitution mutation (EU numbering). In some aspects, the CR₅ consists of the V133E substitution mutation.

In other aspects, the VL2 domain is linked to a CL domain (CL2) and the VH2 is linked to a CH1 domain (CH1₂), wherein the CL2 comprises a charged region (CR₇) and the CH1₂ comprises a charged region (CR₈), and wherein the CR₇ in the CL2 forms a charged pair with the CR₈ in the CH1₂. In some aspects, the CR₇ comprises a basic amino acid residue and the CR₈ comprises an acidic residue. In some aspects, the CR₇ comprises a V133K substitution mutation (EU numbering). In some aspects, the CR₇ consists of the V133K substitution mutation. In some aspects, the CR₈ comprises a S183E substitution mutation (EU numbering). In some aspects, the CR₈ consists of the S183E substitution mutation.

In other aspects, the VL2 domain is linked to a CL domain (CL2) and the VH2 is linked to a CH1 domain (CH1₂), wherein (a) the CL2 comprises one or more mutations at amino acid residues F116, L135, S174, S176, and/or T178 (EU numbering) and (b) the CH1₂ comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH1₂ comprises one or more of the following substitution mutations: A141I, F170S, S181M, S183A, and/or V185A. In some aspects, the CH1₂ comprises the following substitution mutations: A141I, F170S, S181M, S183A, and V185A. In some aspects, the anti-FcRH5 antibody comprises one or more heavy chain constant domains, wherein the one or more heavy chain constant domains are selected from a first CH2 domain (CH2₁), a first CH3 domain (CH3₁), a second CH2 domain (CH2₂), and a second CH3 domain (CH3₂). In some aspects, at least one of the one or more heavy chain constant domains is paired with another heavy chain constant domain. In some aspects, the CH3₁ and the CH3₂ each comprise a protuberance (P₁) or a cavity (C₁), and wherein the P₁ or the C₁ in the CH3₁ is positionable in the C₁ or the P₁, respectively, in the CH3₂. In some aspects, the CH3, and the CH3₂ meet at an interface between the P₁ and the C₁. In some aspects, the CH2₁ and the CH2₂ each comprise (P₂) or a cavity (C₂), and wherein the P₂ or the C₂ in the CH2₁ is positionable in the C₂ or the P₂, respectively, in the CH2₂. In some aspects, the CH2₁ and the CH2₂ meet at an interface between the P₂ and the C₂.

J. Recombinant Methods and Compositions

Bispecific anti-FcRH5/anti-CD3 antibodies of the invention may be produced using recombinant methods and compositions, for example, as described in U.S. Pat. No. 4,816,567. In one embodiment, an isolated nucleic acid encoding an anti-FcRH5 antibody described herein is provided. Such nucleic acid may encode an amino acid sequence comprising the VL and/or an amino acid sequence comprising the VH of the antibody (e.g., the light and/or heavy chains of the antibody). In another embodiment, an isolated nucleic acid encoding an anti-CD3 antibody described herein is provided. Such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such a nucleic acid are provided. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making a bispecific anti-FcRH5/anti-CD3 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of a bispecific anti-FcRH5/anti-CD3 antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

1. Two-Cell Methods for Manufacturing Bispecific Antibodies

In some aspects, an antibody of the invention (e.g., a bispecific anti-FcRH5/anti-CD3 antibody) is manufactured using a method comprising two host cell lines. In some aspects, a first arm of the antibody (e.g., a first arm comprising a hole region) is produced in a first host cell line, and a second arm of the antibody (e.g., a second arm comprising a knob region) is produced in a second host cell line. The arms of the antibody are purified from the host cell lines and assembled in vitro.

2. One-Cell Methods for Manufacturing Bispecific Antibodies

In some aspects, an antibody of the invention (e.g., a bispecific anti-FcRH5/anti-CD3 antibody) is manufactured using a method comprising a single host cell line. In some aspects, a first arm of the antibody (e.g., a first arm comprising a hole region) and a second arm of the antibody (e.g., a second arm comprising a knob region) are produced in and purified from a single host cell line. Preferably, the first arm and the second arm are expressed at comparable levels in the host cell, e.g., are both expressed at a high level in the host cell. Similar levels of expression increase the likelihood of efficient TDB production and decrease the likelihood of light chain (LC) mispairing of TDB components. The first arm and second arm of the antibody may each further comprise amino acid substitution mutations introducing charge pairs, as described in Section IIB (7) herein. The charge pairs promote the pairing of heavy and light chain cognate pairs of each arm of the bispecific antibody, thereby minimizing mispairing.

3. Host Cells

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

K. Immunoconjugates

The invention also provides immunoconjugates comprising a bispecific anti-FcRH5/anti-CD3 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises a bispecific anti-FcRH5/anti-CD3 antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises a bispecific anti-FcRH5/anti-CD3 antibody described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

L. Pharmaceutical Compositions and Formulations

Pharmaceutical compositions and formulations of the anti-FcRH5/anti-CD3 bispecific antibodies can be prepared by mixing such antibodies having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as L-Histidine/glacial acetic acid (e.g., at pH 5.8), phosphate, citrate, and other organic acids; tonicity agents, such as sucrose; stabilizers, such as L-methionine; antioxidants including N-acetyl-DL-tryptophan, ascorbic acid, and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbate 20 or polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### III. Articles of Manufacture

In another aspect of the invention, an article of manufacture containing materials useful for the treatment, prevention, and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-FcRH5/anti-CD3 bispecific antibody described herein. In some aspects, the article of manufacture comprises at least two containers (e.g., vials), a first container holding an amount of the composition suitable for a C1D1 (cycle 1, dose 1) and a second container holding an amount of the composition suitable for a C1D2 (cycle 1, dose 2). In some aspects, the article of manufacture comprises at least three containers (e.g., vials), a first container holding an amount of the composition suitable for a C1D1, a second container holding an amount of the composition suitable for a C1D2, and a third container holding an amount of the composition suitable for a C1D3. In some aspects, the containers (e.g., vials) may be different sizes, e.g., may have sizes proportional to the amount of the composition they contain. Articles of manufacture comprising containers (e.g., vials) proportional to the intended doses may, e.g., increase convenience, minimize waste, and/or increase cost-effectiveness. The label or package insert indicates that the composition is used for treating the condition of choice (e.g., a multiple myeloma (MM), e.g., relapsed or refractory MM, e.g., 4L+R/R MM) and further includes information related to at least one of the dosing regimens described herein. Moreover, the article of manufacture may comprise (a) a first container with a composition contained therein, wherein the composition comprises an anti-FcRH5/anti-CD3 bispecific antibody described herein; and (b) a second container with a composition contained therein, wherein the composition comprises a further cytotoxic or otherwise therapeutic agent. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### IV. Examples

The following are examples of the methods of the invention. It is understood that various other embodiments may be practiced, given the general description provided above, and the examples are not intended to limit the scope of the claims.

### Example 1. Phase I Trial Evaluating the Safety and Efficacy of Escalating Doses of Cevostamab (BFCR4350A) in Patients with R/R MM

GO39775 (NCT03275103) is an open-label, multicenter, Phase I trial evaluating the safety and pharmacokinetics of escalating doses of the anti-FcRH5/anti-CD3 T-cell-dependent bispecific antibody (TDB) cevostamab (BFCR4350A) in approximately 150 patients with relapsed or refractory multiple myeloma for whom no established therapy for MM is appropriate and available or who are intolerant to those established therapies. A dedicated expansion arm to test tocilizumab pretreatment in ameliorating the frequency and/or severity of CRS following treatment with cevostamab (Arm E) is included.

### A. Background

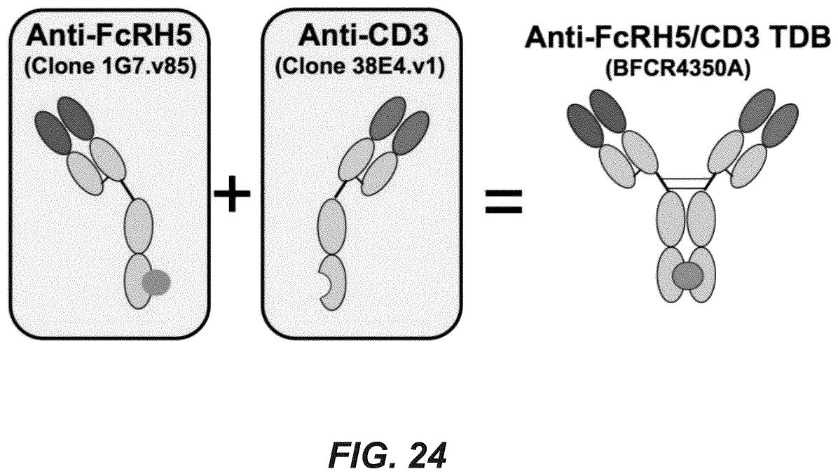

Cevostamab (BFCR4350A) is a humanized, full-length immunoglobulin (Ig) G1 anti-fragment crystallizable receptor-like 5/cluster of differentiation 3 (anti-FcRH5/anti-CD3) T-cell-dependent bispecific antibody (TDB) produced in Chinese hamster ovary cells using knobs-into-holes technology (Atwell et al., J Mol Bio, 270: 26-35, 1997; Spiess et al., Nat Biotechnol, 31(8): 753-758, 2013) (FIG. 24). Cevostamab contains the N297G amino acid substitution in the Fc regions of the KFCR8534A and HCDT4425A half-antibodies based on EU numbering, which results in non-glycosylated heavy chains that have minimal binding to Fcγ receptors (FcγRs) and, consequently, attenuates Fc-effector function.

### B. Inclusion Criteria

This study enrolls patients with a history of R/R MM that is expected to express the FcRH5 antigen and who meet the inclusion and exclusion criteria as outlined below. Confirmation of FcRH5 expression is not required during eligibility screening prior to enrollment, but is evaluated retrospectively, based on the following rationale:
Nonclinical studies have demonstrated that cevostamab is broadly active in cell killing in multiple human MM cell lines and primary human MM plasma cells with a wide range of FcRH5 expression levels, including cells with minimal FcRH5 expression, suggesting that even very low levels of FcRH5 expression may be sufficient for clinical activity (Li et al., Cancer Cell, 31: 383-395, 2017).
FcRH5 is a cell-surface antigen whose expression is restricted to cells of the B lineage, including plasma cells. It is expressed with 100% prevalence on MM samples tested to date (Elkins et al., Mol Cancer Ther, 11: 2222-2232, 2012; Li et al. Cancer Cell, 31: 383-395, 2017).
   Bone marrow samples obtained from all patients are retrospectively analyzed for FcRH5 expression with validation of assays (e.g., quantitative reverse transcription-PCR, immunohistochemistry, and quantitative flow cytometry). These data are used to inform how best to utilize FcRH5 expression screening in subsequent studies.
      Patients must meet the following criteria for study entry:
Age ≥18 years
Eastern Cooperative Oncology Group (ECOG) Performance Status of 0 or 1
Life expectancy of at least 12 weeks
Patients must have R/R MM for which no established therapy for MM is appropriate and available or be intolerant to those established therapies
Adverse events from prior anti-cancer therapy resolved to Grade ≤1, with the following exceptions:
   Any grade alopecia
   Peripheral sensory or motor neuropathy must have resolved to Grade ≤2
Measurable disease defined as at least one of the following:
   Serum monoclonal protein (M-protein) ≥0.5 g/dL (≥5 g/L)
   Urine M-protein ≥200 mg/24 hr
   Serum free light chain (SFLC) assay: Involved SFLCs ≥10 mg/dL (≥100 mg/L) and an abnormal SFLC ratio (<0.26 or >1.65)
Laboratory values as follows:
   Hepatic function
      AST and ALT ≤3×ULN
      Total bilirubin ≤1.5×ULN; patients with a documented history of Gilbert syndrome and in whom total bilirubin elevations are accompanied by elevated indirect bilirubin are eligible.
   Hematologic function (requirement prior to first dose of cevostamab)
      Platelet count ≥50,000/mm³ without transfusion within 14 days prior to first dose of cevostamab
      ANC ≥1000/mm³
      Total hemoglobin ≥8 g/dL
         Patients who do not meet criteria for hematologic function because of MM-related cytopenias (e.g. due to extensive marrow involvement by MM) may be enrolled into the study after discussion with and with the approval of the Medical Monitor. Patients may receive supportive care to meet hematologic function eligibility criteria (e.g., transfusions, G-CSF, etc.).
   Creatinine ≤2.0 mL/dL and creatinine clearance (CrCl) ≥30 mL/min (either calculated or per 24-hr urine collection)
   Serum calcium (corrected for albumin) level at or below Grade 1 hypercalcemia (patient may receive treatment for hypercalcemia to meet eligibility criteria)
For women of childbearing potential: agreement to remain abstinent (refrain from heterosexual intercourse) or use contraceptive measures.
For men: agreement to remain abstinent (refrain from heterosexual intercourse) or use a condom, and agreement to refrain from donating sperm.

C. Exclusion Criteria

Patients who meet any of the following criteria are excluded from study entry:
Pregnant or breastfeeding, or intending to become pregnant during the study or within 3 months after the last dose of study drug.
Women of childbearing potential must have a negative serum pregnancy test result within 7 days prior to initiation of study drug.
Prior use of any monoclonal antibody, radioimmunoconjugate, or antibody-drug conjugate as anti-cancer therapy within 4 weeks before first cevostamab infusion.
Prior treatment with chimeric antigen receptor (CAR) T-cell therapy within 12 weeks before first cevostamab infusion.
Prior treatment with systemic immunotherapeutic agents, including, but not limited to, cytokine therapy and anti-CTLA4, anti-PD-1, and anti-PD-L1 therapeutic antibodies, within 12 weeks or 5 half-lives of the drug, whichever is shorter, before first cevostamab infusion.
Known treatment-related, immune-mediated adverse events associated with prior immunotherapeutic agents as follows:
   Prior PD-L1/PD-1 or CTLA-4 inhibitor: Grade 3 adverse events, with the exception of Grade 3 endocrinopathy managed with replacement therapy
   Grade 1-2 adverse events that did not resolve to baseline after treatment discontinuation
Treatment with radiotherapy, any chemotherapeutic agent, or treatment with any other anti-cancer agent (investigational or otherwise) within 4 weeks or 5 half-lives of the drug, whichever is shorter, prior to first cevostamab infusion.
Autologous stem cell transplantation (SCT) within 100 days prior to first cevostamab infusion.
Prior allogeneic SCT.
Absolute plasma cell count exceeding 500/μL or 5% of the peripheral blood white cells.
Prior solid organ transplantation.
History of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjögren's syndrome, Guillain-Barré syndrome, multiple sclerosis, vasculitis, or glomerulonephritis.
   Patients with a history of autoimmune-related hypothyroidism on a stable dose of thyroid replacement hormone may be eligible for this study.
Patients with history of confirmed progressive multifocal leukoencephalopathy.
History of severe allergic or anaphylactic reactions to monoclonal antibody therapy (or recombinant antibody-related fusion proteins).
Patients with known history of amyloidosis (e.g., positive Congo Red stain or equivalent in tissue biopsy).
Patients with lesions in proximity of vital organs that may develop sudden decompensation/deterioration in the setting of a tumor flare.
   Patients may be eligible after discussion with the Medical Monitor.
History of other malignancy that could affect compliance with the protocol or interpretation of results.
   Patients with a history of curatively treated basal or squamous cell carcinoma of the skin or in situ carcinoma of the cervix are allowed.
Patients with a malignancy that has been treated with curative intent will also be allowed if the malignancy has been in remission without treatment for 2 years prior to first cevostamab infusion. Current or past history of CNS disease, such as stroke, epilepsy, CNS vasculitis, neurodegenerative disease, or CNS involvement by MM.
   Patients with a history of stroke who have not experienced a stroke or transient ischemic attack in the past 2 years and have no residual neurologic deficits as judged by the investigator are allowed.
   Patients with a history of epilepsy who have had no seizures in the past 2 years while not receiving any anti-epileptic medications are allowed.
Significant cardiovascular disease (such as, but not limited to, New York Heart Association Class III or IV cardiac disease, myocardial infarction within the last 6 months, uncontrolled arrhythmias, or unstable angina) that may limit a patient's ability to adequately respond to a CRS event Symptomatic active pulmonary disease requiring supplemental oxygen.
Known active bacterial, viral, fungal, mycobacterial, parasitic, or other infection (excluding fungal infections of nail beds) at study enrollment, or any major episode of infection requiring treatment with IV antibiotics within 4 weeks prior to first cevostamab infusion.
Known or suspected chronic active EBV infection. Guidelines for diagnosing chronic active EBV infection are provided by Okano et al., Am J Hematol, 80: 64-69, 2005.
Recent major surgery within 14 days prior to first cevostamab infusion.
   Protocol-mandated procedures (e.g., bone marrow biopsies) are permitted.
Positive serologic or PCR test results for acute or chronic HBV infection
   Patients whose HBV infection status cannot be determined by serologic test results must be negative for HBV by PCR to be eligible for study participation.
Acute or chronic HCV infection.
   Patients who are positive for HCV antibody must be negative for HCV by PCR to be eligible for study participation.
Known history of HIV seropositivity.
Administration of a live, attenuated vaccine within 4 weeks before first cevostamab infusion or anticipation that such a live attenuated vaccine will be required during the study Received systemic immunosuppressive medications (including, but not limited to, cyclophosphamide, azathioprine, methotrexate, thalidomide, and anti-tumor necrosis factor agents) with the exception of corticosteroid treatment ≤10 mg/day prednisone or equivalent within 2 weeks prior to first dose of cevostamab and, if applicable, tocilizumab premedication prior to first dose of cevostamab.
   Patients who received acute, low-dose, systemic immunosuppressant medications (e.g., single dose of dexamethasone for nausea) may be enrolled in the study after discussion with and approval of the Medical Monitor.
   The use of inhaled corticosteroids is permitted.
   The use of mineralocorticoids for management of orthostatic hypotension is permitted.
   The use of physiologic doses of corticosteroids for management of adrenal insufficiency is permitted.
History of illicit drug or alcohol abuse within 12 months prior to screening, in the investigator's judgment

D. Dosage and Administration: Cevostamab

Flat dosing independent of body weight is used for cevostamab. The dose of cevostamab for each patient depends on their dose level assignment, as described in Example 2.

Cevostamab is directed against the extracellular domains of the FcRH5 and CD3 antigens. Engagement of both arms of anti-FcRH5/anti-CD3 TDB results in T-cell-directed cell killing of FcRH5+ malignant cells for the treatment of MM. Therefore, at pharmacologically active doses, T-cell activation, including cytokine release, is anticipated in the presence of FcRH5+ cells. Consequently, determination of the recommended safe starting dose in this Phase I study (GO39775) employed a minimum anticipated biological effect level (MABEL) approach based on in vitro T-cell activation. The proposed starting dose in patients is a flat dose of 0.05 mg (0.7 μg/kg based on a 70-kg patient) and is supported by in vitro experiments with human peripheral blood mononuclear cells (PBMCs) co-cultured with MOLP-2 cells. The 4-week dose toxicity study in cynomolgus monkey also supports the proposed starting dose for cevostamab.

The estimated Cₘₐₓ at the proposed starting dose is approximately 14 ng/mL (range of 8-25 ng/mL, based on body weight range of 40-120 kg, assuming a 50 mL/kg human volume of distribution to the central compartment). This estimated Cₘₐₓ has a predicted pharmacological activity of approximately 20%-25% based on the 50% effective concentration (EC50) value (58.8±41 ng/mL, and taking into account donor variability) for T-cell activation in the in vitro human PBMC:MOLP-2 co-culture (based on the calculation [C/EC₅₀+C], where C is the estimated concentration at 0.05 mg; Saber et al., Regul Toxicol Pharmacol, 81: 448-456, 2016; Saber et al., Society of Toxicology, abstract 1556, 2017). T-cell activation in the in vitro human PBMC:MOLP-2 co-culture is the most sensitive safety endpoint in the most sensitive assay. Moreover, this projected Cₘₐₓ is lower than EC50 cytokine release in the in vitro human PBMC:MOLP-2 co-culture (minimal cytokine release with high donor to donor variability; EC50 values range from 63.6-289.25 ng/mL). At this estimated Cₘₐₓ, CD3 receptor occupancy is calculated to be 4%, based on the 2.6 nM monovalent dissociation constant (KD) of cevostamab.

The proposed starting dose is supported by the established highest non-severely toxic dose (HNSTD) of 4 mg/kg in the cynomolgus monkey. Based on the Cₘₐₓ achieved in cynomolgus monkey studies (Cₘₐₓ=40.7 μg/mL at 4-mg/kg doses for fractionated dose, and Cₘₐₓ=129 μg/mL), the proposed starting dose of 0.05 mg has a 2900- to 9200-fold safety factor range. The body-weight-normalized dose-based safety factor is 5600 (calculated as follows: Dose_{cynomolgus monkey, HNSTD}/Dose_{human, proposed starting dose}=4 mg/kg/0.7 μg/kg). The pharmacologically active dose of 0.01 mg/kg was also established based on changes in B-cell counts, T-cell activation, and cytokine level increases in cynomolgus monkeys' peripheral blood. The estimated Cₘₐₓ at the proposed starting dose is approximately 10-fold below the observed Cₘₐₓ of 135 ng/mL at the cynomolgus monkey pharmacologically active dose. Cevostamab exhibited potent B-cell killing in cynomolgus monkey in vitro and in vivo, compared to minimal to moderate (20%-40%) in vitro B-cell killing observed with cevostamab in human PBMCs. PK simulations based upon other therapeutic IgG1 antibodies with similar PK characteristics do not suggest clinically meaningful differences in exposure variability following fixed dose or dose adjusted for weight (Bai et al., Clin Pharmacokinet, 51: 119-135, 2012). On the basis of this simulations-based evaluation, fixed doses are proposed for this study. Fixed dosing has been utilized and approved for multiple monoclonal antibodies (e.g., GAZYVA® (obinutuzumab), U.S. Package Insert, Genentech USA, Inc.).

Cevostamab is administered to patients by IV infusion using standard medical syringes and syringe pumps or IV bags where applicable. Compatibility testing has shown that cevostamab is stable in extension sets and polypropylene syringes. The Drug Product is delivered by syringe pump via an IV infusion set or by IV bag infusion, with a final cevostamab volume determined by the dose.

Hospitalization requirements for patients receiving cevostamab are described herein. Cevostamab is administered in a setting with immediate access to trained critical care personnel and facilities equipped to respond to and manage medical emergencies. Alternatively, cevostamab is administered to patients by subcutaneous (SQ or SC) injection.

All cevostamab doses are administered to well-hydrated patients. Corticosteroid premedication consisting of dexamethasone 20 mg IV or methylprednisolone 80 mg IV must be administered 1 hour prior to the administration of each cevostamab dose in Cycle 1 and Cycle 2, or in the subsequent cycle if the patient experienced CRS with the prior dose. Starting in Cycle 3, corticosteroid premedication may be discontinued in patients who did not have CRS in the prior dose. In addition, premedication with oral acetaminophen or paracetamol (e.g., 500-1000 mg) and 25-50 mg diphenhydramine must be administered prior to administration of cevostamab, unless contraindicated. For sites that do not have access to diphenhydramine, an equivalent medication may be substituted per local practice.

Initially, cevostamab is administered over 4 hours (±15 minutes). The infusion may be slowed or interrupted for patients experiencing IRRs. At the end of the cevostamab infusions during Cycle 1, patients are hospitalized. Patients are observed at least 90 minutes for fever, chills, rigors, hypotension, nausea, or other signs and symptoms of IRRs following each subsequent cevostamab infusion. Also, in the absence of IRRs, the infusion time of cevostamab in subsequent cycles may be reduced to 2 hours.

Patients who undergo intrapatient dose escalation should receive the first higher infusion of cevostamab over a minimum of 4 hours.

E. Dosage and Administration: Tocilizumab

Tocilizumab is administered when necessary, as described below. Based on review of available clinical data, it may be required that tocilizumab be administered prior to administration of cevostamab during Cycle 1. In some aspects, tocilizumab is administered to all patients prior to the administration of cevostamab.

CRS is a potentially life-threatening symptom complex, caused by the excessive release of cytokines by immune effector or target cells during an exaggerated and sustained immune response. CRS can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or fatal outcome. Current clinical management focuses on treating the individual signs and symptoms, providing supportive care, and attempting to dampen down the inflammatory response using high-dose corticosteroids. However, this approach is not always successful, especially in the case of late intervention. Moreover, steroids may negatively impact T-cell function, which may diminish the clinical benefit of immune modulating therapies in the treatment of cancer.

CRS is associated with elevations in a wide array of cytokines, including marked elevations in IFN-γ, IL-6, and tumor necrosis factor-alpha (TNF-α) levels. Emerging evidence implicates IL-6 as a central mediator in CRS. IL-6 is a pro-inflammatory multi-functional cytokine produced by a variety of cell types, which has been shown to be involved in a diverse array of physiological processes including T-cell activation.

Regardless of the inciting agent, CRS is associated with high IL-6 levels (Panelli et al., J Transl Med, 2: 17, 2004; Lee et al., Blood, 124: 188-195, 2014; Doessegger and Banholzer, Clin Transl Immunology, 4: e39, 2015), and IL-6 correlates with the severity of CRS with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J Immunol Methods, 434: 1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling. Patients treated with cevostamab who develop severe CRS may benefit from tocilizumab therapy.

On Aug. 30, 2017, the U.S. Food and Drug Administration approved tocilizumab for the treatment of severe or life-threatening CAR-T cell-induced CRS in adults and in pediatric patients 2 years of age and older. Initial clinical data (Locke et al., Blood, 130: 1547, 2017) suggests that tocilizumab prophylaxis may reduce the severity of CAR-T cell-induced CRS by blocking IL-6 receptors from signaling prior to cytokine release. Consequently, tocilizumab premedication may also reduce the frequency or lower the severity of CRS associated with cevostamab. Tocilizumab may be required to be administered as a premedication in Cycle 1 in either treatment arm (i.e., Arm A or Arm B) if there would likely be benefit in further reducing the frequency or severity of CRS, based on the totality of the data with step fractionation. Patients may be administered one or more than one dose of tocilizumab. The tocilizumab label allows up to four doses 8 hours apart for treatment of CRS. CRS treatment may include administration of IV steroids.

F. Disease-Specific Assessments

Patients are evaluated for disease response and progression according to the International Myeloma Working Group (IMWG) response criteria (Table 4) during each cycle of treatment. Cycles of treatment are described in detail in Example 2.

A bone marrow biopsy and aspirate are required prior to C1D1 dosing, between the Cycle 1 target dose infusion day and C2D1, within 7 days prior to or on Cycle 4, and at the time of confirmation of CR or disease progression.

The following myeloma-specific tests are conducted at the beginning of every cycle, starting with C1D1:
Serum protein electrophoresis (SPEP) with serum immunofixation electrophoresis (SIFE)
SFLCs
Quantitative Ig levels
   The following myeloma-specific tests should be performed at screening and as needed to confirm a response:
A 24-hour urine protein electrophoresis (UPEP) with urine immunofixation electrophoresis (UIFE) for M-protein quantitation

The following confirmatory assessments are required for all response categories (stringent complete response (sCR), CR, VGPR, PR, and minimal response (MR)), as defined in Table 4:
If extra-medullary disease was previously present, CT scan or MRI with bi-dimensional measurements to confirm reduction in size per IMWG criteria
If extra-medullary disease was previously present, PET-CT scan, CT scan, or MRI to confirm complete resolution
24-hour UPEP/UIFE is required to confirm VGPR even if a UPEP was not performed at screening.

The following additional samples/assessments are required to confirm a sCR or CR:
SIFE
SFLC
24-hour UPEP/UIFE (performed locally) is required to confirm CR/sCR even if a UPEP was not performed at screening
Bone marrow aspiration and biopsy
If extra-medullary disease was previously present, PET-CT scan, CT scan, or MRI to confirm complete resolution
   To confirm progressive disease, the following are required:
If progressive disease is suspected by rising M-protein, SPEP, UPEP, or SFLC analysis should be obtained on two consecutive assessments in two consecutive cycles.
If progressive disease is suspected on development of new bone lesions or soft tissue plasmacytomas or an increase in size of existing bone lesions or soft tissue plasmacytomas, skeletal survey/CT scan/MRI should be obtained and compared with baseline imaging.
If progressive disease is suspected on hypercalcemia attributed solely to MM, local laboratory results levels of serum calcium should be ≥11 mg/dL and confirmed on a second assessment.

All patients with clinically suspected extra-medullary disease or known extra-medullary disease at the time of screening must undergo imaging during screening to evaluate for the presence/extent of extramedullary disease. This can be performed using PET/CT, CT scan, or whole-body MRI. Patients who are found to have extra-medullary disease undergo repeat imaging (preferably the same modality as performed at screening) every 12 weeks (±7 days). Imaging should also be performed upon clinical suspicion of progressive disease.

A skeletal survey is completed at screening and as clinically indicated. Plain films and CT scans are both acceptable imaging modalities for assessing skeletal disease. Imaging should include the skull, long bones, chest, and pelvis. If plasmacytomas are seen on skeletal survey, bi-dimensional tumor measurements should be recorded. The skeletal survey may be omitted if a PET/CT scan or a low-dose, whole-body CT is performed as part of screening.

TABLE 4
International Myeloma Working Group (IMWG) uniform response criteria (2016)

| | |
|---|---|
| Response Subcategory | Response Criteria |

All response categories require two consecutive assessments made any time before
starting any new therapy

| | |
|---|---|
| Stringent complete | CR as defined below, plus: |
| response (sCR) | Normal FLC ratio and absence of clonal cells in BM by |
| | immunohistochemistry (kappa/lambda ratio ≤4:1 or ≥1:2 for kappa and lambda patients, |
| | respectively after counting ≥100 plasma cells in BM |
| Complete response | No evidence of initial monoclonal protein isotype(s) on immunofixation of |
| (CR) | the serum and urine,^{b} disappearance of any soft tissue plasmacytomas, and ≤5% |
| | plasma cells in BM |
| Very good partial | Serum and urine M-protein detectable by immunofixation but not on |
| response (VGPR) | electrophoresis; or ≥90% reduction in serum M-protein plus urine M-protein |
| | level <100 mg/24 hr |
| Partial response (PR) | ≥50% reduction of serum M-protein and reduction in 24-hour urine M- |
| | protein by ≥90% or to <200 mg/24 hr |
| | If the serum and urine M-protein are unmeasurable, a ≥50% |
| | decrease in the difference between involved and uninvolved FLC |
| | levels is required in place of the M-protein criteria. |
| | If serum and urine M-protein are unmeasurable and serum FLC |
| | assay is also unmeasurable, ≥50% reduction in plasma cells is |
| | required in place of M-protein, provided baseline BM plasma cell |
| | percentage was ≥30% |
| | In addition to the above listed criteria, if present at baseline, a ≥50% |
| | reduction in the size (SPD)^{c} of soft tissue plasmacytomas is |
| | also required. |
| Minimal response (MR) | ≥25% but ≤49% reductions of serum M-protein and reduction in 24-hour |
| | urine M-protein by 50%-89% |
| | In addition to the above criteria, if present at baseline, 25%-49% reduction in |
| | the size (SPD)^{c} of soft tissue plasmacytomas is also required. |
| Stable disease (SD) | Not meeting criteria for MR, CR, VGPR, PR, or PD |
| Progressive disease | Any one or more of the following criteria: |
| (PD) ^{d, e} | Increase of ≥25% from lowest response value in one or more of the following: |
| | Serum M-protein (absolute increase must be ≥0.5 g/dL) |
| | Serum M-protein increase ≥1 g/dL, if the lowest M component was ≥5 g/dL |
| | Urine M-protein (absolute increase must be ≥200 mg/24 hr) |
| | In patients without measurable serum and urine M-protein levels: |
| | the difference between involved and uninvolved FLC levels |
| | (absolute increase must be >10 mg/dL) |
| | In patients without measurable serum and urine M-protein levels |
| | and without measurable disease by FLC: BM plasma cell |
| | percentage irrespective of baseline status (absolute % must be ≥10%)^{b} |
| | Appearance of new lesion(s), ≥50% increase from nadir in SPD of >1 |
| | lesion, or ≥50% increase in the longest diameter of a previous |
| | lesion >1 cm in short axis |
| | ≥50% increase in circulating plasma cells (minimum 200 cells per |
| | microliter) if this is the only measure of disease |
| | Development of new CRAB criteria events |
| Clinical relapse | Requires one or more of the following: |
| | Direct indications of increasing disease and/or end organ |
| | dysfunction (CRAB features) ^{f} related to the underlying clonal |
| | plasma cell proliferative disorder. It is not used in calculation of time |
| | to progression or PFS but is listed here as something that can be |
| | reported optionally or for use in clinical practice. |
| | Development of new soft tissue plasmacytomas or bone lesions |
| | (osteoporotic fractures do not constitute progression) |
| | Definite increase in the size of existing plasmacytomas or bone |
| | lesions. A definite increase is defined as a 50% (and ≥1 cm) |
| | increase as measured serially by the sum of the products of the |
| | cross-diameters of the measurable lesion. |
| | Hypercalcemia >11 mg/dl (2.65 mmol/L) |
| | Decrease in hemoglobin of ≥2 g/dL (1.25 mmol/L) not related to |
| | therapy or other non-myeloma related conditions |
| | Rise in serum creatinine by 2 mg/dl or more (177 μmol/L or more) |
| | from the start of therapy and attributable to myeloma |
| | Hyperviscosity related to serum paraprotein |
| Relapse from CR (to be | Any one or more of the following: |
| used only if the endpoint | Reappearance of serum or urine M-protein by immunofixation or |
| studied is PFS)^{c} | electrophoresis |
| | Development of ≥5% plasma cells in the BM |
| | Appearance of any other sign of progression (i.e., new |
| | plasmacytoma, lytic bone lesion, or hypercalcemia) |

BM = bone marrow; CT = computed tomography; FLC = free light chain; M-protein = monoclonal protein; MRI = magnetic resonance imaging; PET = positron emission tomography; PFS = progression-free survival; SPD = sum of the products of diameters.
^{a} Special attention should be given to the emergence of a different M-protein following treatment, especially in the setting of patients having achieved a conventional CR, often related to oligoclonal reconstitution of the immune system. These bands typically disappear over time, and in some studies, have been associated with a better outcome. Also, appearance of IgGk in patients receiving monoclonal antibodies should be differentiated from the therapeutic antibody.
^{b}In some cases it is possible that the original M-protein light-chain isotype is still detected on immunofixation, but the accompanying heavy-chain component has disappeared; this would not be considered a CR even though the heavy-chain component is not detectable, since it is possible that the clone evolved to one that secreted only light chains. Thus, if a patient has IgA lambda myeloma, then to qualify as a CR there should be no IgA detectable on serum or urine immunofixation; if free lambda is detected without IgA, then it must be accompanied by a different heavy-chain isotype (IgG, IgM, etc.). Modified from Durie et al. 2006. Requires two consecutive assessments to be carried out at any time before the institution of any new therapy (Durie et al. 2015).
^{c}Plasmacytoma measurements should be taken from the CT portion of the PET/CT or MRI scans, or dedicated CT scans where applicable. For patients with only skin involvement, the skin lesions should be measured with a ruler. Measurement of tumor size will be determined by the SPD.
^{d} Positive immunofixation alone in a patient previously classified as achieving a CR will not be considered progression. Criteria for relapse from a CR should be used only when calculating disease-free survival.
^{e} In the case where a value is felt to be a spurious result per investigator discretion (e.g. a possible laboratory error), that value will not be considered when determining the lowest value.
^{f} CRAB features = calcium elevation, renal failure, anemia, lytic bone lesions.

### Example 2. Study Design

i. Description of Study

Patients are enrolled in one of two arms: the single-step dose escalation arm (Arm A) or the multistep dose-escalation arm (Arm B). The study enrolls approximately 50-70 patients in the dose-escalation arms at approximately 20-25 sites globally. Cevostamab is administered in 21-day cycles. Patients with acceptable toxicity and evidence of clinical benefit may continue to receive cevostamab up to a maximum of 17 cycles until disease progression (as determined according to International Myeloma Working Group (IMWG) criteria (Table 4) or unacceptable toxicity, whichever occurs first. An exception is made for patients who undergo intra-patient dose escalation, as is described below; these patients may continue to receive cevostamab up to a maximum of 17 cycles at the new, increased dose until disease progression or unacceptable toxicity, whichever occurs first. Patients who complete 17 cycles of treatment may be eligible for cevostamab re-treatment.

The rationale for limiting the duration of cevostamab treatment to 17 cycles is 3-fold. First, chronic, and/or cumulative toxicity potentially associated with prolonged treatment duration can be minimized. Second, a limited duration of treatment provides an opportunity to assess the duration of response once cevostamab treatment is discontinued. Finally, limiting cevostamab treatment to 17 cycles provides an opportunity to explore the possibility of cevostamab re-treatment in patients who achieve an objective response (PR or CR) or SD with initial cevostamab treatment provided that the criteria outlined above are met.

Patients who complete 17 cycles of study treatment (or re-treatment, if eligible), will continue to have tumor and additional assessments as outlined herein until disease progression, start of new anti-cancer therapy, or withdrawal from study participation, whichever occurs first.

All patients are closely monitored for adverse events throughout the study and for at least 90 days after the last dose of study treatment. Adverse events are graded according to the National Cancer Institute Common Terminology Criteria for Adverse Events, Version 4.0 (NCI CTCAE v4.0), with the exception of cytokine release syndrome (CRS), which is graded according to the Modified Cytokine Release Syndrome Grading System established by Lee et al., Blood, 124: 188-195, 2014 or the updated ASTCT Consensus Grading for Cytokine Release Syndrome established by Lee et al., Biol Blood Marrow Transplant, 25(4): 625-638, 2019 and described in Table 5A. The NCI CTCAE v4.0 CRS grading scale was based on characterizations of CRS following treatment with monoclonal antibodies (Lee et al., Blood, 124: 188-195, 2014). T-cell directed therapies, including bispecifics such as blinatumomab, and adoptive cell therapies, such as engineered T-cells expressing CARs, result in PD profiles of cytokine release from T-cell activation distinct from those associated with conventional monoclonal antibodies. Consequently, the clinical features of CRS as defined by NCI CTCAE v4.0 may not be applicable to those following T-cell directed therapy.

Several alternate grading scales have been proposed and published which are specifically geared toward evaluation of CRS for T-directed therapies (Davila et al., Sci Transl Med, 6: 224ra25, 2014; Lee et al., Blood, 124: 188-195, 2014; Porter et al., Sci Transl Med, 7: 303ra139, 2015). The grading system of Lee et al. is based on CRS arising from treatment with CD19-directed CAR-T cell and blinatumomab. It is a modification of NCI CTCAE v4.0, which provides further diagnostic detail including accounting for transient elevations in liver transaminases that may occur in the setting of CRS. In addition to diagnostic criteria, recommendations on management of CRS based on its severity, including early intervention with corticosteroids and/or anti-cytokine therapy, are provided and referenced in Tables 5A and 5B. Incorporation of the CRS grading scale therefore allows for alignment between reporting and management guidelines that have been published and widely adopted.

TABLE 5A
Cytokine release syndrome grading systems

| | Modified Cytokine Release Syndrome | ASTCT Consensus Grading |
|---|---|---|
| Grade | Grading System | System |
| Grade 1 | Symptoms are not life threatening and | Temperature ≥38° C. |
| | require symptomatic treatment only (e.g. | No hypotension |
| | fever, nausea, fatigue, headache, myalgia, | No hypoxia |
| | malaise) | |
| Grade 2 | Symptoms require and respond to | Temperature ≥38° C.* with |
| | moderate intervention | hypotension not requiring |
| | Oxygen requirement <40%; or | vasopressors and/or^{†} hypoxia |
| | Hypotension responsive to fluids or low | requiring low-flow nasal cannula^{‡} or |
| | dose ^{a} of one vasopressor; or | blow-by |
| | Grade 2 organ toxicity | |
| Grade 3 | Symptoms require and respond to | Temperature ≥38° C.* with |
| | aggressive intervention | hypotension requiring a vasopressor |
| | Oxygen requirement ≥40%; or | with or without vasopressin and/or^{†} |
| | Hypotension requiring high dose ^{b} or | hypoxia requiring high-flow nasal |
| | multiple vasopressors; or | cannula^{‡}, facemask, nonrebreather |
| | Grade 3 organ toxicity or Grade 4 | mask, or Venturi mask |
| | transaminitis | |
| Grade 4 | Life-threatening symptoms | Temperature ≥38° C.* with |
| | Requirement for ventilation support or | hypotension requiring multiple |
| | Grade 4 organ toxicity (excluding | vasopressors (excluding vasopressin) |
| | transaminitis) | and/or^{†} hypoxia requiring positive |
| | | pressure (e.g., CPAP, BiPAP, |
| | | intubation and mechanical ventilation) |
| Grade 5 | Death | Death |

Lee 2014 criteria: Lee et al., Blood, 124: 188-195, 2014.
ASTCT consensus grading: Lee et al., Biol Blood Marrow Transplant, 25(4): 625-638, 2019.
^{a} Low-dose vasopressor: single vasopressor at doses below that shown in Table 5B.
^{b} High-dose vasopressor: as defined in Table 5B.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 5B
High-dose vasopressors
High-Dose Vasopressors (duration ≥3 hours)

| Pressor | Dose |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine |
| | equivalent of ≥10 μg/min ^{a} |
| If on combination or vasopressors | Norepinephrine equivalent |
| (not vasopressin) | of ≥20 μg/min ^{a} |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

ii. Dose Escalation and Expansion Arms

Monoclonal antibodies used for the treatment of hematologic malignancies are administered on schedules based on 3- to 4-week cycles. For PK and safety reasons, the first cycle of treatment is frequently modified in that the antibody is administered more frequently in split or fractionated doses (GAZYVA® (obinutuzumab) U.S. Package Insert, Genentech USA, Inc.). In analogous fashion, the bispecific T-cell engager blinatumomab targeting CD19, which is administered as a continuous IV infusion, employs a step-dosing strategy in the treatment of acute lymphoblastic leukemia (ALL) (BLINCYTO® (blinatumomab) U.S. Package Insert, Amgen, Inc.) and non-Hodgkin's lymphoma (NHL) (Viardot et al., Blood, 127: 1410-1416, 2016). Nonclinical data with cevostamab resulted in acute cytokine release following the first dose and not at all, or to a lesser degree, in subsequent doses. Therefore, the collective nonclinical and clinical data from T-cell-engaging antibodies targeting B-cell malignancies suggest that step dosing has the potential to minimize treatment-emergent toxicity with cevostamab. Cevostamab is therefore administered using a Cycle 1 step-dose schedule as described herein.

The optimal ratio between doses in a step dose approach is unknown, but based on clinical information for other bispecific molecules, a 1 to 3 ratio of C1D1 (cycle 1, day 1) dose to C1D8 (cycle 1, day 8) dose is a rational initial dosing regimen for cevostamab. However, given that the C1D1 dose may be fixed while the C1D8 dose continues to be dose escalated, other ratios between doses may be tested in this study.

The single-step dose-escalation arm (Arm A) of the study assesses the safety, tolerability, and pharmacokinetics of cevostamab administered by IV infusion on Day 1 and Day 8 of the first 21-day cycle, followed thereafter by IV infusion on Day 1 of each 21-day cycle. Enrollment in the multistep dose-escalation arm (Arm B) began after Arm A had completed assessment of at least 10 dose cohorts; Arm B then ran in parallel with Arm A. Arm B assesses the safety, tolerability, and pharmacokinetics of cevostamab administered by IV infusion on Day 1, Day 8 and Day 15 of the first 21-day cycle, followed thereafter by IV infusion on Day 1 of each 21-day cycle. For both Arms A and B, “target dose” refers to the highest dose administered in Cycle 1; this “target dose” is administered on Day 1 of subsequent cycles.

Arm a (Single-Step Dose Escalation Arm)

For Arm A only, to minimize the number of patients exposed to subtherapeutic doses, initially 1 patient was enrolled into each dose-escalation cohort. A conversion to a standard 3+3 design was based on the occurrence of one of the following events:
Observation of a Grade ≥2 adverse event not considered by the investigator to be attributable to another clearly identifiable cause; or
Any DLT is observed in either Window 1 or Window 2.

Dose-escalation cohorts consist of at least 3 patients, unless dose-limiting toxicities (DLTs) are observed in the first 2 patients prior to enrollment of a third patient, according to a standard 3+3 design.

Two dose-limiting toxicity (DLT) assessment windows are utilized, as follows:
The first DLT assessment window (Window 1, step-up DLT window) consists of the period of time between Cycle 1, Day 1 (C1D1) and the initiation of the cevostamab infusion on Cycle 1, Day 8 (C1D8).

The second DLT assessment window (Window 2, target dose DLT window) is defined as a period of 14 days following the initiation of the C1D8 infusion.

Arms C and F (Single-Step Dose Expansion Arms)

Arm C and Arm F are dose-expansion arms to obtain safety, tolerability, pharmacokinetic, and preliminary clinical activity data with single-step cevostamab treatment, based on emergent clinical data from Arm A. Based on data from Arm A, the dose level of 3.6 mg/90 mg was selected for Arm C and the arm was opened.

Arm B (Multistep Dose Escalation Arm)

A multistep dose escalation arm (Arm B) was added to assess the safety, tolerability, and pharmacokinetics of a multistep dosing regimen in Cycle 1. Emerging clinical data indicates that multiple-step dose fractionation is effective in mitigating CRS-related adverse events that may be induced by TDBs (Budde et al., Blood, 132: 399, 2018). The proposed starting doses for the multistep dose-escalation arm are based on available clinical data from Arm A of the study.

The first step-up dose on C1D1 is less than or equal to the highest DLT-cleared C1D1 dose in Arm A.

The second step-up dose on C1D8 may be up to the next permitted dose level from the DLT-cleared C1D1 dose in Arm A based on escalation guidelines. (e.g., if 3.6 mg is the highest cleared Arm A step-up dose with allowance of up to 100% dose increase, the highest permitted starting dose for Arm B C1D8 will be 7.2 mg).

Finally, the Arm B C1D15 target dose starts at the highest DLT-cleared C1D8 dose in Arm A.

Arm B is conducted using a standard 3+3 design. Dose-escalation cohorts consist of at least 3 patients, unless DLTs are observed in the first 2 patients prior to enrollment of a third patient, according to a standard 3+3 design. In Cycle 1, patients in Arm B receive 2 step-up doses and a target dose. These three doses are administered one week apart on Days 1, 8, and 15.

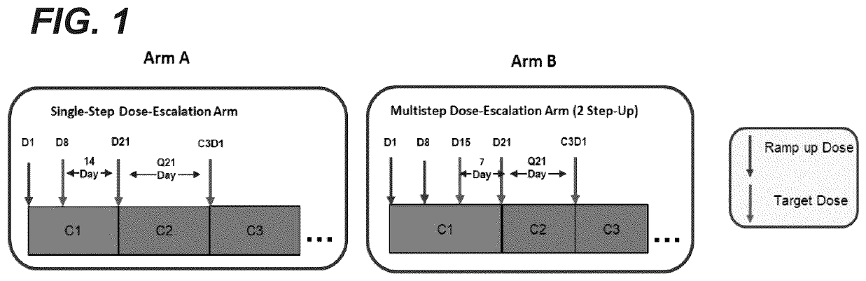

The DLT assessment windows are utilized as follows:
Each step-up dose has a DLT assessment window defined as a period of 7 days following the initiation of the step-up dose. If the Cycle 1, Day 1 or Day 8 step-up dose is less than or equal to a previously cleared Cycle 1, Day 1 or Day 8 step-up dose, respectively, in either Arm A or Arm B, the DLT assessment window is not required.
The target dose DLT assessment window is defined as a period of 7 days following the initiation of the target dose cevostamab infusion.
   Dosing days for the dose-escalation arms are illustrated in FIG. 1.

Arms D and G (Multistep Dose Expansion Arms)

Arm D and Arm G are dose-expansion arms to obtain safety, tolerability, pharmacokinetic, and preliminary clinical activity data with multistep cevostamab treatment at different dose(s), based on emergent clinical data from Arm B.

All Dose-Escalation Arms

The dose-escalation rules outlined above are designed to ensure patient safety while minimizing the number of patients exposed to sub-therapeutic doses of study treatment. For this reason, single-patient dose-escalation cohorts are initially used with dose-escalation intervals not exceeding 200% of the preceding dose level, with conversion to a standard 3+3 dose-escalation design and lower dose-escalation intervals based on rules outlined above.

For each dose-escalation cohort, treatment with the first dose of cevostamab is staggered such that the second patient enrolled in the cohort receives cevostamab at least 72 hours after the first patient receives cevostamab to allow assessment of any severe and unexpected acute or subacute drug or infusion-related toxicities; dosing in subsequent patients in each cohort is staggered by at least 24 hours. Dose-escalation rules are defined below.

Patients who discontinue from the study prior to completing the DLT assessment windows for reasons other than a DLT are considered non-evaluable for dose-escalation decisions and maximum tolerated dose (MTD) assessments, and are replaced by an additional patient at that same dose level. Patients who miss any dose during the DLT assessment windows for reasons other than a DLT are also replaced. Patients who receive supportive care (including radiotherapy) during the DLT assessment windows that confounds the evaluation of DLTs (not including supportive care described below as part of the DLT definition) may be replaced.

Definition of Dose-Limiting Toxicity

For the initial assessment of cevostamab in patients, the interval between repeat dosing is 21 days. As outlined herein, the DLT observation period for dose escalation is the 21-day period following the first dose of cevostamab. In the nonclinical toxicity studies in cynomolgus monkeys, this observation period allowed for adequate recovery from observed toxicities related to cevostamab.

All adverse events, including DLTs, are graded according to NCI CTCAE v4.0 unless otherwise indicated. DLTs are treated according to clinical practice and are monitored through their resolution. All adverse events are considered related to cevostamab unless such events are clearly attributed by the investigator to another clearly identifiable cause (e.g., disease progression, concomitant medication, or pre-existing medical condition).

Decreases in B cells, lymphopenia, and/or leukopenia due to decreases in B cells or T cells are not considered DLTs as they are expected pharmacodynamic (PD) outcomes of cevostamab treatment based on nonclinical testing of this molecule.

A DLT is defined as any of the following adverse events occurring during the DLT assessment windows:

Any Grade 4 or 5 adverse event not considered by the investigator to be attributable to another clearly identifiable cause, with the following exception:
Grade 4 lymphopenia, which is an expected outcome of therapy
Grade 4 neutropenia that is not accompanied by temperature elevation (oral or tympanic temperature of 100.4° F. [38° C.]) and improves to Grade ≤2 (or to ≥80% of the baseline ANC, whichever is lower) within 1 week with or without G-CSF Grade 4 thrombocytopenia that improves to Grade ≤2 (or to ≥80% of the baseline platelet count, whichever is lower) within 1 week without platelet transfusion (unless previously transfusion dependent) and not associated with bleeding that is considered clinically significant by the investigator.

Any Grade 3 hematologic adverse event not considered by the investigator to be attributable to another clearly identifiable cause, with the following exceptions:
Grade 3 lymphopenia, which is an expected outcome of therapy.
Grade 3 neutropenia that is not accompanied by temperature elevation (oral or tympanic temperature of ≥100.4° F. (38° C.)) and improves to Grade ≤2 (or to ≥80% of the baseline ANC, whichever is lower) with or G-CSF within 1 week.

Grade 3 thrombocytopenia that improves to Grade ≤2 (or to ≥80% of the baseline platelet count, whichever is lower) within 1 week without platelet transfusion and is not associated with bleeding that is considered clinically significant by the investigator.

Any Grade 3 non-hematologic adverse event not considered by the investigator to be attributable to another clearly identifiable cause, with the following exceptions:
Grade 3 nausea or vomiting in the absence of premedication or that can be managed with resulting resolution to Grade ≤2 with oral or IV anti-emetics within 24 hours.
Grade 3 nausea or vomiting that requires total parenteral nutrition or hospitalization are not excluded and should be considered a DLT.
Grade 3 fatigue lasting ≤3 days.
Grade 3 laboratory abnormalities that are asymptomatic and resolve to Grade ≤1 or baseline within 7 days.

Any hepatic function abnormality as defined by the following:
AST or ALT >3×the upper limit of normal (ULN) and total bilirubin >2×ULN, with the following exception: any AST or ALT >3×the ULN and total bilirubin >2×ULN where no individual laboratory value exceeds Grade 3 that occurs in the context of Grade ≤2 CRS (as defined by the criteria established by Lee et al., Biol Blood Marrow Transplant, 25: 625-638, 2019; see Table 5A); and resolves to Grade ≤1 within <3 days will not be considered a DLT.
Any Grade 3 AST or ALT elevation with the following exception:
   Any Grade 3 AST or ALT elevation that occurs in the context of Grade ≤2 CRS (as defined by the criteria established by Lee et al., Biol Blood Marrow Transplant, 25: 625-638, 2019 (Table 5A) and resolves to Grade ≤1 within <3 days will not be considered a DLT.

Any Grade 2 neurologic toxicity mapping to a MedDRA High-Level Group Term from the list consisting of cranial nerve disorders (excluding neoplasms), demyelinating disorders, encephalopathies, mental impairment disorders, movement disorders (including parkinsonism), neurological disorders NEC (not elsewhere classified), seizures (including subtypes), cognitive and attention disorders and disturbances, communication disorders and disturbances, delirium (including confusion), and dementia and amnestic conditions that is not considered by the investigator to be attributable to another clearly identifiable cause and that does not resolve to baseline within 72 hours will be considered a DLT.

Grade 1 depressed level of consciousness or Grade 1 dysarthria that is not considered by the investigator to be attributable to another clearly identifiable cause and that does not resolve to baseline within 72 hours will be considered a DLT.

Any grade seizure that is not considered by the investigator to be attributable to another clearly identifiable cause will be considered a DLT.

Dose Escalation Rules

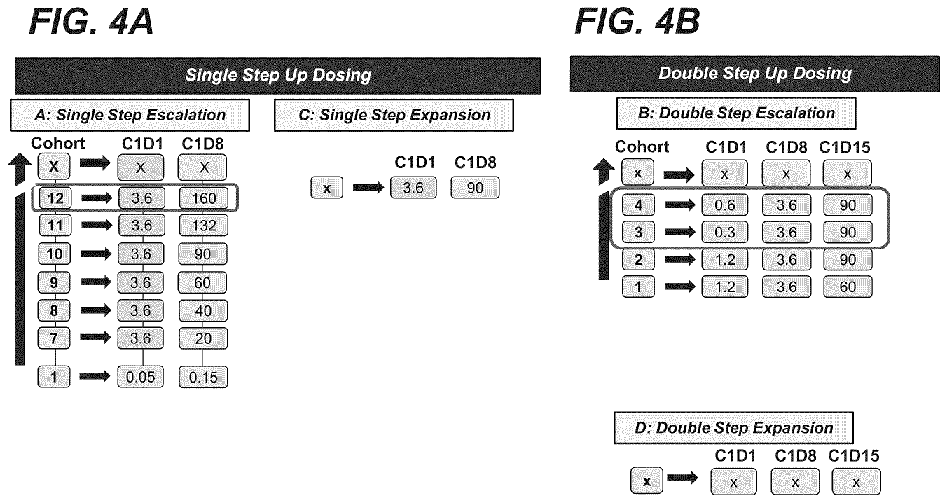

Cevostamab is administered using a step-dose approach in Cycle 1. For Arm A, the initial dose given on C1D1 (cycle 1, day 1) (the step dose) is less than a second dose (target dose) given on C1D8 (cycle 1, day 8). The starting dose of cevostamab was 0.05 mg and 0.15 mg on C1D1 and C1D8, respectively, administered intravenously (FIG. 4A).

Patients are hospitalized during Cycle 1. Treatment-emergent toxicities, notably CRS and neurologic toxicity, have been observed with blinatumomab and CAR-T therapies (Kochenderfer et al., Blood, 119: 2709-2720, 2012; Grupp et al., New Engl J Med, 368:1509-1518, 2013). These toxicities generally occur upon first exposure to the therapeutic agent. While the mechanisms of action of these toxicities are not completely understood, it is believed that they are the result of immune cell activation resulting in inflammatory cytokine release. With CAR-T and blinatumomab, the onset of laboratory and clinical manifestations of cytokine release generally occur within 24 hours of first exposure to the therapeutic agent and substantially decrease in frequency and severity over time (Klinger et al., Blood, 119: 6226-6233, 2012). A similar pattern has been observed with the anti-CD20/CD3 TDB BTCT4465A, with the onset of CRS occurring within 24 hours of the C1D1 dose in the majority of patients that developed CRS. The onset of CRS has correlated well with increases in serum interleukin (IL)-6, which have been observed most frequently 4-6 hours after the completion of C1D1 dosing. Therefore, on the basis of this prior clinical experience, hospitalization is required as described herein.

For Arm B, two step-up doses are given on a weekly basis on Days 1 and 8 followed by administration of the target dose on Day 15. The target dose is administered 7 days after the last step-up dose. The starting dose of cevostamab was 1.2 mg, 3.6 mg, and 60 mg on C1D1, C1D8, and C1D15, respectively, administered intravenously (FIG. 4B). Doses of 0.3 or 0.6 mg, 3.6 mg, and 90 mg on C1D1, C1D8, and C1D15, respectively, administered intravenously, were also tested (FIG. 4B).

The Cycle 2, Day 1 (C2D1) dose must be given a minimum of 14 days after the target dose is given in Cycle 1 for Arm A and a minimum of 7 days after the target dose is given in Cycle 1 for Arm B. Thereafter, cevostamab is administered on Day 1 of a 21-day cycle as described above, but may be given up to ±2 days from the scheduled date (i.e., with a minimum of 19 days between doses) for logistic/scheduling reasons. The C2D1 dose and all subsequent doses are equal to the Cycle 1 target dose unless a dose modification is required or intrapatient dose escalation occurs.

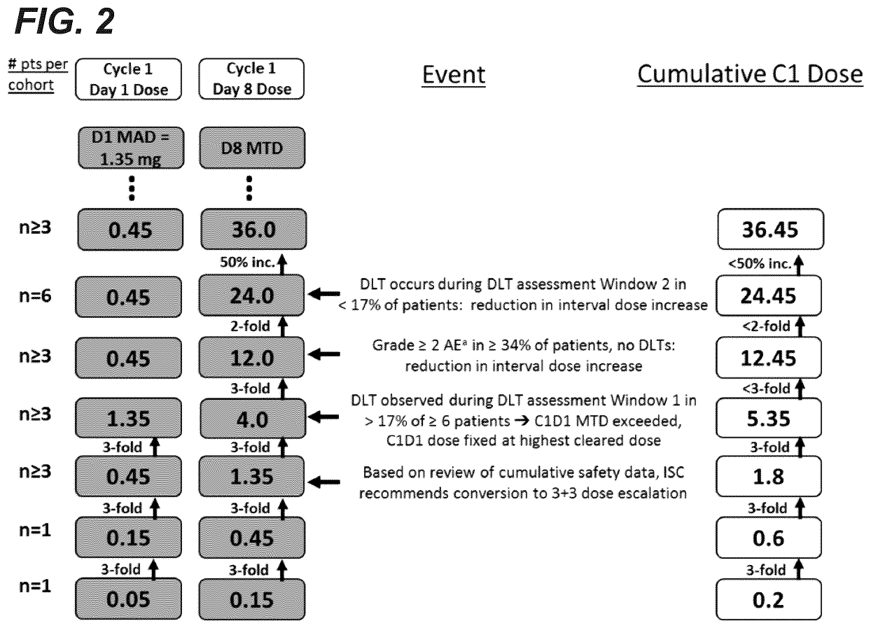

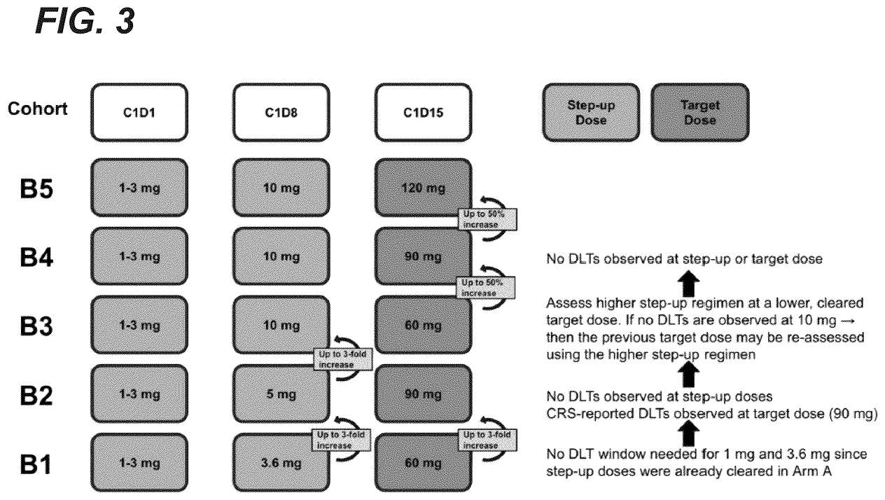

The step-up and target doses may be increased up to a maximum of 3-fold of the preceding dose levels for each successive cohort until a safety threshold (defined as the observation of a Grade ≥2 adverse event not considered by the investigator to be attributable to another clearly identifiable cause in ≥34% of patients is observed) is reached. Once this safety threshold has been met during a DLT window of a given cohort, the corresponding dose may be increased by up to a maximum of 2-fold of the preceding dose for subsequent cohorts (see FIGS. 2 and 3 for illustrative examples). Following the observation of a DLT in ≤17% of ≥6 patients during a DLT window of a given cohort, the corresponding dose may be increased no more than 50% of the preceding dose for subsequent cohorts.

DLT criteria, as defined above, are the same for all DLT assessment windows. The totality of safety data from both arms of the study is considered when making dose escalation decisions. However, for dose escalation decisions, DLTs are counted independently for each study arm. Similarly, the MTD and maximum achieved dose (MAD) for Arms A and B will be determined separately.

Rules for dose escalation of the step-up dose(s) are as follows:

If none of the first 3 DLT-evaluable patients in a given cohort experiences a DLT during the step-up dose DLT window, the step-up dose may be escalated in the next cohort according to the rules described above.

If 1 of the first 3 DLT-evaluable patients experiences a DLT during the step-up dose
DLT window, the cohort is expanded to 6 patients. If there are no further DLTs in the 6 DLT-evaluable patients during the step-up dose DLT window, the step-up dose may be escalated by no more than 50% of the preceding C1D1 dose in subsequent cohorts.

If 2 or more of the first 3 DLT-evaluable patients in a given cohort experience a DLT
during the step-up dose DLT window, the corresponding step-up dose MTD will have been exceeded and escalation at that step-up dose will stop. An additional 3 patients will be evaluated for DLTs using a dosing scheme consisting of the preceding step-up dose level and the highest cleared target dose level, unless 6 patients have already been evaluated at that level.

If the step-up dose level at which the dose MTD is exceeded is 25% higher than the preceding tested step-up dose, additional dose cohorts of at least 6 patients may be evaluated at intermediate step-up dose(s) for evaluation as the MTD.

Rules for dose escalation of the target dose are as follows:
If none of the first 3 DLT-evaluable patients in a given cohort experiences a DLT during the target dose DLT window, enrollment of the next cohort at the next highest dose level for the target dose DLT window may proceed according to the dose-escalation rules outlined above.
If 1 of the first 3 DLT-evaluable patients experiences a DLT during the target dose DLT window, the cohort will be expanded to 6 patients at the same dose level. (Note: if the step-up dose at a given level has been shown to exceed the step-up dose MTD, the additional patients enrolled in the cohort will be enrolled at a lower, previously cleared step-up dose.) If there are no further DLTs in 6 DLT-evaluable patients during the target dose DLT window, enrollment of the next cohort may proceed with the target dose being escalated by no more than 50% of the preceding target dose.

If 2 or more DLT-evaluable patients in a cohort experience a DLT during the target dose DLT window, the target dose MTD will have been exceeded and escalation of the target dose will stop, with the following exception:
If all DLTs experienced at a given target dose were reported as CRS or its symptoms, an additional 3 patients may be evaluated for DLTs by dose escalating the step-up dose(s) (if allowed per criteria above) and using a lower, previously cleared target dose. If all 3 patients do not experience CRS or its symptoms in the new regimen, then the previously tested target dose can be retested using a higher step up regimen and may continue to escalate.
If only CRS-related DLTs are observed at the Arm B target dose, additional step-up regimens may be explored with a lower, previously cleared target dose before declaring MTD for the target dose. If the new step-up regimen is tolerated, the original target dose with CRS-related DLTs may be reassessed.

If the target dose MTD has been exceeded and no escalation of the step-up dose is planned, the following rules will apply:
An additional 3 patients may be evaluated for DLTs using a dosing scheme consisting of the highest cleared step-up dose level and the highest cleared target dose level, unless 6 patients have already been evaluated at that level.
If the target dose MTD is exceeded at any dose level, the highest target dose at which fewer than 2 of 6 DLT-evaluable patients (i.e., <17%) experience a DLT will be declared the target dose MTD.
If the target dose level at which the target dose MTD is exceeded is ≥25% higher than the preceding tested target dose, additional dose cohorts of at least 6 patients may be evaluated at intermediate target dose(s) for evaluation as the MTD.

Additional dose cohorts that assess intermediate dose levels between two dose levels that have been demonstrated to not exceed the MTD may be evaluated to further characterize dose-dependent toxicities. Enrollment of cohorts to evaluate intermediate dose levels may occur concurrently with enrollment of dose-escalation cohorts to identify the MTD.

For each dose-escalation arm, if the target dose MTD is not exceeded at any dose level, the highest doses administered in this study for step-up and target dose in a single cohort will be declared the MADs.

If only CRS-related DLTs are observed at the Arm B target dose, additional step-up regimens may be explored with a lower, previously cleared target dose before declaring MTD for the target dose. If the new step-up regimen is tolerated, the original target dose with CRS-related DLTs may be reassessed.

To acquire additional safety and PD data to better fully inform the recommended Phase II dose, additional patients may be enrolled at a dose levels that have been shown to not exceed the MTD based on the dose-escalation criteria described above, and at which there is evidence of anti-tumor activity and/or PD biomarker modulation. Up to approximately 3 additional patients per dose level may be enrolled. For the purposes of dose-escalation decisions, these patients will not be included as part of the DLT-evaluable population.

Intrapatient Dose Escalation

In dose-escalation Arms A and B only, to maximize the collection of information at relevant doses and minimize the exposure of patients to suboptimal doses of cevostamab, intrapatient dose escalation may be permitted. The dose of cevostamab for an individual patient may be increased to the highest cleared dose level that is tolerated by completed cohorts through at least one cycle of cevostamab administration. Patients are able to undergo intrapatient dose escalation after completing at least two cycles at their originally assigned dose level. Subsequent intrapatient dose escalations may occur after at least one cycle of any subsequently higher cleared dose level without any adverse event that meets the definition of a DLT or necessitates post-administration hospitalization. Because intrapatient dose escalation will be conducted in this manner, additional information regarding step dosing as a mitigation strategy against treatment-emergent toxicity can be acquired.

Once the MTD is declared and the recommended Phase II dose is determined, intrapatient dose escalation directly to the recommended Phase II dose is permitted for patients who remain on study and continue to tolerate cevostamab.

Rules for Continued Dosing Beyond Cycle 1

Patients who do not experience a DLT during the DLT observation period are eligible to receive additional infusions of cevostamab as follows:

Ongoing clinical benefit: Patients must have no clinical signs or symptoms of progressive disease (patients will be clinically assessed for disease progression on Day 1 of each cycle). Patients will also be assessed at the beginning of each cycle for progression based on the International Myeloma Working Group (IMWG) criteria (see Table 4). Patients with solely biochemical disease progression (defined as an increase of monoclonal paraprotein in absence of organ dysfunction and clinical symptoms) and who qualify for intrapatient dose escalation may receive additional infusions. For determining disease progression according to IMWG criteria after a patient has undergone intrapatient dose escalation, baseline will be reestablished at each new dose level assessed for a patient.

Acceptable toxicity: Patients who experience Grade 4 non-hematologic adverse events with the possible exception of Grade 4 tumor lysis syndrome (TLS) should discontinue study treatment and may not be re-treated. Patients who experience Grade 4 TLS may be considered for continued study treatment. All other study treatment-related adverse events from prior study treatment infusions must have decreased to Grade 51 or baseline grade by the next infusion. Exceptions on the basis of ongoing overall clinical benefit may be allowed. Any treatment delay for adverse events not attributed to study treatment may not require study treatment discontinuation. Dose reductions of cevostamab may be allowed if it is determined that clinical benefit may be maintained.

Cevostamab Re-Treatment

Patients who initially respond to cevostamab, but subsequently develop recurrent or progressive disease after the completion of therapy, may benefit from additional cycles of cevostamab treatment. To test this hypothesis, patients are eligible for cevostamab re-treatment as described below. The cevostamab dose and schedule for these patients will be the dose and schedule that has been found to be safe at the time of re-treatment, provided the following criteria are met:
Pertinent eligibility criteria are met at the time that cevostamab treatment is re-initiated. Manageable and reversible immune-related adverse events with initial cevostamab treatment are allowed and do not constitute an exclusionary history of autoimmune disease.
Patients must have had documented objective response (complete response (CR), very good partial response (VGPR), or partial response (PR)) per IMWG criteria at the end of initial cevostamab treatment and for at least one post-treatment tumor assessment after the end of treatment.
Patients must not have experienced Grade 4 non-hematologic adverse events related to study treatment during initial cevostamab treatment.
Patients who experienced Grade 2 or Grade 3 adverse events during initial treatment must have resolved these toxicities to ≤Grade 1.
No intervening systemic anti-cancer therapy was administered between the completion of initial cevostamab treatment and re-initiation of cevostamab treatment.

A repeat bone marrow biopsy and aspirate to assess FcRH5 expression status and the tumor microenvironment must be obtained prior to cevostamab re-treatment.

The schedule of activities for patients who receive cevostamab re-treatment will follow the schedule of activities currently implemented in dose escalation or expansion. Patients who complete 17 cycles of re-treatment will continue to have tumor and additional assessments as outlined herein until disease progression, start of new anti-cancer therapy, or withdrawal from study participation, whichever occurs first.

Pharmacokinetic, Pharmacodynamic, and Anti-Drug Antibody Sampling Schedule

The PK sampling schedule that follows the cevostamab administration is designed to capture cevostamab exposure data at a sufficient number of timepoints to provide a detailed profile of the concentration-time curve. Additionally, the PD sampling schedule is designed to provide a detailed profile of the magnitude and kinetics of T-cell activation, possible peripheral blood B-cell depletion, and cytokine release following cevostamab treatment. These data are used to understand the relationship of dose to exposure and to support PK- and/or PD-based dose selection and schedules of cevostamab administration as single agent and in combinations with other agents used to treat MM. Anti-drug antibodies (ADAs) against cevostamab may have an impact on its benefit-risk profile. Therefore, a risk-based strategy (Rosenberg and Worobec, Biopharm International, 17: 22-26, 2004; Rosenberg and Worobec, Biopharm International, 17: 34-42, 2004; Rosenberg and Worobec, Biopharm International, 18: 32-36, 2005; Koren et al., J Immunol Methods, 333: 1-9, 2008) is utilized to detect and characterize ADA responses to cevostamab. Validated screening and confirmatory assays are used to detect ADA at timepoints before, during, and after cevostamab treatment. In addition, the correlation of ADA responses to relevant clinical endpoints may be assessed.

Biomarker Assessments

Understanding the mechanism of action of cevostamab and identifying prognostic and predictive biomarkers for safety clinical activity in patients with R/R MM forms the underlying rationale for their assessment in this study.

The biomarker sampling schedule (from peripheral blood, and bone marrow biopsies and aspirates) following cevostamab administration is designed to provide a detailed profile of the following:
Time course of cytokine release in relation to cevostamab pharmacokinetics and clinical safety during the DLT observation period. Assessments of cytokine levels beyond the DLT observation period permit correlations with any chronic safety signals observed with chronic cevostamab treatment.
Expression of phenotypic markers of T-cell function and potential markers of resistance to cevostamab therapy. Examples of these include, but are not limited to, markers of T-cell activation and proliferation as well as expression of PD-1 and other inhibitory molecules on T cells.
Dynamic quantitative changes in T-cell, B-cell, and natural killer (NK) cell counts.
Monitoring for minimal residual disease (MRD) and establishing correlations with objective response and survival.

In addition to biomarker sampling, bone marrow biopsies and aspirates are obtained. Evaluating changes to the tumor immune microenvironment is important in understanding the mechanism of action of cevostamab, understanding potential mechanisms of cevostamab resistance, and providing biologic rationale for combinations of cevostamab with other anti-cancer therapies. The sampling schedule is therefore designed to capture quantitative and functional changes in the immune cell infiltrate as well as changes to disease biology using both phenotypic and gene expression assays.

As described herein, patients experiencing disease progression or disease relapse after cevostamab treatment may be eligible for re-treatment. Given that loss of FcRH5 expression after cevostamab treatment is a potential mechanism of resistance to T cell-directed therapies (Topp et al., Lancet Oncol, 16: 57-66, 2011), a repeat biopsy from a safely accessible site should be obtained prior to cevostamab re-treatment to both confirm FcRH5 expression and assess tumor immune status.

QT/QTc Assessment

Assessment for QT/QTc prolongation is based on recommendations of the ICH E14 guideline. Nonclinical studies in cynomolgus monkeys showed tachycardia and consequent decreases in RR, PR, and QT intervals at doses 0.1 mg/kg. Collection of triplicate 12-lead ECGs at pharmacologically matched timepoints and with the option for assessment by a dedicated centralized ECG laboratory allows for an assessment of the relationship between cevostamab exposure and any QT/QTc interval changes.

### Example 3. Assessment of Safety

This is the first study in which cevostamab is administered to humans. Specific anticipated or potential toxicities associated with administration of cevostamab, as well as the measures taken to avoid or minimize such toxicities in this trial, are described below.

i. Dose and Schedule Modifications

Cevostamab dosing (and tocilizumab remedication, if applicable) occurs only if a patient's clinical assessment and laboratory test values are acceptable. Management guidelines, including study treatment dose and schedule modifications for specific adverse events, are described herein. The following guidelines regarding dose and schedule modifications should be followed:

In general, patients receiving cevostamab who experience a Grade 4 adverse event that is not considered by the investigator to be attributable to another clearly identifiable cause should permanently discontinue all study treatment. However, for patients with Grade 4 adverse events of asymptomatic laboratory changes, study treatment may be resumed upon resolution to Grade ≤1.

For patients who experience IRRs with the first dose of cevostamab, or are at increased risk of recurrent IRRs with subsequent doses, the infusion rate should be slowed by 50%. If the patient does not experience IRR with the subsequent dose, the infusion rate may be brought back to the initial rate during the infusion based on the investigator's discretion.

In general, patients who experience either an adverse event that meets the definition of a DLT or other Grade 3 adverse event that is not considered by the investigator to be attributable to another clearly identifiable cause (e.g., disease progression, concomitant medication, or pre-existing medical condition) will be allowed to delay dosing for up to 2 weeks (or longer if approved by the Medical Monitor) in order to recover from the toxicity. Patients may continue to receive additional infusions of cevostamab, provided that the toxicity has resolved to Grade ≤1 (or for laboratory abnormalities, return to ≥80% of the baseline value), within 2 weeks.

A reduced dose for subsequent infusions of cevostamab should be considered. If the intended reduced dose (e.g., to the next highest cleared dose level assessed during dose escalation) is to a dose level where there is no evidence of cevostamab PD activity (e.g., no evidence of changes in serum cytokine levels), the patient may be discontinued from study treatment. Decisions on continued treatment following a DLT or other study treatment-related Grade 3 toxicity should be made following a careful assessment, including in the following scenarios:
If an elevation of AST or ALT >3×ULN and/or total bilirubin >2×ULN, with no individual laboratory value exceeding Grade 3, occurs in the context of Grade ≤2 CRS that lasts <3 days, cevostamab dosing may continue without dose reduction.
Patients with Grade 3 events of anemia if manageable by red blood cell transfusions as per institutional practice may continue without dose reduction.
Patients with Grade 3 or 4 events of thrombocytopenia or neutropenia if manageable by transfusions (platelets) or granulocyte colony-stimulating factor (GCSF) as per institutional practice may continue without dose reduction.

Patients with a Grade 3 or 4 event of neutropenia or thrombocytopenia that is considered due to disease and does not require transfusions or GCSF may continue dosing without dose reduction. Any patient in whom similar toxicity recurs at a reduced dose should be discontinued from further cevostamab treatment.

Patients who do not fulfill the criteria for dosing after the additional 2 weeks have elapsed are discontinued from study treatment (unless a longer dose delay was approved by the Medical Monitor) and are followed for safety outcomes as described below. Exceptions to this on the basis of ongoing clinical benefit may be allowed following investigator assessment of risk versus benefit. In addition, delay of therapy because of toxicities not attributed to study drug may not require discontinuation.

Depending on the length of treatment delay, the patient may be required to repeat step-up dosing. If a patient's dose is delayed more than 2 to 4 weeks beyond their normally scheduled dose, the investigator should consult with the Medical Monitor to determine if repeat step-up dosing is required. If a patient's dose is delayed by more than 4 weeks beyond their normally scheduled dose, repeat step-up dosing is mandatory. Patients will require hospitalization following the first repeat step-up infusion of cevostamab.

ii. Risks Associated with Cevostamab

The mechanism of action of cevostamab is immune cell-activation against FcRH5-expressing cells; therefore, a spectrum of events involving IRRs, target-mediated cytokine release, and/or hypersensitivity with or without emergent ADAs, may occur. Other bispecific antibody therapeutics involving T-cell activation have been associated with IRR, CRS, and/or hypersensitivity reactions.

Based on nonclinical data, cevostamab has the potential to cause rapid increases in plasma cytokine levels. Thus, IRR may be clinically indistinguishable from manifestations of CRS, defined as a disorder characterized by nausea, headache, tachycardia, hypotension, rash, and shortness of breath (NCI CTCAE v.4.0), given the expected human pharmacology of cevostamab, where T-cell engagement with plasma cells and B cells results in T-cell activation and cytokine release. The selection of MABEL as the initial dose of cevostamab and the design of the dose-escalation scheme are specifically intended to minimize risk of exaggerated cytokine release.

To minimize the risk and sequelae of IRR and CRS, cevostamab is administered over a minimum of 4 hours in Cycle 1 in a clinical setting. Corticosteroid premedication must be administered as described in Example 1.

Mild to moderate presentations of IRR and/or CRS may include symptoms such as fever, headache, and myalgia, and may be treated symptomatically with analgesics, anti-pyretics, and antihistamines as indicated. Severe or life-threatening presentations of IRR and/or CRS, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures per institutional practice. Severe CRS may be associated with other clinical sequelae such as disseminated intravascular coagulation, capillary leak syndrome, or MAS. Standard of care for severe or life threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124:188-195, 2014; Maude et al., New Engl J Med, 371: 1507-1517, 2014). The grading of CRS follows the modified grading scale described in Table 5A. As noted in Table 5A, even moderate presentations of CRS in patients with extensive comorbidities should be monitored closely with consideration given to intensive care unit admission and tocilizumab administration. Table 6 provides details about tocilizumab treatment of severe or life-threatening CRS.

TABLE 6
Tocilizumab treatment of severe or life-threatening cytokine release syndrome (CRS)

| | Pre-TCZ | | | | | | |
|---|---|---|---|---|---|---|---|
| | Treatment | | | | | | |
| | (within 24 | TCZ | Post-TCZ Treatment ^{a} | | | | |
| Assessment/Procedure | hours) | Administration | 6 hours | 1 day | 2 days | 3 days | 8 days |

| | |
|---|---|
| TCZ Administration (8 mg/kg) | x |

| | | |
|---|---|---|
| Vital signs ^{b} | x ^{c} | Measure at least every 6 hours until resolution |
| | | to baseline, then every 12 hours through Day 8 ^{c} |
| | | or until discharge from ICU |
| Pressor documentation ^{d} | x ^{c} | Record at least every 6 hours until pressors |
| | | are discontinued ^{c} |
| FiO2 | x ^{c} | Record at least every 6 hours until patient on |
| | | room air ^{c} |
| Pulse oximetry, resting | x ^{c} | Measure at least every 6 hours until resolution |
| | | to baseline, then every 12 hours through Day 8 ^{c} |
| | | or until discharge from ICU |

Local Laboratory Assessments

| | | | | | | |
|---|---|---|---|---|---|---|
| Hematology | x | x | x | x | x | x |
| Liver function tests (AST, ALT, total bilirubin) | x | x | x | x | x | x |
| Serum chemistry and creatine ^{e} | x | x | x | x | x | x |
| CRP, LDH, and serum ferritin | x | x | x | x | x | x |
| Coagulation (aPTT, PT/INR, fibrinogen) | x | x | x | x | x | x |
| Infection workup ^{f} | x | | | | | |

Central Laboratory Assessments

| | | | | | | | |
|---|---|---|---|---|---|---|---|
| Plasma cytokines | x | | x | x | x | x | x |
| Plasma IL-6 PD markers ^{g} | x | x ^{h} | x | x | x | x | x |
| Serum TCZ pharmacokinetics | x | x ^{h} | x | x | x | x | x |

CRP: C-reactive protein; eCRF: electrionic Case Report Form; IL: interleukin; PD: pharmacodynamic; PK: pharmacokinetic; TCZ: tocilizumab.
Note:
Record abnormalities or worsened clinically significant abnormalities on the Adverse Event eCRF.
^{a} If TCZ dose is repeated, follow schedule of activities following the second TCZ dose.
^{b} Includes respiratory rate, heart rate, and systolic and diastolic blood pressure while the patient is in a seated or supine position, and temperature.
^{c} The maximum and minimum values for any 24-hour period should be recorded in the clinical database.
^{d} Document vasopressor type and dose in the concomitant medication eCRF.
^{e} Includes sodium, potassium, chloride, bicarbonate, glucose and blood urea nitrogen
^{f} Includes assessment for bacterial, fungal, and viral infections.
^{g} Includes IL-6, soluble IL-6R, and sgp130.
^{h} Blood draws for serum TCZ PK and plasma IL-6 PD markers will be performed at the end of TCZ infusion, and will be drawn from the arm which was not used to administer TCZ.

iii. Safety Parameters and Definitions

Safety assessments consist of monitoring and recording adverse events, including serious adverse events and adverse events of special interest, performing protocol-specified safety laboratory assessments, measuring protocol-specified vital signs, and conducting other protocol-specified tests that are deemed critical to the safety evaluation of the study.

iv. Adverse Events

According to the ICH guideline for Good Clinical Practice, an adverse event is any untoward medical occurrence in a clinical investigation subject administered a pharmaceutical product, regardless of causal attribution. An adverse event can therefore be any of the following:

Any unfavorable and unintended sign (including an abnormal laboratory finding), symptom, or disease temporally associated with the use of a medicinal product, whether or not considered related to the medicinal product.

Any new disease or exacerbation of an existing disease (a worsening in the character, frequency, or severity of a known condition), excluding the exceptions described herein.

Recurrence of an intermittent medical condition (e.g., headache) not present at baseline

Any deterioration in a laboratory value or other clinical test (e.g., ECG, X-ray) that is associated with symptoms or leads to a change in study treatment or concomitant treatment or discontinuation from study drug.

Adverse events that are related to a protocol-mandated intervention, including those that occur prior to assignment of study treatment (e.g., screening invasive procedures such as biopsies).

v. Serious Adverse Events

A serious adverse event is any adverse event that meets any of the following criteria:
Is fatal (i.e., the adverse event actually causes or leads to death)
Is life threatening (i.e., the adverse event, in the view of the investigator, places the patient at immediate risk of death). This does not include any adverse event that, had it occurred in a more severe form or was allowed to continue, might have caused death.
Requires or prolongs inpatient hospitalization.
Results in persistent or significant disability/incapacity (i.e., the adverse event results in substantial disruption of the patient's ability to conduct normal life functions)
Is a congenital anomaly/birth defect in a neonate/infant born to a mother exposed to study drug
Is a significant medical event in the investigator's judgment (e.g., may jeopardize the patient or may require medical/surgical intervention to prevent one of the outcomes listed above)

The terms “severe” and “serious” are not synonymous. Severity refers to the intensity of an adverse event (e.g., rated as mild, moderate, or severe, or according to NCI CTCAE); the event itself may be of relatively minor medical significance (such as severe headache without any further findings). Severity and seriousness are independently assessed for each adverse event.

Adverse Events of Special Interest

Adverse events of special interest for this study are as follows:

Cases of potential drug-induced liver injury that include an elevated ALT or AST in combination with either an elevated bilirubin or clinical jaundice, as defined by Hy's Law.

Suspected transmission of an infectious agent by the study drug. Any organism, virus, or infectious particle (e.g., prion protein transmitting transmissible spongiform encephalopathy), pathogenic or non-pathogenic, is considered an infectious agent. A transmission of an infectious agent may be suspected from clinical symptoms or laboratory findings that indicate an infection in a patient exposed to a medicinal product. This term applies only when a contamination of the study drug is suspected.

DLTs.

Adverse events of special interest specific to cevostamab:
Grade ≥2 IRR.
Grade ≥2 neurologic adverse event.
Any grade CRS.
Any suspected MAS/HLH.
TLS (Grade ≥3 by definition).
Febrile neutropenia (Grade ≥3 by definition).
Any grade disseminated intravascular coagulation (minimum Grade 2 by definition).
Grade ≥3 AST, ALT, or total bilirubin elevation.
Any adverse event that fulfills protocol-defined DLT criteria.

### Example 4. Statistical Analysis

Descriptive statistics are used to summarize the safety, tolerability, pharmacokinetics, and clinical activity of cevostamab. Data are described and summarized as warranted by number of patients in question. All analyses are based on the safety-evaluable population, defined as all patients who receive any amount of study drug.

Continuous variables are summarized using means, standard deviations, median and ranges; categorical variables will be presented using counts and percentages. All summaries are presented by cohort.

i. Determination of Sample Size

The sample size for this trial is based on the dose-escalation rules described in Example 2. The planned enrollment for the escalation stage (Arms A and B) of this study is approximately 150 patients. The planned enrollment for each expansion arm of this study (Arms C, D, E, F, and G) is approximately 30 patients.

This trial initially utilized single-patient dose-escalation cohorts, but converted to a standard 3+3 design, as discussed above. Table 7 provides the probability of not observing a DLT in 3 patients or observing ≤1 DLT in 6 patients given different underlying DLT rates.

TABLE 7
Probability of observing DLTs with different underlying DLT rates

| True Underlying | Probability of Observing | Probability of |
|---|---|---|
| DLT | no DLT in 3 | Observing ≤1 |
| Rate | Patients | DLT in 6 Patients |
| 0.10 | 0.73 | 0.89 |
| 0.20 | 0.51 | 0.66 |
| 0.33 | 0.30 | 0.36 |
| 0.40 | 0.22 | 0.23 |
| 0.50 | 0.13 | 0.11 |
| 0.60 | 0.06 | 0.04 |

ii. Safety Analyses

The safety analyses include all patients who received any amount of study drug. Safety is assessed through summaries of adverse events, changes in laboratory test results, changes in ECGs, changes in anti-drug antibodies (ADAs), and changes in vital signs. Summaries are presented by cohort and overall. Verbatim descriptions of adverse events are mapped to MedDRA thesaurus terms. All adverse events occurring on or after treatment on C1D1 are summarized by mapped term, appropriate thesaurus levels, and NCI CTCAE toxicity grade. In addition, all serious adverse events, including deaths, are listed separately. DLTs and adverse events leading to treatment discontinuation are also listed separately. Relevant laboratory and vital sign data are displayed by time. Additionally, laboratory data are summarized by NCI CTCAE grade where the grading is available.

iii. Pharmacokinetic Analyses

Individual and mean serum cevostamab concentration versus time data are tabulated and plotted by dose level. The following PK parameters are derived when appropriate, as data allow:
Total exposure (area under the concentration-time curve (AUC))
Maximum observed serum concentration (Cₘₐₓ)
Minimum observed serum concentration (Cₘᵢₙ)
Clearance
Volume of distribution at steady state

Compartmental, non-compartmental, and/or population methods may be considered. Estimates for these parameters are tabulated and summarized (mean, standard deviation, coefficient of variation, median, minimum, and maximum). Other parameters, such as accumulation ratio, half-life, and dose proportionality, are also calculated. Additional PK analyses are conducted as appropriate.

iv. Activity Analyses

Response assessment data and duration of response are summarized for all patients by cohort.

Objective response is defined as a sCR, CR, VGPR, or PR as determined by investigator assessment using IMWG response criteria. Patients with missing or no response assessments are classified as non-responders. The objective response rate is summarized for patients receiving the recommended Phase II dose.

Among patients with an objective response, duration of response is defined as the time from the initial objective response to the time of disease progression or death. If a patient does not experience disease progression or death before the end of the study, duration of response is censored at the day of the last tumor assessment. If no tumor assessments were performed after the time of first objective response, duration of response is censored at the time of first objective response.

v. Immunogenicity Analyses

The numbers and proportions of ADA-positive patients and ADA-negative patients at baseline (baseline prevalence) and after baseline (post-baseline incidence) are summarized. The relationship between ADA status and safety, drug activity, PK, and biomarker endpoints is analyzed and reported via descriptive statistics.

When determining postbaseline incidence, patients are considered to be ADA positive if they are ADA negative or have missing data at baseline but develop an ADA response following study drug exposure (treatment-induced ADA response), or if they are ADA positive at baseline and the titer of one or more postbaseline samples is at least 0.60 titer unit greater than the titer of the baseline sample (treatment-enhanced ADA response). Patients are considered to be post-treatment ADA negative if they are ADA negative or have missing data at baseline and all postbaseline samples are negative, or if they are ADA positive at baseline but do not have any postbaseline samples with a titer that is at least 0.60 titer unit greater than the titer of the baseline sample (treatment unaffected).

### Example 5. Results of Phase I Dose Escalation Study

The ongoing GO39775 Phase I, multicenter, open-label, dose-escalation study described in Examples 1-4 investigated cevostamab (FIG. 24) as a monotherapy in patients with R/R MM for whom no established therapy for MM is appropriate and available or who are intolerant to those established therapies. In this study, cevostamab was administered by intravenous (IV) administration in a step-up dose approach (single step-up dose and double step-up dose regimens) to mitigate cytokine release syndrome (CRS).

Current clinical efficacy data indicate promising clinical activity of cevostamab in both single step-up dose (Cohen et al., Blood, 136 (Supplement 1): 42-43, 2020) and double step-up dose regimens in heavily pretreated R/R MM patients who have exhausted available treatment options. The safety profile of cevostamab is manageable, with CRS as the most frequently reported adverse event (AE). Available efficacy and safety data and clinical pharmacology data of Study GO39775 are provided below.

As of the final clinical cut-off date (CCOD) presented in these Examples, 163 patients had been enrolled in Study GO39775, with 160 patients receiving cevostamab monotherapy in single step-up and double step-up regimens. Overall, for all 163 patients enrolled, the median time on treatment was 51 days (range: 1-703 days) with a median of 3 cycles of treatment (range: 1-34 cycles). Data are shown for the initial treatment phase. As of the CCOD, 1 patient was eligible for re-treatment after completing initial therapy and subsequently relapsing.

Of these 160 patients who received cevostamab monotherapy, 136 patients (85%) were triple-class refractory and received two or more prior lines of therapy and 42 patients (26%) had received prior CAR-T or ADC BCMA-targeting therapy and were triple-class exposed (at least one PI, one IMiD, and an anti-CD38 MAb). Patients' demography and disease characteristics are described in Table 8. All patients were heavily pre-treated, with a median of 6 prior lines of therapy. Moreover, all patients had received prior treatment with a PI and an IMiD, and 141 patients (88%) had received an anti-CD38 MAb. Patients' characteristics were very similar across the different patient populations.

TABLE 8
Summary of Baseline Characteristics of Patients Treated
with Cevostamab in Study GO39775 (ITT population)

| | | | Prior BCMA | | Triple-class | |
|---|---|---|---|---|---|---|
| | Overall | | ADC or CAR-T ^{a} | | Refractory | |
| | N = 160 | | N = 42 | | N = 136 | |
| Median age, years | 64 | (33-82) | 61 | (33-81) | 64 | (33-82) |
| (range) | | | | | | |
| Male sex, n (%) | 93 | (58) | 29 | (69) | 77 | (57) |
| ECOG, n (%) | | | | | | |
| 0 | 60 | (38) | 21 | (50) | 49 | (36) |
| 1 | 99 | (62) | 21 | (50) | 86 | (63) |
| High-risk | 71 | (44) | 19 | (45) | 59 | (43) |
| cytogenetics ^{b}, n (%) | | | | | | |
| Extramedullary | 34 | (21) | 7 | (17) | 30 | (22) |
| disease, n (%) | | | | | | |
| Median time from first | 6.1 | (0.3-22.8) | 7.3 | (1.2-21.8) | 6.8 | (0.3-22.8) |
| myeloma treatment, | | | | | | |
| years (range) | | | | | | |
| Median number of | 6 | (2-18) | 7.5 | (4-18) | 6 | (2-18) |
| prior lines, (range) | | | | | | |
| Prior stem cell | 142 | (89) | 39 | (93) | 120 | (88) |
| transplant, n (%) | | | | | | |
| Triple-class - | 136 | (85) | 39 | (93) | 136 | (100) |
| refractory ^{c}, n (%) | | | | | | |

ADC = antibody-drug conjugate; BCMA = B-cell maturation antigen; CAR-T = chimeric antigen receptor T-cell; CD38 = cluster of differentiation 38; ECOG = Eastern Cooperative Oncology Group; IMID = immunomodulatory agent; ITT = intent-to-treat; MAb = monoclonal antibody; PI = proteasome inhibitor.
^{a} Prior BCMA is defined as patients previously treated with a BCMA-targeting ADC or CAR-T therapy and triple-exposed (to a PI, an IMiD and an aCD38 mAB), excluding patients who were exposed to a bispecific MAb therapy.
^{b} High-risk cytogenetics are defined as 1q21 gain, translocation t(4;14), translocation t(14;16), and deletion 17p. Among all patients (n = 160), 71 (44.4%) had missing or unknown cytogenetics risk and could not be classified.
^{c} Triple-class refractory is defined as patients refractory to an IMiD, a PI, and a CD38 MAb.

Demographics and baseline characteristics were similar between patients receiving the single step-up dose regimen and double step-up dose regimen (Table 9). A total of 54 patients received a prior BCMA-targeting therapy; 51 of those were also exposed to a prior PI, IMiD and an anti-CD38 therapy, and of those 23 had a prior ADC therapy, 28 had a prior CAR-T therapy, and 9 had a prior bispecific mAb.

TABLE 9
Demographics and baseline characteristics (GO39775)

| | Single | | Single | | Double | | Double | | | | | | | |
|---|---|---|---|---|---|---|---|---|---|---|---|---|---|---|
| | Step-Up | | Step-Up | | Step- | | Step- | | | | Clinically | | | |
| | Dose | | Dose | | Up Dose | | Up Dose | | | | Active | | RP2D | |
| | Regimen | | Regimen | | Regimen | | Regimen | | | | Doses | | Regimen | |
| | (Arms | | (Arms | | (Arms | | (Arms | | All | | in | | (Arm | |
| | A + C) | | A + C) | | B + D) | | B + D) | | Patients^{a} | | Arms A + C | | B + D)^{b} | |
| | N = 99 | | N = 85 | | N = 61 | | N = 44 | | N = 160 | | N = 82 | | N = 36 | |
| | Dose (mg) | | | | | | | | | | | | | |
| | 0.05/0.15 | | | | 1.2/3.6/60 | | | | | | | | | |
| | to 3.6/198^{c} | | 3.6/Target | | to 0.3/3.6/160^{d} | | 0.3/3.6/90+ | | | | ≥3.6/20+ ^{e} | | 0.3/3.6/160 | |
| Age, median (range) | 63 | (33-80) | 63 | (33-76) | 64 | (45-82) | 64 | (47-82) | 64 | (33-82) | 62.5 | (33-76) | 64.5 | (47-82) |
| years | | | | | | | | | | | | | | |
| Male sex, n (%) | 60 | (60.6) | 50 | (58.8) | 33 | (54.1) | 22 | (50.0) | 22 | (50.0) | 50 | (61.0) | 18 | (50.0) |
| Race, n (%) | | | | | | | | | | | | | | |
| American Indian or | 2 | (2.0) | 2 | (2.4) | 1 | (1.6) | 1 | (2.3) | 8 | (4.9) | 2 | (2.4) | 1 | (2.8) |
| Alaska Native | | | | | | | | | | | | | | |
| Asian | 7 | (7.1) | 6 | (7.1) | 1 | (1.6) | 1 | (2.3) | 8 | (5.0) | 5 | (6.1) | 0 | |
| Black or African | 8 | (8.1) | 6 | (7.1) | 4 | (6.6) | 3 | (6.8) | 12 | (7.5) | 6 | (7.3) | 3 | (8.3%) |
| American | | | | | | | | | | | | | | |
| Native Hawaiian or | 2 | (2.0) | 2 | (2.4) | 0 | | 0 | | 2 | (1.3) | 2 | (2.4) | 0 | |
| other Pacific Islander | | | | | | | | | | | | | | |
| White | 77 | (77.8) | 66 | (77.6) | 53 | (86.9) | 38 | (86.4) | 130 | (81.3) | 64 | (78.0) | 31 | (86.1) |
| Unknown | 3 | (3.0) | 3 | (3.5) | 2 | (3.3) | 1 | (2.3) | 5 | (3.1) | 3 | (3.7) | 1 | (2.8) |
| BMI, median (range), | 27.71 | | 28.28 | | 27.2 | | 28.25 | | 27.49 | | 28.06 | | 28.79 | |
| kg/m² | (11.3-61.0) | | (11.3 -60.0) | | (16.4-42.3) | | (16.4-42.3). | | (11.3-61.0) | | (11.3-60.0) | | (16.4-42.3) | |
| High-risk cytogenetics, | | | | | | | | | | | | | | |
| n (%) | | | | | | | | | | | | | | |
| Unknown or Missing | 45 | (45.5) | 39 | (45.9) | 26 | (42.6) | 21 | (47.7) | 71 | (44.4) | 36 | (43.9) | 17 | (47.2) |
| cytogenetics | 39 | (39.4) | 32 | (37.7) | 27 | (44.2) | 17 | (38.6) | 66 | (41.3) | 32 | (39.0) | 14 | (38.9) |
| Amplified 1q21 f | 32 | (32.3) | 28 | (32.9) | 15 | (24.6) | 11 | (25.0) | 47 | (29.4) | 25 | (30.5) | 7 | (19.4) |
| Translocation t(4;14) f | 8 | (8.1) | 7 | (8.2) | 5 | (8.2) | 5 | (11.4) | 13 | (8.1) | 6 | (7.3) | 4 | (11.1) |
| Translocation t(11;14) f | 9 | (9.1) | 9 | (10.6) | 5 | (8.2) | 3 | (6.8) | 14 | (8.8) | 9 | (11.0) | 3 | (8.3) |
| Translocation t(14;16) f | 1 | (1.0) | 1 | (1.2) | 0 | | 0 | | 1 | (0.6) | 1 | (1.2) | 0 | |
| Deletion of TP53(17p) f | 15 | (15.2) | 11 | (12.9) | 11 | (18.0) | 9 | (20.5) | 26 | (16.3) | 10 | (12.2) | 9 | (25.0) |
| Baseline ECOG | | | | | | | | | | | | | | |
| performance | | | | | | | | | | | | | | |
| status, n (%) | | | | | | | | | | | | | | |
| 0 | 41 | (41.8) | 38 | (44.7) | 19 | (31.1) | 11 | (25.0) | 60 | (37.7) | 38 | (46.3) | 8 | (22.2) |
| 1 | 57 | (58.2) | 47 | (55.3) | 42 | (68.9) | 33 | (75.0) | 99 | (62.3) | 44 | (53.7) | 28 | (77.8) |
| Extramedullary Disease | | | | | | | | | | | | | | |
| at Screening, n (%) | | | | | | | | | | | | | | |
| n | 99 | | 85 | | 61 | | 44 | | 160 | | 82 | | 36 | |
| Yes | 20 | (20.2) | 17 | (20.0) | 14 | (23.0) | 11 | (25.0) | 34 | (21.3) | 17 | (20.7) | 9 | (25.0) |
| No | 78 | (78.8) | 67 | (78.8) | 46 | (75.4) | 32 | (72.7) | 124 | (77.5) | 64 | (78.0) | 27 | (75.0) |
| Unknown | 1 | (1.0) | 1 | (1.2) | 1 | (1.6) | 1 | (2.3) | 2 | (1.3) | 1 | (1.2%) | 0 | |
| Time since first multiple | 5.7 | | 5.70 | | 7.2 | | 7.30 | | 6.10 | | 5.7 | | 7.5 | |
| | (1.1-22.8) | | (1.1-17.6) | | (0.3-21.8) | | (0.3-21.8) | | (0.3-22.8) | | (1.1-17.6) | | (0.3-21.8) | |
| myeloma therapy, | | | | | | | | | | | | | | |
| median (range) years | | | | | | | | | | | | | | |
| Prior lines of therapy, | 6.00 | | 6.00 | | 6.00 | | 6.00 | | 6.00 | | 6.00 | | 6.00 | |
| | (2.0-14.0) | | (2.0-14.0) | | (2.0-18.0) | | (2.0-18.0) | | (2.0-18.0) | | (2.0-14.0) | | (2.0-14.0) | |
| median (range) | | | | | | | | | | | | | | |
| Refractory to last prior | 87 | (87.9) | 75 | (88.2) | 55 | (90.2) | 41 | (93.2) | 142 | (88.8) | 72 | (87.9) | 33 | (91.7) |
| therapy, n (%) | | | | | | | | | | | | | | |
| Prior triple-class | 86 | (86.9) | 73 | (85.9) | 42 | (95.5) | 42 | (95.5) | 141 | (88.1) | 72 | (87.8) | 35 | (97.2) |
| exposed (IMID, PI, and | | | | | | | | | | | | | | |
| Anti-CD38) | | | | | | | | | | | | | | |
| Triple-class refractory | 82 | (82.8) | 69 | (81.2) | 41 | (93.2) | 41 | (93.2) | 136 | (85.0) | 68 | (82.9) | 34 | (94.4) |
| Penta-refractory (2 | 68 | (68.7) | 58 | (68.2) | 29 | (65.9) | 29 | (65.9) | 109 | (68.1) | 58 | (70.7) | 22 | (61.1) |
| IMiDs, 2 PI, and Anti- | | | | | | | | | | | | | | |
| CD38) | | | | | | | | | | | | | | |
| Prior BCMA therapy^{g} | 31 | (31.3) | 28 | (33.0) | 23 | (37.7) | 16 | (36.3) | 54 | (33.7) | 28 | (34.2)f | 12 | (33.4) |

BCMA = B-cell maturation antigen; BMI = body mass index; CD38 = cluster of differentiation 38; ECOG = Eastern Cooperative Oncology Group; IMID = immunomodulatory drug; PI = proteasome inhibitor; Q3W = every 3 weeks; RP2D = recommended Phase II dose.
^{a} All patients refers to all patients in Arms A-D; data for 3 patients in Arm E are not presented.
^{b} The proposed RP2D and regimen is 0.3/3.6/160 mg Q3W: Cevostamab is administered at 0.3 mg (step-up dose) on Cycle 1 Day 1, 3.6 mg (step-up dose) on Cycle 1 Day 8, and 160 mg (target dose) on Cycle 1 Day 15 and Day 1 of subsequent Q3W cycles.
^{c} Cevostamab is administered on Day 1 (step-up dose) and Day 8 (target dose) of Cycle 1 and on Day 1 (target dose) of subsequent Q3W cycles.
^{d} Cevostamab is administered on Day 1 (step-up dose), Day 8 (step-up dose), and Day 15 (target dose) of Cycle 1 and on Day 1 (target dose) of subsequent Q3W cycles.
^{e} Doses ≥3.6 mg/20 mg, at which objective responses were observed, are considered the clinically active doses.
f Among all patients (n = 160), 72 (45.0%), 66 (41.3%), 75 (46.9%), 69 (43.1%), 51 (31.9%) patients had missing or unknown Amplified 1q21, Translocation t(4;14), Translocation t(11;14), Translocation t(14;16), and Deletion of TP53(17p), respectively and could not be classified.
^{g}Includes patients who received 1 or more prior BCMA therapies.

Efficacy Results

Of all 160 patients treated with the single or double step-up dose regimen, 158 were efficacy evaluable. Efficacy-evaluable patients were defined as patients who received treatment and had a response assessment, who had more than 30 days of treatment exposure, or discontinued cevostamab treatment prior to the 30 days of treatment exposure. Efficacy-evaluable patients who had no response assessments were considered as non-responders.

Clinical activity of cevostamab was seen starting at a target dose (TD) of 20 mg; any dose level with target ≥20 mg was considered to be an active dose. Clinically meaningful response rates were observed. Responses deepened over time and were generally durable. At a median follow-up time of 6.1 months (range 0.2-39.4), the projected median DOR was 15.6 months (95% CI: 6.4, 21.6) (Table 10).

TABLE 10
Summary of Best Overall Responses According to IMWG Response
Criteria for Efficacy-Evaluable Patients treated at Active
Doses and Patients Treated at Target Doses >90 mg

| | | | Prior BCMA ADC | | Triple-Class | |
|---|---|---|---|---|---|---|
| | Overall | | or CAR-T ^{a} | | Refractory ^{b} | |
| Enrolled patients | 160 | | 42 | | 136 | |
| Efficacy-evaluable | 141 | | 39 | | 120 | |
| patients treated | | | | | | |
| at active doses ≥20 mg | | | | | | |
| ORR (%) | 61 | (43.3) | 17 | (43.6) | 50 | (41.7) |
| SCR (%) | 11 | (7.8) | 3 | (7.7) | 10 | (8.3) |
| CR (%) | 3 | (2.1) | 2 | (5.1) | 3 | (2.5) |
| VGPR (%) | 14 | (9.9) | 5 | (12.8) | 13 | (10.8) |
| PR (%) | 33 | (23.4) | 7 | (18.0) | 24 | (20) |
| mDOR, months (95% | 15.6 | (6.4-21.6) | 15.6 | (3.7-NE) | 15.6 | (11.5-NE) |
| CI) | | | | | | |
| Event free rate, % | | | | | | |
| (95% CI) | | | | | | |
| 6 months | 66.4 | (52.6-80.3) | 65.5 | (40.8-90.1) | 68.1 | (52.0-84.3) |
| 12 months | 53.1 | (35.2-71.0) | 65.5 | (40.8-90.1) | 59.6 | (38.6-80.7) |
| Efficacy-evaluable | 59 | | 15 | | 55 | |
| patients treated | | | | | | |
| at >90 mg ^{c} | | | | | | |
| ORR (%) | 31 | (52.5) | 7 | (46.7) | 29 | (52.7) |
| sCR (%) | 4 | (6.8) | 1 | (6.7) | 4 | (7.3) |
| CR (%) | 2 | (3.4) | 1 | (6.7) | 2 | (3.6) |
| VGPR (%) | 7 | (11.9) | 2 | (13.3) | 7 | (12.7) |
| PR (%) | 18 | (30.5) | 3 | (20) | 16 | (29.1) |

ADC = antibody-drug conjugate; BCMA = B-cell maturation antigen; CAR-T = chimeric antigen receptor T-cell; CD38 = cluster of differentiation 38; CR = complete response; IMiD = immunomodulatory drug; IMWG = International Myeloma Working Group; mAb = monoclonal antibody; mDOR = modified duration of response; ORR = objective response rate; PI = proteasome inhibitor; PR = partial response; RP2D = recommended Phase II dose; sCR = stringent complete response; VGPR = very good partial response.
^{a} Prior BCMA is defined as patients previously treated with a BCMA-targeting ADC or CAR-T therapy and triple-exposed (to a PI, an IMiD and an aCD38 mAB).
^{b} Triple-class refractory is defined as patients refractory to an IMiD, a PI, and a CD38 MAb.
^{c} Activity observed in a dose escalation regimen with 23 patients treated with a single step-up dose of 3.6 mg followed by target doses of 132 mg, 160 mg, or 198 mg, and 36 patients treated with double step-up doses at 0.3/3.6/160 mg. This subset of patients represent activity expected with the RP2D at which only 7 post BCMA patients have been treated in the GO39775 study.

Among efficacy-evaluable patients with prior BCMA targeting CAR-T or ADC treated at clinically active doses, the ORR was 44% (17/39 patients), 95% CI: 28-60. At the time of CCOD, 11 of these patients were in continued response. The estimated DOR rate at 6 months was 65.5% (95% CI: 40.8, 90.1). This subgroup of prior BCMA targeting CAR-T or ADC patients had received a median of 7.5 prior lines of therapy, and a high proportion were also triple-class refractory (94%).

Triple-class refractory patients treated at clinically active doses showed an ORR of 42% (50/120 patients), 95% C1: 33-51. At the time of CCOD, 35 of these patients were in continued response. The estimated DOR rate at 6 months was 67.7% (95% C1: 52.1, 83.3). This subgroup of patients had received a median of 6 prior lines of therapy, and a high proportion were also penta-class refractory (81%).

At clinically active doses, efficacy in prior BCMA patients who received an ADC compound or a CAR T-cell product seemed to be similar (47.4% vs 41.7%). Although based on a small sample size, the efficacy observed in patients who received a BCMA-targeting bispecific antibody was limited, with only 1 of 9 patients showing a response.

At the proposed dose and schedule of RP2D, the reported response rate was 42.9% (3/7 patients) and 47.1% (16/34 patients) for patients with prior BCMA-targeting therapies and triple-class refractory patients, respectively.

At doses >90 mg across all arms in Study GO39775, 31 of 59 patients responded for an ORR of 53% (95% CI: 39-66). In a heavily pre-treated patient population, with a median of 6 prior lines of therapy (range: 2-18) and with a median observation time on study of 6.1 months (range: 0.2-39.4), the projected median DOR in 61 responders is 15.6 months (95% CI: 6.4-21.6). These efficacy results compare favorably with the current standard of care or published results in late line R/R MM such as those obtained with Selinexor/dexamethasone, belantamab mafodotin, melflufen or from the MAMMOTH retrospective study of outcome with current standard of care, showing response rates of 26% to 31% and median DORs of 4.4 to 11 months (Chari et al., N Engl J Med, 381: 727-738, 2019; Gandhi et al., Leukemia, 33: 2266-2275, 2019; Lonial et al., Lancet Oncol, 21(2): 207-221, 2020; Richardson et al., J Clin Oncol, 39: 757-767, 2021).

The key efficacy findings from ongoing Study GO39775 are as follows:
The observed ORR across all doses explored was 38.6% (61 out of 158 patients, 95% CI: 30.7, 46.5). Clinical active doses are defined as TD ≥20 mg, dose at which first responses were observed. The ORR at active dose was 43.3% (61 out of 141 patients, 95% CI: 35.0, 51.9).
Of the 61 responders, 21 had ongoing responses at 6 months, and 8 patients had an ongoing response at 12 months. The estimated DOR rate at 6 months was 66% (95% CI: 53, 80) and the estimated median DOR, 15.6 months (95% CI: 6.4-21.6).
In single step-up clinically active doses, the ORR was 45.0% (36/80 patients; 95% CI: 33.5-56.5). The median to time to first response was 29 days (range: 21-105) and response deepened over time, with a median time to best overall response at 51 days (range: 21-323). A dose response relationship was observed with higher activity at TDs >3.6/90 mg showing an ORR of 60.9% (14/23 patients, 95% CI: 38.8, 83.0) as compared with 38.6% (21/57 patients, 95% CI: 25.1, 52.1) at doses of 3.6/20-90 mg. As of the CCOD, the median follow-up time was 9.3 months (range: 0.2-28.5) and the estimated median DOR was 15.6 months (95% C1: 11.5, 21.6).
In double step-up dosing cohorts, the ORR was 41.0% (25/61 patients, 95% CI:27.8-54.1). With a short median follow up time of 3.3 months (range: 0.5-18.5), the best overall response and response ≥VGPR are still preliminary as depth of response for recently enrolled patients might still evolve. The estimated median DOR was 8.3 months (95% CI: 2.3-NE).
At the RP2D (0.3/3.6/160 mg), the ORR was 47.2% (17/36 patients, 95% CI: 30.4, 64.5), with 2.8% (1 patient) achieving a sCR, 2.8% (1 patient) achieving a CR, and 8.3% (3 patients) achieving a VGPR.

Efficacy in Single Step-Up Dose Regimens (Arm a +Arm C)

Of the 80 efficacy-evaluable patients treated at clinically active doses (≥3.6 mg/20 mg) in Arms A and C, 36 patients (45.0%) had objective responses (Table 11). As of the CCOD, the median follow-up time was 9.3 months (range: 0.2-28.6). The median follow-up time of the 36 responders was 11.2 months (range: 2.7-28.6 months), with the median time on treatment of 7.2 months (range: 0.3-30.2 months). The median time to best overall response among responders was 50 days (range: 21-323 days). The estimated median DOR was 15.6 months (95% CI: 11.5, 21.6). At the time of CCOD, 9 of the 36 responders (25%) still remained on treatment.

A dose-response relationship was observed in the dose-escalation cohorts, and higher response rate was reported at TDs >3.6/90 mg with an ORR of 60.9% (95% CI: 38.8, 83.0) as compared with 38.6% (95% CI: 25.1, 52.1) at doses of 3.6/20-90 mg.

TABLE 11
Summary of Best Overall Responses According to IMWG Response Criteria for Efficacy-Evaluable Patients
Receiving ≥3.6/20 mg Doses in Single Step-Up Dose Regimen (Arm A and Arm C) of Study GO39775

| | | | | | | | | | | | | | Clinically | | Doses | |
|---|---|---|---|---|---|---|---|---|---|---|---|---|---|---|---|---|
| | | | | | | | | | | | | | Active | | >90 mg | |
| | | | | | | | | | | | Arm C | | Doses in | | in | |
| | | | | | | | | | | | (Dose | | Arm A + | | Arm A + | |
| Arm A (Dose Escalation at Active Doses)^{a} | | | | | | | | | | | Expansion) | | Arm C | | Arm C | |
| | Dose (mg) | | | | | | | | | | | | | | | |
| | 3.6/(20-90) | | 3.6/132 | | 3.6/160 | | 3.6/198 | | Total | | 3.6/90 | | 3.6/90 | | 3.6/90 | |
| | No. of Patients | | | | | | | | | | | | | | | |
| | N = 27 | | N = 7 | | N = 8 | | N = 8 | | N = 50 | | N = 30 | | N = 80 | | N = 23 | |
| ORR^{b} (%) | 13 | (48.1) | 5 | (71.4) | 7 | (87.5) | 2 | (25.0) | 27 | (54.0) | 9 | (30.0) | 36 | (45.0) | 14 | (60.9) |
| SCR (%) | 4 | (14.8) | 2 | (28.6) | 1 | (12.5) | 0 | | 7 | (14.0) | 3 | (10.0) | 10 | (12.5) | 3 | (13.0) |
| CR (%) | 1 | (3.7) | 0 | | 1 | (12.5) | 0 | | 2 | (4.0) | 0 | | 2 | (2.5) | 1 | (4.3) |
| VGPR | 3 | (11.1) | 2 | (28.6) | 2 | (25.0) | 0 | | 7 | (14.0) | 3 | (10.0) | 10 | (12.5) | 4 | (17.4) |
| (%) | | | | | | | | | | | | | | | | |
| PR (%) | 5 | (18.5) | 1 | (14.3) | 3 | (37.5) | 2 | (25.0) | 11 | (22.0) | 3 | (10.0) | 14 | (17.5) | 6 | (26.1) |
| MR (%) | 4 | (14.8) | 0 | | 0 | | 0 | | 4 | (8.0) | 2 | (6.7) | 6 | (7.5) | 0 | |
| SD (%) | 5 | (18.5) | 1 | (14.3) | 0 | | 5 | (62.5) | 11 | (22.0) | 11 | (36.7) | 22 | (27.5) | 6 | (26.1) |
| PD (%) | 5 | (18.5) | 1 | (14.3) | 1 | (12.5) | 1 | (12.5) | 8 | (16.0) | 7 | (23.3) | 15 | (18.8) | 3 | (13.0) |

| Clinical | 0 | 0 | 0 | 0 | 0 | 0 | | 0 | | 0 |
|---|---|---|---|---|---|---|---|---|---|---|
| Relapse | | | | | | | | | | |
| (%) | | | | | | | | | | |
| Missing | 0 | 0 | 0 | 0 | 0 | 1 | (3.3) | 1 | (1.3) | 0 |

or NE
(%)

CR = complete response; IMWG = International Myeloma Working Group; MR = minimal response; NE = not evaluable; ORR = objective response rate; PD-progressive disease; PR = partial response; sCR = stringent complete response; SD = stable disease; VGPR = very good partial response.
Note:
Non-response includes MR, SD, PD, clinical relapse, or missing/NE.
^{a}No objective responses were observed at doses <3.6 mg/20 mg (0.05 mg/0.15 mg to 3.6 mg/10.8 mg) in Arm A.
^{b}ORR is defined as the proportion of patients who achieved sCR, CR, VGPR, or PR as determined by investigator assessment according to the IMWG response criteria.

Efficacy in Double Step-Up Dose Regimens (Arm B+Arm D)

Of the 61 efficacy-evaluable patients in Arms B and D, 25 patients (41.0%) had objective responses (Table 12). As of the CCOD, the median follow-up time was 3.3 months (range: 0.5-18.5). The median follow-up time of the 25 responders was 3.3 months (range: 1.6-18.2 months) and all but one remained on treatment for a median time on treatment of 1.5 months (range: 0-12.0 months). At the time of CNOD, 18 responders (72.0%) remained on treatment.

In Study GO39775, for the 36 patients who received cevostamab at the RP2D (0.3/3.6/160 mg), the ORR was 47.2% (17 patients), with 2.8% (1 patient) achieving a sCR, 2.8% (1 patient) achieving a CR, and 8.3% (3 patients) achieving a VGPR. Based on the short follow up time for the double step-up arms, the reported rates of VGPR or better response might be underestimated.

TABLE 12
Summary of Best Overall Responses According to IMWG Response Criteria for Efficacy-Evaluable
Patients in Double Step-Up Dose Regimen Arms (Arm B and Arm D) of Study GO39775

| | | | | | | | | | | | | | | | Clinically | | | |
|---|---|---|---|---|---|---|---|---|---|---|---|---|---|---|---|---|---|---|
| | | | | | | | | | | | | | | | Active | | Proposed | |
| | | | | | | | | | | | | | Arm D | | Doses in | | RP2D | |
| | | | | | | | | | | | | | (Dose | | Arm B + | | and | |
| | Arm B (Dose Escalation) | | | | | | | | | | | | Expansion) | | Arm D | | Regimen^{a} | |
| | Dose (mg) | | | | | | | | | | | | | | | | | |
| | 1.2/3.6/60 | | 1.2/3.6/90 | | 0.3/3.6/90 | | 0.6/3.6/90 | | 0.3/3.6/160 | | Total | | 0.3/3.6/160 | | ≥3.6/20 | | 0.3/3.6/160 | |
| | No. of Patients | | | | | | | | | | | | | | | | | |
| | N = 6 | | N = 3 | | N = 8 | | N = 8 | | N = 5 | | N = 30 | | N = 31 | | N = 61 | | N = 36 | |
| ORR^{b} (%) | 1 | (16.7) | 2 | (66.7) | 3 | (37.5) | 2 | (25.0) | 2 | (40.0) | 10 | (33.3) | 15 | (48.4) | 25 | (41.0) | 17 | (47.2) |
| SCR (%) | 0 | | 0 | | 0 | | 0 | | 0 | | 0 | | 1 | (3.2) | 1 | (1.6) | 1 | (2.8) |
| CR (%) | 0 | | 0 | | 0 | | 0 | | 1 | (20.0) | 1 | (3.3) | 0 | | 1 | (1.6) | 1 | (2.8) |
| VGPR | 0 | | 1 | (33.3) | 0 | | 0 | | 0 | | 1 | (3.3) | 3 | (9.7) | 4 | (6.6) | 3 | (8.3) |
| (%) | | | | | | | | | | | | | | | | | | |
| PR (%) | 1 | (16.7) | 1 | (33.3) | 3 | (37.5) | 2 | (25.0) | 1 | (20.0) | 8 | (26.7) | 11 | (35.5) | 19 | (31.1) | 12 | (33.3) |
| MR (%) | 0 | | 0 | | 0 | | 0 | | 0 | | 0 | | 3 | (9.7) | 3 | (4.9) | 3 | (8.3) |
| SD (%) | 4 | (66.7) | 1 | (33.3) | 4 | (50.0) | 2 | (25.0) | 2 | (40.0) | 13 | (43.3) | 9 | (29.0) | 22 | (36.1) | 11 | (30.6) |

| | | | | | | | | | | | | | | | | |
|---|---|---|---|---|---|---|---|---|---|---|---|---|---|---|---|---|
| PD (%) | 1 | (16.7) | 0 | 1 | (12.5) | 3 | (37.5) | 0 | 5 | (16.7) | 3 | (9.7) | 8 | (13.1) | 3 | (8.3) |

| | | | | | | | | | |
|---|---|---|---|---|---|---|---|---|---|
| Clinical | 0 | 0 | 0 | 0 | 0 | 0 | 0 | 0 | 0 |
| Relapse | | | | | | | | | |
| (%) | | | | | | | | | |
| Missing | 0 | 0 | 0 | 0 | 0 | 0 | 0 | 0 | 0 |
| or NE | | | | | | | | | |
| (%) | | | | | | | | | |

CR = complete response; IMWG = International Myeloma Working Group; MR = minimal response; NE = not evaluable; ORR = objective response rate; PD-progressive disease; PR = partial response; RP2D = recommended Phase II dose; sCR = stringent complete response; SD = stable disease; VGPR = very good partial response.
Note:
Non-response includes MR, SD, PD, clinical relapse, or missing/non-evaluable.
^{a}The RP2D and regimen is 0.3/3.6/160 mg Q3W: Cevostamab is administered at 0.3 mg (step-up dose) on Cycle 1 Day 1, 3.6 mg (step-up dose) on Cycle 1 Day 8, and 160 mg (target dose) on Cycle 1 Day 15 and Day 1 of subsequent Q3W cycles.
^{b}ORR is defined as the proportion of patients who achieved sCR, CR, VGPR, or PR as determined by investigator assessment according to the IMWG response criteria.

Safety Results

The clinical safety data presented in Table 13 include data from 160 safety-evaluable patients (defined as patients who received cevostamab treatment) in Study GO39775: 68 patients in Arm A and 31 patients in Arm C (single step-up dose regimen) and 30 patients in Arm B and 31 patients in Arm D (double step-up dose regimen). Clinical safety data were also presented for patients treated at the proposed RP2D and regimen (0.3 mg/3.6 mg/160 mg) and for patients treated at clinically active doses in Arm A (≥3.6 mg/20 mg).

TABLE 13
Overview of adverse events in GO39775 (Safety-Evaluable Patients)

| | Single Step- | | Double Step- | | | | Recommended | |
|---|---|---|---|---|---|---|---|---|
| | Up Dose | | Up Dose | | | | Phase II | |
| | Regimen | | Regimen | | | | Dose and | |
| | (Arms A + C) | | (Arms B + D) | | All Patients ^{a} | | Regimen^{b} | |
| | N = 99 | | N = 61 | | N = 160 | | N = 36 | |
| Dose (mg) | 0.05/0.15 to | | 1.2/3.6/60 to | | | | 0.3/3.6/160 | |
| | 3.6/198 ^{c} | | 0.3/3.6/160 ^{d} | | | | | |
| Total number of patients | 98 | (99.0) | 61 | (100) | 159 | (99.4) | 36 | (100) |
| with at least one AE (%) | | | | | | | | |
| Total number of events | 1083 | | 675 | | 1758 | | 364 | |
| Total number of deaths (%) | 43 | (43.4) | 10 | (16.4) | 53 | (33.1) | 3 | (8.3) |

| | | | | |
|---|---|---|---|---|
| Total number of patients | 0 | 0 | 0 | 0 |
| withdrawn from the | | | | |
| study due to an AE (%) | | | | |

Total number of patients with:

| | | | | | | | | |
|---|---|---|---|---|---|---|---|---|
| AE with fatal outcome^{b} (%) | 18 | (18.2) | 6 | (9.8) | 24 | (15.0) | 2 | (5.6) |
| SAE (%) | 57 | (57.6) | 32 | (52.5) | 89 | (55.6) | 19 | (52.8) |
| Related SAE (%) | 24 | (24.2) | 16 | (26.2) | 40 | (25.0) | 10 | (27.8) |
| Grade ≥3 AE (%) | 76 | (77.6) | 42 | (68.9) | 118 | (74.2) | 25 | (69.4) |
| Related Grade ≥3 AE (%) | 49 | (50.0) | 27 | (44.3) | 76 | (47.8) | 14 | (38.9) |
| AE leading to treatment | 12 | (12.1) | 4 | (6.6) | 16 | (10.0) | 3 | (8.3) |
| withdrawal (%) | | | | | | | | |
| AE leading to dose | 32 | (32.3) | 18 | (29.5) | 50 | (31.3) | 11 | (30.6) |
| modification/interruption (%) | | | | | | | | |
| Related AE | 94 | (94.9) | 57 | (93.4) | 151 | (94.4) | 33 | (91.7) |

AE = adverse event; ASTCT = American Society for Transplantation and Cellular Therapy; CRS = cytokine release syndrome; NCI CTCAE = National Cancer Institute Common Terminology Criteria for Adverse Events; Q3W = every 3 weeks; RP2D = recommended Phase II dose; SAE = serious adverse event.
Note:
Investigator text for AEs encoded using MedDRA version 24.0. Only treatment emergent AEs are displayed. Percentages are based on N in the column headings.
Note:
Toxicity grade of CRS was evaluated by ASTCT 2019 criteria, while all other non-CRS events were assessed by NCI CTCAE grading criteria v4.
^{a} “All Patients” refer to all patients in Arms A-D, data for the 3 patients in Arm E are not presented in this document.
^{b}The proposed RP2D and regimen is 0.3/3.6/160 mg Q3W: Cevostamab is administered at 0.3 mg (step-up dose) on Cycle 1 Day 1, 3.6 mg (step-up dose) on Cycle 1 Day 8, and 160 mg (target dose) on Cycle 1 Day 15 and Day 1 of subsequent Q3W cycles.
^{c} Cevostamab is administered on Day 1 (step-up dose) and Day 8 (target dose) of Cycle 1 and on Day 1 (target dose) of subsequent Q3W cycles.
^{d} Cevostamab is administered on Day 1 (step-up dose), Day 8 (step-up dose), and Day 15 (target dose) of Cycle 1 and on Day 1 (target dose) of subsequent Q3W cycles.
e Includes deaths attributed to progression of cancer that occurred during the protocol-specified AE reporting period (i.e., 90 days after the last dose of study treatment or until the initiation of another systemic anti-cancer therapy, whichever occurred first), which were reportable as SAEs with fatal outcome.

Overall, the majority of the reported AEs were of low grade and reversible. CRS was the most frequently reported AE among treated patients. The maximum tolerated dose (MTD) was not reached at the time of CCOD. The safety profile of cevostamab is currently manageable and will continue to be further characterized.

The safety profile between patients treated with single step-up dose and double step-up dose regimen the was generally similar. Differences were noted in the CRS/ICANS profile, as described herein. At the 0.3/3.6/160 mg double step-up dose regimen and at clinically active doses, the safety profile was consistent with that in the overall safety-evaluable population of the study. Across the clinically active dose ranges, there are no trends towards TD-dependent toxicity.

The triple-class refractory patients as well as the patients who have received a prior PI, IMiD, anti-CD38 MAb, and BCMA-targeting therapy had a similar safety profile compared to the overall 160 patients in Study GO39775, as summarized in Table 14.

The most frequently reported AEs reported in these populations compared to the overall 160 patients in Study GO39775 are described in Table 15. Though the subset of patients with prior BCMA is small, similar trends are seen in the AEs in this and the triple class refractory population compared to the overall 160 patients in Study GO39775.

TABLE 14
Overview of Adverse Events in GO39775 in
Overall Safety-Evaluable, Prior BCMA, and

| | | | | | Triple-Class | |
|---|---|---|---|---|---|---|
| | | | Prior BCMA | | Refractory | |
| | All Patients ^{a} | | Population | | Population | |
| | N = 160 | | N = 42 | | N = 136 | |
| Total number of patients | 159 | (99.4) | 42 | (100) | 136 | (100) |
| with at least one AE (%) | | | | | | |
| Total number of events | 1758 | | 514 | | 1469 | |
| Total number of deaths | 53 | (33.1) | 11 | (26.2) | 47 | (34.6) |
| (%) | | | | | | |
| Total number of patients | 0 | | 0 | | 0 | |
| withdrawn from the | | | | | | |
| study due to an AE (%) | | | | | | |

Total number of patients with at least one:

| | | | | | | |
|---|---|---|---|---|---|---|
| AE with fatal outcome^{b} | 24 | (15.0) | 7 | (16.7) | 21 | (15.4) |
| (%) | | | | | | |
| SAE (%) | 89 | (55.6) | 21 | (50.0) | 74 | (53.2) |
| Related SAE (%) | 40 | (25.0) | 11 | (26.2) | 29 | (21.3) |
| Grade ≥3 AE (%) | 118 | (74.2) | 30 | (71.4) | 10 | (73.5) |
| Related Grade ≥3 AE | 76 | (47.8) | 21 | (50.0) | 61 | (44.9) |
| (%) | | | | | | |
| AE leading to treatment | 16 | (10.0) | 7 | (16.7) | 13 | (9.6) |
| withdrawal (%) | | | | | | |
| AE leading to dose | 50 | (31.3) | 14 | (33.3) | 40 | (29.4) |
| modification/interruption | | | | | | |
| (%) | | | | | | |
| Related AE | 151 | (94.4) | 41 | (97.6) | 128 | (94.1) |

AE = adverse event; MedDRA = Medical Dictionary for Regulatory Activities; NCI CTCAE = National Cancer Institute Common Terminology Criteria for Adverse Events; Q3W = every 3 weeks; SAE = serious adverse event.
Note:
Investigator text for AEs encoded using MedDRA version 24.0. Only treatment emergent AEs are displayed. Percentages are based on N in the column headings.
Note:
Toxicity grade of Cytokine Release Syndrome (CRS) was evaluated by ASTCT 2019 criteria, while all other non-CRS events were assessed by NCI CTCAE grading criteria v4.
^{a} “All Patients” refer to all patients in Arms A-D, data for the 3 patients in Arm E are not presented in this document.
^{b}Includes deaths attributed to progression of cancer that occurred during the protocol-specified AE reporting period (i.e., 90 days after the last dose of study treatment or until the initiation of another systemic anti-cancer therapy, whichever occurred first), which were reportable as SAEs with fatal outcome.

TABLE 15
Frequency ≥15% of Adverse Events in Study
GO39775 in Overall Safety-Evaluable, Prior
BCMA, and Triple-Class Refractory Population

| | | |
|---|---|---|
| | Prior BCMA | Triple-Class |
| All Patients | Population | Refractory Population |
| N = 160 | N = 42 | N = 136 |

Patients (%) with:

| | | | | | | |
|---|---|---|---|---|---|---|
| CRS | 128 | (80.0) | 39 | (92.9) | 111 | (79.9) |
| Anemia | 51 | (31.9) | 13 | (31.0) | 44 | (31.7) |
| Diarrhea | 42 | (26.3) | 9 | (21.4) | 36 | (26.5) |
| Cough | 37 | (23.1) | 11 | (26.2) | 33 | (24.3) |
| Nausea | 35 | (21.9) | 10 | (23.8) | 29 | (21.3) |
| Neutropenia | 29 | (18.1) | 9 | (21.4) | 22 | (16.2) |
| Infusion Related | 28 | (17.5) | 11 | (26.2) | 22 | (16.2) |
| Reaction (IRR) | | | | | | |
| Fatigue | 26 | (16.3) | 9 | (21.4) | 24 | (17.6) |
| Pyrexia | 25 | (15.6) | 8 | (19.0) | 20 | (14.7) |
| Aspartate | 25 | (15.6) | 11 | (26.2) | 21 | (15.4) |
| Aminotransferase | | | | | | |
| Increased | | | | | | |
| Hypomagnesemia | 25 | (15.6) | 8 | (19.0) | 21 | (15.4) |
| Alanine | 24 | (15.0) | 11 | (26.2) | 23 | (16.9) |
| Aminotransferase | | | | | | |
| increased | | | | | | |
| Neutrophil Count | 24 | (15.0) | 8 | (19.0) | 20 | (14.7) |

Decreased

The key safety findings from ongoing Study GO39775 are as follows:
The overall incidence rate of AEs was 99.4% (159 patients). The most frequently reported AEs are CRS (80.0%), anemia (31.9%), diarrhea (26.3%), cough (23.1%), nausea (21.9%), neutropenia (18.1%), and infusion-related reaction (17.5%).
Fatal AEs (not including Grade 5 AEs of disease progression) were reported in 5 patients (3.1%). Causes of death include respiratory failure in 3 patients, acute kidney injury, and HLH. HLH was the only death considered related to cevostamab treatment.
Serious AEs (not including Grade 5 AEs of disease progression) were reported in 70 patients (43.8%). The most frequently reported SAEs are CRS (13.8%) and pneumonia (6.3%).
Grade ≥3 AEs (not including Grade 5 AEs of disease progression) were reported in 99 patients (61.9%). The most frequently reported Grade ≥3 AEs are anemia (21.9%), neutropenia (16.3%), and neutrophil count decreased (13.8%).
AEs leading to withdrawal of cevostamab were reported in 16 patients (10%). A total of 6 patients (4.4%) withdrew from AEs related to study treatment. AEs leading to dose modification of cevostamab were reported in 5 patients (3.1%).
At the RP2D, 36 patients have been treated and the most frequently reported AE was CRS (80.6%).

Timing of CRS and Hospitalization Requirement

In the GO39775 study, patients are required, as per protocol, to be hospitalized for a minimum of 72 hours during all cycle 1 doses in order to ensure rapid detection and management of CRS. This decision was made, for the safety of the patients, before any clinical data were available. Hospitalization has proven to be an effective risk mitigation measure in detecting and treating CRS quickly; however, the requirement of keeping patients in the hospital for 48 hours regardless of whether they develop CRS or not, and regardless of how quickly they recover, adds an undue burden to patients and hospitals. Based on the available data in the ongoing Study GO39775, the current hospitalization requirement may be reduced to a minimum of 24 hours of hospitalization after the completion of infusion for C1D1 and 48 hours of hospitalization after the completion of infusion for C1D8 and C1D15; if the duration of CRS extends beyond the 24 or 48 hours, patients remain in hospital and the event is considered a serious adverse event (SAE) due to prolonged hospitalization. Patients may be discharged after 24 hours (C1D1) or 48 hours (C1D8, C1D15) if they meet all of the following criteria: no evidence of ongoing CRS; no evidence of neurological toxicity; vitals and oxygen saturation return to baseline; abnormal laboratory values attributed to cevostamab are improving towards normal or baseline.

Patients who do not experience IRR or CRS with the Cycle 1 TD will not require hospitalization for the next dose on C2D1 and subsequent doses. Hospitalization for subsequent doses is considered for individual patients based on how they tolerated the initial doses in Cycle 1.

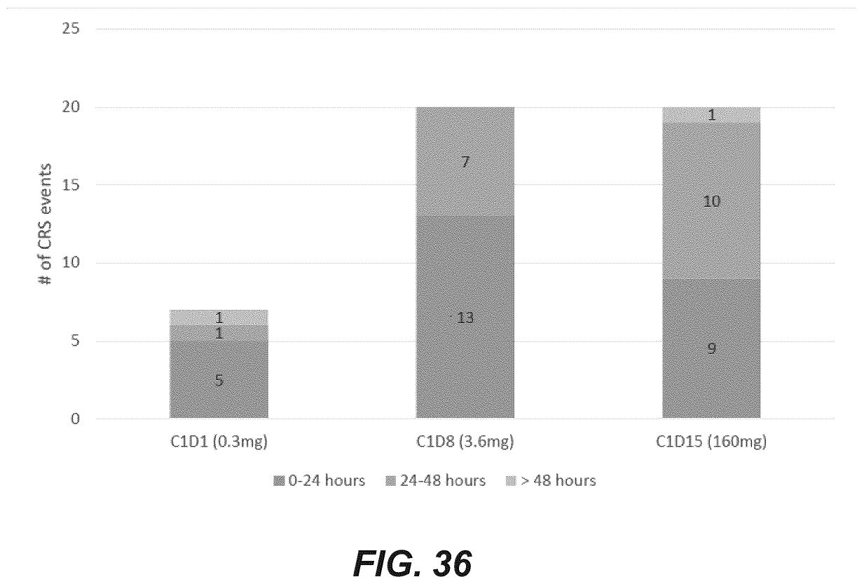

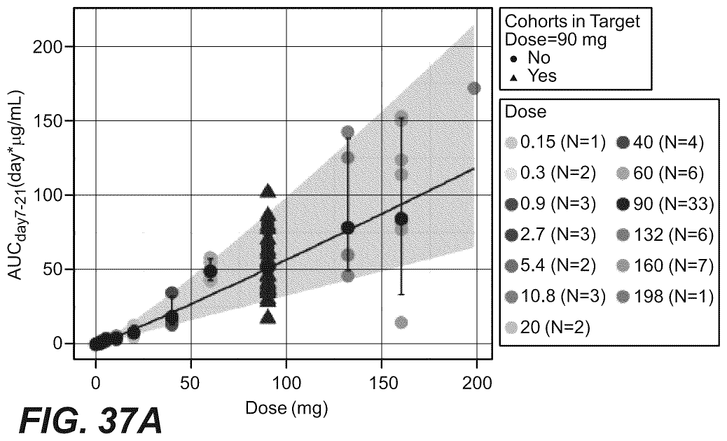

At the first step-up dose of the RP2D (0.3 mg, C1D1), the rate of CRS was low (7 out of 36 patients, 19.4%), all CRS events were Grade 1, no ICANS symptoms were reported, and all but 2 patients (both experiencing symptoms limited to fever and chills) had an onset within 24 hours (see FIG. 36). No intervention with fluids or oxygen was required and only 2 of the 7 patients reporting CRS were treated with tocilizumab and/or steroids for prolonged fever. All events resolved by the following day (within 24 hours of onset).

At the second step up dose of the RP2D (3.6 mg, C1D8), all CRS events had an onset within 48 hours post infusion. At the first TD of the RP2D (160 mg, C1D15), all CRS events except for one had an onset within 48 hours post-infusion. This patient reported cough, dyspnea, dysphonia, and hypoxia requiring low flow oxygen onset at 56 hours post infusion at C1D15. All events resolved and the patient went on to get further cycles without recurrence.

### Example 6. Results at Intermediate Data Cut-Off Points

Arm A (Single-Step Dose Escalation Arm) at First Data Cut-Off

At a first data cut-off, 51 patients had been enrolled into Arm A. Patients had a median age of 62.0 years (range: 33-80 years). 28 patients had high-risk (HR) cytogenetics (1q21, t(4;14), t(14;16) or del(17p)).

The median number of prior lines of therapy was 6 (range: 2-15). Prior treatments included:
proteasome inhibitors (PIs) (all patients; 94.1% refractory);
immunomodulatory drugs (IMiDs) (all patients, 98.0% refractory);
anti-CD38 monoclonal antibodies (mAbs) (40 patients (78.4%); 92.5% refractory);
CAR-T cells, T-cell engaging bispecific antibodies (bsAbs), or antibody-drug conjugates (ADCs) (12 patients (23.5%)); and
autologous stem cell transplant (44 patients, 86.3%).

34 patients (66.7%) were triple-class refractory (≥1 PI, ≥1 IMiD, and ≥1 anti-CD38 mAb), and 48 patients (94.1%) were refractory to their last therapy.

The dose escalation study followed a typical 3+3 design. Patients in Cohort 1 were treated with an 0.05 mg step dose on C1D1 (cycle 1, day 1) and an 0.15 mg target dose on C1D8 (cycle 1, day 8) and beyond (FIG. 4A). No DLTs or activity were observed in Cohorts 2-6. At Cohort 7 (C1D1: 3.6 mg; C1D8: 20 mg), objective response of partial response (PR) or higher was first observed. Activity continued to be observed as the dose was escalated up to the highest cleared dose at C1D1: 3.6 mg; C1D8: 132 mg (FIG. 4A). Cevostamab was thus observed to be active at target doses of 20 mg and higher. 34 patients were treated at an effective dose (3.6/20 mg to 3.6/90 mg). Baseline demographics for these patients are shown in Table 16.

Cohort 10 of Arm A was treated with 3.6 mg and 90 mg cevostamab on C1D1 and C1D8, respectively, administered intravenously (FIG. 4A).

TABLE 16
Baseline patient demographics for Arm A

| Baseline Characteristic | N = 34 |
|---|---|
| Age, median [range] | 62 [33-75] |
| Prior lines of treatment, median [range] | 6 [2-15] |
| Prior IMID, n (%), refractory [%] | 34 (100%) [98%] |
| Prior PI, n (%), refractory [%] | 34 (100%) [94%] |
| Prior Dara, n (%), refractory [%] | 25 (73%) [92%] |
| Prior ASCT, n (%) | 29 (85%) |
| Refractory to last line, n (%) | 32 (94%) |
| Triple class [PI, IMID, aCD38] refractory, n (%) | 23 (66%) |
| High risk cytogenetics, n (%)* | 19 (56%) |

*High risk cytogenetics by central assessment: [1q21, t(4;14), t(14;16), or del(17p)]

i. Efficacy

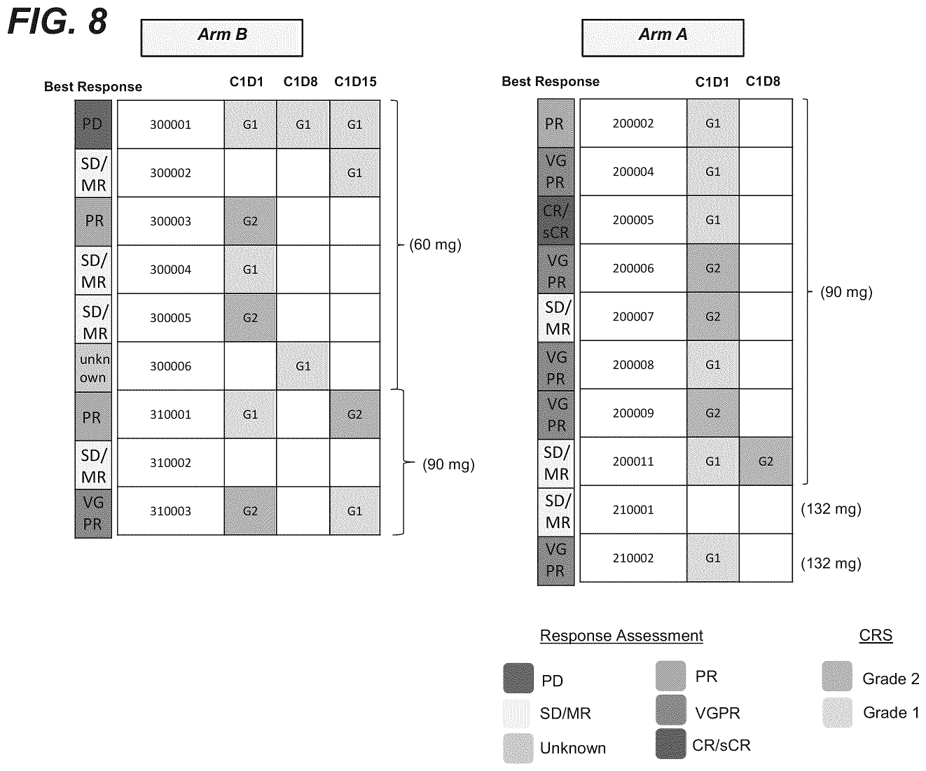

At the cut-off used in this Example, 46 of the 51 patients were evaluable for efficacy. Responses were observed at and above the 3.6/20 mg dose level in 15 of 29 patients (51.7%) (Table 17 and FIG. 8). Responses included 3 stringent complete responses (sCRs), 3 complete responses (CRs), 4 very good partial responses (VGPRs), and 5 partial responses (PRs) (Table 17). At the active dose level and above, responses were observed in patients with HR cytogenetics (9/17), triple-class refractory disease (10/20), and prior exposure to anti-CD38 mAbs (11/22), CAR-Ts (2/3), or ADCs (2/2). 6 of 15 patients have been responding for more than 6 months at cut-off. Responses were observed across a range of FcRH5 expression levels, including in patients with lower levels of expression.

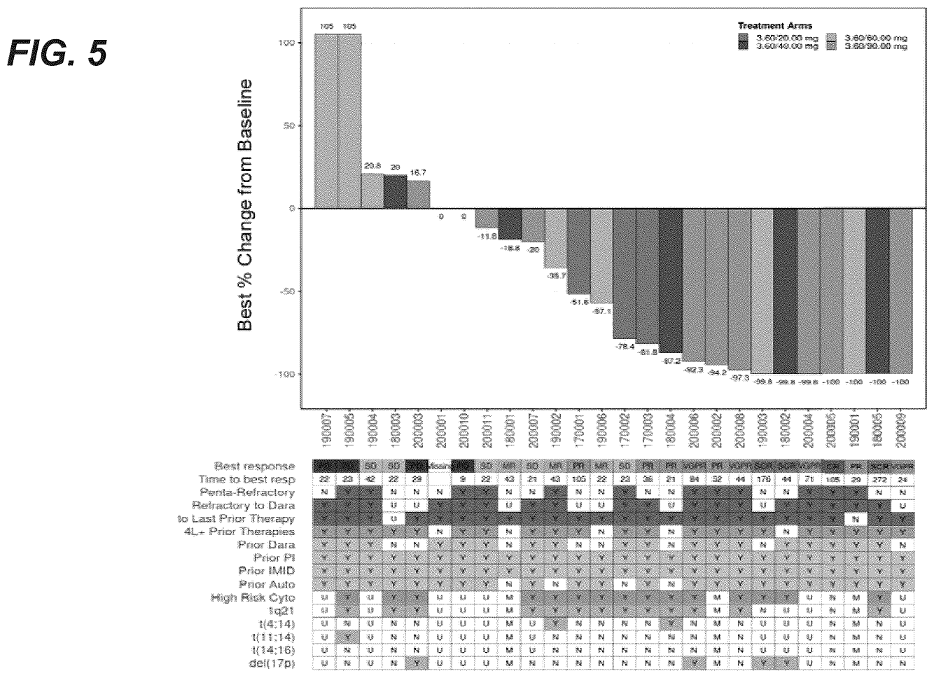

Most patients treated in the single-step fractionation arm have received six or more prior lines of therapy, as shown in FIG. 5, e.g., have received a proteasome inhibitor (PI), IMiD, and/or anti-CD38 therapy (e.g., daratumumab). ORR was similar in patients who had received prior daratumumab (50%, 11/22) (FIG. 5). Responses were also seen in patients who had previously received an antibody-drug conjugate targeting B-cell maturation antigen (BCMA-ADC (2/3 patients)) or chimeric antigen receptor T cell (CAR-T) therapy (2/5 patients). Efficacy was observed regardless of high risk cytogenetics, number of prior lines, previous therapies used, or other demographic stratifications.

Six of eleven patients in Cohort 10 had an objective response: one patient had a partial response (PR), four patients had a very good partial response (VGPR), and one patient had a complete response (CR). The time to first response varied among patients. The majority of patients who responded to treatment had PR or better within the first three cycles of treatment.

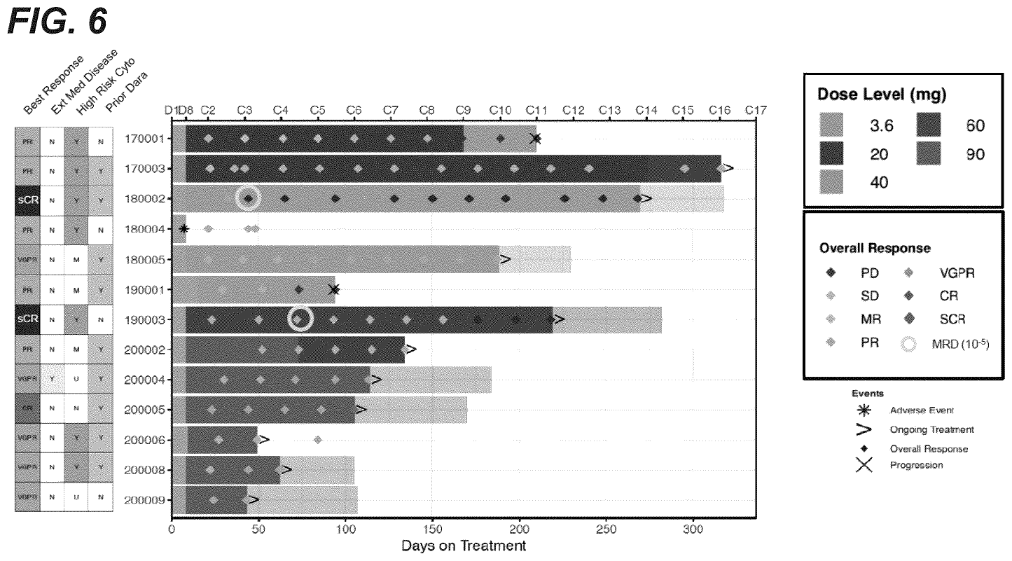

FIG. 6 shows timelines of treatment for each of thirteen patients who showed a response to cevostamab therapy. Six of the thirteen responders have maintained response for over six months, and several are approaching one year on treatment.

TABLE 17
Summary of best overall response by investigator assessment at the active
dose level (3.26/20 mg and above) in single-step dose escalation arm

| | Cohort 7 | Cohort 8 | Cohort 9 | Cohort 10 | Cohort 11 | Total of | |
|---|---|---|---|---|---|---|---|
| | 3.6/ | 3.6/ | 3.6/ | 3.6/ | 3.6/ | Cohorts | Total of |
| | 20 mg | 40 mg | 60 mg | 90 mg | 132 mg | 7-11 | All Cohorts |
| | N = 3 | N = 6 | N = 7 | N = 11 | N = 7 | N = 34 | N = 51 |
| ORR ¹ (%) | 2 (66.7) | 4 (66.7) | 1 (14.3) | 6 (54.5) | 5 (71.4) | 18 (52.9) | 18 (35.3) |
| SCR (%) | — | 2 (33.3) | 1 (14.3) | — | 1 (14.3) | 4 (11.8) | 4 (7.8) |
| CR (%) | — | — | — | 1 (9.1) | 1 (14.3) | 2 (5.9) | 2 (3.9) |
| VGPR (%) | — | — | — | 4 (36.4) | — | 4 (11.8) | 4 (7.8) |
| PR (%) | 2 (66.7) | 2 (33.3) | — | 1 (9.1) | 3 (42.9) | 8 (23.5) | 8 (15.7) |
| MR/SD/PD | 1 (33.3) | 2 (33.3) | 6 (85.7) | 4 (36.3) | 2(28.5) | 16 (47.0) | 33 (64.7) |
| (%) | | | | | | | |

¹ patients with best overall response of sCR, CR, VGPR or PR by IMWG uniform response criteria 2016; ORR, overall response rate; CR, complete response; PR, partial response; sCR, stringent CR; VGPR, very good PR; MR, minimal response; SD, stable disease; PD, progressive disease.

ii. Safety

Median follow-up for safety was 6.2 months (range: 0.2-26.3 months). Almost all patients (49/51) had ≥1 treatment-related adverse event (AE). The most common treatment-related AE was cytokine release syndrome (CRS), as defined by the criteria established by Lee et al., Blood, 124: 188-195, 2014 (Table 5A). 42 of 46 patients (91%) treated with clinically active doses of cevostamab (≥3.6 mg/20 mg) experienced CRS (Tables 18-20).

TABLE 18
Frequency and grade of CRS

| | | All Safety | | | | |
|---|---|---|---|---|---|---|
| | | Evaluable at | | | | |
| | | Clinically | All Safety | | | Arm C |
| | | Active Dose | Evaluable at | Arm A | Arm B | (Single- |
| | All Safety | Levels | 3.6 mg/90 mg | (Single- | (Double- | Step) |
| | Evaluable | (≥3.6 mg/20 mg) | Dose | Step) | Step) | 3.6 mg/90 mg |
| | n = 72 | n = 46 | n = 23 | n = 52 | n = 9 | n = 11 |
| Any Grade | 57 (79.2%) | 42 (91%) | 22 (96%) | 39 (75%) | 8 (88.9%) | 10 (91%) |
| Grade 1 | 29 (40.3%) | 20 (43%) | 10 (43%) | 20 (38%) | 4 (44.4%) | 5 (45.4%) |
| Grade 2 | 26 (36.1%) | 20 (43%) | 11 (49%) | 18 (35%) | 4 (44.4%) | 4 (36%) |
| Grade 3 | 2 (2.8%)* | 2 (4.3%)* | 1 (4.3%)* | 1 (2%)* | 0 | 1 (9%)* |
| Grade 4 | 0 | 0 | 0 | 0 | 0 | 0 |
| Grade 5 | 0 | 0 | 0 | 0 | 0 | 0 |

*Grade 3 as assessed per Lee et al., Blood, 124: 188-195, 2014 (Table 5A); Grade 1 and Grade 2 if assessed per ASTCT Grading Scale (2019) (Table 5A).

TABLE 19
Frequency and grade of CRS in Cycle 1 after
Day 1 step-up doses in Arms A, B, and C

| | Step-up Doses | | | | | | |
|---|---|---|---|---|---|---|---|
| | | 0.1 mg | 0.3 mg | 0.9 mg | 1.2 mg* | 1.8 mg | 3.6 mg |
| Grade | 0.05 mg | (n = 3) | (n = 4) | (n = 3) | (n = 9) | (n = 3) | (n = 49) |
| 1 | 0 | 1 | 0 | 0 | 3 | 1 | 22 |
| 2 | 0 | 0 | 0 | 1 | 3 | 0 | 15 |
| 3 | 0 | 0 | 0 | 0 | 0 | 0 | 2\hat{ } |
| Total | 0 | 1 (33%) | 0 | 1 (33%) | 6 (66%) | 1 (33%) | 39 (80%) |

*first step up dose in Arm B.
\hat{ }Grade 3 as assessed per Lee et al., Blood, 124: 188-195, 2014 (Table 5A) due to elevation in liver function tests (LFTs); one would be Grade 1 and one would be Grade 2 if assessed per ASTCT Grading Scale (2019) (Table 5A).

TABLE 20
Frequency and grade of CRS in Cycle 1 after Day 8 target doses in Arms A and C

| | Target Doses* | | | | | | | |
|---|---|---|---|---|---|---|---|---|
| | 0.3 mg | 2.7 mg | 5.4 mg | 20 mg | 40 mg | 60 mg | 90 mg | 132 mg |
| Grade | (n = 3) | (n = 3) | (n = 3) | (n = 3) | (n = 6) | (n = 7) | (n = 23) | (n = 7) |
| 1 | 1 | 1 | 1 | 1 | 0 | 0 | 2 | 2 |
| 2 | 0 | 1 | 0 | 0 | 3 | 1 | 4 | 0 |
| 3 | 0 | 0 | 0 | 0 | 0 | 0 | 0 | 0 |
| Total | 1 (33%) | 2 (66%) | 1 (33%) | 1 (33%) | 3 (50%) | 1 (14%) | 6 (26%) | 2 (29%) |

*Doses: 0.15 (n = 1), 0.90 (n = 4), 10.8 (n = 3) not in table as only doses with CRS were included.

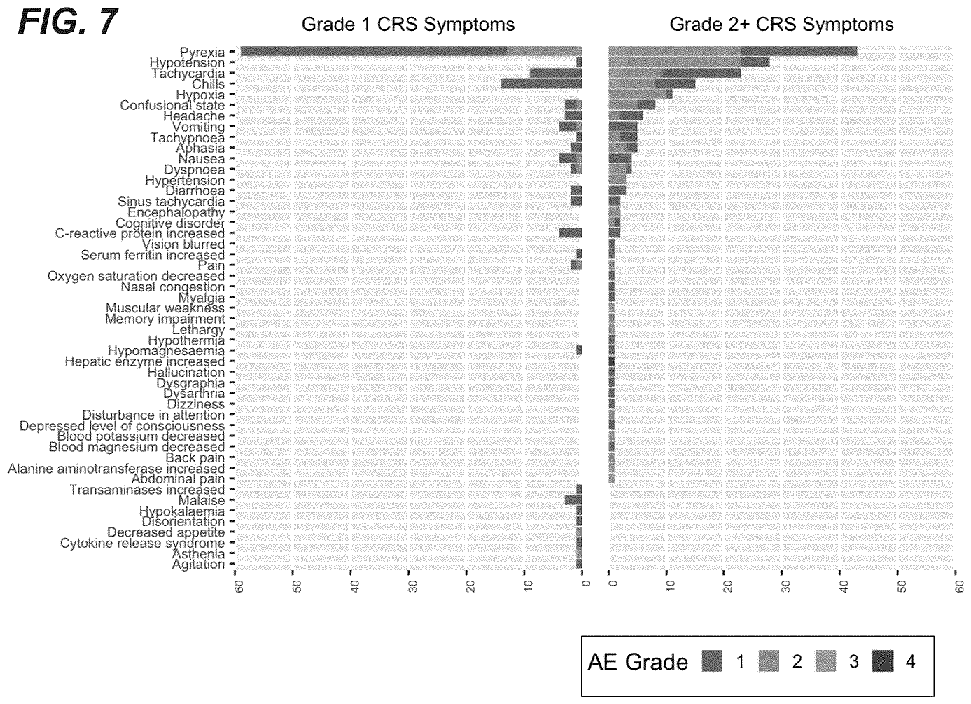

CRS was Grade 1 in 20 patients (43%), Grade 2 in 20 patients (43%), and Grade 3 in 2 patients (4.3%) (both due to transient transaminase elevation that fully resolved). Clinical symptoms of Grade 1 and Grade 2 CRS are shown in FIG. 7. Clinical symptoms of Grade 1 PRS were primarily due to fever (pyrexia) and are generally treatable with antipyretics and do not require urgent intervention. Grade 2 events do not require an intensive care unit (ICU) level of care. Management of Grade 2 hypotension was predominantly limited to administration of intravenous fluids (IVF). One patient received a single low dose vasopressor prior to receiving tocilizumab. Grade 2 hypoxia was managed with standard supplementary oxygen. No patient has required hi-flow oxygen or mechanical ventilation. No Grade 4 or Grade 5 CRS events were observed.

CRS was most common in cycle 1 (C1) (38 patients) and was uncommon or absent in subsequent cycles (4 patients). CRS was reversible with standard of care treatment, steroids, or tocilizumab if clinically warranted. Most CRS events (49/58, 84.5%) resolved within 2 days. 18 of 38 patients (47.3%) with CRS received tocilizumab and/or steroids.

Other treatment-related AEs occurring in ≥5 patients were neutropenia and lymphocyte count decreased (6 patients each, 11.8%); aspartate aminotransferase increased; and platelet count decreased (5 patients each, 9.8%). Treatment-related Grade 3-4 AEs (20 patients, 39.2%) occurring in ≥3 patients were lymphocyte count decreased (6 patients, 11.8%); neutropenia (5 patients, 9.8%); anemia; and platelet count decreased (3 patients each, 5.9%). No treatment-related Grade 5 (fatal) AEs were observed. Treatment-related AEs leading to withdrawal of treatment were uncommon (1 patient, 2.0%). One dose-limiting toxicity (DLT) was observed in the 3.6/90 mg cohort, but the maximum tolerated dose (MTD) was not reached.

In Cohort 10 (patients treated with 3.6 mg and 90 mg cevostamab on C1D1 and C1D8, respectively), a 54.5% ORR was observed (6/11 patients were responders): one patient had a partial response (PR), four patients had a very good partial response (VGPR), and one patient had a complete response (CR) (Table 17). CRS was observed in 96% of patients (22/23). CRS was Grade 1 in 10 patients (43%), Grade 2 in 11 patients (49%), and Grade 3 in 1 patient (4.3%). No significant chronic adverse events (AE) were observed in the patients identified as responders. Thus, BFCR4305A is tolerable and drives meaningful deep responses in a population with high unmet need using the 3.6/90 mg dosing regimen.

No clear dose-dependent increase in CRS was observed across dose levels. Non-CRS adverse events (AEs) occurred sporadically at different dose levels with no pattern or dose dependency. At the 90 mg dose, grade 3 (G3) pneumonia, pneumonitis (n=1) and general malaise requiring dose reduction (n=1) were observed. At the 132 mg dose, G2 foot blisters (n=1) and malaise and diarrhea requiring dose reduction (n=1) were observed.

Cevostamab PK was linear across the active dose levels tested, and the estimated half-life was supportive of the Q3W dosing regimen.

iii. Conclusions

Cevostamab monotherapy demonstrates promising activity in heavily pre-treated R/R MM, with deep and durable responses observed in patients with HR cytogenetics, triple-class refractory disease, and/or prior exposure to anti-CD38 mAbs (e.g., daratumumab), CAR-Ts, or ADCs, thereby establishing FcRH5 as a novel target in MM. Toxicity was manageable, with C1 single step-up dosing effectively mitigating the risk for severe CRS and allowing escalation to clinically active doses.

Arm A (Single-Step Dose Escalation Arm) at Second Data Cut-Off

At a second cut-off, 53 patients had been enrolled into Arm A. Baseline characteristics for these patients are provided in Table 21.

TABLE 21
Baseline characteristics for Arm A at second data cut-off

| N (%) unless stated | N = 53 | |
|---|---|---|
| Age in years, median (range) | 62 | (33-80) |
| Male | 31 | (59) |
| High-risk cytogenetics* | 28 | (53) |
| Extramedullary disease at screening | 9 | (17) |
| Time since first multiple myeloma | 5.7 | (1.2-22.8) |
| therapy in years, median (range) | | |
| Number of prior lines of therapy, | 6 | (2-15) |
| median (range) | | |
| Prior PI | 53 | (100) |
| Prior IMiD | 53 | (100) |
| Prior anti-CD38 antibody | 43 | (81) |
| Prior anti-BCMA^{‡} | 11 | (21) |
| Prior bispecific antibody | 2 | (4) |
| Prior ADC | 9 | (17) |
| Prior CAR-T | 6 | (11) |
| Prior ASCT | 47 | (89) |
| Refractory to prior PI | 50 | (94) |
| Refractory to prior IMID | 52 | (98) |
| Refractory to prior anti-CD38 antibody | 41 | (77) |
| Triple-class refractory | 38 | (72) |
| Penta-drug refractory | 24 | (45) |
| Refractory to last prior therapy | 50 | (94) |

*1q21 gain, 21/53 (40%); t(4:14), 5/53 (9%); t(14;16), 0/53; del(17p), 10/53 (19%);
*PI, proteasome inhibitor; IMiD, immunomodulatory drug; ADC, antibody-drug conjugate; CAR-T, chimeric antigen receptor T cell therapy; ASCT, autologous stem cell transplant; BCMA, B-cell maturation antigen;
^{‡}CAR-T, 6/11; ADC, 5/11.

i. Safety

Median follow-up was 8.1 months (range: 0.2-30.4). 28 patients experienced serious AEs. 13 of these patients experienced treatment-related events; for 6 of these patients, the event was CRS.

Five patients (9%) experienced AEs leading to withdrawal. For two of these patients, the AE was treatment-related. One patient experienced pneumonitis, and one patient experienced meningitis.

Seven patients (13%) experienced Grade 5 AEs, which included malignant neoplasm progression (5 patients) and respiratory failure (2 patients). No treatment-related grade 5 events were observed.

One patient (2%) experienced a DLT of Gr 3 pneumonia in the 3.6/90 mg cohort; MTD was not reached. Adverse events are summarized in Table 22.

TABLE 22
Frequency and grade of adverse events
for Arm A at second data cut-off

| | | |
|---|---|---|
| | All Gr | All Gr 3-4 |
| N (%) | (N = 53) | (N = 53) |

Hematologic AEs (≥15%)

| Anemia | 15 | (28) | 10 | (19) |
|---|---|---|---|---|
| Thrombocytopenia | 9 | (17) | 7 | (13) |
| Neutropenia | 9 | (17) | 8 | (15) |
| Platelet count decreased | 8 | (15) | 6 | (11) |
| Lymphocyte count decreased | 8 | (15) | 8 | (15) |
| Non-hematologic AEs (≥15%) | | | | |
| Cytokine release syndrome | 40 | (76) | 1 | (2) |
| Hypomagnesemia | 15 | (28) | 0 | |
| Diarrhea | 15 | (28) | 1 | (2) |
| Infusion-related reaction | 12 | (23) | 0 | |
| Hypokalemia | 11 | (21) | 2 | (4) |
| Hypophosphatemia | 10 | (19) | 5 | (9) |
| Nausea | 10 | (19) | 0 | |
| Fatigue | 9 | (17) | 2 | (4) |
| AST increased | 8 | (15) | 1 | (2) |

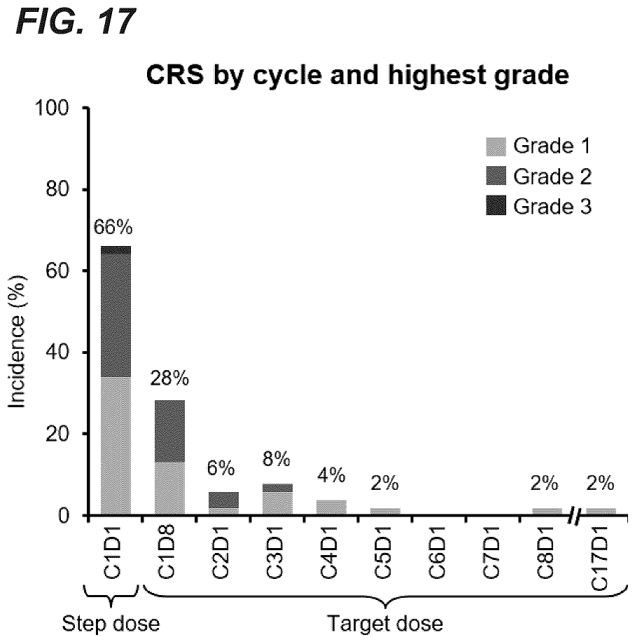

CRS events occurring in five or more patients were pyrexia (39 patients, 74%), hypotension (16 patients, 30%), tachycardia (14 patients, 26%), chills (8 patients, 15%), confusional state (7 patients, 13%), and hypoxia (5 patients, 9%). Neurological events occurring in two or more patients were confusional state (7 patients, 13%), headache (4 patients, 8%), aphasia (3 patients, 6%), and cognitive disorder (2 patients, 4%). All events occurred in the setting of CRS and resolved with CRS resolution. All CRS events resolved with standard of care (tocilizumab, 13 patients (33%); steroids, 9 patients (23%)). CRS events are summarized in Table 23 and FIG. 17.

TABLE 23
Frequency and grade of CRS events
for Arm A at second data cut-off

| N (%) unless stated | N = 53 | |
|---|---|---|
| Any CRS event* | 40 | (76) |
| Grade 1 | 18 | (34) |
| Grade 2 | 21 | (40) |
| Grade 3 | 1 | (2)^{†} |
| Median time to onset, hours (range) | 6-12 | (0-55)^{‡} |
| Any neurological event | 15 | (28) |
| Grade 1 | 10 | (19) |
| Grade 2 | 5 | (9) |
| Median time to onset, hours (range) | x-x | (x-x) |

CRS was assessed by Lee et al., Blood, 124: 188-195, 2014 criteria.
^{†}Due to transient Gr 4 transaminase elevation;
^{‡}missing CRS onset time was imputed with 23:59:59.

ii. Efficacy

51 of 53 patients were efficacy evaluable No response was observed in the ≤3.6/10.8 mg cohorts. ORR in the ≥3.6 mg/20 mg cohorts (defined as the best response of PR, VGPR, CR or sCR by IMWG Uniform Response Criteria 2016) was 53% (18/34) in all patients; 42% (7/17) in penta-drug refractory patients; and 63% (5/8) in patients with prior anti-BCMA exposure.

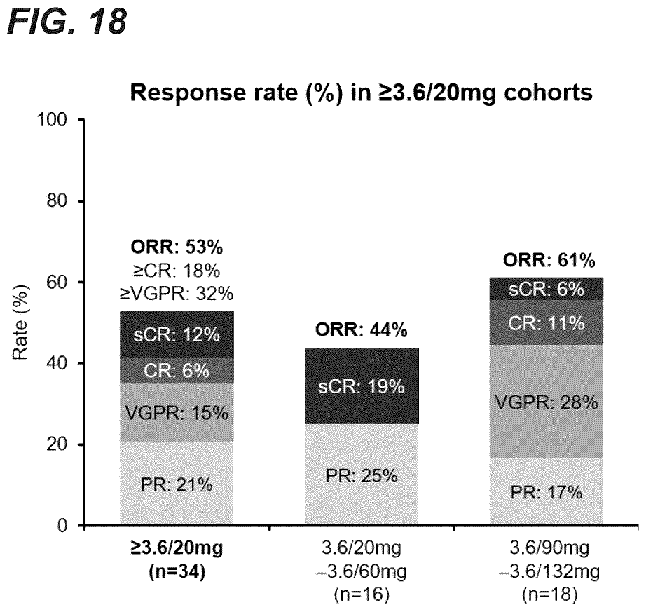

The median time to first response was 29.5 days (range: 21-105 days). The median time to best response was 57.5 days (range: 21-272). Responses were observed irrespective of level of target expression. MRD negativity by next-generation sequencing (NGS) (<10⁻⁵) in 6/7 evaluable patients with ≥VGPR. Response rates are summarized in FIG. 18.

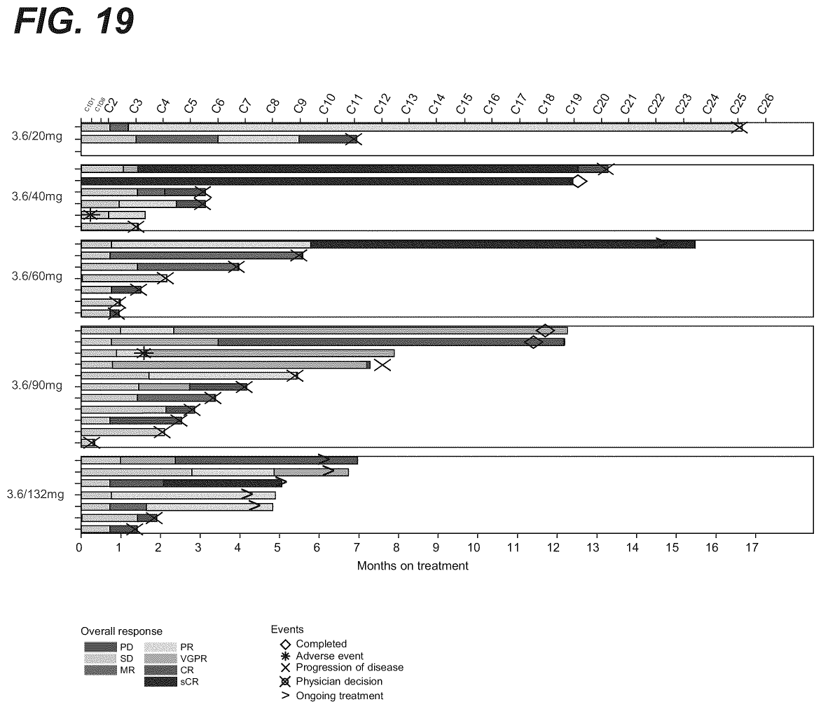

Median length of follow-up in responders was 10.3 months (range: 2.7-19.5). Eight patients had a duration of response of 6 months or longer, and four patients showed durable responses off-treatment. Two of these patients completed 17 cycles of treatment, and two discontinued treatment prematurely due to AEs (FIG. 19).

iii. Conclusions

The data collected at the second data cut-off indicate that the cevostamab safety profile is manageable, with C1 single step-up dosing effectively mitigating the risk for severe CRS. CRS in 76% of patients (40/53), but was Grade 3 in only 2% (1 patient).

Cevostamab was found to be highly active in heavily pre-treated RR/MM patients, with 53% ORR in the ≥3.6 mg/20 mg cohorts (32% with ≥VGPR); 42% ORR in penta-drug refractory patients; 63% ORR in patients with prior anti-BCMA; MRD negativity by next-generation sequencing (NGS) (<10⁻⁵) in 6/7 evaluable patients with ≥VGPR; and response irrespective of level of target expression.

Arm B (Multistep Dose Escalation Arm)

Cohort 1 of Arm B was treated with 1.2 mg, 3.6 mg, and 60 mg cevostamab on C1D1, C1D8, and C1D16, respectively, administered intravenously (IV). Cohort 2 of Arm B was treated with 1.2 mg, 3.6 mg, and 90 mg cevostamab on C1D1, C1D8, and C1D16, respectively (IV). Cohorts 3 and 4 of Arm B were treated with 0.3 or 0.6 mg, 3.6 mg, and 90 mg cevostamab on C1D1, C1D8, and C1D16, respectively (IV) (FIG. 4B). Arm D was opened as an expansion of Arm B.

i. Safety and Efficacy of 1.2 mg Double-Step Dose

Nine patients were treated with the 1.2 mg double-step dose: six patients received 1.2/3.6/60 mg, and three received 1.2/3.6/90 mg. Eight of the nine patients had CRS in the first cycle; six of those eight patients had CRS (Grade 1 or Grade 2) at the first 1.2 mg dose (FIG. 8; Table 18). Grade 2 CRS was also observed at the C1D15 target dose (FIG. 8). Double-step fractionation did not prevent patients from developing CRS at the target dose on C1D15. The severity of CRS at the 1.2 mg dose was not superior to that at the 3.6 mg dose tested in the single-step fractionation arms (Arm A and Arm C).

ii. Additional Arm B Cohorts

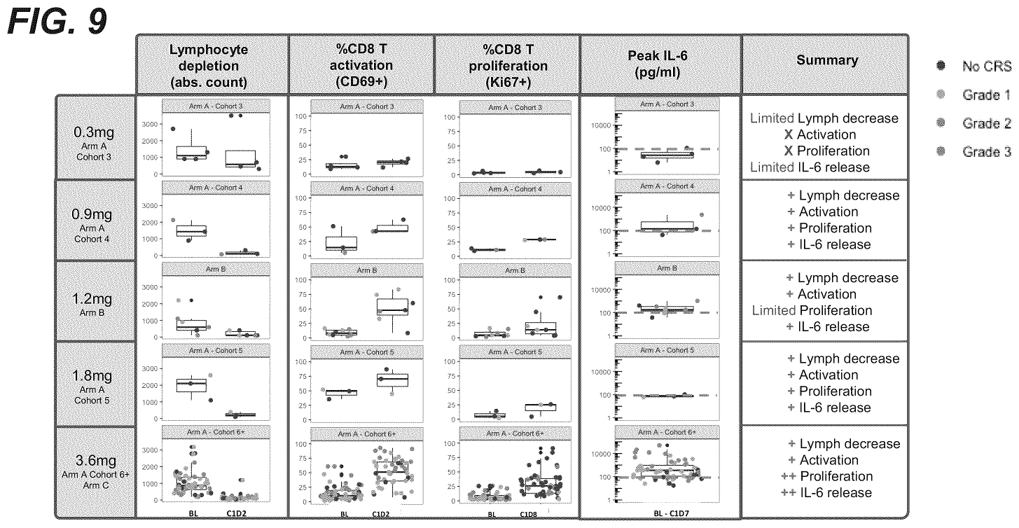

Additional cohorts 3 and 4 were opened to investigate step-up doses lower than 1.2 mg. An initial dose of 0.3 mg was selected as the lowest dose based on the observations that this dose had minimal pharmacodynamic (PD) activation, i.e., limited T-cell activation/proliferation (FIG. 9) and that some biological effect of cevostamab was observed at this dose. Initial doses of 0.05 mg and 0.15 mg were also tested.

Arm C (Single-Step Dose Expansion Arm)

Arm C was opened as an expansion of Arm A. The first cohort of Arm C was treated with 3.6 mg and 90 mg cevostamab on C1D1 and C1D8, respectively, administered intravenously (FIG. 4A). 31 patients had been enrolled at the data cut-off.

i. Safety

CRS incidence and severity in Arm C were consistent with Arms A and B (Tables 18, 19, and 24). Predictors of response analyses were performed; no significant predictors of Grade 2+ CRS were identified in multivariable analysis. No additional safety signals were observed.

TABLE 24
CRS profile in Arms A, B, and C

| | Arm A* n = 38 | | Arm B n = 9 | | Arm C n = 29 | |
|---|---|---|---|---|---|---|
| Any CRS | 34 | 89% | 7 | 78% | 26 | 90% |
| C1D1 | 32 | 84% | 6 | 67% | 24 | 83% |
| C1D8 | 8 | 21% | 2 | 22% | 8 | 28% |
| C1D15 | — | — | 5 | 56% | — | — |
| C2D1+ | 3 | 8% | 1 | 11% | 2 | 7% |

| | Arm A* n = 38 | | Arm B n = 9 | | Arm C n = 30 | |
|---|---|---|---|---|---|---|
| No CRS | 6 | 16% | 1 | 11% | 4 | 13% |
| Grade 1 | 12 | 32% | 4 | 44% | 13 | 43% |
| Grade 2 | 19 | 50% | 4 | 44% | 11 | 37% |
| Grade 3 | 1 | 3% | — | — | 2 | 7% |

*Arm A cohorts with step dose 3.6 mg
** Based on Lee et al., Blood, 124: 188-195, 2014 (Table 5A)

ii. Efficacy

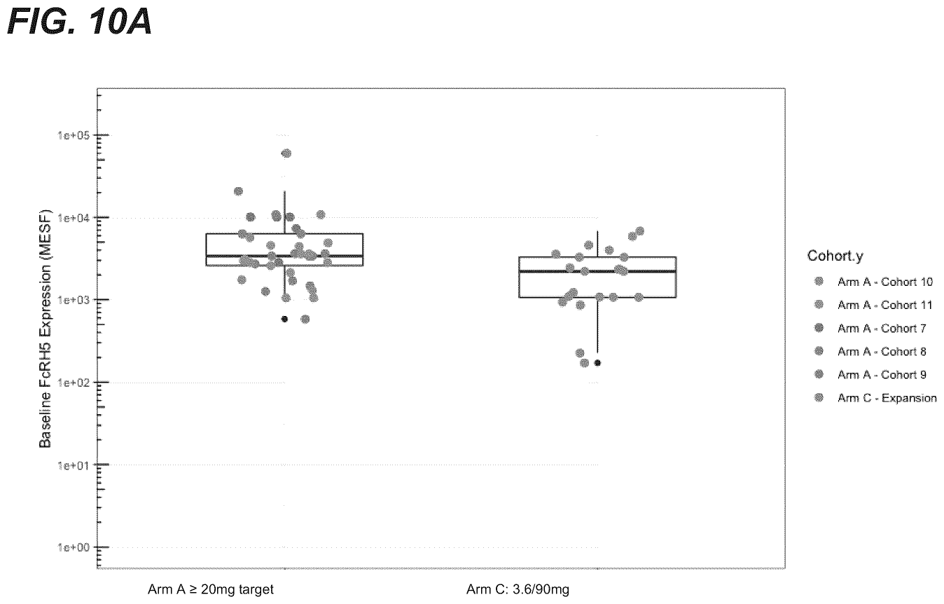

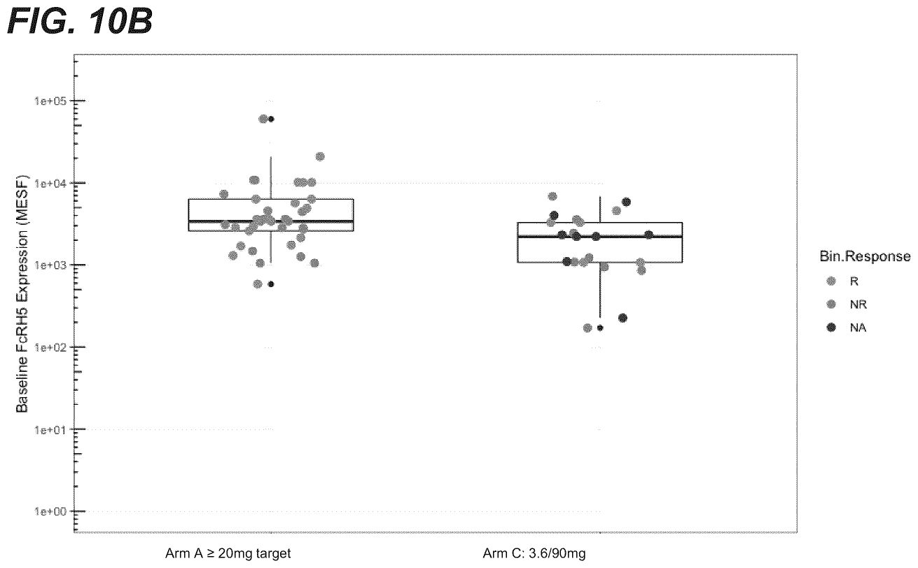

No obvious differences in demographic data between Arm A and Arm C were identified. Baseline FcRH5 expression between the two patient populations was comparable (FIGS. 10A and 10B). As in Arm A, responding patients showed deep responses, and multiple very good partial responses or complete responses (VGPR/CR) were observed. Responses were observed across the FcRH5 expression spectrum, including in patients with low expression (FIG. 10B).

Results at Third Data Cut-Off

As of a third clinical cutoff date (CCOD), cevostamab monotherapy continued to show clinically meaningful activity and manageable safety in patients with heavily pre-treated relapsed/refractory multiple myeloma (R/R MM). This example presents updated safety and efficacy data from a larger cohort of patients, including results comparing Cycle (C) 1 single step-up and double step-up dosing for the mitigation of cytokine release syndrome (CRS).

i. Methods

Cevostamab (intravenous infusion) was administered in 21-day cycles, as described above. In the single step-up cohorts, the step dose (0.05-3.6 mg) was given on C1 Day (D) 1 and the target dose (0.15-198 mg) on C1D8. In the double step-up cohorts, the step doses were given on C1D1 (0.3-1.2 mg) and C1D8 (3.6 mg) and the target dose (60-160 mg) on C1D15. In both regimens, the target dose was given on D1 of subsequent cycles. Cevostamab was continued for a total of 17 cycles, unless progressive disease or unacceptable toxicity occurred. CRS was reported using ASTCT criteria (Lee et al., Biol Blood Marrow Transplant, 25: 625-638, 2019) (Table 5A).

ii. Results

At the third data cut-off, 160 patients had been enrolled (median age: 64 years, range: 33-82 years; male: 58.1%); 21.3% of patients had extramedullary disease. The median number of prior lines of therapy was 6 (range: 2-18). Most patients (85.0%) were triple-class refractory (PI, IMiD, anti-CD38 antibody). 28 patients (17.5%) had received >1 prior CAR-T, 13 patients (8.1%) had received ≥1 prior BsAb, 27 patients (16.9%) had received ≥1 prior antibody-drug conjugate (ADC), and 54 patients (33.8%) had received ≥1 prior anti-BCMA targeting agent.

Median follow-up in exposed patients was 6.1 months. Almost all had ≥1 adverse event (Table 25). The most common adverse event was cytokine release syndrome (CRS) (128/160 patients [80.0%]; Grade [Gr] 1: 42.5%; Gr 2: 36.3%; Gr 3: 1.3%). Immune effector cell-associated neurotoxicity syndrome (ICANS) associated with CRS was observed in 21 patients (13.1%) and in 34/211 (16.1%) CRS events (Gr 1: 8.5%; Gr 2: 6.2%; Gr 3: 1.4%). Most CRS events occurred in Cycle 1 (87.2%), arose within 24 hours of cevostamab administration (70.5%), and resolved within 48 hours of onset (83.4%). In the patients with CRS, tocilizumab was used for CRS management in 43.8% of patients and steroids in 25.8% of patients (both agents were used in 18.0% of patients). In single step-up dose-escalation (68 patients), 3.6 mg was chosen as the most effective C1D1 single step-up dose for limiting CRS in Cycle 1, with no target dose-dependent increase in the rate or severity of CRS observed after the C1D8 administration. Likewise, in double step-up dose-escalation (30 patients), 0.3/3.6 mg was identified as the preferred C1D1/C1D8 DS dose for limiting CRS in Cycle 1. Notably, the overall rate of CRS was lower in the patients who received the 0.3/3.6 mg/target double step-up regimen than in those who received the 3.6 mg/target single step-up regimen (77.3% [34/44] vs. 88.2% [75/85], respectively). The rate of ICANS associated with CRS was also lower in the 0.3/3.6 mg/target double step-up cohort than in the 3.6 mg/target SS cohort (4.5% [2/44] vs. 21.2% [18/85], respectively).

TABLE 25
Adverse event summary

| | Any AE | | Any Gr 3-4 AE | |
|---|---|---|---|---|
| N (%) of patients | (N = 160) | | (N = 160) | |
| Any AE* | 159 | (99.4) | 94 | (58.8) |
| Cytokine release syndrome | 128 | (80.0) | 2 | (1.3)^{‡} |
| Infections (SOC) | 68 | (42.5) | 30 | (18.8) |
| Neurological/Psychiatric (SOC) | 65 | (40.6) | 6 | (3.8)^{‡} |
| Anemia | 51 | (31.9) | 35 | (21.9)^{‡} |
| Diarrhea | 42 | (26.3) | 1 | (0.6)^{‡} |
| Cough | 37 | (23.1) | 0 | |
| Nausea | 35 | (21.9) | 0 | |
| Neutropenia | 29 | (18.1) | 26 | (16.3) |
| Infusion-related reaction | 28 | (17.5) | 0 | |
| Fatigue | 26 | (16.3) | 3 | (1.9)^{‡} |
| Aspartate aminotransferase increased | 25 | (15.6) | 10 | (6.3) |
| Hypomagnesaemia | 25 | (15.6) | 1 | (0.6)^{‡} |
| Pyrexia | 25 | (15.6) | 0 | |
| Neutrophil count decreased | 24 | (15.0) | 22 | (13.8) |
| Alanine aminotransferase increased | 24 | (15.0) | 11 | (6.9)^{‡} |
| Any serious AE | 89 | (55.6) | | |
| Any TR serious AE^{†} | 40 | (25.0) | | |
| Any Gr 5 (fatal) AE | 24 | (15.0)** | | |
| Any TR Gr 5 (fatal) AE^{†} | 1 | (0.6)^{††} | | |
| Any AE leading to withdrawal | 16 | (10.0) | | |
| of cevostamab | | | | |
| Any TR AE leading to withdrawal | 7 | (4.4) | | |
| of cevostamab^{†} | | | | |

*listed preferred terms are those with ≥15% incidence;
^{†}AE considered related to cevostamab by the investigator;
^{‡}Gr 3 only;
**acute kidney injury, n = 1; hemophagocytic lymphohistiocytosis, n = 1; malignant neoplasm progression, n = 17; plasma cell myeloma, n = 1; progressive disease, n = 1; respiratory failure, n = 3;
^{††}hemophagocytic lymphohistiocytosis, n = 1.
AE, adverse event; SOC, System Organ Class; TR, treatment-related.

At the data cut-off, 158/160 patients were efficacy evaluable. In dose-escalation, responses were observed at the 20-198 mg target dose levels, and data suggested a target dose-dependent increase in clinical efficacy. Median time to response was 29 days (range: 20-179 days). Two dose-expansion cohorts were opened: overall response rate (ORR) was higher at the 160 mg dose level (54.5%, 24/44 patients) than at the 90 mg dose level (36.7%, 22/60). At target dose levels >90 mg, ORRs in patients with prior exposure to CAR-Ts, BsAbs, ADCs, and anti-BCMA targeting agents were 44.4% (4/9 patients), 33.3% (3/9 patients), 50.0% (7/14 patients), and 36.4% (8/22 patients) respectively. Median follow-up among all responders (n=61) was 8.1 months; estimated median duration of response was 15.6 months (95% CI: 6.4, 21.6).

iii. Conclusions

Cevostamab monotherapy continues to show clinically meaningful activity in a large cohort of patients with heavily pre-treated R/R MM, with a target dose-dependent increase in ORR, but no increase in CRS rate. Responses appear durable, and are observed in patients with prior exposure to CAR-Ts, BsAbs, and ADCs. Compared with single step-up dosing, double step-up dosing at the 0.3/3.6 mg level appears to be associated with a trend for an improved C1 safety profile.

### Example 7. Tocilizumab for Treatment of CRS

Tocilizumab was found to be highly effective at treating FcRH5 TDB-mediated CRS. At the data cutoff used in this Example, of the 82 patients in the GO39775 trial who had CRS (data includes Arm A cohorts with 3.6 mg step doses, Arm B, and Arm C), 25 patients received tocilizumab to treat the CRS. 5 of these patients had Grade 1 CRS, 17 had Grade 2 CRS, and 3 had Grade 3 CRS. In 19 patients, CRS symptoms resolved within 1 day, and in 5 patients, symptoms resolved within 3 days. 24 of the 25 patients continued with cevostamab dosing in the next cycle. Early intervention with tocilizumab helps to limit progression of CRS to higher grades (e.g., Grade 3 or higher).

67 patients in Arm A or Arm C were administered a 3.6 mg dose on C1D1. 56 (84%) patients had CRS at C1D1: 45% were Grade 1 (n=30), 36% were Grade 2 (n=24), and 4% were Grade 3 (n=3). 16 (24%) patients had CRS at C1D8: 10% G1 (n=7), 13% G2 (n=9). 3 patients did not have CRS on C1D1; 3 patients did not have C1D8 dose due to withdrawal and PD; 2 patients had repeat dose of 3.6 mg on C1D8 and no additional CRS after C1D1. The decreased incidence of CRS on C1D8 suggests that step dose C1D1 is mitigating CRS (Tables 18, 19, and 24).

Most cases of CRS resolved with one dose of tocilizumab. As of the second data cut-off, only four patients have required two doses within 24 hours. No patients have required more than 2 doses to treat a CRS event.

A single dose of tocilizumab is not expected to have high impact on safety profile. A risk of neutropenia was identified with chronic administration of tocilizumab. In cycle 1, a subset of patients developed neutropenia that was transient, reversible, and responsive to growth factor support. No signal of more severe or persistent neutropenia has been observed in GO39775 patients who received tocilizumab as compared to patients who did not receive tocilizumab.

Preliminary data suggest no difference in response rates in patients who have received tocilizumab as compared to patients who have not received tocilizumab. 9/22 patients who received tocilizumab to treat CRS (all arms; efficacy evaluable) had a response.

### Example 8. Arm E: Tocilizumab Prophylaxis Arm

Arm E is a dose-expansion arm to investigate the use of tocilizumab pretreatment to potentially mitigate the frequency and/or severity of CRS events associated with cevostamab treatment, based on emergent clinical data from Arms A, B, and C.

Approximately 30 patients are enrolled into Arm E at the single-step cevostamab dose regimen of 3.6 mg/90 mg. Cevostamab dosing is performed as described above, and existing steroid premedication during C1 is continued as described above. All patients in Arm E will receive a single dose of 8 mg/kg tocilizumab IV (maximum 800 mg) 2 hours prior to the Cycle 1 Day 1 dose of cevostamab as premedication. Patients who weigh less than 30 kg will receive a dose of 12 mg/kg.

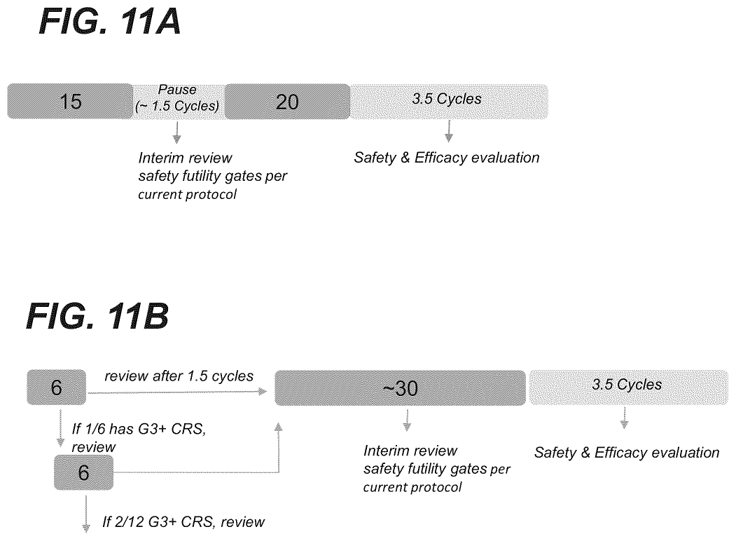

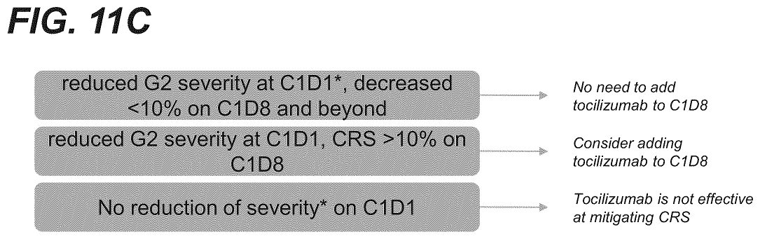

If the initial data demonstrate an acceptable safety and efficacy profile in mitigating CRS on C1D1 but patients are experiencing CRS in subsequent doses, then an additional dose of 8 mg/kg tocilizumab (maximum 800 mg) may be instituted as premedication for subsequent Cycle 1 dose(s) of cevostamab (FIG. 11C). Additionally, based on emerging data from Arm E, tocilizumab premedication may be instituted for Cycle 1 cevostamab doses for other treatment arms. Administration prior to the C1D1 dose may additionally mitigate CRS at the C1D8 dose, as the receptor occupancy (RO) for 8 mg/kg tocilizumab dose was >99% following the first dose in RA patients over a 4-week dosing interval (Xu et al., Arthritis Rheumatol, 71 (suppl. 10), 2019).

Enrollment of the first 3 patients in Arm E is staggered such that the respective C1D1 treatments are administered ≥72 hours apart. Initially, a 6+6 safety run-in is tested (FIG. 11B). Safety signals (e.g., worsening of CRS profile (Grade 3+ CRS) or overlapping toxicities) are assessed, and the arm may be expanded to enroll about 30 patients. Alternatively, an experimental design including a pause for safety review is used (FIG. 11A).

Breakthrough CRS is managed per existing protocols. Additional institutional management guidelines (e.g., guidelines for tocilizumab refractory CRS) may be used if CRS does not resolve.

For all patients, tocilizumab should be administered when indicated as described in the protocol for CRS that occurs following the cevostamab dose.

The primary study objectives are assessment of Grade 2+ CRS incidence in patients treated with tocilizumab prophylaxis with cevostamab and assessment of the safety profile of tocilizumab prophylaxis with cevostamab. The impact of tocilizumab prophylaxis on efficacy (e.g., ORR, DoR) is also assessed.

Exploratory biomarkers (e.g., PK/PD relationship with IL6, sIL6R, and PD biomarkers (e.g., lymphocyte transient decrease, T cell activation and proliferation)) are assessed. Biomarker sampling timepoints are as described above; minor adjustments may be made. An additional measurement of IL6 and other cytokine levels is taken before tocilizumab infusion. Additional flow cytometry measurements are taken before tocilizumab infusion, at C2D2 and at C3D1.

### Example 9. Assessment of Biomarkers

The Phase I dose-escalation study (GO39775; NCT03275103) investigated the pharmacodynamics (PD) of cevostamab monotherapy in patients with relapsed/refractory (R/R) multiple myeloma (MM). Early PD changes in T-cell activation, proliferation, and cytokine production were detected and confirm the mode of action of cevostamab, support Cycle 1 (C1) C1 step-up dosing for CRS mitigation in R/R MM, and offer insight into markers that may predict response. The data show that at the end of C1, higher peripheral CD8⁺ T-cell expansion and TILs may be observed in responding patients than in non-responding patients.

i. Assessment Methods and Patient Population

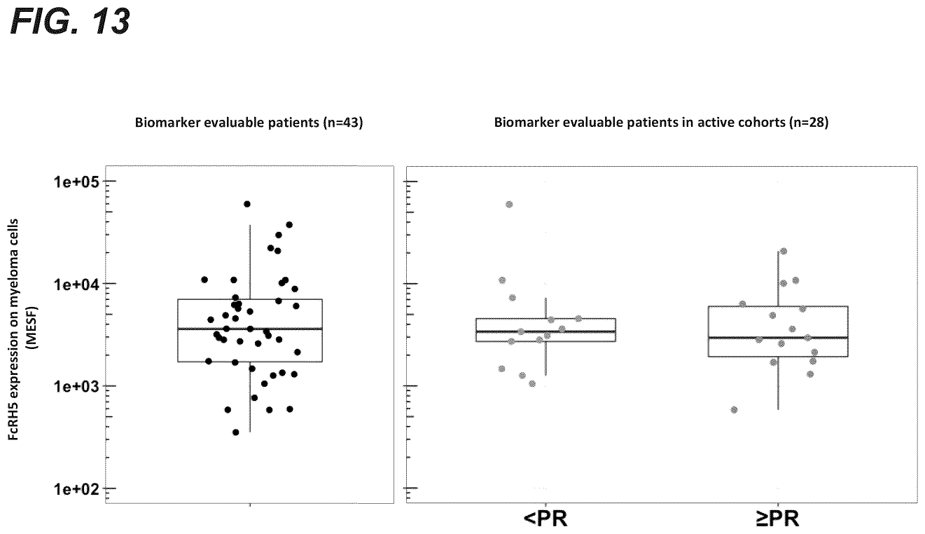

Pharmacodynamic changes in peripheral blood were assessed at baseline and at multiple timepoints within C1 by whole blood flow cytometry, plasma cytokine electrochemiluminescence, and digital ELISA. Tumor biomarkers were assessed at baseline at before Cycle 2 (C2) by bone marrow biopsy dual CD138/CD8 immunohistochemistry staining and bone marrow aspirate flow cytometry. At the cut-off date used in this Example, 43 of 53 patients (Examples 1-4 and 6) were biomarker evaluable, including up to 33 patients treated at clinically active doses (at or above doses of 3.6 mg on Cycle 1, Day 1 and 20 mg on Cycle 1, Day 8). FcRH5 expression on myeloma cells was detected in all biomarker-evaluable patients (FIG. 13). A wide range of FcH5 expression levels was detected by flow cytometry on myeloma cells in bone marrow aspirates. Data suggest that response to cevostamab was observed irrespective of FcRH5 levels in patients.

ii. T-Cell Activation and Proliferation

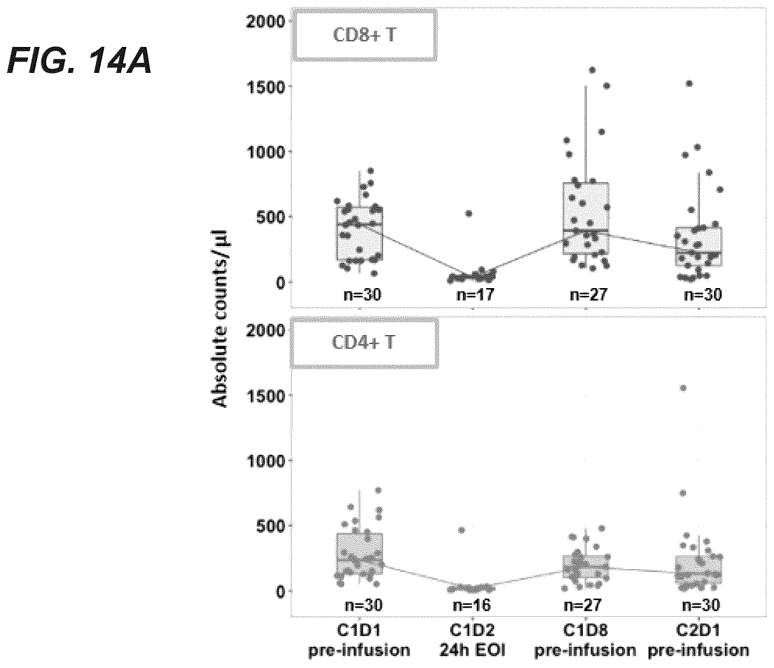

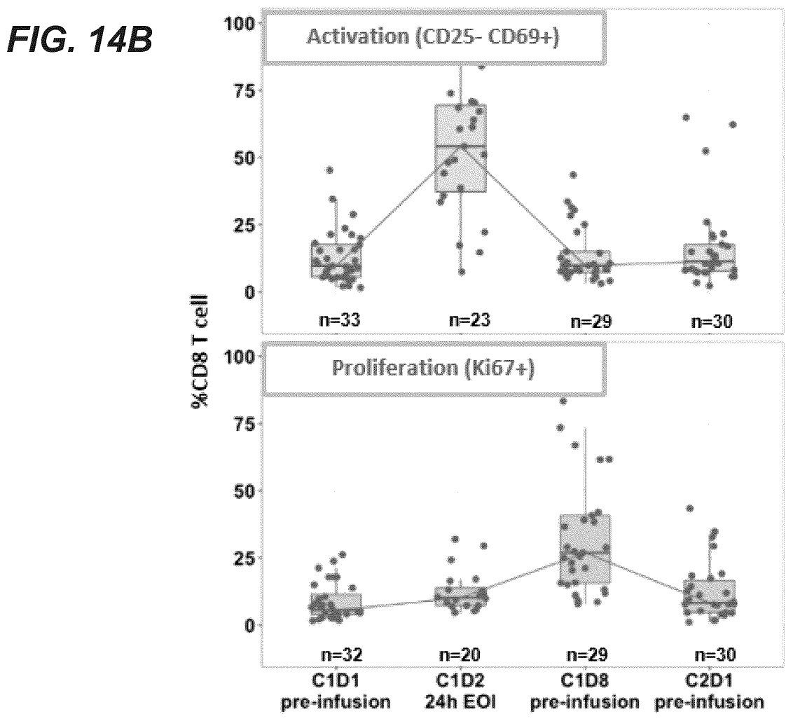

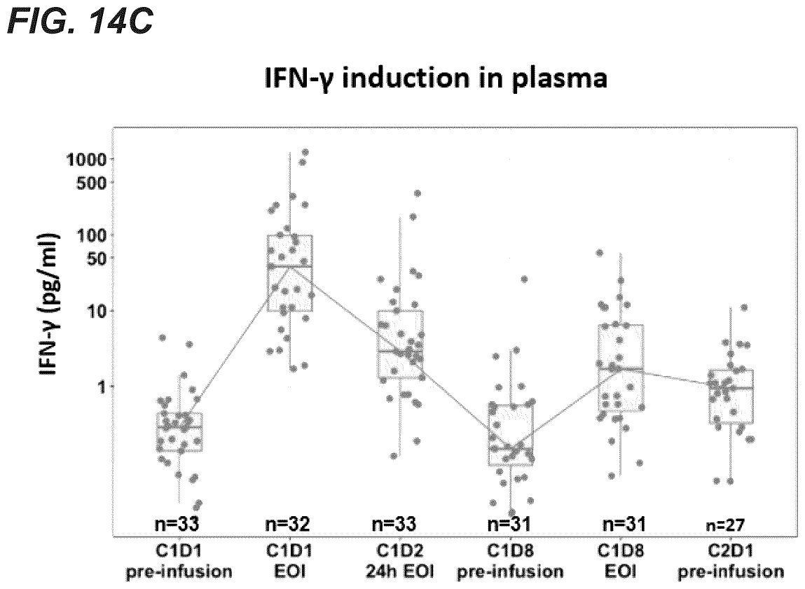

Transient T-cell reduction was observed in peripheral blood (PB) at 24 hours after the end of the C1D1 infusion, with recovery by C1D8 (FIG. 14A). Dose-dependent PD changes in peripheral blood were observed 24-192 hours after the C1D1 infusion. Variable reduction in circulating T cells was observed 24 hours after the 0.3-1.8 mg C1D1 doses, while robust reduction was observed after the 3.6 mg C1D1 dose, with recovery by C1D8 (192 hours). T-cell activation was detected 24 hours post-infusion by upregulation of CD69 in CD8+ and CD4+ T cells (FIG. 14B) and elevation of IFN-γ in plasma (median increase of about 150-fold from baseline) (FIG. 14C), while T-cell proliferation (Ki67+) peaked by C1D8. At the 3.6 mg C1D1 dose, CD8+ T-cell activation and proliferation were up to 20-fold higher than at baseline (FIG. 14B). IFN-γ induction was detected at end of infusion (EOI) on C1D1 and C1D8, with the C1D1 elevation being greater than the C1D8 (target dose) elevation (FIG. 14C).

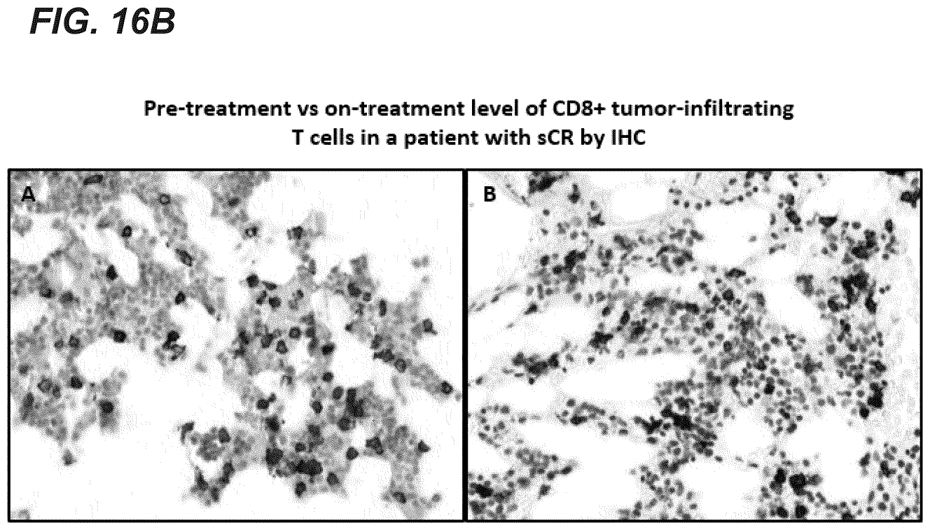

Data indicate that patients who respond to cevostamab may have more pronounced T-cell expansion in peripheral blood by the end of Cycle 1, irrespective of baseline CD8+ T-cell levels during the first week of C1 (FIG. 16A).

Analysis of the subset of patients with paired bone marrow biopsies (n=19 patients) revealed that levels of CD8⁺ tumor infiltrating T-cells (TILs) were higher on treatment (timepoints between Cycle 1, Day 9 and Cycle 1, Day 21) in responding patients compared to non-responding patients (FIGS. 16A and 16B).

iii. Cytokine Production

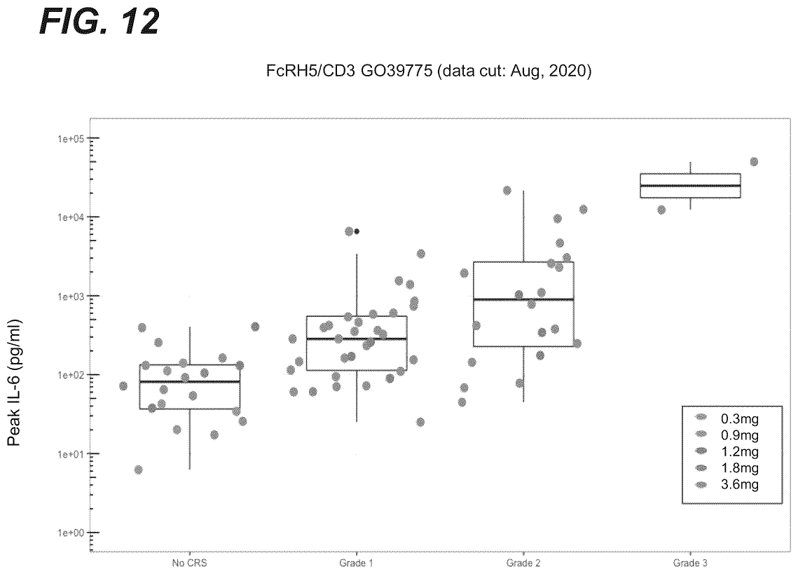

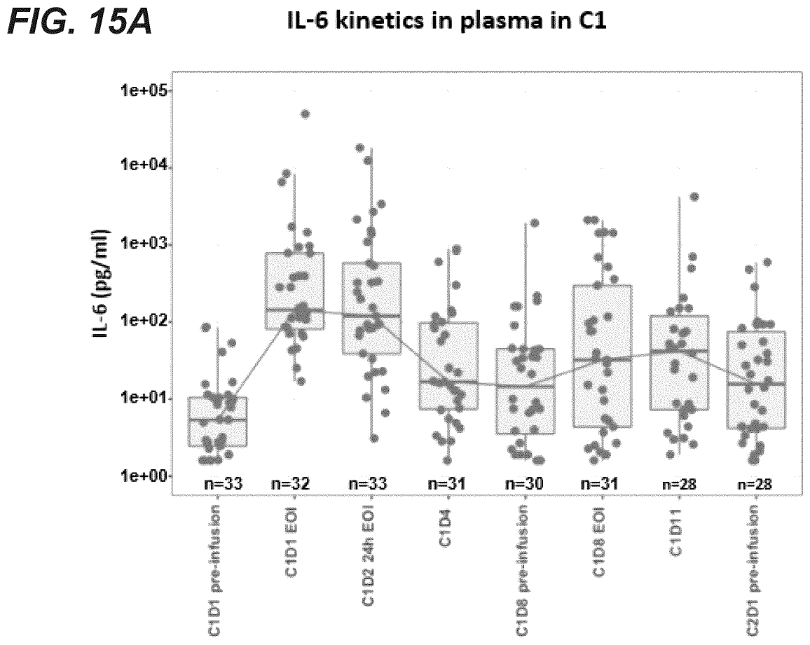

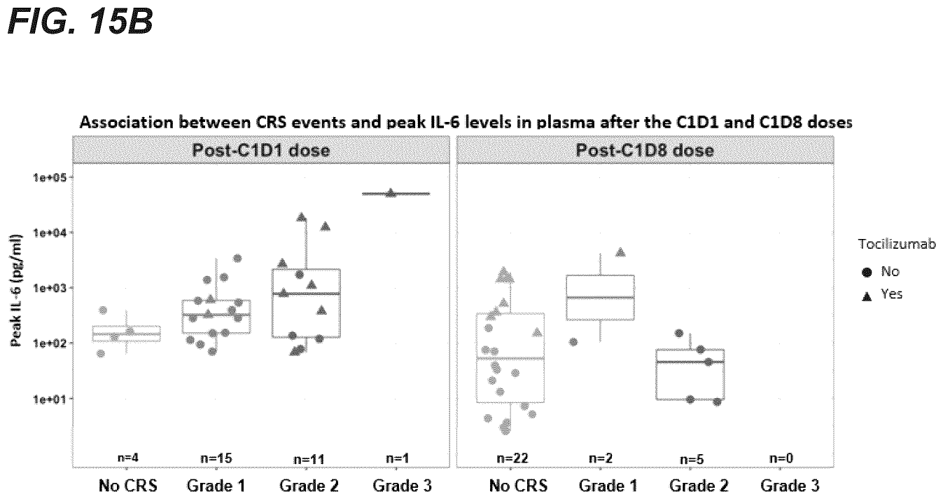

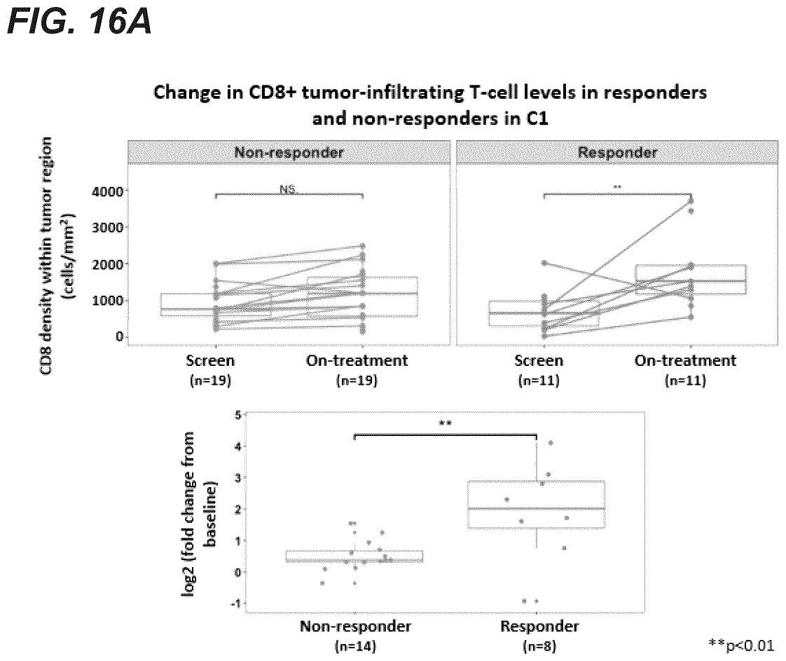

Minimal elevation of IL-6 was observed post-infusion in patients who received sub-efficacious doses, while more consistent increases (≥100 μg/ml) were observed at clinically active doses (3.6 mg/20 mg dose and above). At clinically active doses, IL-6 elevation was detected at EOI on C1D1 and C1D8, with the C1D1 elevation being greater than the C1D8 (target dose) elevation (FIG. 15A). IL-6 levels peaked within 24 hours of the C1D1 dose, and the kinetics of IL-6 increase were associated with the onset of risk for CRS at the C1D1 3.6 mg step-up dose, but not at the C1D8 target dose (20-132 mg) (FIGS. 12 and 15B). Patients who received tocilizumab as a part of CRS treatment are indicated in FIG. 15B. Tocilizumab has previously been shown to increase soluble IL-6 in plasma due to the formation of tocilizumab-soluble IL-R complexes (Nishimoto et al., Blood, 112: 3959-394, 2008). Step-up dosing mitigated the risk for severe CRS, as evidenced by lower IL-6 levels after the C1D8 target dose compared to the 3.6 mg C1D1 step dose in 27/33 patients (82%) (see Example 6). No Grade 3 CRS events were observed at the C1D8 dose, despite an up to 36-fold increase in dose relative to the C1D1 dose.

iv. Pharmacokinetics

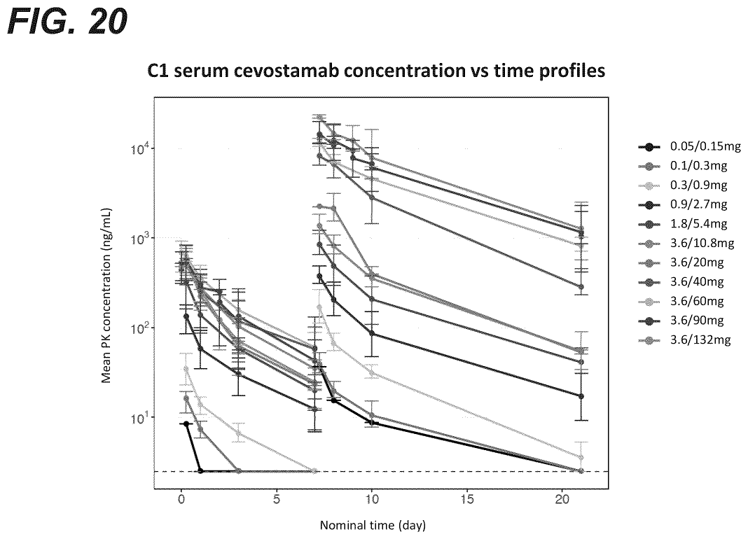

PK behavior of cevostamab was supportive of the Q3W dosing regimen. Serum concentrations of cevostamab peaked after infusion and declined in a multi-exponential fashion (FIG. 20). A generally linear increase in exposure (Cₘₐₓ and AUC) with increasing dose across the range 0.9/2.7 mg to 3.6/132 mg was observed. There was evidence of target-mediated drug disposition leading to rapid clearance at lower dose levels (0.05/0.15 mg-0.3/0.9 mg).

### Example 10. Additional Formulations

i. Overview

During clinical development, additional formulations and vial configurations of cevostamab are used, as outlined in Tables 26 and 27. Nominal content of formulation components for each vial configuration of cevostamab are provided in Table 26.

TABLE 26
Overview of Cevostamab Drug Product Formulation Development

Concentration

| | 20 mg/mL | 3mg/mL |
|---|---|---|
| Description | 40 mg/vial | 90 mg/vial |
| Cevostamab (mg) | 40 | 90.0 |
| L-Histidine (mg) | 6.21 | 93.0 |
| Glacial Acetic Acid (mg) | 1.56 | 24.0 |
| Sucrose (mg) | 164 | 2466 |
| Polysorbate 20 (mg) | 0.40 | 9.00 |
| L-Methionine (mg) | 1.49 | 44.7 |
| N-Acetyl-DL-Tryptophan | 0.148 | 2.22 |
| (mg) | | |
| Water for Injection (mL) | QS to 2.00 | QS to 30.0 |
| Primary Packaging | 6 ml vials | 50 ml vials |
| Configuration | | |

TABLE 27
Overview of Cevostamab Drug Product Configurations

| | 20 mg/mL | 20 mg/mL | 3 mg/mL | 3 mg/mL |
|---|---|---|---|---|
| Component | Toxicology | Clinical | Clinical | Clinical |
| Vial | 6 mL Type I Glass | 6 mL Type I Glass | 2 mL Type I Glass | 50 mL Type I Glass |
| Stopper | 20 mm butyl | 20 mm butyl rubber | 13 mm butyl rubber | 20 mm butyl rubber |
| | rubber fluororesin | fluororesin | fluororesin | fluororesin |
| | laminated, serum- | laminated, serum- | laminated, serum- | laminated, serum- |
| | type, | type, | type, | type, |
| | USP/Ph. Eur. | USP/Ph. Eur. | USP/Ph. Eur. | USP/Ph. Eur. |
| Cap | 20 mm aluminum | 20 mm aluminum | 13 mm aluminum | 20 mm aluminum |
| | seal with plastic | seal with plastic flip- | seal with plastic flip- | seal with plastic flip- |
| | flip-off cap | off cap | off cap | off cap |
| Fill Volume | 2 mL | 2 mL | 0.4 mL | 20 L |

ii. Components of the Drug Product

Drug Substance

Cevostamab is the only active ingredient in the drug product. The drug substance manufacturing process, testing procedures, and release criteria used to control the drug substance are given in the corresponding drug substance sections. The drug product cevostamab, polysorbate 20, and methionine concentrations in the drug substance are altered during drug product manufacturing through a dilution step. No incompatibility exists between the excipients in the formulation and the active drug, as demonstrated by the drug substance and drug product stability data.

Excipients

The 3 mg/mL and 20 mg/mL drug products are formulated with the same buffer and excipients at a target pH of 5.8. The 3 mg/mL drug product is formulated with a greater amount of polysorbate 20 and methionine than in the 20 mg/mL drug product, as described below. The rationale for all formulation excipients is listed below and is the same for both 3 mg/mL and 20 mg/mL formulations.

L-Histidine/Glacial Acetic Acid [5.8]
Function: Buffer to maintain solution pH at 5.8.
Concentration: 20 mM in drug substance and drug product.

L-histidine provides buffering capacity at target pH 5.8. A L-histidine concentration of 20 mM was shown to be sufficient to maintain the formulation pH through the manufacturing of the drug product, as well as during storage of the drug substance and drug product.

The total concentration of the buffering system (histidine acetate) is 20 mM.

Sucrose
Function: Tonicity agent.
Concentration: 240 mM in drug substance and drug product.

A sucrose concentration of 240 mM is sufficient to achieve isotonicity and provide stability for the drug substance and drug product.

Polysorbate 20
Function: Surfactant to prevent losses due to surface adsorption as well as to minimize the potential formation of soluble aggregates and/or insoluble proteinaceous particles.
Concentration: 0.2 mg/mL in drug substance and 1.2 mg/mL in drug product.

A polysorbate 20 level of 0.2 mg/mL in the drug substance and 1.2 mg/mL in the drug product was shown to be sufficient to protect cevostamab against stresses that may occur during processing (e.g., freezing and thawing), handling, and storage and in-use administration.

L-Methionine
Function: Stabilizer.
Concentration: 5 mM L-methionine in the drug substance and 10 mM L-methionine in drug product.

An L-methionine drug substance concentration of 5 mM and drug product concentration of 10 mM are sufficient to provide stability for the cevostamab drug substance and drug product.

N-Acetyl-DL-Tryptophan
Function: Anti-oxidant.
Concentration: 0.3 mM N-acetyl-DL-tryptophan in drug substance and drug product.

An N-acetyl-DL-tryptophan concentration of 0.3 mM is sufficient to provide stability for the cevostamab drug substance and drug product.

iii. Drug Product

Formulation Development

A single-dose formulation designed as a solution for intravenous (IV) infusion or subcutaneous (SC) injection was developed for the initiation of Phase 1 cevostamab clinical trials. The drug substance and drug product were composed of 50 mg/mL and 20 mg/mL cevostamab, respectively, in 20 mM L-histidine acetate, 240 mM sucrose, 5 mM L-methionine, 0.3 mM N-acetyl-DL-tryptophan, 0.2 mg/mL polysorbate 20, pH 5.8. The protein concentration in drug product differs from that of drug substance due to a dilution step during drug product manufacturing.

A 3 mg/mL drug product formulation was developed to enable delivery of a wider dose range expected during subsequent clinical trials as an IV infusion. This drug product formulation is composed of 3 mg/mL cevostamab in 20 mM L-histidine acetate, 240 mM sucrose, 10 mM L-methionine, 0.3 mM N-acetyl-DL-tryptophan, and 1.2 mg/mL polysorbate 20, pH 5.8. The formulation differs from drug substance due to a dilution step, which alters protein, L-methionine and polysorbate 20 concentrations during dilution to drug product. The drug substance composition was not altered during the development of the 3 mg/mL drug product.

All the excipients and excipient concentrations of the 20 mg/mL and 3 mg/mL formulations are the same, with the exception of the polysorbate 20 and L-methionine. The surfactant concentration was determined based on studies designed to determine stability of diluted drug product into saline-containing IV bags. Based on the results of this study, 1.2 mg/mL polysorbate 20 was found to be sufficient to ensure drug product stability and was therefore selected for the 3 mg/mL drug product formulation. L-methionine was added to the drug product formulation as a stabilizer. Formulation development studies demonstrated that 10 mM L-methionine was sufficient to ensure stability of the 3 mg/mL drug product formulation. Formulation studies were performed to demonstrate acceptable stability in the 3 mg/mL cevostamab drug product.

Based on the results from these formulation development studies, a liquid formulation consisting of 3 mg/mL cevostamab in 20 mM histidine acetate, 240 mM sucrose, 10 mM L-methionine, 0.3 mM N-Acetyl-DL-Tryptophan, 1.2 mg/mL polysorbate 20, with a target pH of 5.8 was selected as the drug product formulation.

For the initiation of the clinical studies, cevostamab 40 mg/vial (20 mg/mL) was used. Current patients are transitioned to and new patients begin using the newly developed 1.2 mg/vial and 60 mg/vial drug product.

Physicochemical and Biological Properties

All characterization testing was performed on the drug substance. Extended characterization of drug product lots are provided in Table 28 below.

TABLE 28
Extended Characterization of Cevostamab Drug Product Batches

| Analytical Procedure | Batch 1 | Batch 2 |
|---|---|---|
| Mass Spectrometry-Based | <0.5 | <0.5 |
| Anti-CD3 Homodimer (%) | | |
| T-Cell Activation Assay (%) | <0.5 | <0.5 |

The formulation remains stable at the recommended storage conditions of 2° C.-8° C. when protected from light.

There was no increase in visible or subvisible particles (≥10 μm and ≥25 μm) at the recommended storage temperature (2° C.-8° C.), as shown by the 3 mg/mL drug product representative stability study.

Subvisible particles ≥2 μm and ≥5 μm in size (in addition to ≥10 μm and ≥25 μm, which are part of the control system) are monitored using the light-obscuration method through development. These evaluations are conducted as part of extended characterization performed at the time of drug product release and during stability.

Intravenous (IV)

An in-line filter (0.2 μm) is used for administration of clinical material at this stage of development, as a measure of precaution.

iv. Manufacturing Process Development

Changes in the drug product manufacturing process are highlighted in Table 29. No changes have been made to the manufacturing process of the drug substance. The drug product manufacturing process for BFCR350A is a standard, aseptic manufacturing procedure. For the 40 mg/vial drug product (DP), thawed drug substance is diluted with formulation buffer to 20 mg/mL followed by processing through bioburden reduction and sterile filtration steps. Next, 2 mL of diluted solution is filled into 6-mL glass vials, stoppered, capped, labeled and packaged. For the 1.2 mg/vial and 60 mg/vial DP, thawed drug substance is diluted to 3 mg/mL with a dilution buffer, followed by processing through bioburden reduction and sterile filtration steps. Next, 0.4 mL of diluted solution is filled into 2-mL glass vials or 20 mL diluted solution is filled into 50-mL vials. Vials are then stoppered, capped, labeled, and packaged.

TABLE 29
Comparison of the Manufacturing Process
of BFCR430A 20 mg/mL DP and 3 mg/mL DP

| Process | | |
|---|---|---|
| Step | Cevostamab Solution for Injection in Vial | Cevostamab Solution for Injection in Vial |
| 1 | Thaw the drug substance solution | Thaw the drug substance solution |
| 2 | Prepare buffer solution | Prepare buffer solution |
| 3 | Mix drug substance solution with buffer | Mix drug substance solution with buffer |
| | solution to obtain a bulk drug product | solution to obtain a bulk drug product |
| | solution with a concentration of | solution with a concentration of 3 mg/ml |
| | 20 mg/mL cevostamab | cevostamab |
| 4 | Bioburden reduction filtration and sterile | Bioburden reduction filtration and sterile |
| | filtration (in-line filtration) | filtration (in-line filtration) |
| 5 | Aseptically fill into 6-ml vials | Aseptically fill into 2-mL or 50-mL vials |
| 6 | Close vials with stoppers | Close vials with stoppers |
| 7 | Cap with aluminum seal | Cap with aluminum seal |

### Example 11. FcRH5 Target Expression in Patients with Relapsed/Refractory (R/R) Multiple Myeloma (MM) Treated with Cevostamab in an Ongoing Phase I Dose Escalation Study

Cevostamab showed promising activity and manageable toxicity as monotherapy in the ongoing cevostamab Phase 1 dose escalation study (GO39775) enrolling late-line R/R MM patients (Examples 1-8). Responses were observed in patients with prior exposure to standard and emerging therapies (including anti-BCMA) and high-risk cytogenetics.

In this Example, the relationships between baseline (pre-treatment) FcRH5 expression and baseline patient and disease characteristics (e.g., prior therapy, cytogenetic risk status), and between baseline FcRH5 expression and response to cevostamab monotherapy, are explored in GO39775.

Bone marrow aspirates (BMA) were collected prior to cevostamab treatment. FcRH5 cell surface expression on myeloma cells was assessed using flow cytometry and the levels of expression (reported as molecules of equivalent soluble fluorochrome (MESF)) were compared between patients by prior therapy and stratified by cytogenetic risk status (determined by fluorescence in situ hybridization (FISH)).

As of the CCOD used in this Example, 53 patients (median age: 62.0 years; range: 33-80) had enrolled in the study. The median number of prior lines of therapy was 6 (range: 2-15). Prior treatments included proteasome inhibitors (PI) (100%; 94.1% refractory); immunomodulatory drugs (IMiD) (100%; 98.0% refractory); anti-CD38 mAbs (81%; 92% refractory); and autologous stem cell transplant (86%). Overall, 72% of patients were triple-class refractory (≥1 PI, ≥1 IMiD, and ≥1 anti-CD38 mAb), and 94% of patients were refractory to their last therapy (Table 21).

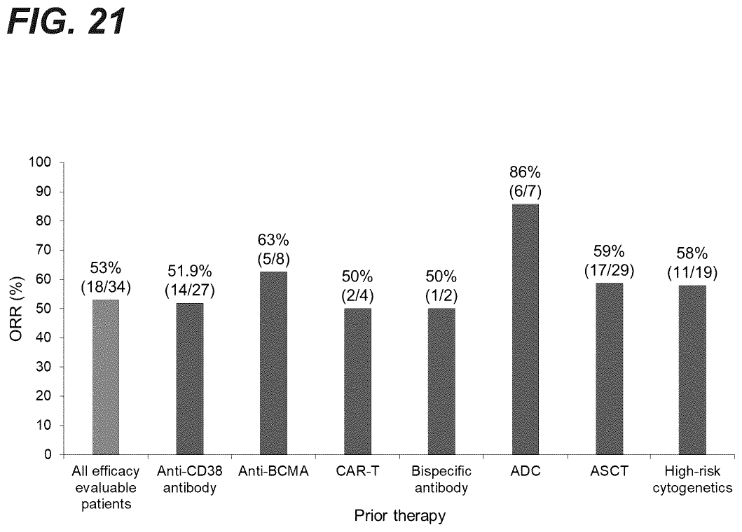

ORRs at the active dose level by prior therapy were generally consistent. In patients who received ≥3.6 mg/20 mg doses of cevostamab, the overall response rate (ORR) was 53% (18/34); response was consistent in patents with exposure to prior daratumumab and anti-BCMA agents (FIG. 21). Prior therapy and the number of lines of treatments do not appear to affect FcRH5 expression.

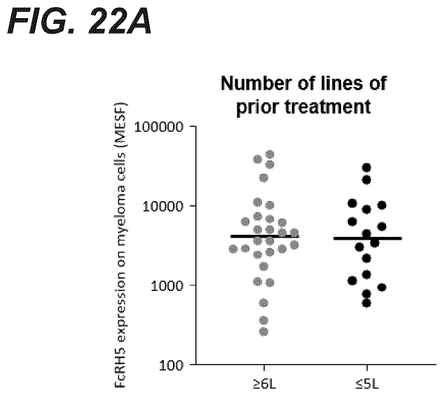

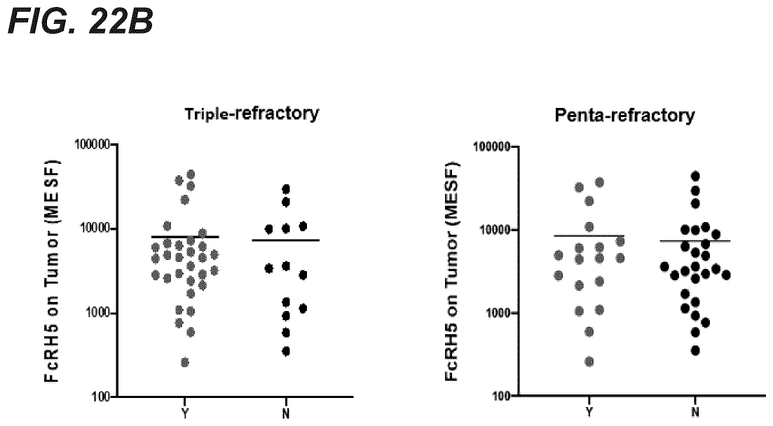

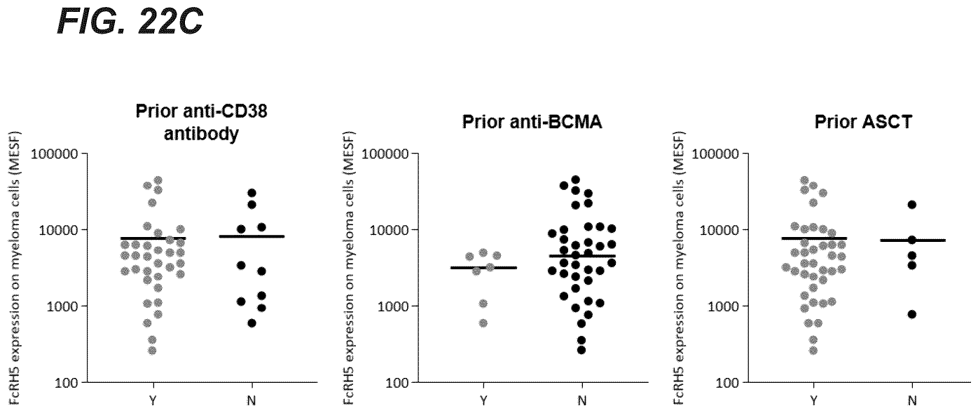

FcRH5 expression was detected on myeloma cells from the bone marrow of all patients with adequate BMA samples for biomarker evaluation at baseline (n=44). Response to cevostamab was observed irrespective of FcRH5 expression levels in patients with R/R MM assessed to date; no obvious relationship between response to cevostamab and baseline FcRH5 expression level was observed at the active dose level (FIG. 13). FcRH5 expression did not appear to be affected by the number of lines or types of prior therapies, including prior anti-BCMA agents (FIGS. 22A-22C).

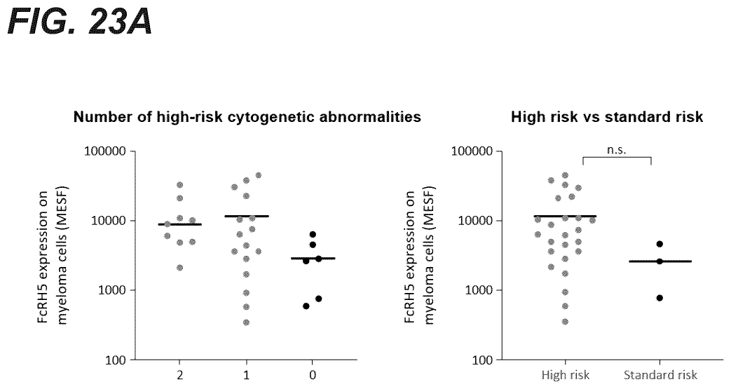

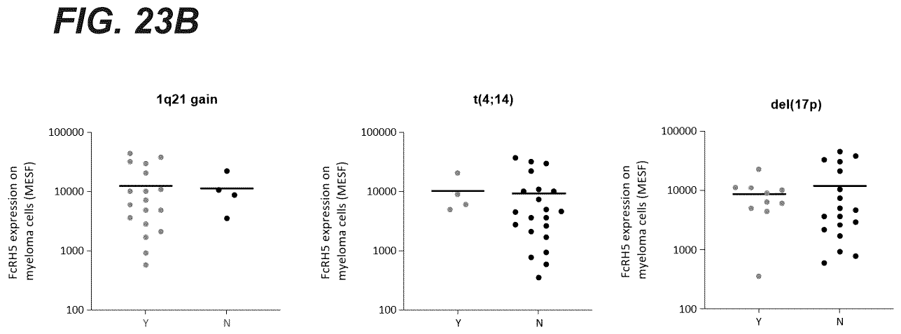

There is a trend towards higher FcRH5 expression levels in the cytogenetics high-risk patients (FIGS. 23A-23C). Of the patients with evaluable samples for cytogenetics (n=28), 25 patients were high risk (HR; defined as presence of one or more of the following abnormalities: 1q21, t(4;14), t(4,16) or del(17p)) and 3 were standard risk (SR). Baseline FcRH5 expression stratified by cytogenetic risk showed a trend towards higher expression in patients with HR cytogenetics; median MESF was 6329 (minimum: 352; maximum: 44409) in HR patients and 2591 (minimum: 766; maximum: 4560) in SD patients (FIG. 23A). MESF was 8839 (range: 2137-32381) in patients with two cytogenetic abnormalities (n=9), 5379 (range: 352-44409) in patients with one cytogenetic abnormality (n=16), and 2591 (range: 766-4560) in patents without cytogenetic abnormalities (n=3) (FIG. 23A). Expression levels were consistent in patients with and without 1q21 gain, t(4,14) vs no t(4,14), and del(17p) vs no del(17p) (FIG. 23B). No patients with t(14;16) were detected to date. No clear correlations were observed between response to cevostamab in the active dose cohort and baseline expression levels of FcRH5.

These data further confirm FcRH5 as a promising target for MM therapeutics.

### Example 12. Rationale for Doses and Indications

Dose

Based on the totality of clinical safety and efficacy, pharmacokinetic (PK) and pharmacodynamic (PD) data, and PK-PD/exposure-response (E-R) analyses generated in the GO39775 study, 0.3/3.6/160 mg (double step-up doses of 0.3 mg on Cycle 1 Day 1 and 3.6 mg on Cycle 1 Day 8, followed by a target dose (TD) of 160 mg on Cycle 1 Day 15 and Day 1 of subsequent Q3W cycles) is recommended as a cevostamab monotherapy dosing regimen for patient having R/R MM. These doses and schedule were selected not only to ensure that overall safety and CRS are manageable, but to also enable patients to safely receive the TD that drives higher, deeper, and durable responses.

In the ongoing GO39775 study, cevostamab demonstrated a positive benefit/risk profile in a heavily pre-treated MM population (median 6 prior lines of therapy; Table 9). At TDs ≥20 mg, at which objective responses were observed (clinically active doses), cevostamab had an overall response rate (ORR) of 43.3% and responses were shown to be durable with a median duration of response (DOR) of 15.6 months (95% CI: 6.4, 21.6). The most frequently reported adverse event (AE), CRS (graded by the American Society for Transplantation and Cellular Therapy (ASTCT) 2019 criteria (Lee et al., Biol Blood Marrow Transplant, 25: 625-638, 2019)), was effectively managed with the use of step-up dosing to limit the frequency of severe CRS; rates of Grade 3 CRS were low (1.3% overall), and no Grade 4 or 5 CRS events occurred. CRS events predominantly occurred during Cycle 1 while patients were under inpatient observation, enabling prompt identification and management of CRS. Non-CRS AEs also predominately occurred in early cycles and no cumulative toxicity was observed. Overall, cevostamab had a clear benefit/risk for heavily pre-treated MM patients, with strong evidence of clinical activity coupled with a manageable safety profile.

Limiting the rates of severe CRS and ensuring that patients can safely escalate to clinically active doses was a key objective of the Phase 1 study. To this end, both single and double step-up dosing regimens were tested in escalation and expansion. As further detailed below, clinical safety, PK, and PD data identified the 3.6 mg single step-up and 0.3/3.6 mg double step-up regimens for further evaluation in dose expansion. Data from the Phase 1 study demonstrate that both single step-up and double step-up regimens effectively limited the rate of severe CRS. However, the totality of data also indicates that the proposed double step-up regimen further improved the CRS profile of cevostamab compared with the single step-up regimen. Lower CRS rates were observed with the 0.3/3.6 mg/TD double step-up doses (77.3%) versus 3.6 mg/TD single step-up dose (88.2%). Notably, the rates of neurological symptoms consistent with immune-effector cell associated neurotoxicity syndrome (ICANS) accompanying CRS were considerably lower with the 0.3/3.6 mg/TD double step-up doses (4.5%) compared with the 3.6 mg/TD single step-up dose (21.2%) (Table 30). Given these meaningful improvements in CRS profile and neurological symptoms consistent with ICANS, the 0.3/3.6 mg double step-up is recommended.

TABLE 30
Summary of Neurological Symptoms Consistent
with ICANS in Single Step-Up and Double Step-Up
Regimens Irrespective of Target Dose

| | Single Step-Up | | Double Step-Up | |
|---|---|---|---|---|
| | Arms A + C | | Arms B + D | |
| | (3.6/TD) | | (0.3/3.6/TD) | |
| | N = 85 | | N = 44 | |
| Total number of patients with at | 22 | (25.9%) | 6 | (13.6%) |
| least one event, n (%) | | | | |
| ICANS accompanying CRS (s/s | 18 | (21.2%) | 2 | (4.5%) |
| of CRS) | | | | |
| ICANS like NAEs (not reported | 4 | (4.7%) | 4 | (9.1%) |
| as s/s of CRS) | | | | |
| Recurring symptoms or AEs | 9 | (10.6%) | 0 | (0.0%) |

AE = adverse event; CRS = cytokine release syndrome; ICANS = immune-effector cell associated neurotoxicity syndrome; NAE = neurological adverse event; s/s = signs/symptoms; TD = target dose.

Data from the Phase 1 study support the proposed 160 mg cevostamab TD based upon improvements in the response rates compared to lower TDs while maintaining a tolerable safety profile. Cevostamab has been tested across a wide range of TDs (0.15-198 mg), with initial clinical activity being observed at the 20 mg dose. Data from this dose escalation showed an increasing response rate with increasing TDs, independent of single vs. double step-up regimen, and thus expansion arms were opened at both 90 mg and 160 mg to confirm the dose-response relationship. Consistent with the results from dose escalation, a higher ORR was observed at the 160 mg TD (54.5%) compared with the lower 90 mg TD (36.7%). Exposure-response (E-R) and Population Pharmacokinetics-Tumor Growth Inhibition (PopPK-TGI) analyses for efficacy confirmed the observed dose-response relationship, with a significant improvement in the probability of both the ORR and ≥VGPR rates with an increasing TD (range tested: 0.15-198 mg). Similar ORR and ≥VGPR rates were predicted at matched TD levels for single step-up and double step-up doses using the PopPK-TGI model.

Importantly, safety and tolerability at the 160 mg dose is comparable to other lower active doses tested. No significant positive E-R relationships were observed between increasing exposure and the risk of key safety events across the TDs tested (0.15-198 mg). Across active TDs, Grade ≥3 AE rates in later cycles remained low and, similarly, chronic cumulative toxicity has not been observed. Taken together, cevostamab was shown to be well tolerated across all TDs and, supported by the evidence that increasing exposure improves the probability of obtaining a clinical response, it is believed that that the 160 mg TD maximizes the benefit/risk ratio for patients.

In summary, data from the initial Phase 1 GO39975 study demonstrate that cevostamab offers a positive benefit/risk for patients with late-line MM.

A. 0.3/3.6 mg Cycle 1, Day 1/Cycle 1, Day 8 (C1D1/C1D8) Double-Step is Recommended for Cycle 1 Step-Up Doses

Emerging data with T-cell engaging bispecific therapies demonstrate that step-up dosing is an effective method to mitigate CRS, but the optimal number of step-up doses and the mechanism by which step-up dosing limits CRS are not known. In Study GO39775, both single and double step-up regimens were tested to inform the selection of RP2D with the goals of:
1. Limiting the rates of severe, Grade ≥3 CRS;
2. Limiting the majority of CRS events to the first cycle when patients are hospitalized for observation and allowing prompt intervention if needed; and
3. Enabling the safe administration of higher TDs to provide anti-myeloma activity.

Both single and double step-up regimens effectively limited the severity of CRS and enabled patients to safely receive TDs up to 198 mg (maximum administered dose to date). Grade 3 CRS events were observed in 1.3% of patients on study. and no Grade 4 or Grade 5 CRS events were reported. Only 1 of 160 patients discontinued cevostamab treatment due to CRS. Similarly, CRS events with both step-up regimens were primarily observed during the initial cycle and occurred while patients were hospitalized for observation. In this regard, both step-up regimens enabled the safe management of CRS.

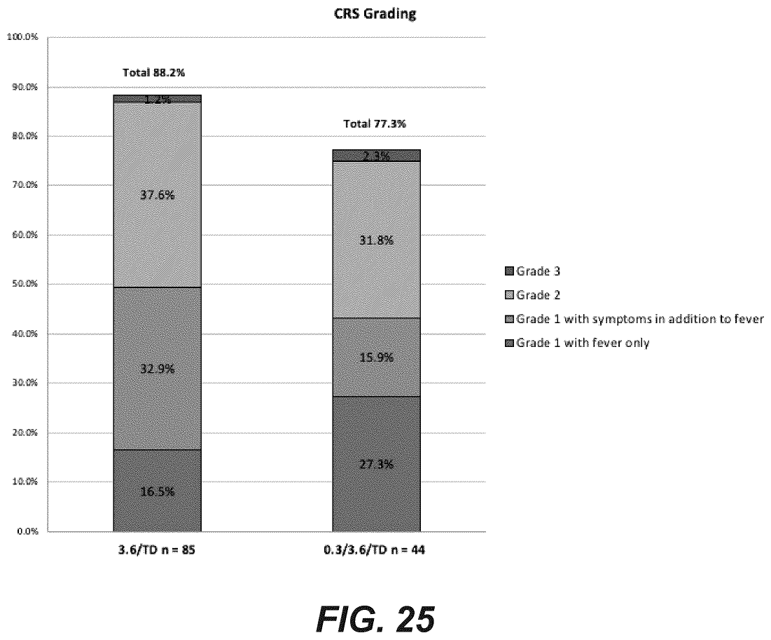

While either step-up regimen effectively limits severe CRS, the totality of data suggests that the 0.3/3.6 mg double step-up regimen further improves the CRS profile compared to the 3.6 mg single step-up regimen. As detailed below, among double step-up regimens, the 0.3/3.6 mg doses were identified as the optimal double step-up regimen that limited CRS during step dosing while still enabling safe administration of the TDs. Further testing of this double step-up regimen in dose expansion demonstrated improvement in the overall CRS profile as compared to the 3.6 mg single step-up regimen: not only was the overall rate of CRS lowered from 88.2% to 77.3%, but the Grade 1 CRS symptom profile was improved (FIG. 25).

Neurological symptoms are another important concern with T-cell engaging therapies. In GO39775, neurological symptoms that the investigator attributed to CRS were captured as signs and symptoms of CRS. Any neurological symptoms not attributed to CRS were captured as an adverse event. For completeness and to not miss any potential signals, all of these symptoms/adverse events were reviewed for neurological symptoms that would be consistent with immune-effector cell associated neurotoxicity syndrome (ICANS) as defined in Lee et al., Biol Blood Marrow Transplant, 25(4): 625-638, 2019.

Neurological symptoms consistent with ICANS that were reported as a symptom of CRS are referred to as ICANS accompanying CRS, as these are thought to be immune effector cell-associated. Neurological events reported as AEs that are symptoms consistent with the definition of ICANS were reported but not all of these were due to immune effector cell activation, but may be due to other causes (e.g. underlying disease, concomitant medications, subdural hematoma). These are referred to as ICANS-like NAEs.

The 0.3/3.6 mg double step-up regimen appears to limit the occurrence of neurological symptoms consistent with ICANS (i.e., both ICANS accompanying CRS and ICANS-like NAEs). With 3.6 mg single step-up dosing, 18 of 85 patients (21.2%) experienced ICANS accompanying CRS (Table 24); importantly, all events were Grade 1 or Grade 2 events, with the exception of one reversible Grade 3 event, indicating that ICANS accompanying CRS is manageable with single step-up dosing. With the 0.3/3.6 mg double step-up regimen, the rate of ICANS accompanying CRS was notably lower, with only 2 of 44 patients (4.5%) experiencing these symptoms. For both patients, the ICANS accompanying CRS were limited to Grade 1 and did not reoccur with subsequent doses. Taken together, neurological symptoms consistent with ICANS were limited in severity and were manageable with either step-up approach. However, the trends towards lower rates of ICANS accompanying CRS with 0.3/3.6 mg/TD double step-up doses favor this regimen.

Differences in the observed CRS profiles between step-up regimens are unlikely to be due to variability in CRS interventions (Table 31) or baseline demographics (Table 9), which were similar between regimen groups. Given that the 0.3/3.6 mg double step-up regimen improves various aspects of the CRS and ICANS profile as compared to the single step-up dose, this approach is proposed to limit the CRS incidence for treatment of prior BCMA and triple-refractory MM.

TABLE 31
Management of Cytokine Release Syndrome Events in Study GO39775

| | Single Step- | | | | |
|---|---|---|---|---|---|
| | Up Dose | Double Step-Up Dose | | Proposed | |
| | Regimen^{a} | Regimen^{b} | | RP2D^{c} | All Patients^{d} |
| | N = 85 | N = 17 | N = 44 | N = 36 | N = 160 |
| Dose (mg) | 3.6/TD | 0.6-1.2/3.6/TD | 0.3/3.6/TD | 0.3/3.6/160 | 128 |
| Total patients | 75 | 14 | 34 | 29 | 128 |
| with CRS | | | | | |

Patients treated with:

| Tocilizumab | 31 | (41.3%) | 9 | (64.3%) | 16 | (47.1%) | 16 | (55.2%) | 56 | (43.8%) |
|---|---|---|---|---|---|---|---|---|---|---|
| Steroids | 18 | (24.0%) | 5 | (35.7%) | 9 | (26.5%) | 9 | (31.0%) | 33 | (25.8%) |
| Tocilizumab or | 37 | (49.3%) | 10 | (71.4%) | 18 | (52.9%) | 18 | (62.1%) | 66 | (51.6%) |
| steroids | | | | | | | | | | |
| Tocilizumab | 12 | (16.0%) | 4 | (28.6%) | 7 | (20.6%) | 7 | (24.1%) | 23 | (18.0%) |
| and steroids | | | | | | | | | | |
| Fluids | 24 | (32.0%) | 7 | (50%) | 7 | (20.6%) | 6 | (20.7%) | 39 | (30.5%) |
| Vasopressor(s) | 1 | (1.3%) | 0 | | 0 | | — | | 1 | (0.8%) |
| Low flow O₂ | 11 | (14.7%) | 6 | (42.8%) | 9 | (26.5%) | 9 | (31.0%) | 26 | (20.3%) |

| | | | | | | | | |
|---|---|---|---|---|---|---|---|---|
| High flow O₂ | 0 | 0 | 1 | (2.9%) | 1 | (3.4%) | 1 | (0.8%) |
| ICU | 0 | 0 | 1 | (2.9%) | 1 | (3.4%) | 1 | (0.8%) |

admittance

CRS = cytokine release syndrome; ICU = intensive care unit; RP2D = recommended Phase II dose; TD = target dose; Q3W = every 3 weeks.
Note:
Percentages are based on the number of patients who experienced CRS in each column.
^{a}Cevostamab is administered on Day 1 (step-up dose) and Day 8 (target dose) of Cycle 1 and on Day 1 (target dose) of subsequent Q3W cycles.
^{b}Cevostamab is administered on Day 1 (step-up dose), Day 8 (step-up dose), and Day 15 (target dose) of Cycle 1 and on Day 1 (target dose) of subsequent Q3W cycles.
^{c}The proposed RP2D and regimen is 0.3/3.6/160 mg Q3W: Cevostamab is administered at 0.3 mg (step-up dose) on Cycle 1 Day 1, 3.6 mg (step-up dose) on Cycle 1 Day 8, and 160 mg (target dose) on Cycle 1 Day 15 and Day 1 of subsequent Q3W cycles.
^{d}All Patients refers to all patients in Arms A-D. Data for the 3 patients in Arm E are not presented.

Overall CRS Profile of Recommended 0.3/3.6 mg Double Step-Up Regimen was Shown to be Tolerable, Manageable, and Reversible

C1D1 doses of 0.3 mg, 0.6 mg, and 1.2 mg were tested in Arm B. Initial biomarker data suggested that doses from 0.3 mg to 1.2 mg were associated with lower PD markers than the 3.6 mg dose; PD was not observed at doses <0.3 mg. It was observed that the 1.2 mg dose reduced CRS at 3.6 mg dose, but overall C1 rates of CRS are unchanged, and that the 0.6 mg dose was insufficient to mitigate CRS at 3.6 mg but was high enough to induce C1D1 CRS. These data suggest that C1D1 dose shapes the overall C1 CRS profile across a narrow dose range. C1D1 doses associated with maximal T-cell activation are most likely to reduce CRS rates on subsequent doses, but also lead to higher grade CRS and IL-6 release. C1D1 doses that elicit sub-maximal T-cell activation while limiting IL-6 may improve the overall CRS profile.

The clinical data generated to date indicate that the overall CRS profile of the proposed 0.3/3.6 mg double step-up regimen, irrespective of the TD, is well tolerated. A total of 60 CRS events were reported in 34 of 44 treated patients (77.3%) with the 0.3/3.6 mg double step-up regimen. The most frequently reported symptoms (≥10%) associated with CRS at this regimen included fever (75% of patients), hypoxia (27.3%), chills (25%), tachycardia (18.2%), and hypotension (15.9%).

With the 0.3/3.6 mg double step-up regimen, 95% of CRS events occurred within 48 hours post dosing (and all but 2 events had onset within 24 hours), which falls within the protocol-specified hospitalization window. CRS events beyond the first cycle are infrequent, with 90.0% of CRS events occurring within the initial cycle. In the limited cases where CRS events occurred after Cycle 1, CRS events were mostly Grade 1, and no patient on study experienced Grade ≥3 CRS events occurring after Cycle 1. The predictable onset of CRS as well as the mandatory Cycle 1 inpatient observations for each step-up dose and the initial TD have also ensured that CRS events are promptly identified and managed in the inpatient setting.

As detailed above, ICANS accompanying CRS were infrequent, reversible and limited to Grade 1 severity with the 0.3/3.6 mg double step-up regimen. Most CRS events with the 0.3/3.6 mg double step-up regimen were Grade 1 or Grade 2 and were reversible with either supportive care (acetaminophen, fluids, low-flow oxygen), or tocilizumab and/or corticosteroids. A total of 34 patients with this regimen experienced CRS, of whom 18 (52.9%) were treated with tocilizumab or steroids and 7 (20.6%) with both for CRS (Table 25). One patient experienced Grade 3 CRS at the TD due to rapid onset of hypoxia requiring high flow oxygen. The patient was admitted to the intensive care unit (ICU) and treated with tocilizumab with prompt improvement in oxygenation. CRS fully resolved within 48 hours and the patient continued the study and has not experienced any additional CRS events. No other patients experienced Grade 3 CRS with the 0.3/3.6 double step-up regimen. CRS including neurological symptoms consistent with ICANS with the 0.3/3.6 mg double step-up doses were manageable, reversible, and in all but one patient limited to Grade ≤2.

In summary, the totality of safety data generated to date indicates that the 0.3/3.6 mg double step-up regimen enables the safe administration of cevostamab and ensures that CRS events were manageable and reversible.

B. Target Dose of 160 mg is Recommended Based on Positive Dose Response for Clinical Activity and Lack of Target Dose Dependent Toxicity

The proposed TD of 160 mg is recommended based on the clear benefit/risk assessment by using a combination of efficacy, safety, PK, PD and PK-PD/E-R analyses. TDs have been assessed in dose escalation studies across a broad range (0.15-198 mg) and a maximum tolerated dose (MTD) has not been reached. An initial expansion arm was opened at the 90 mg TD to generate additional safety/efficacy data at the single step-up regimen while dose escalation was continued in parallel. While data from the Study GO39775 Arm C expansion at 90 mg showed a positive benefit/risk ratio, emerging data from the ongoing dose escalation indicated that TDs greater than 90 mg could further increase ORR while maintaining a similar safety profile. For this reason, an additional dose expansion at the 160 mg dose was completed. As detailed below, the totality of data collected to date demonstrates a consistent safety profile across all TDs and that higher TDs enhance clinical activity and patients' chances of obtaining deep responses.

Target Dose Safety

Across all TDs tested, the safety profile remained consistent and manageable. A total of 160 safety-evaluable patients in Arms A-D received a median of 3.5 (range: 1-34) treatment cycles and were followed up for a median 6.1 (range: 0.2-39.4) months. No cumulative or late-onset toxicities have been identified to date. Rates of both overall AEs and CRS were highest in the initial treatment cycle and then remained low across later cycles, and showed no trend of worsening with higher TDs. Discontinuations of cevostamab due to AEs were infrequent (16/160 patients, 10.0%), were not driven by any single preferred term, and were not TD-dependent, supporting the tolerability of cevostamab treatment at the recommended TD of 160 mg.

The clinical safety data and PK-PD/exposure-safety analyses demonstrate that the increasing TDs (0.15-198 mg) did not demonstrate a significant positive relationship in the overall AE risk regardless of the step-up doses tested.

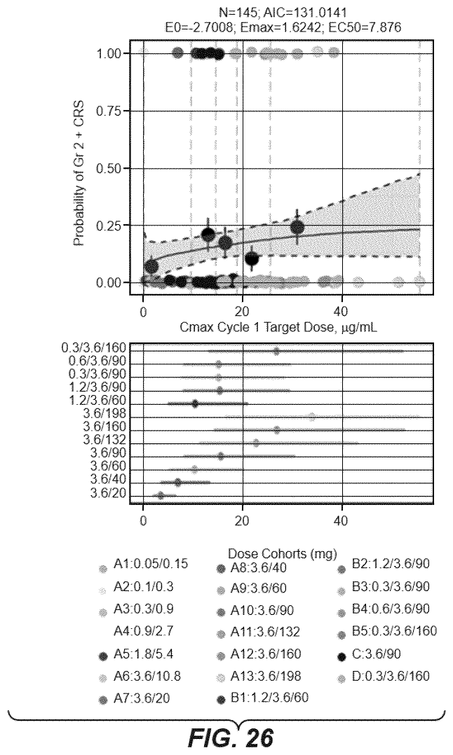

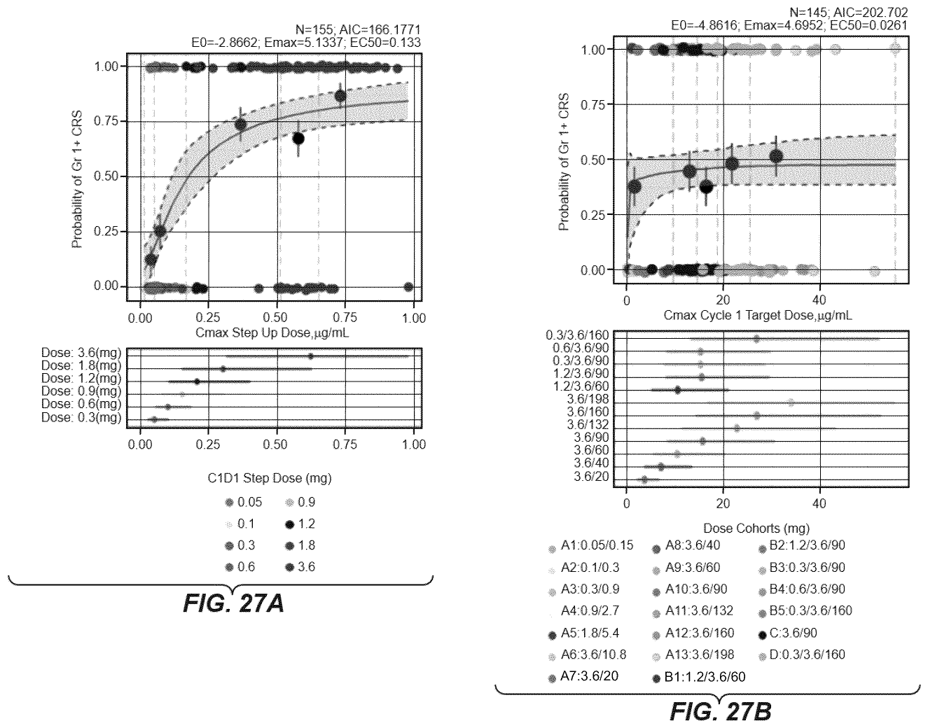

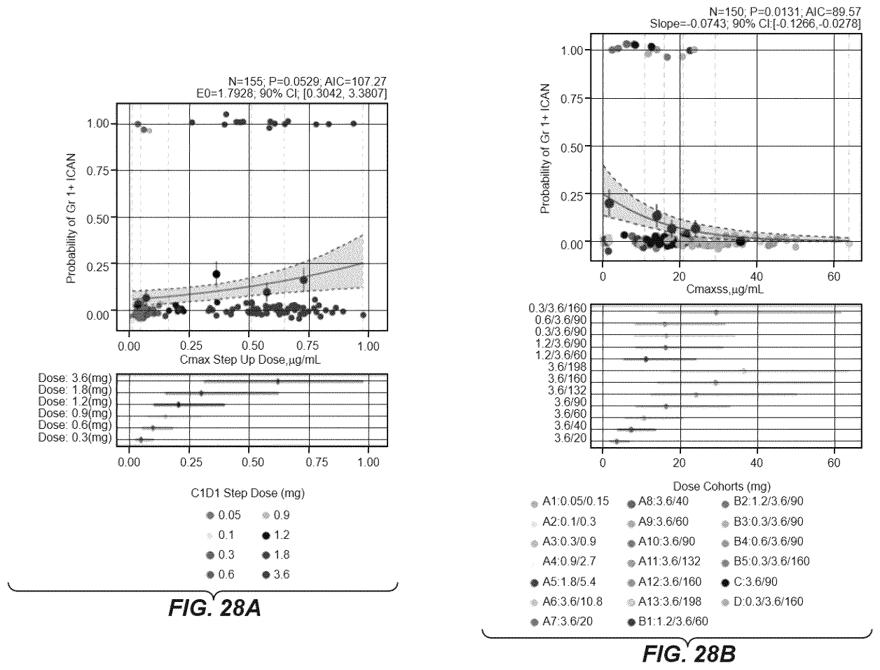

Exposure-safety analyses showed that there were no significant positive relationships between the TD exposures and the following key safety events based on the pooled data from the single step-up and double step-up dosing regimens: Grades ≥1 and ≥2 CRS (FIGS. 27A and 27B and FIG. 26); Grades ≥1 neurological symptoms consistent with ICANS (FIGS. 28A and 28B); Grades ≥3 cytopenias (neutropenia, anemia and thrombocytopenia); Grade ≥2 infusion related reactions (IRRs); Grade ≥2 infections; and any pooled Grade ≥3 AEs.

Exposure-Response Analysis

Clinical efficacy data indicate that increases in TDs are associated with an improved probability of achieving objective response and very good partial response or better (≥VGPR). As discussed previously, 160 mg TD resulted in an improved ORR compared to the lower TDs (see Table 32).

TABLE 32
Responders and ≥VGPR Rates Observed at Target
Doses of 90 mg and 160 mg (Study GO39775)

| | Target Dose (90 mg) | Target Dose (160 mg) |
|---|---|---|
| | N = 60 | N = 44 |
| Responders | 22 (36.7%) | 24 (54.6%) |
| ≥VGPR | 12 (20.0%) | 9 (20.5%) |

≥VGPR = very good partial response or better.
Note:
Patients receiving a target dose of 90 or 160 mg, regardless of single step or double step, were aggregated for response analysis.

E-R and the Population Pharmacokinetics-Tumor Growth Inhibition (PopPK-TGI) analyses confirmed the observed clinical dose-response.

The PopPK-TGI evaluation showed an improvement in the predicted response estimates at the higher TDs tested. To aid in further refining understanding of the nature of the exposure-efficacy relationship of cevostamab, an evaluation using a PopPK-TGI model of M-protein was performed. Since this model relates the longitudinal time course of PK and M-protein data using a population modeling approach, it can be leveraged to account for differences in dose levels and schedules on the treatment response. Consistent with the observed clinical response data (Tables 32, 11, and 12), the model predicted an improvement in the response estimates for ORR and ≥VGPR at the higher TDs tested. At the proposed TD of 160 mg, the predicted response rates appear to approach a plateau (Table 33). The ORR and ≥VGPR predictions from this evaluation demonstrate reasonable concordance with the observed clinical data. Of note, similar (<5% difference) ORR and ≥VGPR rates were predicted at matched TD levels for single step-up and double step-up doses (Table 29). This suggests that choice of step dose regimen does not strongly influence the probability of response. This similarity in the model-predicted ORR at the same TD level is apparent in the observed clinical data at the TDs with a considerable sample size (i.e., for the 90 mg TD: ORR for single step-up was 37% for N=41 patients; and ORR for double step-up was 37% for N=19 patients).

TABLE 33
Comparison of Model-Predicted Best ORR and ≥VGPR
Rates at Target Doses of 90 mg and 160 mg

| | PopPK-TGI Prediction | | E-R Prediction^{a} | E-R Prediction^{a} |
|---|---|---|---|---|
| | Median (90% CI) | | Median (90% CI) | Median (90% CI) |
| Target Dose (mg) | Single Step-Up | Double Step-Up | AUCₛₛ | C_{min, ss} |

Best ORR

| | | | | | | | | |
|---|---|---|---|---|---|---|---|---|
| 90 | 44% | (26-60%) | 40% | (26-56%) | 37% | (29-45%) | 36% | (26-47%) |
| 160 | 50% | (34-66%) | 46% | (30-62%) | 54% | (46-63%) | 53% | (44-62%) |

≥VGPR Rate

| | | | | | | | | |
|---|---|---|---|---|---|---|---|---|
| 90 | 24% | (8-38%) | 22% | (6-36%) | 21% | (15-28%) | 19% | (12-27%) |
| 160 | 30% | (10-42%) | 26% | (10-42%) | 30% | (22-39%) | 29% | (21-38%) |

AUCₛₛ = area under the concentration-time curve at steady-state; C_{min, ss} = trough concentration at steady-state; E-R = exposure-response; ORR = objective response rate; PopPK = population pharmacokinetics; TGI = tumor growth inhibition; VGPR = very good partial response.
^{a}Results of the logistic regression modeling for exposure-efficacy analyses using pooled data from the single step-up and double step-up dosing regimen for the ORR and ≥VGPR endpoints.

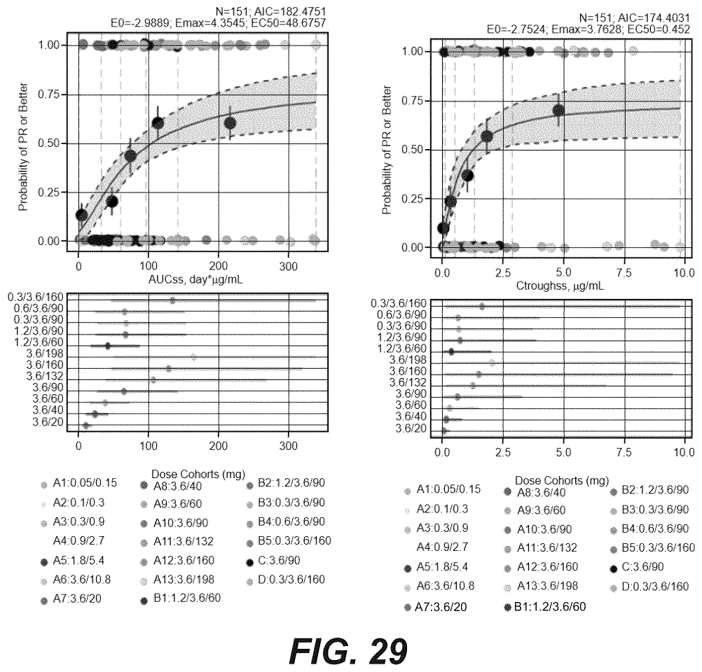

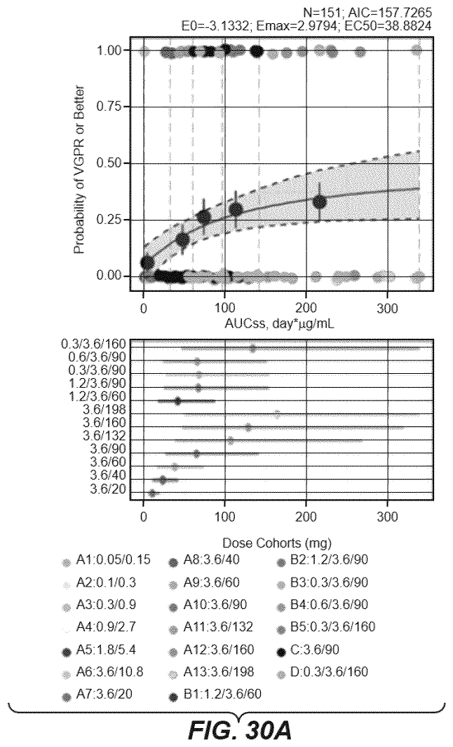

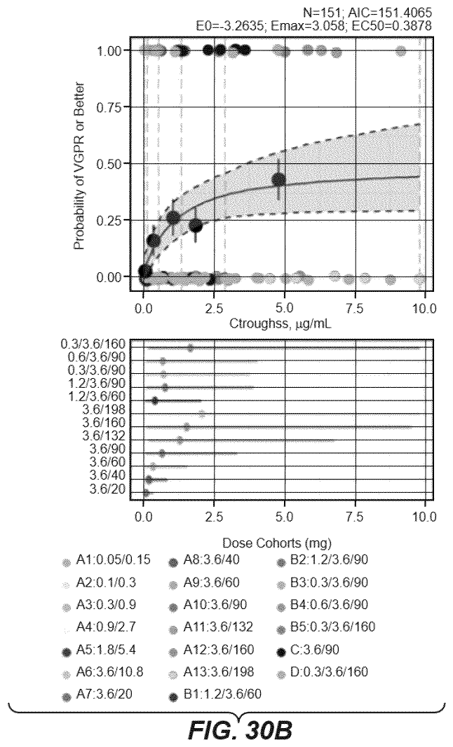

Based on E-R analyses across the range of tested TDs (0.15 mg-198 mg), the ORR and ≥VGPR significantly increased with an increase in cevostamab exposure (AUCₛₛ and C_{min,ss}) (FIGS. 29, 30A, and 30B). The nature of these E-R relationships appeared to follow an Eₘₐₓ model with the TD of 160 mg approaching a plateau for these clinical endpoints using pooled data from the single step-up and double step-up regimen, suggesting that TDs of 198 mg or higher may not lead to a substantial improvement in the response rates. A pooled E-R evaluation was performed given that these empirical models cannot distinguish the impact of dosing schedules on the clinical endpoints. The estimated response rates using the E-R models are consistent with the PopPK-TGI estimates (Table 12) and the ORR and ≥VGPR rates from these E-R models demonstrate reasonable concordance with the observed clinical data.

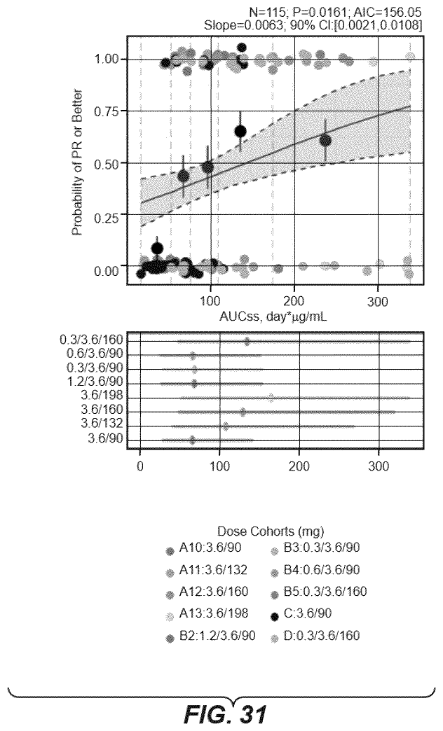

Furthermore, as a sensitivity analysis, E-R analyses performed for the TD ranges of 90 mg-198 mg between AUCₛₛ and ORR preserved the statistically significant improvements in ORR with increasing exposures (FIG. 31), confirming the significant improvement in the response rates at the 160 mg TD compared to the lower TD cohorts.

Based on the E-R evaluation using C_{min,ss} as the exposure metric, at TDs ≥160 mg, the trough concentrations were shown to approach a near maximal effect (i.e., EC₉₀) for cevostamab in patients with MM. The model-derived 90% maximal effect (EC₉₀) for C_{min,ss} was 4.4 μg/mL. Of note, this E-R derived clinical EC₉₀ estimate was comparable with the observed ex vivo maximum EC₉₀ (2.7 μg/mL; range: 0.03 μg/mL-2.7 μg/mL). The value was generated from an ex vivo T-cell dependent cytotoxicity assay on patient-derived primary myeloma cell evaluated by co-culturing human myeloma bone marrow mononuclear cells (N=4) with CD8+ T-cells isolated from healthy donor and varying concentrations of cevostamab (Li et al., Cancer Cell, 31: 383-395, 2017). The maximum observed EC90 is likely to be a relevant pharmacological target given the uncertainty around the effector-to-T-cell ratio in the bone marrow, particularly for patients with high tumor burden, thereby supporting a need for higher TDs in patients with MM.

In conclusion, higher TDs within the tested range improve the probability of clinical responses without an apparent increased risk for adverse events as compared to lower TDs. Both the E-R analyses and PopPK-TGI evaluation confirm that a TD of 160 mg improves the probability of patients achieving an objective response and ≥VGPR compared to lower doses and demonstrate reasonable concordance in the prediction of these responses. Moreover, similar ORR and ≥VGPR rate estimates were predicted at the same TD level for the single step-up and double step-up dosing schedules based on the PK-TGI evaluation. Therefore, the proposed TD of 160 mg is recommended based on the positive benefit/risk assessment using a combination of efficacy, safety, PK, PD and PK-PD/E-R analyses.

Rationale for Selection of the 0.3 mg and 3.6 mg as Step-Up Doses

A final consideration is whether different step-up dose levels could potentially improve the overall CRS profile of cevostamab relative to the 0.3/3.6 mg step levels. Based on the totality of clinical efficacy and safety, PD and the PK-PD/E-R analyses, it is believed that additional changes to either the Cycle 1, Day 1 (C1D1) and/or Cycle 1, Day 8 (C1D8) step-up doses are unlikely to improve the cevostamab CRS profile.

Selection of 0.3 mg as the C1D1 Double Step-Up Dose

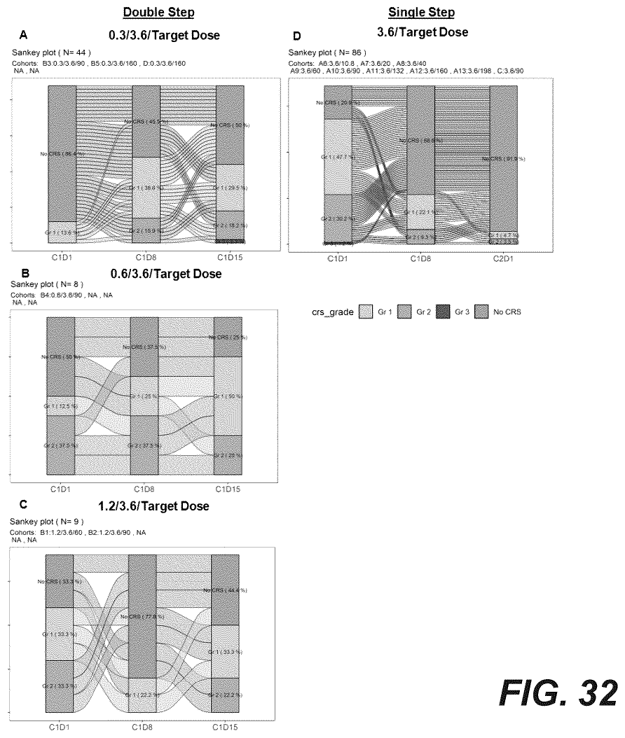

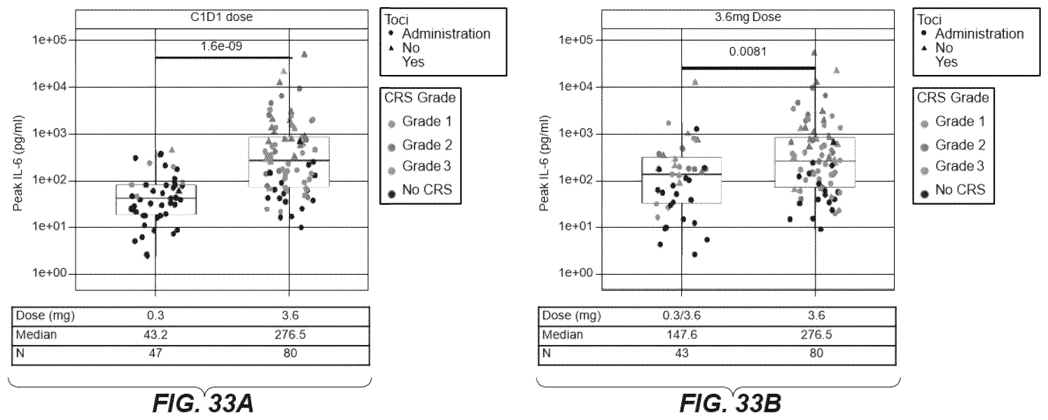

The 0.3 mg dose is considered the optimal Cycle 1, Day 1 (C1D1) dose in the double step-up regimen based on its ability to lower the CRS rate at the second step while also limiting the overall C1D1 CRS rate and severity. Rates of CRS at the subsequent C1D8 3.6 mg dose were lower (54.5% N=44) compared with CRS rates after C1D1 3.6 mg dose in the single step-up regimen (80.0%, N=85) (FIG. 32). Similarly, the 0.3 mg C1D1 dose appears to reduce not only the overall CRS rate after a 3.6 mg dose, but also the rate of Grade ≥2 CRS seen after a 3.6 mg dose (15.9% double step-up, 30.6% single step-up).

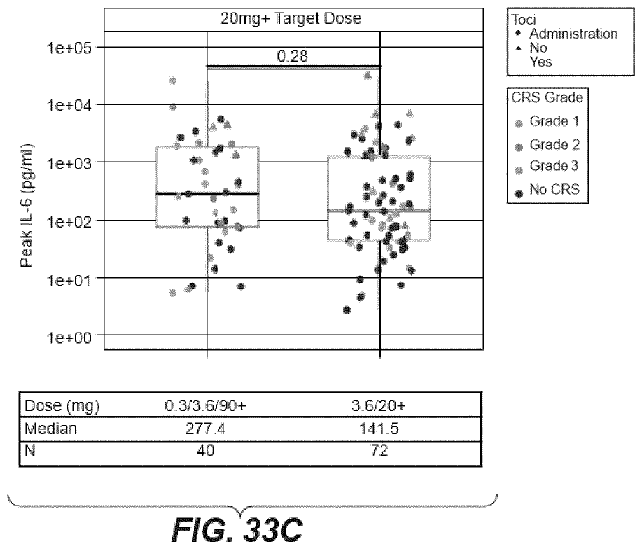

Consistent with this effect, PD data indicate that peak IL-6 levels following the 3.6 mg dose are lower when preceded by the 0.3 mg as compared to the 3.6 mg step dose administered on C1D1 (FIGS. 33A-33C). The overall rates of CRS following the initial 0.3 mg dose itself are low and limited to Grade 1. Taken together, the clinical and PD data indicate that the 0.3 mg dose accomplishes the goal of blunting CRS at the subsequent C1D8 dose.

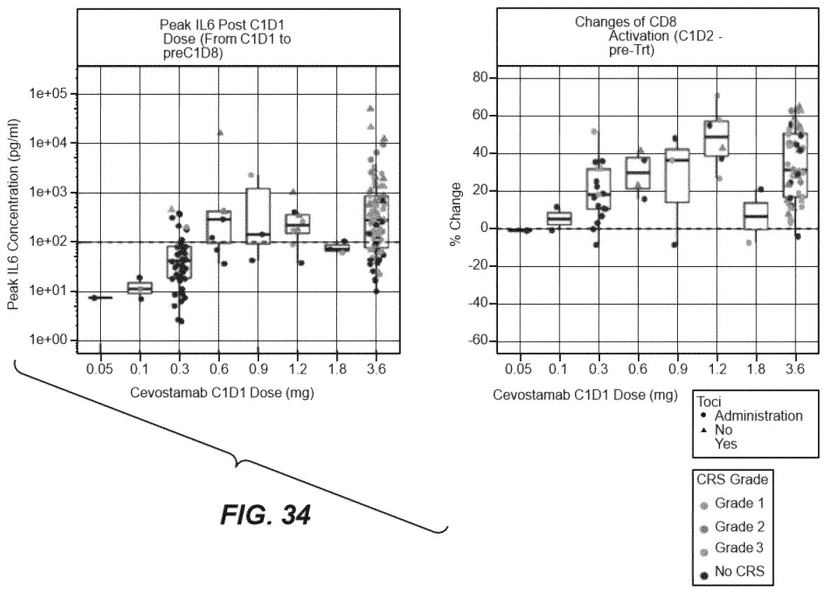

Clinical and PK-PD/E-R analyses do not support either increasing or decreasing the C1D1 dose. Doses of 0.6 and 1.2 mg were tested as potential C1D1 doses. Rates of CRS including Grade ≥2 CRS events were higher following the 0.6 and 1.2 mg C1D1 dose (FIG. 32) compared to the 0.3 mg dose. Additionally, peak IL-6 levels after the 0.6 and 1.2 mg C1D1 doses were higher than levels after the 0.3 mg C1D1 dose, and were similar to IL-6 levels after the 3.6 mg C1D1 dose, consistent with the observed increased risk of CRS at these higher doses (FIG. 34), and suggesting that increasing the C1D1 dose above 0.3 mg would worsen the safety profile.

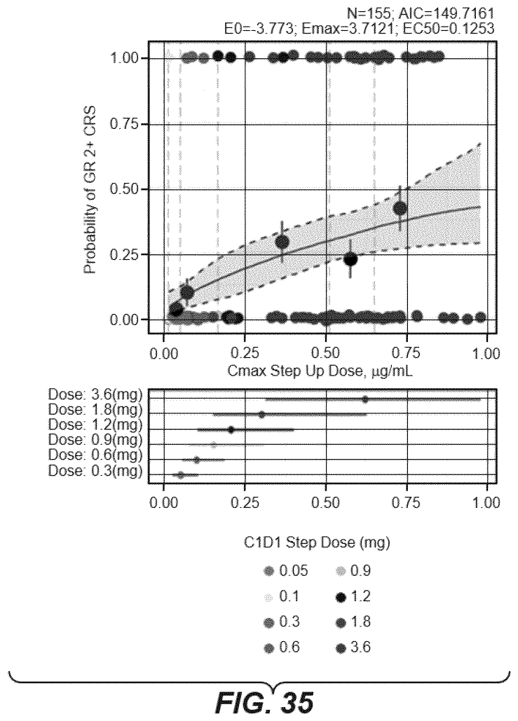

On the other hand, reduction of the C1D1 dose is also not warranted because PD data indicate that C1D1 doses below 0.3 mg are not associated with T-cell activation, suggesting that these doses may be too low to blunt CRS at subsequent doses (FIG. 34). As mentioned previously, the observed safety profile with the 0.3 mg dose is acceptable with low rates of CRS and no observed Grade 2 events. These findings are consistent with the E-R analyses where the 0.3 mg step-up dose led to a significant reduction in the occurrence of Grade ≥2 CRS (FIG. 35) compared to the 3.6 mg as the C1D1 dose. Furthermore, no significant positive E-R relationships were seen for key AEs (other than CRS) and cevostamab Cₘₐₓ after the C1D1 step dose (0.05-3.6 mg), suggesting little benefit would be achieved by further lowering the first dose below 0.3 mg. In summary, clinical data and PK-PD/E-R analyses support 0.3 mg as the optimal C1D1 dose.

Selection of 3.6 mg as the Cycle 1, Day 8 (C1D8) Double Step-Up Dose

The second step-up dose of 3.6 mg was selected based on the totality of data in both single step-up and double step-up dose escalation arms and is supported by quantitative systems pharmacology (QSP) modeling. In both single and double step-up dosing, the 3.6 mg step dose was effective at limiting the frequency of CRS and Grade ≥2 CRS at higher TDs on Cycle 1, Day 8 (C1D8) or Cycle 1, Day 15 (C1D15) (TDs from 10.8 to 198 mg) (FIG. 16).

Adjustment of the C1D8 3.6 mg dose is not expected to reduce overall rates of CRS. Increasing the dose is likely to increase the incidence of CRS after C1D8, while lowering the C1D8 dose may shift CRS to the TD. Tested doses below 3.6 mg (0.6 mg and 1.2 mg) when administered on C1D1 were associated with rates of Grade 2 CRS events similar to that following the 3.6 mg step up dose (Table 34, FIG. 32). For the C1D1 step doses of 0.6 mg and 1.2 mg, the peak IL-6 concentrations were similar to the 3.6 mg dose (FIG. 34). Similarly, T-cell activation was comparable between the 0.6, 1.2, and 3.6 mg doses (FIG. 34). Taken together, the PD and clinical data suggest the 0.6 and 1.2 mg doses are associated with similar risks of CRS as the recommended 3.6 mg C1D8 second step-up dose. Thus, lowering the C1D8 dose has a low likelihood of improving the overall CRS profile.

TABLE 34
Events of Cytokine Release Syndrome by Severity in Study GO39775 -
Double Step-Up Dose Regimen (Arms B + D, Safety-Evaluable Patients)

| | 0.3/3.6/Target Dose | | | | | | | | | | | |
|---|---|---|---|---|---|---|---|---|---|---|---|---|
| | | | | Any | 0.6/3.6/Target Dose (N = 8) | | | | 1.2/3.6/Target Dose (N = 9) | | | |
| Dose | 0.3 | 3.6 | 90-160 | time | | | | Any | | | | Any |
| (mg) | (N = 44) | (N = 44) | (N = 42) | (N = 44) | 0.6 | 3.6 | 90 | time | 1.2 | 3.6 | 60-90 | time |

Patients (%) with highest CRS grade:

| | | | | | | | | | | | | |
|---|---|---|---|---|---|---|---|---|---|---|---|---|
| Any | 7 (15.9) | 24 (54.5) | 24 (57.1) | 34 (77.3) | 4 (50.0) | 5 (62.5) | 6 (75.0) | 6 (75.0) | 6 (66.7) | 2 (22.2) | 5 (55.6) | 8 (88.9) |
| Grade | | | | | | | | | | | | |
| Grade 1 | 6 (13.6) | 17 (38.6) | 13 (31.0) | 19 (43.2) | 1 (12.5) | 2 (25.0) | 4 (50.0) | 1 (12.5) | 3 (33.3) | 2 (22.2) | 3 (33.3) | 3 (33.3) |
| Grade 2 | 0 | 7 (15.9) | 9 (21.4) | 14 (31.8) | 3 (37.5) | 3 (37.5) | 2 (25.0) | 5 (62.5) | 3 (33.3) | 0 | 2 (22.2) | 5 (55.6) |
| Grade 3 | 0 | 0 | 1 (2.4) | 1 (2.3) | 0 | 0 | 0 | 0 | 0 | 0 | 0 | 0 |
| Grade 4 | 0 | 0 | 0 | 0 | 0 | 0 | 0 | 0 | 0 | 0 | 0 | 0 |
| Not | 1 (2.3) | 0 | 1 (2.4) | 0 | 0 | 0 | 0 | 0 | 0 | 0 | 0 | 0 |
| evaluable | | | | | | | | | | | | |
| by | | | | | | | | | | | | |
| ASTCT | | | | | | | | | | | | |

ASTCT = American Society for Transplantation and Cellular Therapy; CRS = cytokine release syndrome; D = Day; NCI CTCAE = National Cancer Institute Common Terminology Criteria for Adverse Events.
Note:
Toxicity grade of CRS events were evaluated by ASTCT 2019 criteria, either as collected or derived programmatically, while the signs and symptoms of CRS were evaluated by NCI CTCAE grading criteria v4.

Given that alternative C1D8 doses other than 3.6 mg were not tested in the double step regimen in study GO39775, an in silico evaluation using an exploratory QSP model was performed to assess the impact of alternative C1D8 doses (0.3-40 mg) on the risk of CRS for the 0.3/C1D8/160 mg double step regimen. Consistently, the QSP model predicted that an adjustment of the C1D8 3.6 mg dose is not expected to meaningfully reduce rates of overall CRS (Grade ≥1 CRS and Grade ≥2 CRS) compared to the 0.3/3.6/160 mg double step-up dose regimen (FIG. 32), despite shifting the Grade ≥1 CRS dynamics at the individual C1D8 and C1D15 doses. A lower C1D8 dose (<3.6 mg) reduced the Grade ≥1 CRS risk at the C1D8 dose but increased the Grade ≥1 CRS risk at the C1D15 dose. In contrast, a higher C1D8 (>3.6 mg) dose was predicted to increase the Grade ≥1 CRS risk at the C1D8 dose despite lowering the Grade ≥1 CRS at C1D15 dose. The Grade ≥2 CRS risk at both C1D8 and C1D15 doses for the altered C1D8 doses were predicted to be similar to the 3.6 mg C1D8 doses. In summary, the exploratory QSP model simulations support that altering the C1D8 dose has low likelihood of improving the overall CRS profile.

Taken together, the observed clinical and PD data, along with QSP modeling, demonstrate that the 3.6 mg step-up dose safely enables patients to step-up to TDs that will drive clinical activity.

In summary, the data generated in the Phase 1 GO39975 study demonstrate that cevostamab delivers a positive benefit/risk for patients with late-line MM. Based upon extensive dose finding, dosing regimens have been identified that enable patients to safely receive clinically effective doses of cevostamab while limiting the risk of severe CRS.

### Example 13. IL-6 Release and CD8+ T Cell Activation

Peak IL-6

Elevated IL6 is a pronounced and causative factor of CRS. PD data demonstrated that 0.3 mg is the lowest C1D1 dose with marginal T-cell activation and minimal IL-6 elevations compared to ≥0.6 mg step doses tested in C1D1 in both treatment schedules. This is consistent with the lowest rates of Grade ≥1 CRS observed at the 0.3 mg C1D1 dose, suggesting that doses higher than 0.3 mg on C1D1 will not improve CRS profiles further.

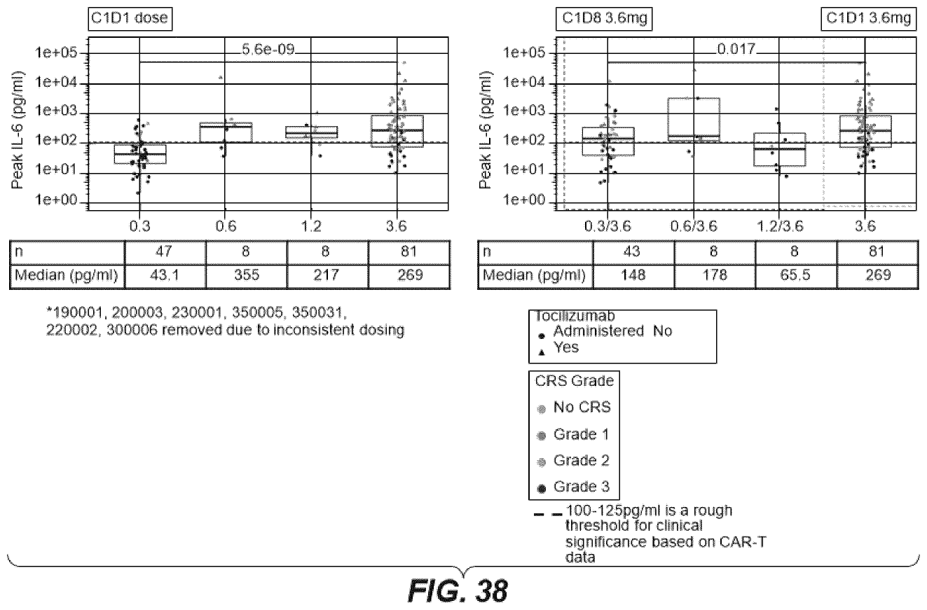

As is shown in FIG. 38, the 0.3 mg C1D1 dose of cevostamab used in the double step-up method was associated with lower IL-6 release compared to doses 20.6 mg. The median peak IL-6 level (highest measured or reported IL-6 value taken during the time period following a dose of the bispecific antibody) was lower following the 0.3 mg dose compared to doses 20.6 mg, despite similar CD8+ T cell activation profiles between these doses, and average peak IL-6 in patients treated with the 0.3 mg dose was below a 100-125 pg/mL threshold for clinical significance (FIG. 38). At the 3.6 mg dose, comparable median peak IL-6 levels were observed when preceded by either the 0.3 or 0.6 mg dose. Double step dosing median peak IL-6 levels were shown to be lower compared to single-step levels.

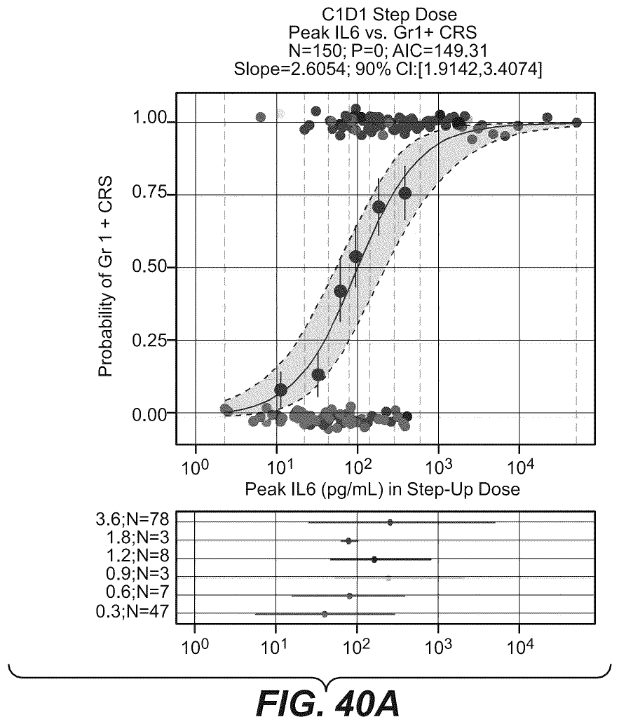

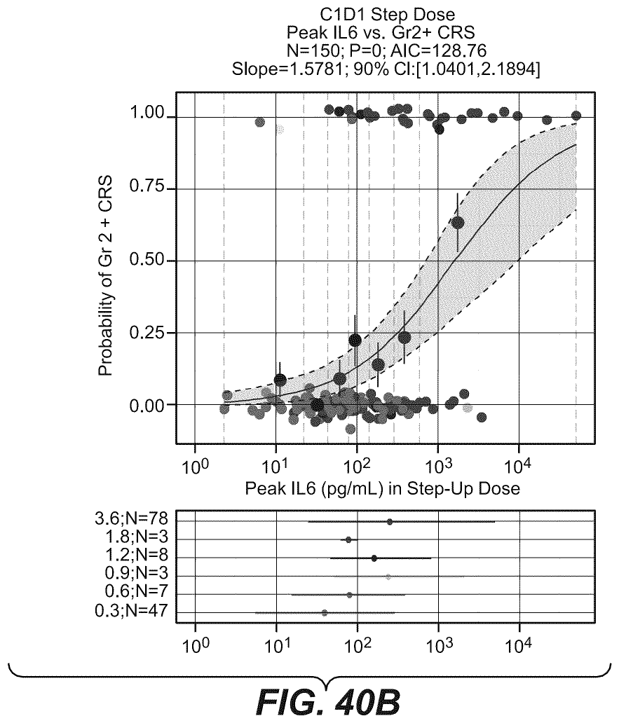

Following the C1D1 step dose, a statistically significant relationship was observed between peak IL-6 concentrations and the probability of grades ≥1 and ≥2 CRS (FIGS. 40A and 40B).

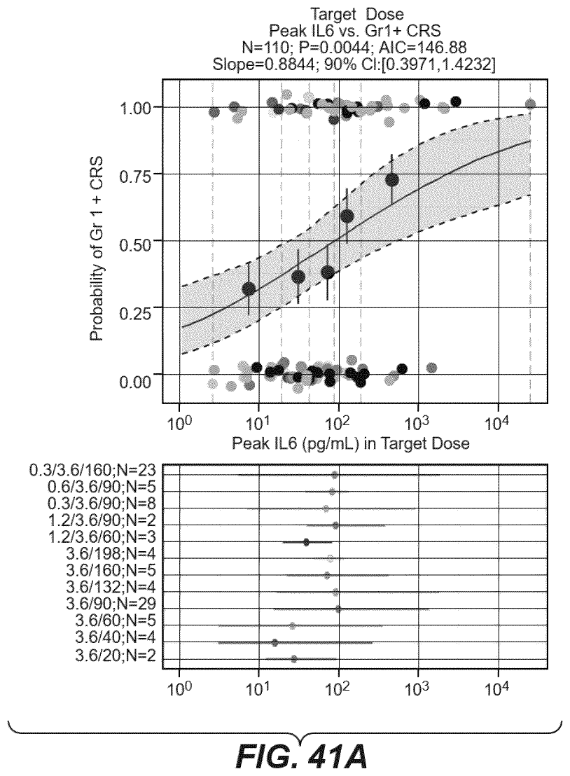

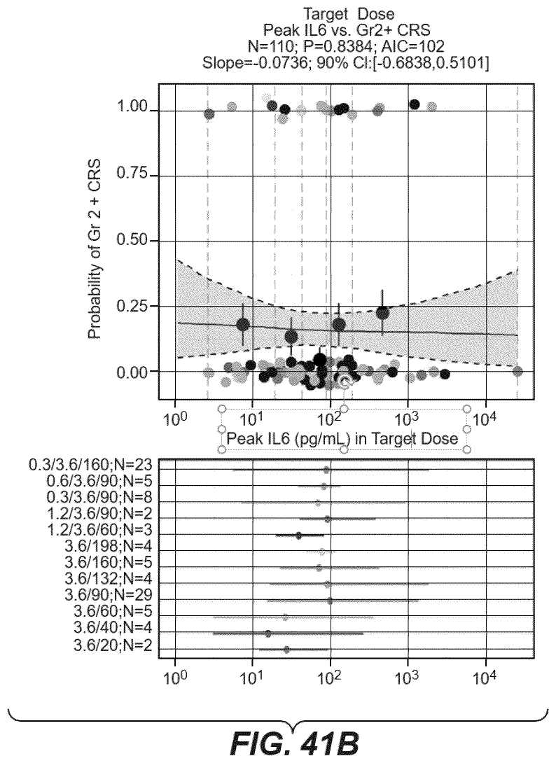

Following administration of the target dose, a significant trend was observed for peak IL-6 and probability of Grade ≥1 CRS events; no apparent trend was noted for peak IL-6 and probability of Grade ≥2 CRS events (FIGS. 41A and 41B).

A pharmacokinetic-pharmacodynamic (PK-PD) evaluation was performed to evaluate the relationship between the peak IL-6 concentrations and cevostamab exposures following the administration of step-up and TDs. For the step-up doses, linear regression analyses were performed to evaluate the relationship between the step-up dose exposures (step-up dose Cₘₐₓ and AUC_{0-7d}) and the peak IL6 concentrations from the time of administration of the step-up dose on Cycle 1 Day 1 until the time of the administration of subsequent step-up/TDs in Cycle 1 using pooled data from single step-up and double step-up dosing regimen. In addition, linear regression analyses were performed to evaluate the relationship between the TD exposures (TD Cₘₐₓ and AUC_{7-21d/14-21d}) and peak IL-6 concentrations following the administration of the TDs on Day 8 or Day 15 in Cycle 1 until the administration of subsequent TDs using pooled data from the single step-up and double step-up dosing regimens.

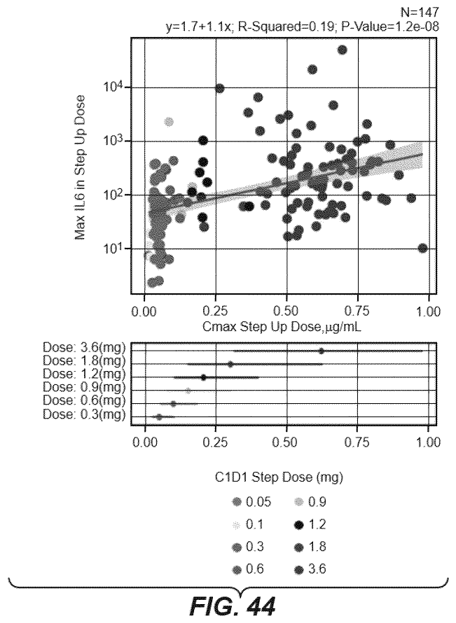

As shown in FIG. 44, statistically significant elevations in the peak IL-6 levels were observed following the step dose exposures at the range of tested step-up doses of 0.05 mg-3.6 mg.

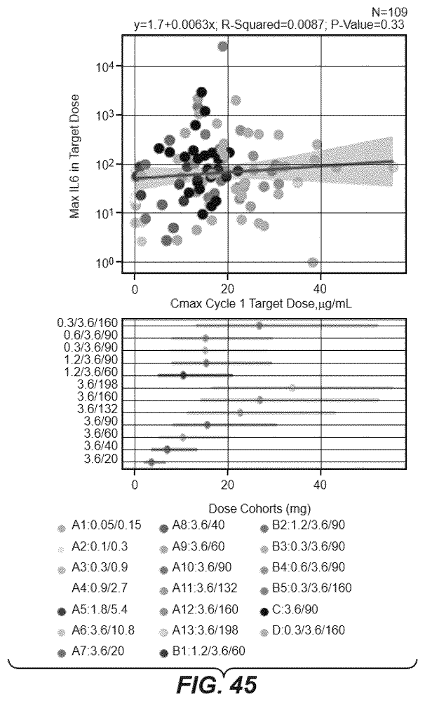

Of note, as shown in FIG. 45, no significant trends were noted for the peak IL6 levels following the TD exposures.

CD8+ T-Cell Activation

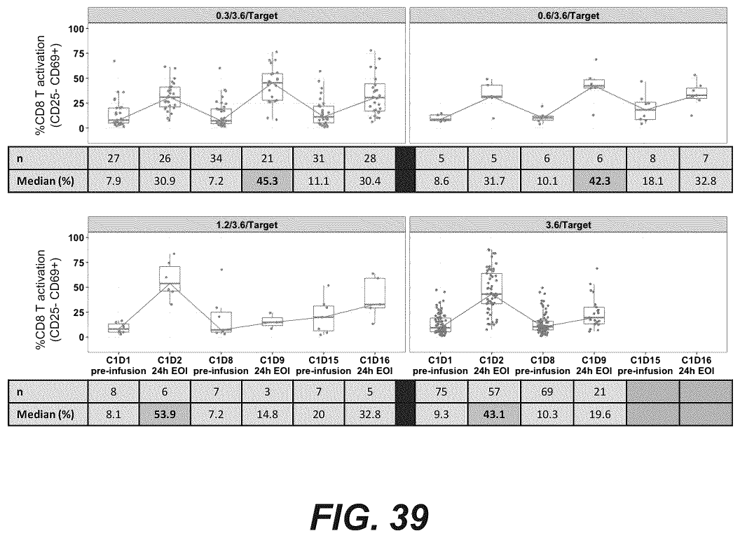

Peak CD8+ T cell activation was delayed at the lower step-up doses (0.3 mg and 0.6 mg) suggesting biological changes with regards to pharmacodynamics (PD) (FIG. 39). This CD8+ T cell activation PD data suggests not all double step (DS) dosing regimens are equivalent. In 0.3 mg and 0.6 mg C1D1 step up doses, the highest activation levels are observed on C1D9, whereas a 1.2 mg dose results in peak levels on C1D2, similar to 3.6 mg in single step (SS) dosing. Median percent CD8+ T-cell activation following the 3.6 mg dose in single-step-up dosing (C1D2) and double step-up dosing (C1D9, in 0.3 mg and 0.6 mg) were comparable.

C1D1 doses of 0.3 mg and 0.6 mg do not blunt T cell activation at subsequent doses.

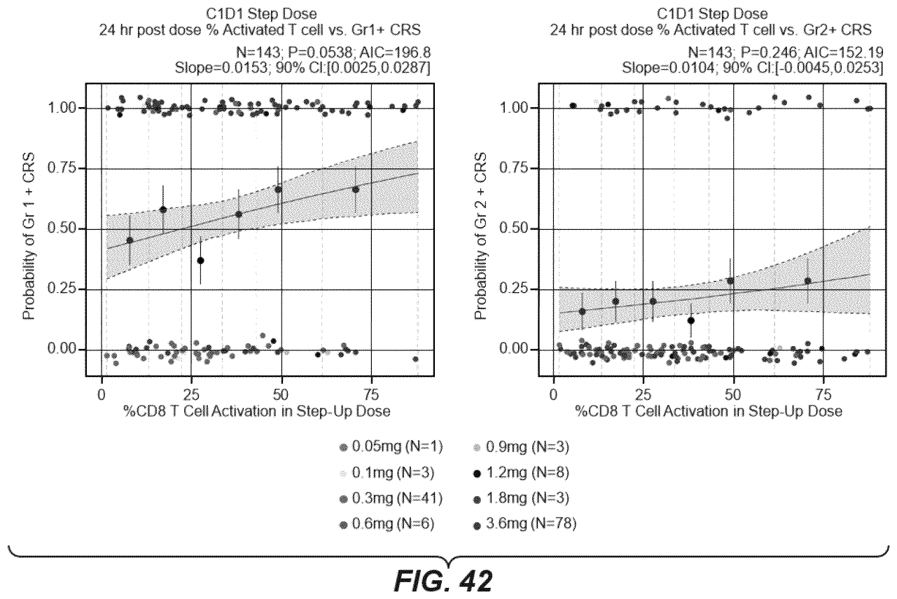

Although positive trends were observed for grade ≥1 CRS, no statistically significant relationship was observed between the percentage of CD8+ T-cell activation and the probability of grades ≥1 and ≥2 CRS following the C1D1 step dose (FIG. 42). T-cell activation was evaluated at 24 hours post-dose.

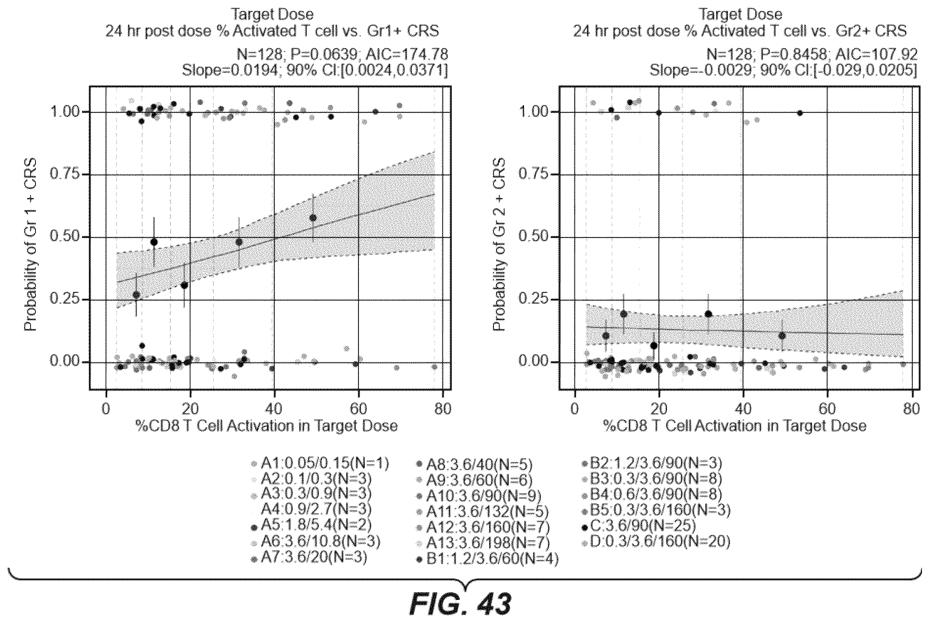

Similarly, although positive trends were observed for grade ≥1 CRS, no statistically significant relationship was observed between the percentage of CD8+ T-cell activation and the probability of grades ≥1 and ≥2 CRS following the target dose (FIG. 43). T-cell activation was evaluated at 24 hours post-dose.

### Example 14. Clinical Pharmacology

Clinical Pharmacokinetics

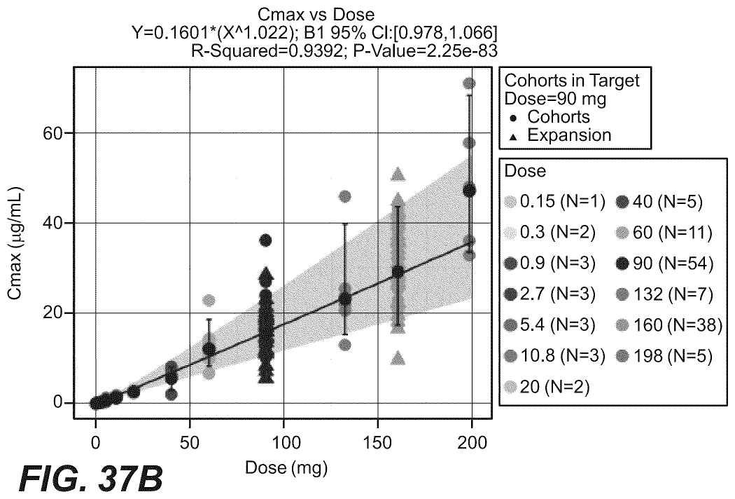

Following the administration of the TD on Cycle 1 Day 8 or Day 15, the cevostamab exposures (Cₘₐₓ and AUC) generally increased with an increase in dose across the dose cohorts tested in Study GO39775 (FIGS. 37A and 37B). Furthermore, proportional increases in Cₘₐₓ and AUC were apparent with an increase in the TDs for both the single step-up and double step-up dosing regimen.

The majority of the responders achieved their initial responses when the drug concentrations had reached the steady-state, i.e., by the end of cycle 4 (day 84) following the Q3W administration of cevostamab in both the single step-up and double step-up doing schedules.

The exposure-efficacy analyses across the range of tested TDs (0.15 mg-198 mg) indicated a statistically significant increase in the best clinical ORR and ≥VGPR rates with increasing cevostamab exposures (AUCₛₛ and C_{min,ss}) using pooled data from the single step-up and double step-up dosing regimen. At the TD of 160 mg, the steady-state cevostamab exposures were shown to approach a plateau for both the ORR and ≥VGPR endpoints.

Furthermore, as a sensitivity analysis, an E-R evaluation was performed for the TD ranges of 90 mg-198 mg between the cevostamab exposure (AUCₛₛ) and ORR. Interestingly, a statistically significant increase in the probability of ORR was observed with increasing target exposures (FIG. 31), confirming the improvement in ORR at the proposed dose of 160 mg as the proposed RP2D.

Taken together, the clinical data and the E-R analyses show a significant improvement in the ORR and ≥VGPR rates with increasing doses/exposures with the exposures at the TD of 160 mg approaching a plateau for the clinical endpoints in both the single step-up and double step-up dosing regimen. Furthermore, based on the sensitivity analysis, a significant improvement in ORR was observed at the TD range of 90 mg-198 mg in both the single step-up and double step-up regimen. In addition, the trough concentrations (C_{min,ss}) at TD of 160 mg approach the E-R model estimated clinical EC₉₀ for C_{min,ss} (4.4 μg/mL). Interestingly, this clinical EC90 was comparable with the ex vivo max. EC90 (2.7 μg/mL) generated from the ex vivo T-cell dependent cytotoxicity assay performed using frozen purified bone marrow mononuclear cells from patients with multiple myeloma. Consistent with the E-R evaluation for efficacy, improved response estimates were predicted at the higher TDs tested using a PK-TGI model. Furthermore, similar (<5% difference) ORR estimates were predicted at the same TD level for both the single step-up and double step-up dosing schedules.

In conclusion, the higher TDs tested maximize the probability of clinical responses, with the TD of 160 mg approaching a plateau for the clinical endpoints.

Exposure-Safety Relationship of Cevostamab for CRS Events

Exposure-safety analyses showed no evidence of an apparent relationship between the TD exposures (TD Cₘₐₓ and AUC_{7-21d/14-21d}) and the frequency of Grades ≥1 and ≥2 CRS following the administration of the TD on Cycle 1 Day 8 or Cycle 1 Day 15 until the end of treatment across the dose levels tested (0.15 mg-198 mg) in both the single step-up and double step-up dosing schedules. However, statistically significant trends were observed between cevostamab step dose exposures (step-up dose Cₘₐₓ and AUC_{0-7d}) and the frequency of the Grades ≥1 and ≥2 CRS events across the range of tested step-up doses (0.05 mg-3.6 mg) in both the single step-up and double step-up dosing schedules. These findings indicate that the step-up doses of 3.6 mg or 0.3/3.6 mg adequately cap the overall acute CRS risks while maximizing the safety margin to allow further escalation of the TDs of cevostamab up to 198 mg.

### CONCLUSIONS

Taken together, the safety, PK, PD, and the PK-PD/E-R analyses continue to support the safety of risk mitigation through the step dose approach, which resulted in a lack of E-R relationship for the frequency of all CRS, Grades ≥2 CRS, and Grades ≥1 ICANS despite the dose escalation of the TDs up to 198 mg administered on Cycle 1 Day 8/Day 15, Cycle 2 Day 1, and every 3 weeks thereafter in both the single step-up and double step-up dosing schedules. This suggests that 3.6 mg or 0.3/3.6 mg doses adequately cap the overall acute safety risks (CRS and ICANS) and maximize the safety margin for TDs up to 198 mg. Moreover, the 0.3 mg dose led to a significant reduction in the peak IL6 levels compared to the 3.6 mg step dose when administered on C1D1 which is consistent with the E-R analyses wherein the 0.3 mg step-up dose led to a significant reduction in the occurrence of Grades ≥2 CRS events.

Furthermore, no significant positive E-R relationship was observed for cevostamab step dose and TD exposures and the other key AEs of concern, i.e., Grades ≥3 cytopenias (neutropenia, anemia and thrombocytopenia), Grade ≥2 IRRs, Grade ≥2 infections and Grade ≥3 any pooled AEs for the single step-up and double step-up schedules.

In conclusion, the TDs (up to 198 mg) do not change the overall risk profile of cevostamab regardless of the step doses tested. The addition of the 0.3 mg dose prior to the 3.6 mg dose demonstrates additional improvements in the CRS and ICANS profile as compared to the single step-up 3.6 mg regimen.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-121. (canceled)

122. A method of treating a subject having a multiple myeloma (MM) comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.01 mg to about 2.9 mg, the C1D2 is between about 3 mg to about 19.9 mg, and the C1D3 is between about 20 mg to about 600 mg.

123. The method of claim 122, wherein:
(a) the C1D1 is between about 0.1 mg to about 1.5 mg; the C1D2 is between about 3.2 mg to about 10 mg; and the C1D3 is between about 80 mg to about 300 mg; and/or
(b) the dosing regimen further comprises a second dosing cycle comprising a single dose (C2D1) of the bispecific antibody, wherein the C2D1 is equal to or greater than the C1D3 and is between about 20 mg to about 600 mg.

124. The method of claim 123, wherein:
(a) the C1D1 is about 0.3 mg; the C1D2 is about 3.6 mg; and the C1D3 is about 160 mg;
(b) the C2D1 is (i) between about 80 mg to about 300 mg or (ii) about 160 mg;
(c) the length of the first dosing cycle is 21 days;
(d) the length of the second dosing cycle is 21 days; and/or
(e) the MM is a relapsed or refractory (R/R) MM.

125. The method of claim 124, wherein:
(I)
(a) the method comprises administering to the subject the C1D1, the C1D2, and the C1D3 on or about Days 1, 8, and 15, respectively, of the first dosing cycle;
(b) the method comprises administering to the subject the C2D1 on or about Day 1 of the second dosing cycle; and/or
(c) the dosing regimen comprises one or more additional dosing cycles; and/or
(II)
(a) the median peak IL-6 level in a population of subjects treated according to the method does not exceed 125 pg/mL between the C1D1 and the C1D2;
(b) the median peak IL-6 level in a population of subjects treated according to the method does not exceed 125 pg/mL between the C1D2 and the C1D3;
(c) the median peak IL-6 level in a population of subjects treated according to the method does not exceed 125 pg/mL following the C1D3; and/or
(d) the peak level of CD8+ T cell activation in the subject in the first dosing cycle occurs between the C1D2 and the C1D3.

126. The method of claim 125, wherein the dosing regimen comprises:
(a) four additional dosing cycles, wherein the length of each of the four additional dosing cycles is 21 days; or
(b) up to 17 additional dosing cycles, wherein the length of each of the additional dosing cycles is 21 days.

127. The method of claim 126, wherein:
(a) the four additional dosing cycles each comprise a single dose of the bispecific antibody, wherein the single dose is between about 80 mg to about 300 mg, and wherein the method comprises administering to the subject the single dose on or about Day 1 of each of the four additional dosing cycles; or
(b) the up to 17 additional dosing cycles each comprise a single dose of the bispecific antibody, wherein the single dose is between about 80 mg to about 300 mg, and wherein the method comprises administering to the subject the single dose on or about Day 1 of each of the up to 17 additional dosing cycles.

128. A method of treating a subject having a MM comprising administering to the subject a bispecific antibody that binds to FcRH5 and CD3 in a dosing regimen comprising at least a first dosing cycle, wherein the first dosing cycle comprises a first dose (C1D1), a second dose (C1D2), and a third dose (C1D3) of the bispecific antibody, wherein the C1D1 is between about 0.2 mg to about 0.4 mg, the C1D2 is greater than the C1D1, and the C1D3 is greater than the C1D2.

129. The method of claim 128, wherein:
(a) the C1D1 is about 0.3 mg;
(b) the C1D2 is (i) between about 3 mg to about 19.9 mg, (ii) between about 3.2 mg to about 10 mg, or (iii) about 3.6 mg;
(c) the C1D3 is (i) between about 20 mg to about 600 mg, (ii) between about 80 mg to about 300 mg, or (iii) about 160 mg; and/or
(d) the dosing regimen further comprises a second dosing cycle comprising a single dose (C2D1) of the bispecific antibody, wherein the C2D1 is equal to or greater than the C1D3 and is between about 20 mg to about 600 mg.

130. The method of claim 129, wherein:
(a) the C2D1 is (i) between about 80 mg to about 300 mg, or (ii) about 160 mg;
(b) the length of the first dosing cycle is 21 days;
(c) the length of the second dosing cycle is 21 days; and/or
(d) the MM is a relapsed or refractory (R/R) MM.

131. The method of claim 130, wherein:
(I)
(a) the method comprises administering to the subject the C1D1, the C1D2, and the C1D3 on or about Days 1, 8, and 15, respectively, of the first dosing cycle;
(b) the method comprises administering to the subject the C2D1 on or about Day 1 of the second dosing cycle; and/or
(c) the dosing regimen comprises one or more additional dosing cycles; and/or
(II)
(a) the median peak IL-6 level in a population of subjects treated according to the method does not exceed 125 pg/mL between the C1D1 and the C1D2;
(b) the median peak IL-6 level in a population of subjects treated according to the method does not exceed 125 pg/mL between the C1D2 and the C1D3;
(c) the median peak IL-6 level in a population of subjects treated according to the method does not exceed 125 pg/mL following the C1D3; or
(d) the peak level of CD8+ T cell activation in the subject in the first dosing cycle occurs between the C1D2 and the C1D3.

132. The method of claim 131, wherein the dosing regimen comprises:
(a) four additional dosing cycles, wherein the length of each of the four additional dosing cycles is 21 days; or
(b) up to 17 additional dosing cycles, wherein the length of each of the additional dosing cycles is 21 days.

133. The method of claim 132, wherein:
(a) the four additional dosing cycles each comprise a single dose of the bispecific antibody, wherein the single dose is between about 80 mg to about 300 mg, and wherein the method comprises administering to the subject the single dose on or about Day 1 of each of the four additional dosing cycles; or
(b) the up to 17 additional dosing cycles each comprise a single dose of the bispecific antibody, wherein the single dose is between about 80 mg to about 300 mg, and wherein the method comprises administering to the subject the single dose on or about Day 1 of each of the up to 17 additional dosing cycles.

134. A method of treating a subject having a multiple myeloma (MM) comprising administering to the subject a bispecific antibody that binds to FcRH5 and CD3 in a dosing regimen comprising at least a first dosing cycle, wherein the first dosing cycle comprises a first dose (C1D1) and a second dose (C1D2) of the bispecific antibody, wherein the C1D1 is between about 0.5 mg to about 19.9 mg and the C1D2 is between about 20 mg to about 600 mg.

135. The method of claim 134, wherein:
(a) the C1D1 is between about 1.2 mg to about 10.8 mg and the C1D2 is between about 80 mg to about 300 mg; and/or
(b) the dosing regimen further comprises a second dosing cycle comprising a single dose (C2D1) of the bispecific antibody, wherein the C2D1 is equal to or greater than the C1D2 and is between about 20 mg to about 600 mg.

136. The method of claim 135, wherein:
(a) the C1D1 is about 3.6 mg and the C1D2 is about 198 mg;
(b) the C2D1 is (i) between about 80 mg to about 300 mg or (ii) about 198 mg;
(c) the length of the first dosing cycle is 21 days;
(d) the length of the second dosing cycle is 21 days; and/or
(e) the MM is a relapsed or refractory (R/R) MM.

137. The method of claim 136, wherein:
(a) the method comprises administering to the subject the C1D1 and the C1D2 on or about Days 1 and 8, respectively, of the first dosing cycle;
(b) the method comprises administering to the subject the C2D1 on or about Day 1 of the second dosing cycle; and/or
(c) the dosing regimen comprises one or more additional dosing cycles.

138. The method of claim 137, wherein:
(a) the dosing regimen comprises one to 17 additional dosing cycles;
(b) the length of each of the additional dosing cycles is 21 days; and/or
(c) each of the one or more additional dosing cycles comprises a single dose of the bispecific antibody on Day 1 of the one or more additional dosing cycles.

139. The method of claim 122, wherein the bispecific antibody comprises:
(a) an anti-FcRH5 arm comprising a first binding domain comprising the following six hypervariable regions (HVRs):
(i) an HVR-H1 comprising the amino acid sequence of RFGVH (SEQ ID NO: 1);
(ii) an HVR-H2 comprising the amino acid sequence of VIWRGGSTDYNAAFVS (SEQ ID NO: 2);
(iii) an HVR-H3 comprising the amino acid sequence of HYYGSSDYALDN (SEQ ID NO:3);
(iv) an HVR-L1 comprising the amino acid sequence of KASQDVRNLVV (SEQ ID NO: 4);
(v) an HVR-L2 comprising the amino acid sequence of SGSYRYS (SEQ ID NO: 5); and
(vi) an HVR-L3 comprising the amino acid sequence of QQHYSPPYT (SEQ ID NO: 6), and/or
(b) an anti-CD3 arm comprising a second binding domain comprising the following six HVRs:
(i) an HVR-H1 comprising the amino acid sequence of SYYIH (SEQ ID NO: 9);
(ii) an HVR-H2 comprising the amino acid sequence of WIYPENDNTKYNEKFKD (SEQ ID NO: 10);
(iii) an HVR-H3 comprising the amino acid sequence of DGYSRYYFDY (SEQ ID NO: 11);
(iv) an HVR-L1 comprising the amino acid sequence of KSSQSLLNSRTRKNYLA (SEQ ID NO: 12);
(v) an HVR-L2 comprising the amino acid sequence of WTSTRKS (SEQ ID NO: 13); and
(vi) an HVR-L3 comprising the amino acid sequence of KQSFILRT (SEQ ID NO: 14).

140. The method of claim 122, wherein the bispecific antibody comprises:
(a) an anti-FcRH5 arm comprising a first binding domain comprising (i) a heavy chain variable (VH) domain comprising an amino acid sequence having at least 95% sequence identity to the amino acid sequence of SEQ ID NO: 7; (ii) a light chain variable (VL) domain comprising an amino acid sequence having at least 95% sequence identity to the amino acid sequence of SEQ ID NO: 8; or (iii) a VH domain as in (i) and a VL domain as in (ii); and/or
(b) an anti-CD3 arm comprising a second binding domain comprising (i) a VH domain comprising an amino acid sequence having at least 95% sequence identity to the amino acid sequence of SEQ ID NO: 15; (ii) a VL domain comprising an amino acid sequence having at least 95% sequence identity to the amino acid sequence of SEQ ID NO: 16; or (iii) a VH domain as in (i) and a VL domain as in (ii).

141. The method of claim 140, wherein:
(a) the first binding domain comprises a VH domain comprising an amino acid sequence of SEQ ID NO: 7 and a VL domain comprising an amino acid sequence of SEQ ID NO: 8; and/or
(b) the second binding domain comprises a VH domain comprising an amino acid sequence of SEQ ID NO: 15 and a VL domain comprising an amino acid sequence of SEQ ID NO: 16.

142. The method of claim 122, wherein the bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein:
(a) the H1 comprises the amino acid sequence of SEQ ID NO: 35;
(b) the L1 comprises the amino acid sequence of SEQ ID NO: 36;
(c) the H2 comprises the amino acid sequence of SEQ ID NO: 37; and
(d) the L2 comprises the amino acid sequence of SEQ ID NO: 38.

143. The method of claim 122, wherein:
(a) the bispecific antibody comprises an aglycosylation site mutation; and/or
(b) the bispecific antibody is a monoclonal antibody, a humanized antibody, a chimeric antibody, or an antibody fragment that binds FcRH5 and CD3.

144. The method of claim 143, wherein:
(a) the aglycosylation site mutation reduces effector function of the bispecific antibody;
(b) the aglycosylation site mutation is a substitution mutation; and/or
(c) the antibody fragment is selected from the group consisting of Fab, Fab′-SH, Fv, scFv, and (Fab′)₂ fragments.

145. The method of claim 122, wherein:
(a) the bispecific antibody is a full-length antibody;
(b) the bispecific antibody is an IgG antibody; and/or
(c) the bispecific antibody comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain.

146. The method of claim 145, wherein:
(a) the IgG antibody is an IgG₁ antibody;
(b) at least one of the one or more heavy chain constant domains is paired with another heavy chain constant domain;
(c) the CH3₁ and CH3₂ domains each comprise a protuberance or cavity, and wherein the protuberance or cavity in the CH3₁ domain is positionable in the cavity or protuberance, respectively, in the CH3₂ domain; and/or
(d) the CH2₁ and CH2₂ domains each comprise a protuberance or cavity, and wherein the protuberance or cavity in the CH2₁ domain is positionable in the cavity or protuberance, respectively, in the CH2₂ domain.

147. The method of claim 146, wherein:
(a) the CH3₁ and CH3₂ domains meet at an interface between the protuberance and cavity; and/or
(b) the CH2₁ and CH2₂ domains meet at an interface between said protuberance and cavity, wherein the anti-FcRH5 arm comprises the protuberance and the anti-CD3 arm comprises the cavity.

148. The method of claim 147, wherein a CH3 domain of the anti-FcRH5 arm comprises a protuberance comprising a T366W amino acid substitution mutation (EU numbering) and a CH3 domain of the anti-CD3 arm comprises a cavity comprising T366S, L368A, and Y407V amino acid substitution mutations (EU numbering).

149. The method of claim 122, wherein:
(a) the bispecific antibody is administered to the subject by intravenous infusion;
(b) the bispecific antibody is administered to the subject subcutaneously;
(c) the bispecific antibody is administered to the subject as a monotherapy; or
(d) the bispecific antibody is administered to the subject as a combination therapy with one or more therapeutic agents, wherein the bispecific antibody is administered to the subject:
(i) concurrently with one or more additional therapeutic agents;
(ii) prior to the administration of one or more additional therapeutic agents; or
(iii) subsequent to the administration of one or more additional therapeutic agents.

150. The method of claim 149, wherein the one or more additional therapeutic agents comprise:
(a) an effective amount of tocilizumab;
(b) an effective amount of pomalidomide, daratumumab, or a B-cell maturation antigen (BCMA)-directed therapy; or
(c) an effective amount of:
(i) a corticosteroid;
(ii) acetaminophen or paracetamol; and/or
(iii) diphenhydramine.

151. The method of claim 150, wherein:
(a) tocilizumab is administered to the subject by intravenous infusion;
(b) the subject weighs:
(i) ≥100 kg, and tocilizumab is administered to the subject at a dose of 800 mg;
(ii) ≥30 kg and <100 kg, and tocilizumab is administered to the subject at a dose of 8 mg/kg; or
(iii) <30 kg, and tocilizumab is administered to the subject at a dose of 12 mg/kg; and/or
(c) tocilizumab is administered to the subject 2 hours before administration of the bispecific antibody.

152. The method of claim 122, wherein the subject has a cytokine release syndrome (CRS) event, and the method further comprises administering to the subject an effective amount of tocilizumab to treat the CRS event while suspending treatment with the bispecific antibody.

153. The method of claim 152, wherein:
(a) the CRS event does not resolve or worsens within 24 hours of treating the symptoms of the CRS event, the method further comprising administering to the subject one or more additional doses of tocilizumab to manage the CRS event; and/or
(b) tocilizumab is administered intravenously to the subject as a single dose of about 8 mg/kg.

154. The method of claim 150, wherein:
(a) the corticosteroid:
(i) is methylprednisolone administered at a dose of about 80 mg or dexamethasone administered at a dose of about 20 mg; and/or
(ii) is administered intravenously to the subject;
(b) the acetaminophen or paracetamol:
(i) is administered at a dose of between about 500 mg to about 1000 mg; and/or
(ii) is administered orally to the subject; and/or
(c) the diphenhydramine:
(i) is administered at a dose of between about 25 mg to about 50 mg; and/or
(ii) is administered orally to the subject,

155. The method of claim 124, wherein:
(a) the individual has received at least three prior lines of treatment for the MM or at least four prior lines of treatment for the MM; and/or
(b) individual has been exposed to a prior treatment comprising a proteasome inhibitor, an immunomodulatory drug (IMiD), and/or an anti-CD38 therapeutic agent.

156. The method of claim 155, wherein:
(a) the proteasome inhibitor is bortezomib, carfilzomib, or ixazomib;
(b) the IMiD is thalidomide, lenalidomide, or pomalidomide;
(c) the anti-CD38 therapeutic agent is an anti-CD38 antibody; or
(d) the individual has been exposed to a prior treatment comprising an anti-SLAMF7 therapeutic agent, a nuclear export inhibitor, a histone deacetylase (HDAC) inhibitor, an autologous stem cell transplant (ASCT), a bispecific antibody, an antibody-drug conjugate (ADC), a CAR-T cell therapy, or a BCMA-directed therapy.

157. The method of claim 156, wherein:
(a) the anti-CD38 antibody is daratumumab, MOR202, or isatuximab;
(b) the anti-SLAMF7 therapeutic agent is an anti-SLAMF7 antibody;
(c) the nuclear export inhibitor is selinexor;
(d) the HDAC inhibitor is panobinostat; or
(e) the BCMA-directed therapy is an antibody-drug conjugate targeting BCMA.


SEQUENCE LISTING (38 sequences)

SEQ ID NO: 1 - protein, 5 aa, synthetic construct
RFGVH

SEQ ID NO: 2 - protein, 16 aa, synthetic construct
VIWRGGSTDYNAAFVS

SEQ ID NO: 3 - protein, 12 aa, synthetic construct
HYYGSSDYALDN

SEQ ID NO: 4 - protein, 11 aa, synthetic construct
KASQDVRNLVV

SEQ ID NO: 5 - protein, 7 aa, synthetic construct
SGSYRYS

SEQ ID NO: 6 - protein, 9 aa, synthetic construct
QQHYSPPYT

SEQ ID NO: 7 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 8 - protein, 107 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIK

SEQ ID NO: 9 - protein, 5 aa, synthetic construct
SYYIH

SEQ ID NO: 10 - protein, 17 aa, synthetic construct
WIYPENDNTKYNEKFKD

SEQ ID NO: 11 - protein, 10 aa, synthetic construct
DGYSRYYFDY

SEQ ID NO: 12 - protein, 17 aa, synthetic construct
KSSQSLLNSRTRKNYLA

SEQ ID NO: 13 - protein, 7 aa, synthetic construct
WTSTRKS

SEQ ID NO: 14 - protein, 8 aa, synthetic construct
KQSFILRT

SEQ ID NO: 15 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 17 - protein, 30 aa, synthetic construct
EVQLVESGPGLVKPSETLSLTCTVSGFSLT

SEQ ID NO: 18 - protein, 14 aa, synthetic construct
WVRQPPGKGLEWLG

SEQ ID NO: 19 - protein, 32 aa, synthetic construct
RLTISKDNSKNQVSLKLSSVTAADTAVYYCSN

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WFQQKPGKAPKLLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGFTFT

SEQ ID NO: 26 - protein, 14 aa, synthetic construct
WVRQAPGQGLEWIG

SEQ ID NO: 27 - protein, 32 aa, synthetic construct
RVTITADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQSPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - DNA, 5620 bp, Homo sapiens
  source 1..5620: mol_type = unassigned DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 34 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 35 - protein, 450 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 219 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC